(19) United States
(12) Patent Application Publication
(10) Pub. No.: US 2025/0145946 A1
(43) Pub. Date: May 8, 2025

(54) GENERATION OF PURE RETINAL CELLS FROM HUMAN PLURIPOTENT STEM CELLS

(71) Applicant: Wisconsin Alumni Research Foundation, Madison, WI (US)

(72) Inventors: Su-Chun Zhang, Madison, WI (US); Yunlong Tao, Madison, WI (US)

(21) Appl. No.: 18/940,042

(22) Filed: Nov. 7, 2024

Related U.S. Application Data
(60) Provisional application No. 63/596,926, filed on Nov. 7, 2023.

(51) Int. Cl. C12N 5/079; C12N 5/0797

(52) U.S. Cl. CPC C12N 5/0618; C12N 5/0623; C12N 2501/155; C12N 2501/999; C12N 2506/03; C12N 2506/08

(57) ABSTRACT

Provided herein are methods for producing neuroectoderm and retinal progenitor cells from pluripotent stem cells, and compositions of retinal progenitor cells produced thereby.

### STATEMENT REGARDING FEDERALLY FUNDED RESEARCH OR DEVELOPMENT

This invention was made with government support under National Institute of Child Health and Human Development (NICHD) (U54 HD090256 and HD106197). The government has certain rights in the invention.

### BACKGROUND OF THE DISCLOSURE

During early neural development, the most anterior neural plate is segmented into three subregions: telencephalon, eye field and diencephalon (Diacou et al., 2022, Prog Retin Eye Res. 91:101093; Giger & Houart, 2018, Front Neurosci 12:87). The eye field, located at the medial portion of the anterior neural plate, is defined by the expression of a group of eye field transcription factors (EFTFs) that include PAX6, RAX, OTX2, LHX2, SIX3 and SIX6 (Zuber et al. 2003, Development 130:5155-5167). The EFTFs are not uniquely expressed in eye field cells and many of them are also expressed by cells in the telencephalon and diencephalon regions (adjunctive subregion) (Maden, 2002, Nat Rev Neurosci 3:843-853; Niehrs, 2004, Nat Rev Genet 5:425-434; Mason, 2007, Nat Rev Neurosci 8:583-596; Bielen & Houart, 2014, Dev Neurobiol 74:772-780); for instance, PAX6, OTX2, LHX2 and SIX3 expression is found in the prospective telencephalon while OTX2, LHX2, SIX3, RAX and SIX6 are expressed in the prospective diencephalon (Lagutin et al., 2003, Genes Dev 17:368-379; Ando et al., 2005, Dev Biol 287:456-468; Georgala et al., 2011, Dev Neurobiol 71:690-709; Hoch et al., 2015, Cell Rep 12:482-494; Matsuo et al., 1995, Genes Dev 9:2646-2658; Orquera et al. 2016, Dev Biol 416:212-224; Jean et al., 1999, Mech Dev 84:31-40). The regulatory mechanisms of EFTFs by morphogens that delineate the eye field from neighboring regions remain elusive.

Eye field formation accompanies regional patterning along the anterior-posterior axis in the neural plate. Inhibitors of the Wnt pathway are present in both telencephalon and eye field subregions in the anterior neural plate to repress posterization (Niehrs et al., 2001, Int J Dev Biol 45:237-240; Houart et al., 2002, Neuron 35:255-265; Tendeng. & Houart, 2006, Gene Expr Patterns 6:761-771). It remains unknown how the eye field and telencephalic regions are further subdivided (Esteve & Bovolenta, 2006, Curr Opin Neurobiol 16:13-19). Due to the challenges in accessing those subregions within the neural plate at such an early stage, there have been only limited studies investigating the signals specifying eye field formation. Studies in a Xenopus frog model have suggested that fibroblast growth factor (FGF) and ephrin signaling pathways regulate cell movements to the eye field (Moore et al., 2004, Dev Cell 6:55-67) while the non-canonical WNT pathway promotes cell coherence within the eye field and represses the Wnt/beta-catenin pathway (Cavodeassi et al., 2005, Neuron 47:43-56). Rostral paraxial mesoderm has been shown to inhibit normal eye development through BMPs in chicken embryo (Teraoka et al., 2009, Dev Biol 330, 389-398). A zebrafish study demonstrated that BMP pathway acts as a repressor of the eye field fate by inhibiting Rx3 (RAX homolog) hence preventing the telencephalon from acquiring the eye identity (Bielen & Houart, 2012, Dev Cell 23:812-822), suggesting the negative role of BMP in the eye field formation. However, these findings have not been validated in other vertebrates including humans due to technical difficulties in distinguishing cell movement during gastrulation. Consequently, the molecular pathways and their interactions that underlie the specification of the eye-field or retinal cells from the prospective telencephalic cells in humans remains unclear.

Human pluripotent stem cells (hPSCs) have the potential to be used as an advantageous model to reveal aspects of early development, including eye-field/retinal specification. They may also be promising source for generating retinal cells to treat retina diseases. Given the neuroectodermal origin of retinas, most of the current methods to generate retinal cells have involved guiding hPSCs to neuroepithelia through embryoid body formation followed by spontaneous differentiation to retinal cells in the form of optic cup-like structures in a suspension culture which are then manually selected (Zhao et al., 2017, Development 144:1368-1381; Zhong et al., 2014, Nat Commun 5:4047; Capowski et al., 2019, Development 146; Gonzalez-Cordero et al., 2017, Stem Cell Reports 9:820-837). However, such approaches are inefficient, primarily due to the lack of understanding how the early neuroepithelia are specified to the eye field cells and subsequent retina progenitors.

There remains a need in this art for methods to produce retinal cells for treating retina degenerative diseases, as well as methods for using such cells for these treatments and isolated populations of retinal cells for such uses.

### SUMMARY OF THE DISCLOSURE

Disclosed herein are methods for generating pure retinal progenitor cells (RPCs) and their progenies, and isolated populations of such cells produced thereby. Elucidated herein is a previously unappreciated role of BMP in specifying early (primitive) neuroepithelia to the eye-field/retinal fate through identification of differential molecular pathways in neuroepithelial and retinal scRNA-Seq datasets, followed by serial molecular interventions during the neuroepithelia-to-retina transition. Generation of pure retinal progenitor cells (RPCs) and their progenies using chemically defined conditions without any need for manual selection is a consequence of this understanding of molecular pathways underlying the transition from neuroepithelia to eye field-like cells disclosed herein. Further disclosed is the use of EFTFs, including PAX6 wherein expression thereof is mediated by BMP.

Also provided herein are methods for producing neuroectoderm cells from human pluripotent stem cells (hPSCs) comprising the steps of treating hPSCs in cell culture in vitro with inhibitors of BMP or TGF-beta, alone or in combinations thereof.

The RPCs produced by the methods disclosed herein are advantageously derived from neuroectoderm cells produced from human pluripotent stem cells (hPSCs). Accordingly, provided herein are methods for producing primitive neuroectoderm cells (PNCs) from human pluripotent stem cells (hPSCs).

Also provided herein are methods for producing RPCs from PNCs comprising the steps of treating the PNCs in cell culture in vitro with BMP2, BMP4, or BMP7.

Also provided herein are methods for producing RPCs from human pluripotent stem cells (hPSCs) comprising the steps of treating hPSCs in cell culture with a TGF-beta inhibitor for 5 days followed by treatment with BMP activator from day 6 to day 20.

Also provided herein are compositions of retinal progenitor cells, including cell populations comprising 50-100% RPCs, produced by the disclosed methods.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

The disclosure will be better understood and features, aspects, and advantages other than those set forth above will become apparent when consideration is given to the following detailed description thereof. Such detailed description refers to the following figures.

FIGS. 1a-1j show differential gene expression between retinal and cortical progenitors. FIG. 1a illustrates a diagram of the presumptive eye field, telencephalon and diencephalon in the anterior neural plate. FIG. 1b illustrates a diagram of the strategy to identify retina specific genes or pathways from cortical progenitors. SOX2-expressing progenitor cells were selected from both cortical and retina organoids and then integrated into one dataset for downstream bioinformatic analysis. FIG. 1c shows uniform manifold approximation and projection (UMAP) clustering of retina progenitors isolated from a single cell RNA-Seq dataset generated from a retina organoid (day 25-day 35). The retina progenitor markers were demonstrated. FIG. 1d shows UMAP clustering of cortical progenitors isolated from a cortical organoid dataset (day 35). FIG. 1e shows UMAP of the integrated single cell data from the progenitors of cortical and retina organoids in FIGS. 1c-1d. The composition of each sample in the UMAP was demonstrated by splitting them. FIG. 1f is a pie chart showing the proportion of the clusters in FIG. 1e; and FIG. 1g shows the ratio of cortical and retina cells in each cluster. FIG. 1h is a volcano plot illustrating the differentially regulated genes between retina and cortical progenitors in the cell population of Neural Pro 1-5 in FIG. 1e. FIG. 1i is a gene ontology analysis of the biological process and molecular function using the up-regulated genes in retina progenitors (fold change>2, p-value<0.05). Only top 10 most significant gene ontology terms were plotted in the panel. FIG. 1j shows BMP pathway gene expression in both retina and cortical cell single cell datasets FIGS. 1c-1d.

FIGS. 2a-2j show regulation of retinal differentiation by BMPs. FIG. 2a is a diagram of the experiment design to investigate the role of WNT, BMP and TGF-beta in retina differentiation. FIG. 2b is shows the results of qPCR analysis of retinal differentiation-associated genes under treatment with inhibitors of WNT (IWR1), BMP (DMH1) or TGF-beta (SB). FIG. 2c shows expression of the retinal progenitor marker VSX2 at different time points along the neural differentiation pathway under treatment using SB with or without DMH1. FIG. 2d shows a diagram of the experiment design in FIGS. 2e-2h to study the role of BMP inhibition at different stages of retina development. FIG. 2e shows UMAP clustering of the differentiating cells under the treatment using SB with and without DMH1 during day 0-day 6. Single cells from each condition were demonstrated separately by the UMAP clustering. FIG. 2f shows quantification of the cluster proportion and the ratio of cells from each condition among all the clusters in FIG. 2e. FIG. 2g shows a violin plot of selected gene expression between SB and SB+DMH1 in each cluster in FIG. 2e. FIG. 2h shows UMAP clustering of differentiating cells at day-12 treated with or without DMH1 during day 6-12 of neural differentiation. Cells were pretreated with SB from day 0-day 5 as illustrated in FIG. 2d. Cells from control and DMH1 conditions were demonstrated separately. FIG. 2i shows quantification of the cluster proportion and the ratio of cells from each condition among all the clusters in FIG. 2h. FIG. 2j illustrates a violin plot of selected gene expression between Control and DMH1 in each cluster shown in FIG. 2h.

FIGS. 3a-3e illustrate temporal and dose-dependent effects of BMP on retina differentiation. FIG. 3a is a diagram of the experimental design to investigate the role of BMP in retinal differentiation. FIG. 3b shows qPCR analysis of the expression levels of retina-associated genes in Control, SB and DMH1 conditions shown in FIG. 3a. FIG. 3c shows expression of definitive neuroepithelial marker SOX1 at different time points along neural differentiation in cells treated with SB or BMP2. FIG. 3d shows a diagram of the experimental design to identify the time point when BMP pathway starts to regulate retinal differentiation (left panel), and gene expression of the retinal progenitor marker VSX2 and definitive neuroepithelial marker SOX1 by qPCR (right panel). B4 means the treatment with BMP2 starting from day 4. FIG. 3e shows a diagram of the experiment design to examine the effect of BMP2 doses on retina differentiation (left panel), and the gene expression of VSX2 and SOX1 by qPCR (right panel).

FIGS. 4a-4k illustrates the interaction between PAX6 and BMP during retina differentiation. FIG. 4a shows gene expression levels of PAX6 and VSX2 in day-20 differentiating cells derived from wildtype and PAX6 knock-out (KO) cell lines. FIG. 4b shows a whole mount immunostaining of VSX2 and PAX6 in day 20 organoids generated from wildtype and PAX6 KO cell lines. FIG. 4c illustrates expression levels of BMP pathway genes in day 20 differentiating cells derived from wildtype and PAX6 KO cell lines. FIG. 4d shows a diagram of the PAX6 isoforms and the construction strategies of generating PAX6 KO, ABKO and DKO cell lines in hPSCs. FIG. 4e shows a principal component analysis of wildtype, PAX6 KO, PAX6 ABKO and PAX6 DKO RNA-Seq replicates. FIG. 4f is a Venn diagram analysis of the down-regulated genes in groups of PAX6 KO vs wildtype, PAX6 ABKO vs wildtype and PAX6 DKO vs wildtype. The number of genes and the percentages are labeled in the respective areas. FIG. 4g shows a heatmap and hierarchical clustering analysis of all the DEGs collected from pairwise comparison of PAX6 KO vs wildtype, PAX6 ABKO vs wildtype and PAX6 DKO vs wildtype. FIG. 4h shows a gene ontology analysis of the biological processes enriched in the 117 shared genes in FIG. 4f. FIG. 4i shows relative expression levels of retina specific markers and BMP pathway genes in day 20 progenitors derived from wildtype, PAX6 KO, PAX6 ABKO and PAX6 DKO cell lines. FIG. 4j is a Venn diagram of target genes of PAX6A and PAX6D revealed by ChIP-Seq data. FIG. 4k illustrates interaction between PAX6 and BMP in regulating retina specification. PAX6 regulates BMP signaling by directly modulating transcription of BMPR1B, which renders eye field cells responsive for BMP signaling. BMP activation then specifies eye field cells towards neural retina fate.

FIGS. 5a-5g show EFTF expression. FIG. 5a illustrates the experiment design for snRNA-Seq analysis. FIG. 5b shows UMAP clustering of differentiating cells sequentially treated with SB and BMP2 at day 12 of differentiation as illustrated in FIG. 5a. FIG. 5c-5d show violin (5c) and dot (5d) plots of retina differentiation-related genes in each cluster to show their expression levels and ratios. FIG. 5e shows UMAP clustering of differentiating cells treated with BMP2 or not during day 6-day 12 at day 12 of neural differentiation. The cells were pretreated with SB from day 0-day 5 as shown in FIG. 5a, wherein cells from control and BMP2 conditions was demonstrated separately. FIG. 5f shows quantification of the cluster proportion and the ratio of cells from each condition among all the clusters in FIG. 5e. FIG. 5g shows a violin plot of selected genes in each cluster between BMP2 and Control.

FIGS. 6a-6j show generation of retinal cells by regulating TGF-beta and BMP signaling. FIG. 6a shows a diagram of the retina differentiation protocol. FIG. 6b shows bright field microscopy of day-20 retina organoids generated by sequential treatment of SB and BMP2. FIG. 6c shows whole mount staining for VSX2 and PAX6 in the retina organoids at day 20. Scale bar, 50 μm. FIG. 6d shows immunostaining of photoreceptor markers CRX and OPSIN in the sections of retina organoids at day 60. Scale bar, 25 μm. FIG. 6e shows UMAP clustering of differentiating cells sequentially treated with SB and BMP2 at day 30 of the differentiation as illustrated in FIG. 6i. RA was administrated at day 20 to promote retinogenesis. FIGS. 6f-6g show violin (6f) and dot (6g) plots of retina differentiation-related genes in each cluster to show their expression levels and ratios. FIG. 6h shows integration of snRNA-Seq data in FIG. 6h and scRNA-Seq dataset from human fetal retina at day 59. FIG. 6i is a feature plot of retina progenitor markers (RAX and VSX2) and neuronal marker (DCX) in FIG. 6h. FIG. 6j shows a violin plot of major retina lineage markers in the retina clusters. The corresponding percentage of cells from each sample in each cluster were demonstrated as bar graph.

FIGS. 7a-7f show identified retina specific genes and pathways. FIGS. 7a-7b shows the retina and cortical progenitor markers in single cell RNA-Seq datasets from retina (FIG. 7a) and cortical (FIG. 7b) organoids. FIG. 7c shows clustering based on the cell cycle status G1, S and G2M in the integrated single cell datasets from retina and cortical progenitors in FIG. 1e. FIG. 7d is a dot plot of selected genes in each cluster of the integrated single cell datasets from retina and cortical progenitors in FIG. 1e. FIGS. 7e-7f show bioinformatic analysis of enriched pathways (FIG. 7e) and associated mouse phenotypes (FIG. 7f) in the up-regulated genes in retina progenitors.

FIGS. 8a-8c show the role of BMP in retina development. FIGS. 8a-8b show flow cytometry analyses of the differentiating cells by genes SOX2, SOX1 and PAX6. FIG. 8c shows qPCR analysis of SOX2, PAX6 and RAX expression at different time points along neural differentiation in the differentiating cells treated with SB or SB+DMH1.

FIGS. 9a-9d show snRNA analysis of neural induction at day 6. FIG. 9a is a heatmap of the cluster genes in the differentiating cells treated with SB or SB+DMH1 at day 6. FIG. 9b shows a volcano plot of differentially regulated genes between SB and SB+DMH1 conditions. fold change cutoff is 2. FIG. 9c shows a violin plot of the selected differentially regulated genes in each cluster between SB and SB+DMH1 condition. FIG. 9d shows a violin plot of the BMP pathway targeted genes in each cluster between SB and SB+DMH1 condition.

FIGS. 10a-10b shows snRNA-Seq analysis of retina differentiation at day 12. FIG. 10a shows a heatmap of cluster genes in the differentiating cells treated with or without DMH1 at day 12. FIG. 10b shows a violin plot of BMP pathway related genes in each cluster between Control and DMH1 conditions at day 12.

FIGS. 11a-11e shows retinal fate from primitive neuroepithelial cells. FIG. 11a shows expression of retina differentiation related genes PAX6, SIX6 and RAX at different timepoints along neural differentiation in cells treated with BMP2 from day 6. FIG. 11b shows a diagram of the experiment design to study the time point when BMP pathway starts to regulate retina differentiation (left panel), and gene expression of retina progenitor markers PAX6, RAX and SIX6 by qPCR (right panel). FIG. 11c shows a diagram of the experiment design to study the dose effect of BMP2 on retina differentiation (left panel), and gene expression of PAX6, RAX and SIX6 by qPCR (right panel). FIG. 11d shows immunostaining of VSX2 and PAX6 in differentiating cells after sequentially treatment with SB and BMP2. Scale bar, 50 μm. FIG. 11e shows the evaluation of different BMP ligands in regulating retina differentiation by examining retina development-related gene expression.

FIG. 12 shows a heatmap of cluster markers shown in FIG. 5a.

FIGS. 13a-13b shows BMP2 regulation of retina differentiation. FIG. 13a shows a volcano plot of differentially regulated genes between BMP2 and Control. FIG. 13b shows a feature plot of MITF and VSX2 among single cell datasets from differentiating cells treated with BMP2, Control or DMH1 conditions.

FIG. 14 shows retina differentiation in multiple hPSC lines. FIG. 14 shows an immunostaining for VSX2 and PAX6 in retina progenitors derived from H9, IMR-90-4 and WC30 human PSC lines. Scale bar, 50 μm.

### DETAILED DESCRIPTION OF THE DISCLOSURE

For the purposes of explicating and understanding the principles of this disclosure, reference is made to embodiments and specific language used to describe the same. The skilled artisan will nevertheless understand that no limitation of the scope of the disclosure is thereby intended, such alteration and further modifications of the disclosure as illustrated herein, being contemplated as would be understood by one skilled in the art to which the disclosure relates.

### Definitions

As used herein, articles “a” and “an” are intended to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the therapeutically beneficial result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by a person of ordinary skill in the art to which this disclosure belongs.

The terms “express” or “expression” refer to transcription and translation of a nucleic acid coding sequence resulting in production of the encoded polypeptide. “Express” or “expression” also refers to antigens that are expressed on cell surfaces.

As used herein, the term “subject” refers to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The subject also and specifically can be a human patient that is at risk for, or suffering from, one or more retinal diseases or disorders. The human subject can be of any age (e.g., an infant, child, or adult).

The term “construct” refers to an artificially designed segment of DNA that can be used to incorporate genetic material into a target cell.

The term “sequence identity” refers to the number of identical or similar nucleotide bases on a comparison between a test and reference oligonucleotide or nucleotide sequence. Sequence identity can be determined by sequence alignment of a first nucleic acid sequence to identify regions of similarity or identity to second nucleic acid sequence. As described herein, sequence identity is generally determined by alignment to identify identical residues. Matches, mismatches, and gaps can be identified between compared sequences by techniques known in the art. Alternatively, sequence identity can be determined without taking into account gaps as the number of identical positions/lengths of the total aligned sequence×100. In one embodiment, the term “at least 90% sequence identity to” refers to percent identities from 90 to 100%, relative to the reference nucleotide sequence. Identity at a level of 90% or more is indicative of the fact that, assuming for exemplary purposes a test and reference polynucleotide sequence length of 100 nucleotides are compared, no more than 10% (i.e., 10 out of 100) of the nucleotides in the test oligonucleotide differ from those of the reference oligonucleotide. Differences are defined as nucleic acid substitutions, insertions, or deletions.

The term “media” or “medium,” as used herein, refers to a gel or liquid that contains nutrients sufficient to facilitate growth of one or more cells, particularly human cells and specifically human iPSCs and differentiated species produced therefrom as disclosed herein.

The term “chemically defined,” as used herein related to growth media, refers to a growth medium suitable for in vitro cell culture, wherein all components and concentrations thereof in the medium are known.

The term “progenitor cells,” as used herein, refers to cells descending from stem cells that can be further differentiated, e.g., retinal progenitor cells as disclosed herein.

The term “stem cell” as used herein is a cell which is undifferentiated. Such a cell can undergo differentiation when submitted to one or more stimuli. The stimuli might be physical, mechanical, electrical, chemical, biochemical, biological or a combination of any of the latter. By stem cell, it is to be understood here either pluripotent or multipotent cells, and either embryonic or induced.

As used herein, the term “pluripotent stem cells” appropriate for use according to a method of the invention are cells having the capacity to differentiate into cells of all three germ layers. Pluripotent stem cells (PSCs) suitable for the differentiation methods disclosed herein include, but are not limited to, human embryonic stem cells (hESCs), human induced pluripotent stem cells (hiPSCs) non-human primate embryonic stein cells (nhpESCs), non-human primate induced pluripotent stem cells (nhpiPSCs). As used herein, “embryonic stem cells” or “ESCs” mean a pluripotent cell or population of pluripotent cells derived from an inner cell mass of a blastocyst.

As used herein, the term “human pluripotent stem cell” will be understood to include cells derived from human embryonic stem cells (hESCs) or induced pluripotent stem cells (iPSCs), in particular such cells that are mammalian cells and particularly human cells.

As used herein, the term “induced pluripotent stem cells” or “iPS cells” or “iPSCs” mean a pluripotent cell or population of pluripotent cells that may vary with respect to their differentiated somatic cell of origin, that may vary with respect to a specific set of potency-determining factors and that may vary with respect to culture conditions used to isolate them, but nonetheless are substantially genetically identical to their respective differentiated somatic cell of origin and display characteristics similar to pluripotent stem cells, such as ESCs, as described herein. Induced pluripotent stem cells, however, are not immediately derived from embryos. As used herein, “not immediately derived from embryos” means that the starting cell type for producing iPS cells is a non-embryonic, non-pluripotent cell, such as a multipotent cell or terminally differentiated cell, such as somatic cells obtained from a post-natal individual.

As used herein, the term “retinal progenitors” or “retinal progenitor cells” (RPCs) relates to cells presenting at least one of (i) retinal multipotency (ii) the expression of various retinal progenitor markers, (iii) a mitotic capacity.

As used herein, the term “early neuroectoderm cells” or “primitive neuroectoderm cells” refer to an early or primitive neuroepithelial stage in the first week that express PAX6 but not SOX1.

As used herein, the term “late neuroectoderm cells” or “definitive neuroectoderm cells” refer to a late or definitive neuroepithelial stage in the second week that express PAX6 and SOX1 in an embryoid body culture system. Definitive neuroectoderm cells are the cells that form the neural tube in vivo.

The terms “pure” and “purity,” as used herein, refer to the proportion of desired cells from the final isolated cells.

The term “composition,” as used herein, refers to a mixture of one or more types of cells.

The term “enriched,” as used herein, when used regarding genes identified in metabolic or developmental pathways, refers to the pathways or gene ontologies are overrepresented in a group of genes more than would be expected at random.

The term “enriched population,” as used herein, refers to increased percentage of the target cell types in a population.

The term “low cell quality,” as used herein, refers to low detection of RNA, the presence of dead cells, or potential doublets. The standard quality control used refers to selecting single cells with more than 300 unique genes (nFeature) and less than 15% or 20% mitochondrial gene reads (percent.mt) for follow-up analysis. Additional procedures were used to exclude low quality cells and doublets in each sample (nFeature<6,000 and nCount<20,000).

In some embodiments, human pluripotent stem cells (hPSCs) are treated with inhibitors of cellular gene expression for certain developmentally relevant genes.

In some embodiments, the inhibitors are BMP or TGF-beta.

In some embodiments, the BMP inhibitor is DMH1.

In further embodiments, neuroectoderm cells treated with the BMP inhibitor produce definitive neuroectoderm cells.

In even further embodiments, treatment with the BMP inhibitor induces PAX6 and SOX1 expression which is characteristic for definitive neuroectoderm cells.

In some embodiments, the TGF-beta inhibitor is chosen from, but not limited to, A 77-01, A 83-01, AZ 12799734, D 4476, disitertide, galunisertib, GW 788388, IN 1130, LY 2109761, R 268712, RepSox, SB 431542, SB 505124, SB 525334, SD 208, or SM 16 (Bio-techne Corporation), or any other suitable TGF-beta inhibitor.

In some embodiments, the TGF-beta inhibitor is SB 431542.

As used herein, the skilled worker will understand the terms set forth below to have their corresponding meanings, wherein BMP is bone morphogenic protein, and BMP2, BMP4, and BMP7 are variant species of BMP; see, Reddi & Reddi, 2009, “Bone morphogenetic proteins (BMPs): from morphogens to metabologens.” Cytokine & Growth Factor Reviews 20:341-2; Human Gene Name Committee (HUGO) reference HGNC 1067.

DMH1 is a BMP (ALK2) inhibitor that is commercially available from Stem Cell Technologies (Cat. No. 73632) having the structure:

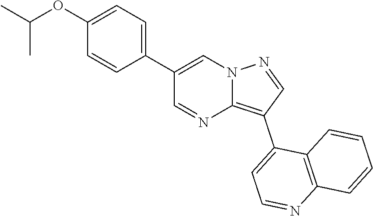 [SMILES: CC(C)Oc1ccc(-c2cnc3c(-c4ccnc5ccccc45)cnn3c2)cc1]

TGF-beta is transforming growth factor beta, identified by OMIM 190180 and UniProt P01137; Human Gene Name Committee (HUGO) reference HGNC 11766.

TGF-beta inhibitors as set forth herein are identified as follows: A 77-01 is a TGF-beta inhibitor commercially available from MedChem Express (Cat. No. 6712) having the structure:

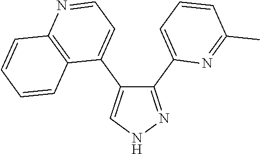 [SMILES: Cc1cccc(-c2n[nH]cc2-c2ccnc3ccccc23)n1]

A 83-01 is a TGF-beta inhibitor commercially available from Stem Cell Technologies (Cat. No. 100-1041) having the structure:

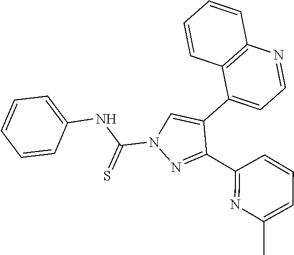 [SMILES: Cc1cccc(-c2nn(C(=S)Nc3ccccc3)cc2-c2ccnc3ccccc23)n1]

AZ 12799734 is a TGF-beta inhibitor commercially available from biotechne tocris (Cat. No. 6870) having the structure:

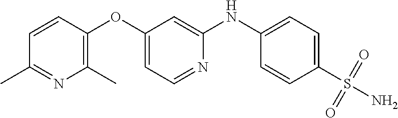 [SMILES: Cc1ccc(Oc2ccnc(Nc3ccc(S(N)(=O)=O)cc3)c2)c(C)n1]

D 4476 is a TGF-beta (ALK-5) inhibitor commercially available from Selleckchem Cat. No. 301836-43-1) having the structure:

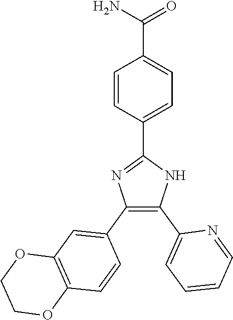 [SMILES: NC(=O)c1ccc(-c2nc(-c3ccc4c(c3)OCCO4)c(-c3ccccn3)[nH]2)cc1]

Disitertide is a peptidic TGF-beta inhibitor commercially available from MedChemExpres (Cat. No. HY-P0118).

Galunisertib is an experimental TGF-beta inhibitor developed by Eli Lilly & Co. having the structure:

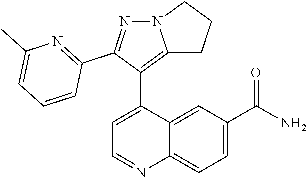 [SMILES: Cc1cccc(-c2nn3c(c2-c2ccnc4ccc(C(N)=O)cc24)CCC3)n1]

GW 788388 is a TGF-beta (ALK-5) inhibitor commercially available from ApexBio (Cat. No. A8301) having the structure:

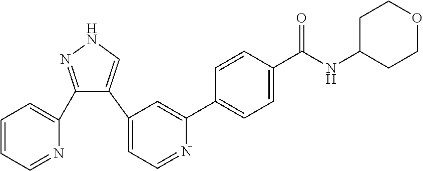 [SMILES: O=C(NC1CCOCC1)c1ccc(-c2cc(-c3c[nH]nc3-c3ccccn3)ccn2)cc1]

IN 1130 is a TGF-beta (ALK-5) inhibitor commercially available from MedChemExpress (Cat. No. HY-18758) having the structure:

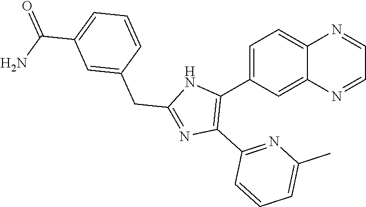 [SMILES: Cc1cccc(-c2nc(Cc3cccc(C(N)=O)c3)[nH]c2-c2ccc3nccnc3c2)n1]

LY 2109761 is a TGF-beta (ALK-5) inhibitor commercially available from Selleckchem Cat. No. S2704) having the structure:

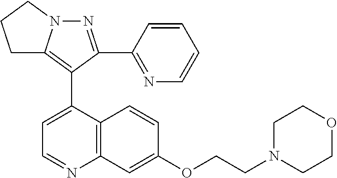 [SMILES: c1ccc(-c2nn3c(c2-c2ccnc4cc(OCCN5CCOCC5)ccc24)CCC3)nc1]

R 268712 is a TGF-beta (ALK-5) inhibitor commercially available from MedChemExpress (Cat. No. HY-12953) having the structure:

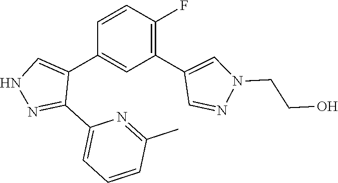 [SMILES: Cc1cccc(-c2n[nH]cc2-c2ccc(F)c(-c3cnn(CCO)c3)c2)n1]

RepSox is a TGF-beta (ALK-5) inhibitor commercially available from Selleckchem Cat. No. S7223) having the structure:

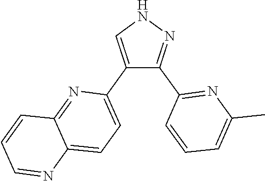 [SMILES: Cc1cccc(-c2n[nH]cc2-c2ccc3ncccc3n2)n1]

SB 431542 is a TGF-beta (ALK-5) inhibitor commercially available from ApexBio (Cat. No. A8249) having the structure:

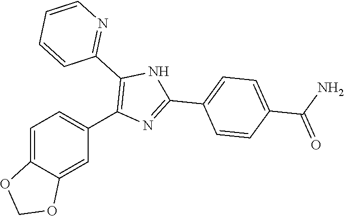 [SMILES: NC(=O)c1ccc(-c2nc(-c3ccc4c(c3)OCO4)c(-c3ccccn3)[nH]2)cc1]

SB 505124 is a TGF-beta (ALK-4, ALK-5) inhibitor commercially available from Selleckchem Cat. No. S2186) having the structure:

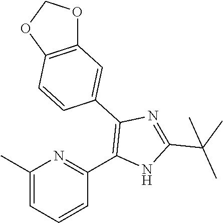 [SMILES: Cc1cccc(-c2[nH]c(C(C)(C)C)nc2-c2ccc3c(c2)OCO3)n1]

SB 525334 is a TGF-beta (ALK-5) inhibitor commercially available from Selleckchem Cat. No. S1476) having the structure:

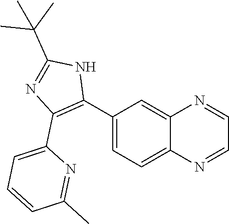 [SMILES: Cc1cccc(-c2nc(C(C)(C)C)[nH]c2-c2ccc3nccnc3c2)n1]

SD 208 is a TGF-beta (ALK-5) inhibitor commercially available from ApexBio (Cat. No. A3808) having the structure:

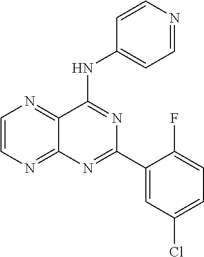 [SMILES: Fc1ccc(Cl)cc1-c1nc(Nc2ccncc2)c2nccnc2n1]

SM 16 is a TGF-beta (ALK-5/ALK-4) inhibitor commercially available from MedChemExpress (Cat. No. HY-111482) having the structure:

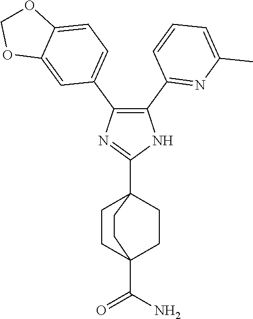 [SMILES: Cc1cccc(-c2[nH]c(C34CCC(C(N)=O)(CC3)CC4)nc2-c2ccc3c(c2)OCO3)n1]

In further embodiments, neuroectoderm cells treated with the TGF-beta inhibitor produce definitive primitive cells (PNCs).

In even further embodiments, treatment with the TGF-beta inhibitor induces PAX6 and SOX1 expression.

In some embodiments PNCs express PAX6, but not SOX1 expression.

In some embodiments, the hPSCs and inhibitors were incubated in chemically defined media for up to 6 days.

In some embodiments, hPSCs are treated simultaneously with a BMP inhibitor and a TGF-beta inhibitor.

In some embodiments, PNCs are treated in cell culture in vitro with BMP2, BMP4, or BMP7 for about 20 days. Treating PNCs with BMP2, BMP4, or BMP7 is effective for producing retinal progenitor cells.

In some embodiments, hPSCs are treated with BMP and TGF-beta for 5 to 6 days. In some embodiments, PNCs are produced by treating the cells with BMP2.

In some embodiments, BMP2 is administered in a concentration of 2-10 ng/ml.

In some embodiments, PNCs are treated with BMP2 after day 4 and before day 7 of cell culture.

In some embodiments, retinal progenitor cells are produced from hPSCs treated with a TGF-beta inhibitor for 5 days followed by treatment with BMP activator from day 6 to day 20.

In some embodiments, the BMP activator is BMP2.

In some embodiments, the concentration of BMP2 is 10 ng/mL.

In some embodiments, the composition of retinal progenitor cells are at least 50% pure.

### Overview

The retina originates from the eye field that is situated between the prospective telencephalon and diencephalon at the most anterior neural plate. How the eye-field cells are specified from the early neuroectoderm remains an enigma, though BMP inhibition was proposed as a driving force from animal studies (Teraoka et al., 2009, Dev Biol 330, 389-398; Bielen & Houart, 2012, Dev Cell 23:812-822). As disclosed herein, informatics analysis comparing the human retinal vs. cerebral cortical progenitors revealed active expression of the BMP pathway genes in retinal progenitors but not in cortical progenitors, suggesting a need of BMP activation for retinal (eye field) specification from the early neuroectoderm instead. Indeed, blockade of BMP signaling committed the early ectodermal cells to the definitive neuroectoderm fate while removal of BMP inhibition endowed the early (primitive) neuroectoderm cells for retinal differentiation. Further BMP activation converted the primitive neuroepithelia to eye-field-like cells and then RPCs. This was achieved through cross regulation between the BMP pathway and the EFTFs at multiple stages. The findings set forth herein renewed the concept on human eye field specification and subsequent RPC differentiation, which led to the establishment of a novel system to generate pure RPCs.

Neuroectoderm induction during gastrulation is orchestrated by inhibition of the TGF-beta/BMP and WNT pathways and activation of the FGF signaling in a temporal-spatial manner (Hemmati-Brivanlou, A et al., 1997, Cell 88, 13-17; Munoz-Sanjuan, et al., 2002, Nat Rev Neurosci 3, 271-280; Harland, R., 2000, Curr Opin Genet Dev 10, 357-362; Heeg-Truesdell, E. et al., 2006, Dev Biol 298, 71-86; Delaune, E., et al., 2005, Development 132, 299-310; Linker, C. et al., 2004, Development 131, 5671-5681). Provided herein also is the evidence that induction of neuroepithelia from hPSCs undergoes two identifiable stages, an early or primitive neuroepithelial stage in the first week that express PAX6 but not SOX1 and a late or definitive neuroepithelial stage in the second week that express PAX6 and SOX1 in an embryoid body culture system (Li, X. J. et al., 2005, Nat.Biotechnol. 23, 215-221; Pankratz, M. T. et al., 2007, Stem Cells 25, 1511-1520; Zhang, X. et al., 2010, Cell Stem Cell 7, 90-100). The result set forth herein confirmed that two-stage neural induction using a monolayer culture platform under TGF-beta inhibition. Results further showed that additional inhibition of the BMP pathway committed the primitive neuroectoderm cells to the definitive neuroectoderm cells, consistent with previous observation (Chambers, S. M. et al., 2009, Nat.Biotechnol. 27, 275-280), which further explained why “dual-smad inhibition” blocked RPC specification. This finding contradicted hypotheses in the prior art that BMP inhibition is required to remove repressive signaling on Rx3 (RAX homolog) expression in the prospective eye field cells (Bielen & Houart, 2012, Dev Cell 23:812-822). Cellular and scRNA-Seq analysis on the differentiating cells demonstrated that BMP inhibition was not required for inducing early primitive neuroepithelia nor eye field cells. In fact, BMP inhibition hindered the specification of RPCs from the primitive neuroepithelia due to the progression to the dorsal telencephalon cells, as indicated by the expression of GLI3, LHX2, PAX6, SIX3 and OTX2 but not RAX. Such a phenomenon suggested a need of a signal to trigger the eye field cells and/or RPC specification from the primitive neuroepithelia. As disclosed herein, treatment with multiple BMP ligands converted the early neuroepithelia to RPCs, as indicated by expression of RAX, PAX6, SIX6, VSX2 and MITF. This was similar to an observation in mice that knocking out BMP receptors conditionally in developing retina cells by Six3Cre resulted in reduced growth of embryonic retina (Murali, D. et al., 2005, Development 132, 913-923) and in hPSC differentiation that a transient high dose of BMP enhances the generation of retinal cells (Capowski et al., 2019, Development 146; Kuwahara, A. et al., 2015, Nat Commun 6, 6286; Harkin, J. et al., 2024, Proc Natl Acad Sci USA 121, e2317285121), although when and how BMPs work in these studies was not documented. The results set forth herein further showed that the effects of BMP treatment occurred only on the primitive neuroepithelia but not before (day 4) or after (day 7). These results further showed that during early development, TGF-beta inhibition allowed specification of the primitive neuroectoderm, which is conducive for specification of both the definitive neuroectoderm and the eye field in the anterior neural plate. Blockade of BMP committed the primitive neuroectoderm to the definitive neuroectoderm whereas activation of BMP permits or triggered the specification of the eye field to generate RPCs.

It is well established in animals (Hogan, B. L., et al., 1988, Development 103 Suppl, 115-119; Hill, R. E. et al., 1991, Nature 354, 522-525; Grindley, J. C., et al., 1995, Development 121, 1433-1442; Hogan, B. L. et al., 1986, J Embryol Exp Morphol 97, 95-110) and hPSCs (Tao et al., 2020, EMBO reports 21) that EFTFs, especially PAX6, are required for neuroretina development. Indeed, knock-out of PAX6, either the PAX6AB KO, retina specific D isoform (PAX6D KO), or both blocked the generation of RPCs despite treatment with BMPs, demonstrating the necessity of PAX6 in mediating the effect of BMPs. Furthermore, PAX6 modulated BMP signaling by regulating BMPR1B transcription during retina specification, as indicated by ChIP-Seq analysis, validating direct targeting of BMPR1B by both PAX6A and PAX6D. These findings suggested a cross signaling between BMPs and PAX6 in regulating eye field or RPC specification. Considering the differential roles of PAX6 isoforms in neuroectoderm and neuroretina specification as well as the spatio-temporal effects of BMPs in these processes, such cross-talks can ensure generation of accurate cell types in these neighboring areas.

Developmental principles are the guideline for hPSC differentiation (Tao, Y. & Zhang, S.C., 2016, Cell Stem Cell 19:573-586). Without the knowledge of how the (early) neuroectoderm is converted into the eye-field cells, current retina differentiation technology employs the embryoid body method to generate the retina lineage cells (Meyer, J. S. et al., 2009, Proc Natl Acad Sci USA 106, 16698-16703; Zhang, S. C., et al., 2001, Nat Biotechnol 19, 1129-1133 (2001), which relies on spontaneous differentiation and yields heterogeneous populations comprising of large populations of cerebral cells besides retinal cells. Hence, manual selection is often necessary to separate the retinal cells from the telencephalic cells thanks to the characteristic optic cup-like morphology (Zhao et al., 2017, Development 144:1368-1381; Zhong et al., 2014, Nat Commun 5:4047; Capowski et al., 2019, Development 146; Gonzalez-Cordero et al., 2017, Stem Cell Reports 9:820-837).

The identification of differential BMP effects in the process of eye-field formation as set forth herein provides a robust system to generate RPCs through generation of the primitive neuroepithelia by TGF-β inhibition followed by eye-field specification and RPC differentiation by BMP activation. This method enables generation of a nearly pure population of RPCs and their differentiated progenies in either a monolayer or 3D culture platform, validated by scRNA-Seq. It is superior to many existing protocols (Harkin, J. et al., 2024, Proc Natl Acad Sci USA 121, e2317285121; Meyer, J. S. et al., 2009, P Natl Acad Sci USA 106, 16698-16703; Mellough, C. B. et al., 2012, Stem Cells 30, 673-686, Nakano, T. et al., 2012, Cell Stem Cell 10, 771-785; Reichman, S. et al., 2014, Proc Natl Acad Sci USA 111, 8518-8523; Zhong, X. F. et al., 2014, Nat Commun 5; Kuwahara, A. et al., 2015, Nat Commun 6) thanks to the directed differentiation with defined conditions that is built upon the well characterized underlying molecular pathway (Table 1). Such a system will facilitate research on the development and pathogenesis of the retinas as well as developing therapeutics for retinal diseases.

Provided herein are therapeutic compositions and methods for producing neuroectoderm and retinal progenitor cells from pluripotent stem cells. Retinal progenitors are known in the art to be capable of giving rise to multiple retina progenies (final differentiated cells) such as ganglion cells and photoreceptor cells. Progenitor cells can be used to treat eye diseases because the cells have the potential to become effective cells, wherein later stage progenies fare desirable for treating degenerative retinal diseases such as glaucoma, age-related macular degeneration (AMD) and retinitis pigmentosa.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The Examples set forth herein incorporate and rely on certain experimental and preparatory methods and techniques preformed as exemplified herein.

### Materials and Methods

hPSC Culture and Retina Differentiation

hPSCs (H9, IMR-90-4 and WC30) were maintained under E8/feeder-free culture condition and passaged at 1:20 ratio with Rock inhibitor. After each new passage, the cells were fed with E8 medium for another 1˜2 days to reach 30-40% confluence. To initiate retina differentiation, culture medium was replaced with Retina Induction Medium (RIM) containing Neural induction medium (NIM) comprising DMEM/F12 media containing 1% N2 and 1× nonessential amino acids (NEAA) supplemented with 10 μM SB431542 (TGF inhibitor, Stemgent, 04-0010-10); this new passage day was counted as day 0 (DO) of retina differentiation. Cells were then fed daily with RIM, or every other day based on the cell density. On day 4, the cells were dissociated by ReLeSR (STEM CELL) or EDTA and re-suspended in the fresh RIM containing 0.5 micromolar (μM) Rock inhibitor. Cell clusters were thereafter transferred to a cell culture flask and cultured in suspension. On day 5, the resulting cell spheres were fed with RIM without SB431542. On day 6, the cell spheres were fed with Retina Specification Medium (RSM) containing NIM supplemented with 10 ng/ml BMP2 (R&D SYSTEMS, 355-BM) every other day till day 20. Typical optical cup structures can be observed as early as day 15; BMP2 can be replaced by BMP4 or BMP7. At day 20, the spheres were cultured in Transition medium (DMEM/F12: Neurobasal (1:1) with 1% N2, 2% B27, 1×NEAA and 1×Glutamax) until analysis.

### Single Nucleus Sequencing and Data Analysis

Differentiating cells at multiple time points (day 0-day 12) indicated below were digested and collected for nucleus isolation and library construction (Tao et al., 2024, Nat. Biotech. 12:1404-1416; https://doi.org/10.1038/s41587-023-01977-4). All libraries prepared in this way were processed by NovaSeq. Alignment of raw sequencing reads, and generation of feature barcode matrices were performed using Cellranger (7.1.0). Seurat (4.1.3) was used to process feature barcode matrices and analyze snRNA-Seq data32. All samples were processed under standard quality control metrics. Single cells with more than 300 unique genes (nFeature) and less than 15% or 20% mitochondrial gene reads (percent.mt) were selected for follow-up analysis. Additional procedures were used to exclude low quality cells and doublets in each sample (nFeature<6,000 and nCount<20,000). In day 30 retina organoid samples, retinoic acid (RA) was used to accelerate retinogenesis from day 20. Early exposure to RA can caudalize progenitors towards the spinal cord region of the neural tube. Cells were filtered by removing HOXB3- and HOXB4-expressing cells to exclude possible caudalized cells in this sample. After quality control, all snRNA-Seq data were normalized using the SCTransform (v2) function in Seurat with mitochondria genes regressed out (Choudhary, S. et al., 2022, Genome Biol 23, 27). PCA and UMAP reduction, gene feature plots were done by using Seurat as well.

### RNA-Seq and Bioinformatics Analysis

Bulk RNA-Seq was performed as previously described (Tao et al., 2020, EMBO reports 21: e50000). RNA-Seq data were processed following quality control, mapping, and analysis of transcripts using FastQC, Trimmomatic, HISAT2, FeatureCount, and DESeq2 pipelines in R. All gene ontology and pathway related analysis were performed using ToppGene (https://toppgene.cchmc.org/). Heatmap and hierarchical clustering of differentially regulated genes in wildtype, PAX6 KO, PAX6AB KO and PAX6D KO were processed by Morpheus (https://software.broadinstitute.org/morpheus/). Gene overlapping was analyzed by Venny 2.1 (https://bioinfogp.cnb.csic.es/tools/venny/)

qPCR Analysis

Total RNA was isolated with the RNeasy Plus Mini Kit according to the manufacturer's instructions. For quantitative PCR (qPCR), cDNA was synthesized using PrimeScript RT Reagent Kit (Takara). qPCR was performed using iTaq Universal SYBR Green Supermix (Bio-Rad). GAPDH gene was used as an internal control to equalize cDNA.

### Immunocytochemistry and Flow Cytometry

Immunocytochemistry was performed as described previously (Huang et al., 2016, Sci Rep 6:32600; Tao et al., 2021, Nat Med 27:632-639). In brief, cells on coverslips were fixed in 4% neutral-buffered paraformaldehyde (PFA) for 10 min at room temperature. Following rinsing with phosphate-buffered saline (PBS), coverslips were incubated in 0.2% Triton X-100 (in PBS) for 10 min followed by incubation in a solution of 10% donkey serum (in PBS) at room temperature for 1 h. Coverslips were then incubated with primary antibodies diluted in 5% donkey serum in 0.1% Triton X-100 (in PBS) at 4° C. overnight, followed by incubation with fluorescently conjugated secondary antibodies at room temperature for 30 min. Nuclei were stained with Hoechst stain. Images were collected with a Nikon A1 laser-scanning confocal microscope.

Flow cytometry was performed following manufacturer's instruction using Transcription Factor Buffer Set which is designed for transcription factor staining. Briefly, single cells were prepared using TrypLE Express Enzyme and fixed in the fixation buffer provided in the kit at 2-8° C. for 45 min. After 3 washings with the permeable buffer, the primary antibodies were added to cells for 45 min at 2-8° C. in a light-tight box. The cells were washed 3 times before incubation with fluorescently conjugated secondary antibodies for 45 mins at 2-8° C. in a light-tight box. After 3 times of washing, cells were suspended in the washing buffer and analyzed by flow cytometry (BD LSR or BD LSRII). Data analysis was performed using FlowJo.

Antibodies used were: SOX2 (R&D Systems AF2018), SOX1 (R&D Systems AF3369), PAX6 Alexa Fluor 488 (BD Biosciences Cat #561664), SOX2-V450 (BD Biosciences Cat #561610), SOX1-PE (BD Biosciences Cat #561592), PAX6 (Covance Cat #PRB-278P), VSX2 (Exalpha, Cat #X1180P), CRX (Abnova, Cat #H00001406-M02), OPSIN (MilliporeSigma, Cat #AB5407).

### Statistics

Data were presented as mean±SEM unless specified in the legends. Statistical analyses were performed using GraphPad Prism 5 or Microsoft Office Excel. The methods used to assess the significance were specified in the figure legends.

### Data Availability

The raw snRNA-seq datasets are available at the Gene Expression Omnibus with accession number GSE250547 (GSM7981367, GSM7981368, GSM7981369, GSM7981370, GSM7981371, GSM7981372). The raw data for RNA-Seq and ChIP-Seq are from GSE128141.

### Example 1

BMP Pathway Genes were Differentially Expressed in Cortical and Retinal Progenitors

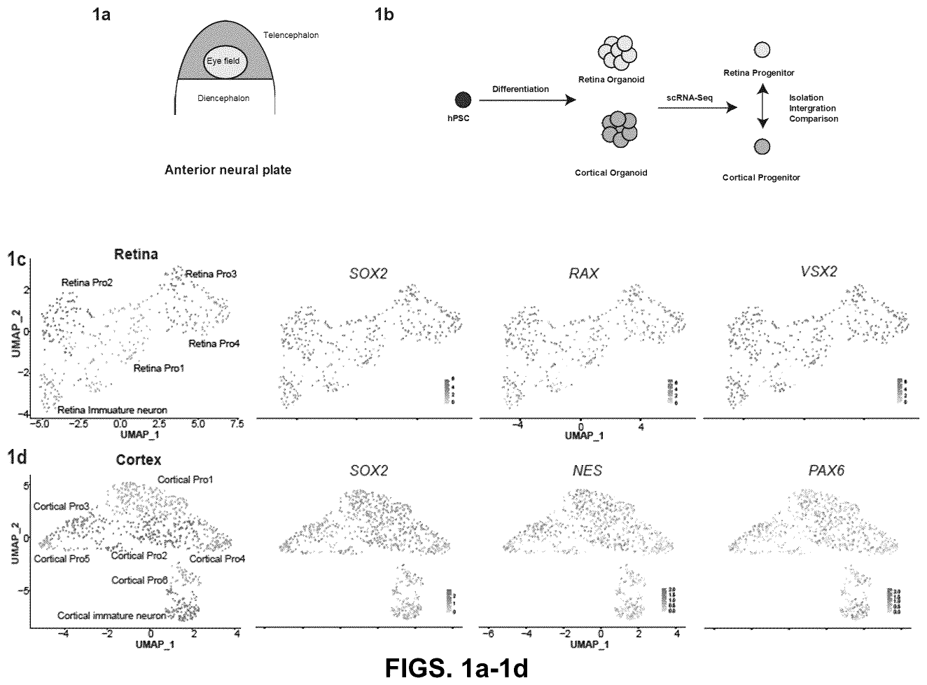

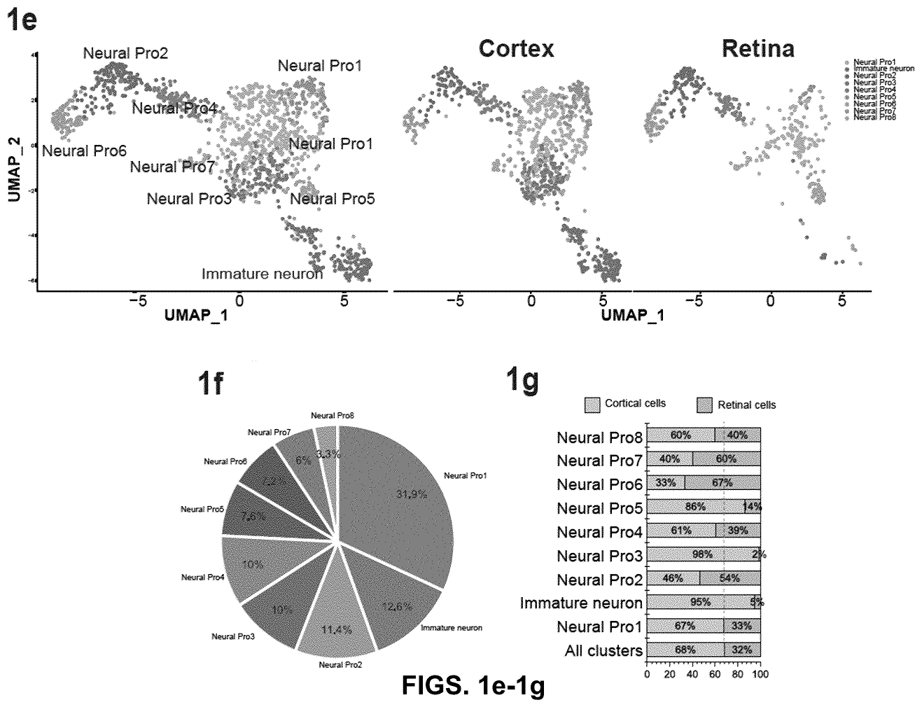

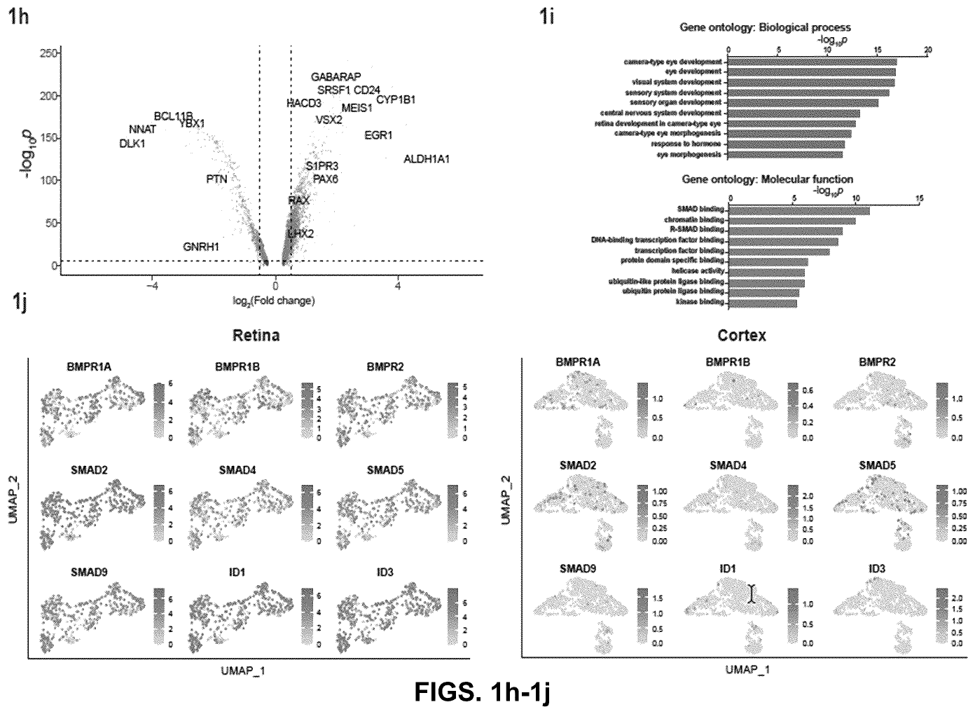

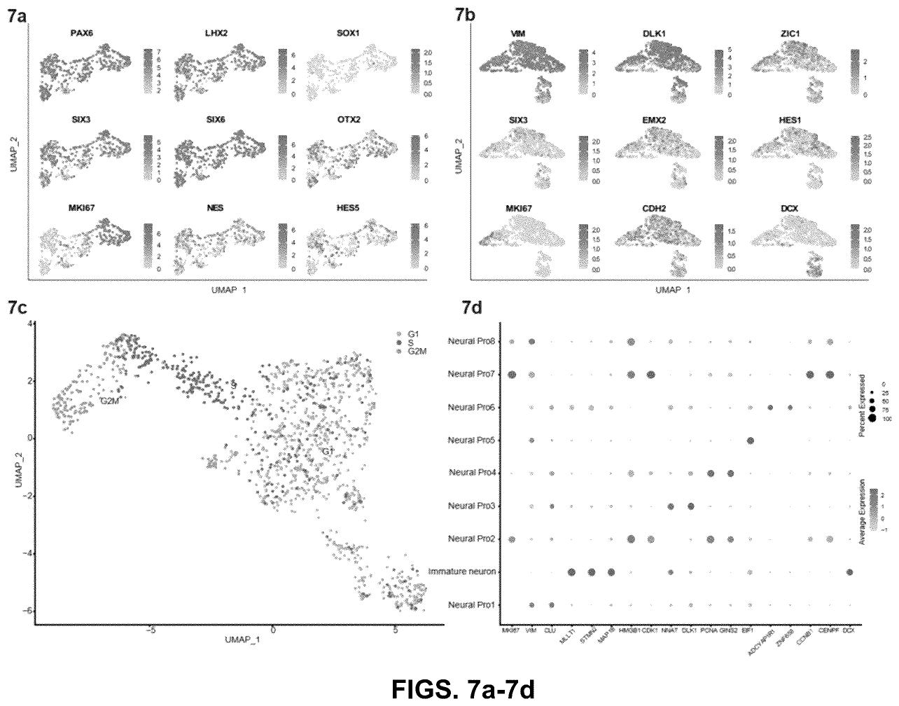

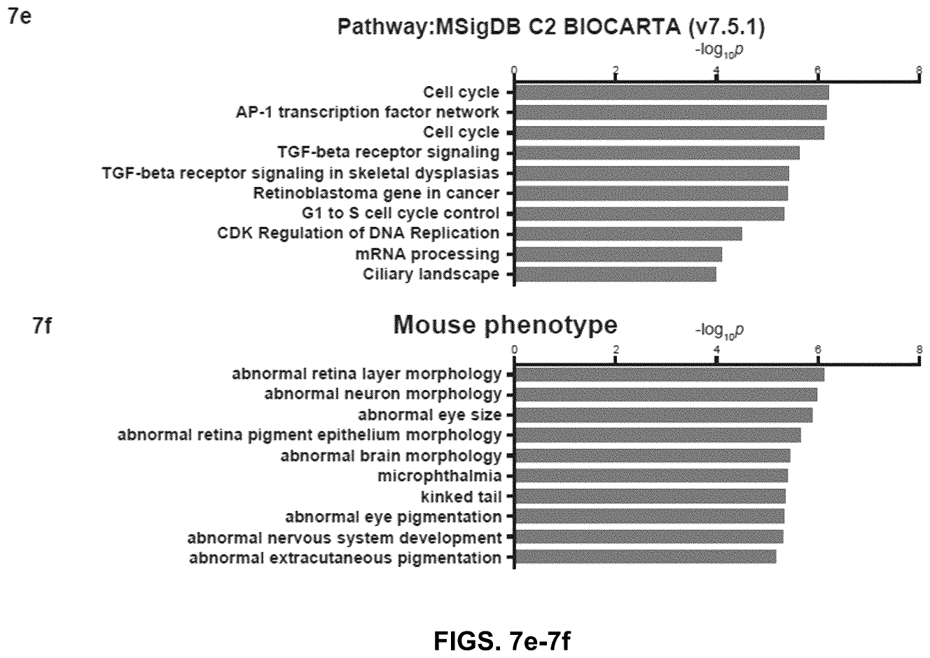

The eye field and telencephalon are adjacent in the most anterior neural plate (FIG. 1a). To identify the extrinsic and intrinsic regulators involved in the eye field specification, gene expression profiles of these two neighboring tissues (the cortical and the retinal progenitor cells) were compared during development at the single-cell level. Progenitor cells were collected by filtering SOX2-positive cells from both cortical and retinal single cell RNA-Seq (scRNA-Seq) datasets (GSE106245 and GSE122783) generated from hPSC-derived organoids (Mao, X. et al., 2019, Stem Cell Reports 13:747-760; Fiddes, I. T. et al., 2018, Cell 173:1356-1369) (FIG. 1b). Cell identities were confirmed by examining their conserved markers. Retinal progenitors were found to be positive for PAX6, RAX, LHX2, OTX2, SIX3, SIX6 and VSX2, which are well-known retinal markers, but negative for SOX1, a definitive neuroepithelial marker (Tao, Y. et al., 2020, EMBO Rep 21, e50000)) (FIGS. 1c, 7a). Cortical neural progenitors were positive for PAX6, NES, OTX2, LHX2, FOXG1, DLK1 but negative for RAX, VSX2 and SIX6 (FIG. 1d and FIG. 7b).

Integration of cortical and retinal progenitors by Seurat (Stuart, T. et al., 2019, Cell 177:1888-1902) revealed that progenitors from the retina and the cortex organoid merged in multiple clusters, indicating very similar gene expression profiles (FIG. 1e-1g). These clusters were well separated by their cell cycle status (FIG. 7c), suggesting that the main variables contributing to clustering were from cell cycle genes. Nine clusters were identified in the integrated single cell dataset (FIG. 1e-1g and FIG. 7d). Eight of them were progenitor cells expressing SOX2, VIM and MKI67 while one cluster was DCX-expressing newborn immature neurons, which was predominantly comprised of cells from the cortical organoid (95%, FIG. 1g). Retinal and cortical specific genes were identified by comparing gene expression between the two different progenitors among all the progenitor clusters (Neural Pro1-5) (Table 1, FIG. 1h). Table 1 shows the percentage of cells expressing the gene in retina cells (Pct.1), the percentage of cells expressing the gene in cortical cells (Pct.2), and the fold change (FC) for the expression levels of the gene in retina versus cortical cells. Well-known retinal markers such as VSX2, RAX, SIX6 were confirmed in the list as the top differentially expressed genes in retinal progenitors (FIG. 1h). Gene ontology (GO) analysis of the up-regulated genes (Fold change>2, p-value<0.05) revealed that biological processes such as “camera-type eye development,” “eye development,” “visual system development,” “sensory system development,” “retina development in camera-type eye,” and “eye morphogenesis” were highly enriched in retinal progenitors (FIG. 1i), further confirming their neural retina identity. Those genes were highly associated with mouse phenotypes relating to retina development such as “abnormal retina layer morphology,” “abnormal eye size,” “microphthalmia,” and “abnormal eye pigmentation” (FIG. 7f).

Analysis of molecular functions of the up-regulated genes revealed enrichment in “SMAD binding,” “R-SMAD binding,” and “transcription factor binding.” Pathway analysis using MSigDB C2 BIOCARTA (v7.5.1) showed that “TGF-beta receptor signaling” was highly enriched in retinal cells (FIG. 7e). Genes involved in the BMP pathways (BMPRIA/B, BMPR2, SMAD2/4/5/9 and ID1/3) were differentially expressed between these two types of progenitors (FIG. 1j), suggesting an important role of the BMP pathway in retinal differentiation.

### Example 2

### BMP Inhibition Blocked Retinal Specification

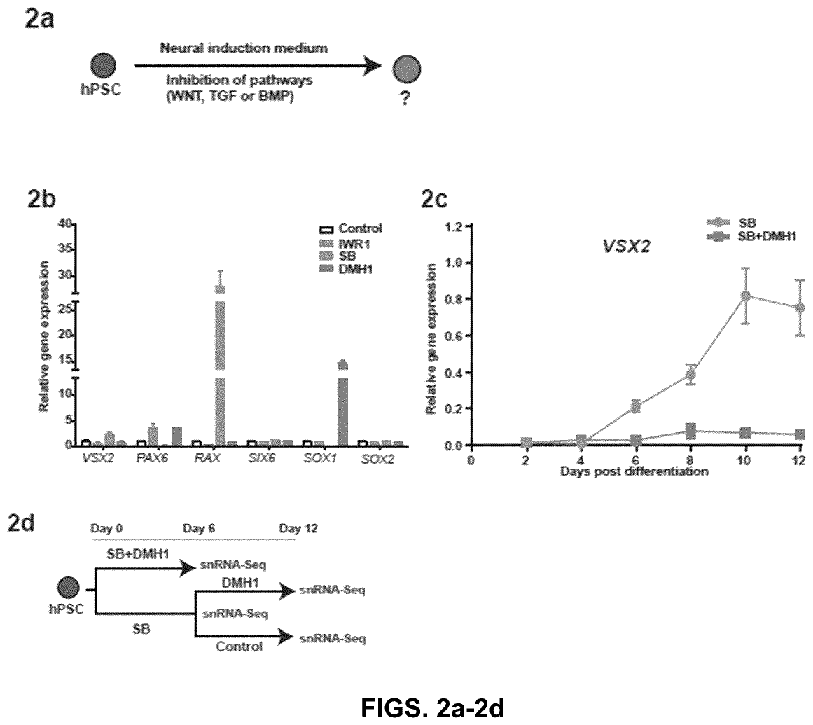

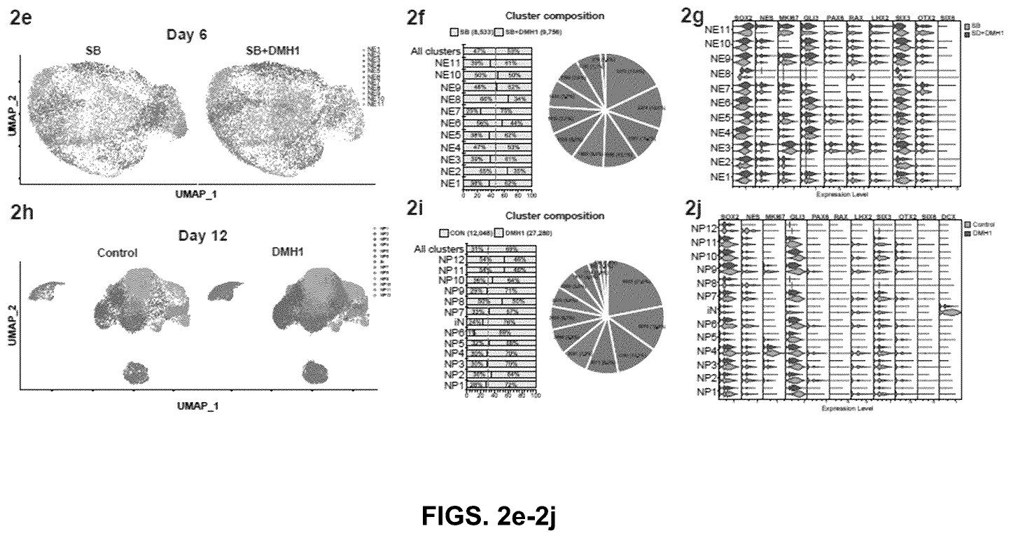

In order to understand whether activation of the BMP pathway was required for switching the neuro/ectoderm to their retinal fate (because inhibition of TFG-beta, BMP, and/or WNT promotes neuroectoderm specification from hPSCs), the next step was to find how regulation of these pathways affected retinal specification. H9 human embryonic stem cells (hESCs) were differentiated to neuroepithelia by culture in chemically defined media as disclosed above and then treated with inhibitors against BMP (DMH1), TGF-beta (SB-431542, SB), or WNT (IWR1) pathways separately for 6 days during the neural induction process (FIG. 2a). Neural genes including PAX6 and SOX1 were efficiently induced by the BMP inhibitor in neural induction medium (DMEM with N₂ supplement). However, retinal lineage-specific markers including RAX and VSX2 did not show much change compared to control. TGF-beta inhibition alone had the highest expression level of RAX and VSX2 but low expression of SOX1 (FIG. 2b), the definitive neuroepithelial marker not expressed by retinal progenitors (Tao, Y. et al., 2020, EMBO reports 21; Kamachi, Y., et. al., 1998, Development 125:2521-2532; Chen, J. et al., 2017, Int J Dev Neurosci 60:94-102). Inhibition of WNT pathway genes did not have much effect on retinal differentiation. These results suggested that BMP inhibition committed the cells to definitive neuroepithelia as indicated by SOX1 expression, whereas TGF-beta blockade induced primitive but not definitive neuroepithelia, which is accompanied by retinal differentiation.

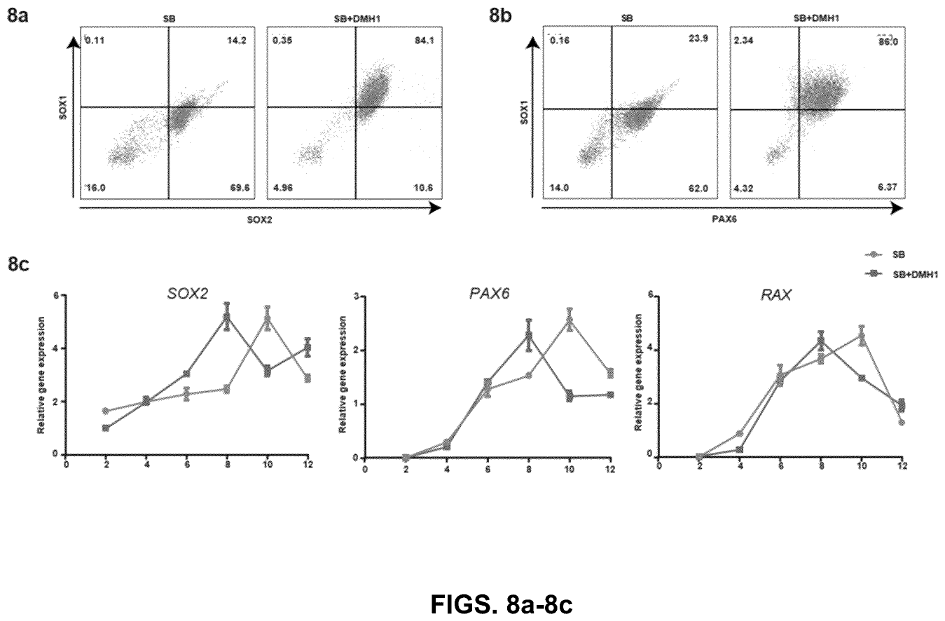

Due to the lack of retinal differentiation resulting from BMP inhibition, expression of retina genes was examined at multiple time points in SB and SB plus dorsomorphin homolog 1 (SB+DMH1), which was a well-established dual-SMAD inhibition condition for neural conversion (Chambers, S. M. et al., 2009, Nat Biotechnol 27:275-280). Retinal progenitor specific marker VSX2 was completely blocked by BMP inhibition when compared to the SB condition (FIG. 2c) while other general neural makers such as SOX2 and PAX6 were not affected (FIG. 8b). Because SOX1 expression was known to be absent in retinal progenitors (also shown in FIG. 7a), expression of SOX2 and SOX1 presented a simple determining marker for cortical vs. retinal progenitors. Over 80% of the differentiating cells were SOX1 and SOX2 double positive cells (definitive for neuroepithelial cells) under dual-SMAD inhibition conditions at day 12 while only 14% of cells in the SB condition were positive for expression of both these genes (FIG. 8a). These results indicated that BMP inhibition increased generation of definitive neuroepithelial cells while inhibiting retinal differentiation.

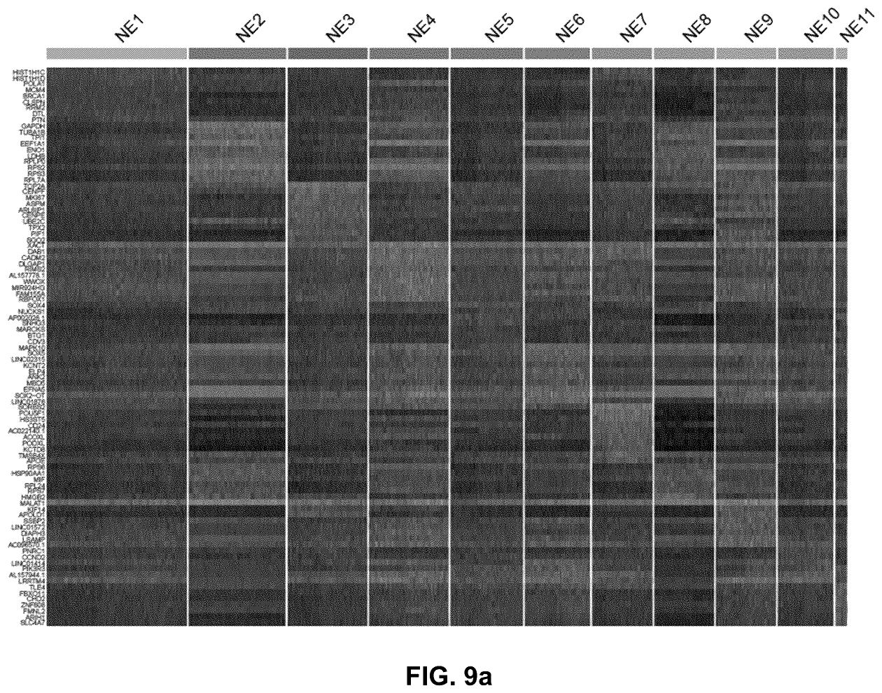

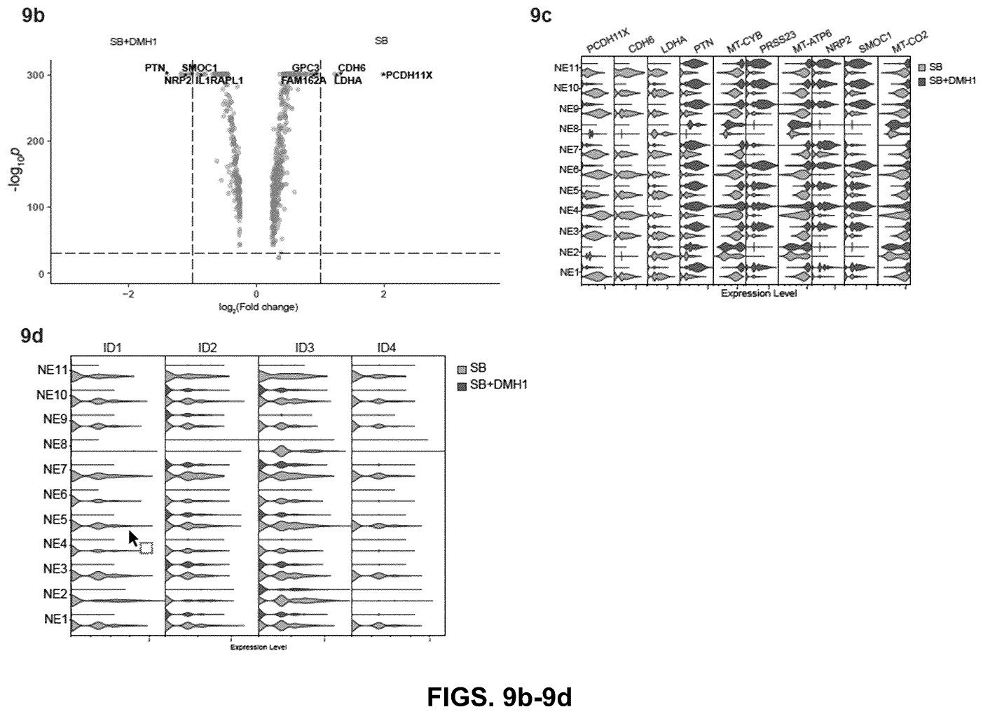

Regulation of retinal differentiation by the BMP pathway was also assessed. Single nucleus RNA-seq (snRNA-Seq) assays were performed to profile and compare the differentiating cells under the SB or SB+DMH1 conditions (FIG. 2d). On day 6, after filtering low quality and dead cells, 8,533 and 9,756 cells in the SB and SB+DMH1 groups were collected, respectively. Uniform manifold approximation and projection (UMAP) clustering identified 11 clusters in both conditions (FIG. 2e). Cell composition in the differentiating cells was very similar among these clusters except cluster neuroepithelial 2 (NE2) and 7 (NE7) (FIGS. 2f-2g). The percentage of cells from different conditions in other clusters was roughly equal to their ratios in the total cells. Cells in most of the clusters expressed neural progenitor makers such as SOX2, NES, MKI67 and GLI3 and EFTFs such as PAX6, RAX, LHX2, SIX3 and OTX2 (FIG. 2g), but not SIX6 in either condition at this time point. RAX expression was slightly lower in most clusters and was absent in the NE8 cluster under SB+DMH1 conditions, suggesting BMP inhibition in the first week already inhibited RAX expression to some degree. Among all these clusters, very few differentially expressed genes (DEGs) were identified between the two conditions (FIGS. 9b-9c). Expression of BMP pathway downstream genes such as ID1/2/3/4 was largely abolished under SB+DMH1 conditions (FIG. 9d), indicating effective BMP inhibition by DMH1. These data indicated that TGF-beta inhibition alone was sufficient for inducing primitive neuroectoderm or ectoderm within 6 days, while additional BMP inhibition committed the cells to the definitive neuroectoderm, impairing retinal differentiation.

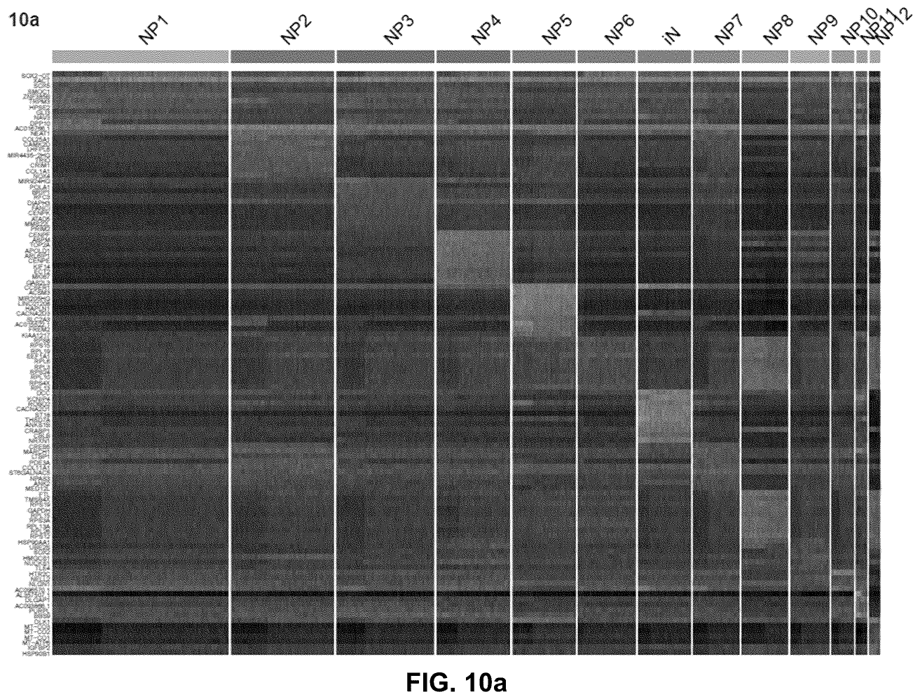

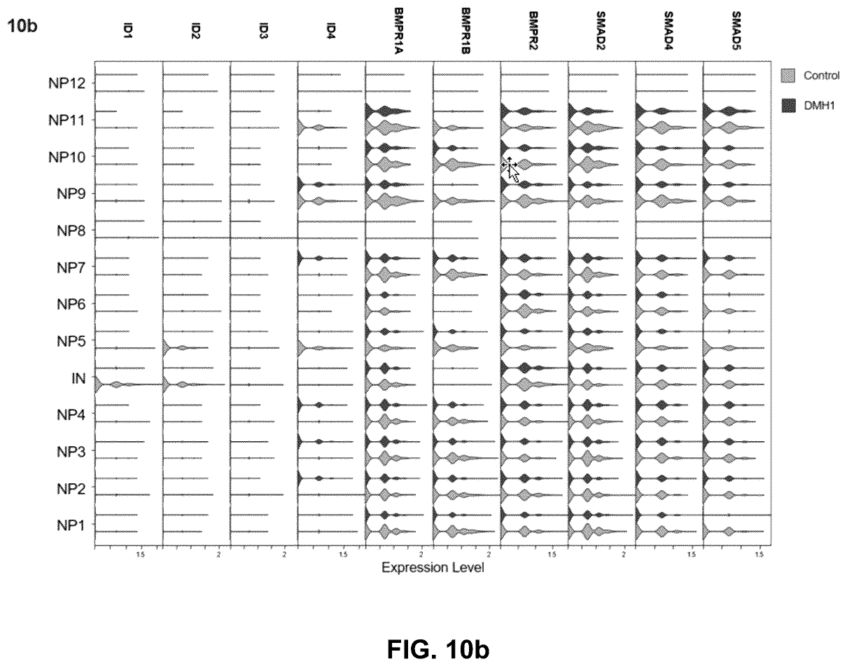

To verify this observation, cells were differentiated with only SB for the first 6 days to generate primitive neuroepithelial cells and then how the BMP pathway regulated retinal differentiation was investigated by treating the cells with or without DMH1 at day 6 (FIG. 2d). Cell nuclei were collected on day 12 for snRNA-Seq to compare differentiating cells in the control (medium without inhibitors) and under DMH1 conditions. After quality control, 12,048 and 27,280 high quality cells were collected from cultured treated under each of these culture conditions, respectively, for snRNA-Seq analysis (results shown in FIGS. 2h-2i). UMAP clustering identified 13 clusters (FIGS. 2h-2j, FIG. 10a). Most of these clusters expressed SOX2, NES, GLI3 and SIX3. One cluster of cells (immature neurons, iN) expressing DCX that began to appear in cells treated under both conditions suggesting an ongoing neurogenesis before day 12. Ratios of cells from each condition in most clusters were close to their ratios in the total cells except in cluster neural progenitor 6 (NP6), 11 (NP11) and 12 (NP12). MKI67 and NES expression levels in progenitor clusters were much lower in cells cultured under DMH1 condition, indicating that BMP inhibition impaired cell proliferation in these differentiating cells. EFTFs such as PAX6, LHX2, SIX3 and OTX2 were much lower or even absent in the differentiating cells under DMH1 treatment. GLI3 was also downregulated by DMH1 treatment, suggesting alteration in cellular identity along the dorsal-ventral axis. Retinal-specific genes RAX and SIX6 were not expressed by any of the clusters, which was consistent with qPCR data showing a down-regulation trend of RAX after day 6 (FIG. 2c). BMP target genes such as ID1/2/4 were expressed at much lower levels in these cells, confirming the effect of DMH1 treatment (FIG. 10b). These results demonstrated that BMP inhibition in the later stage (after primitive neuroepithelial cells) blocked expression of EFTFs (FIG. 2c).

The retina originates from the eye field that is situated between the prospective telencephalon and diencephalon at the most anterior neural plate. The informatics analysis comparing the human retinal vs. cerebral cortical progenitors revealed an active expression of the BMP pathway genes in retinal progenitors but not in cortical progenitors, suggesting a need of BMP activation for retinal (eye field) specification from the early neuroectoderm instead. Indeed, blockade of BMP signaling commits the early ectodermal cells to the definitive neuroectoderm fate while removal of BMP inhibition endows the early (primitive) neuroectoderm cells for retinal differentiation. Further BMP activation converts the primitive neuroepithelia to eye-field-like cells and then RPCs. This is achieved through cross regulation between the BMP pathway and the EFTFs at multiple stages. These findings provided a pathway for establishing a novel system to generate pure RPCs.

### Example 3

BMP Specified the Neural Retinal Fate from Primitive Neuroepithelial Cells

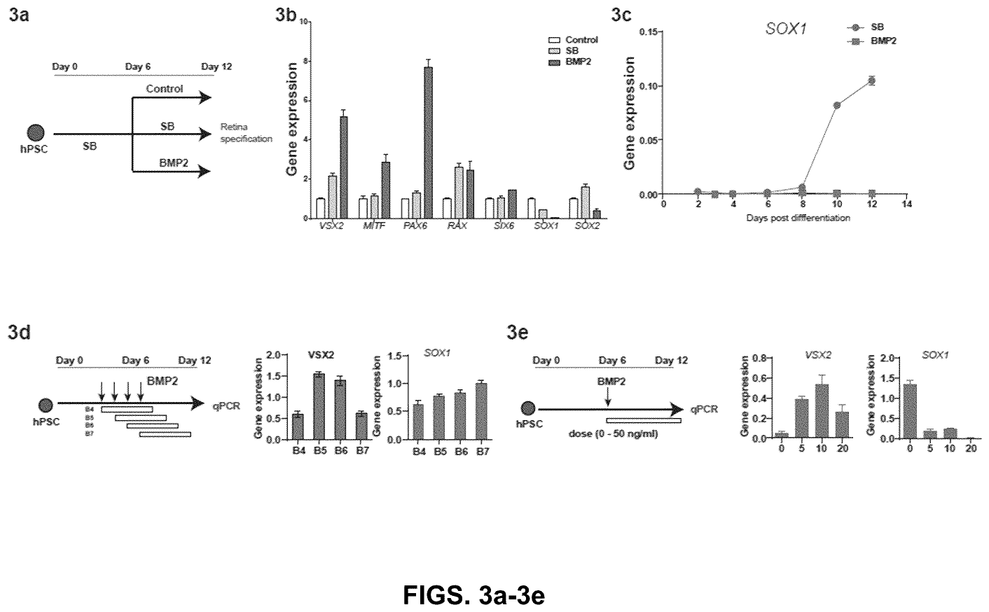

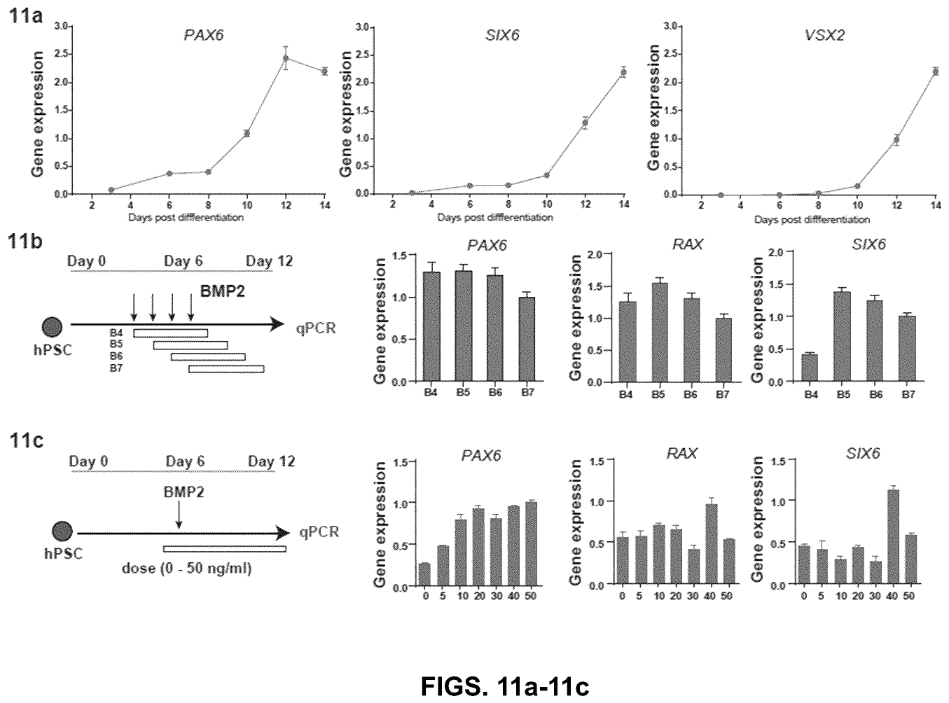

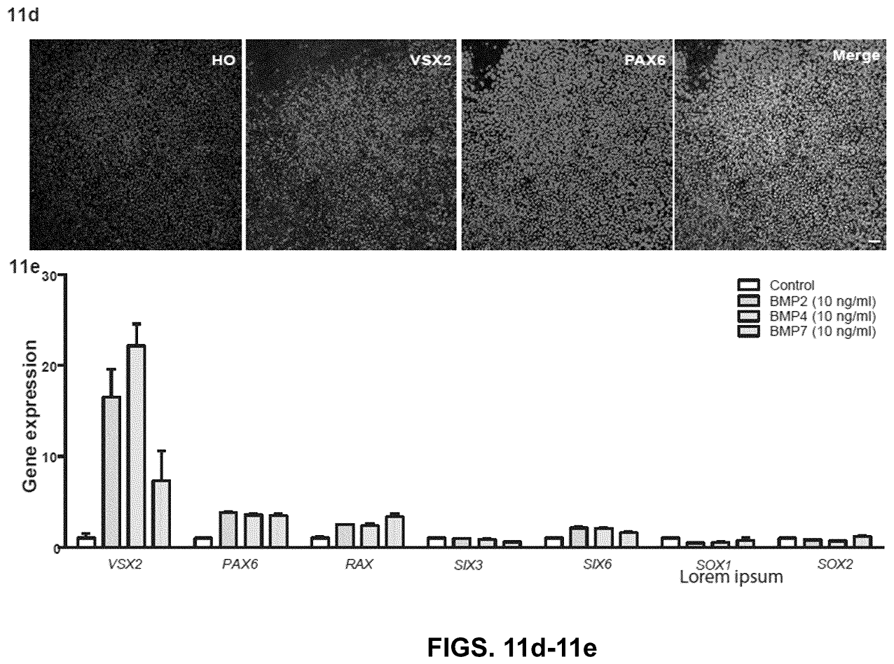

The finding that BMP inhibition blocked EFTF expression raised the question of whether BMP was sufficient for retinal differentiation. To determine whether this was the case, cells were treated with BMP2 (2 ng/ml) on day 6 and collected on day 12 (FIG. 3a). BMP2 strongly enhanced expression of retinal lineage genes such as VSX2, RAX, SIX6 and PAX6 while repressing definitive neuroepithelial marker SOX1 (FIG. 3b). The expression pattern of SOX1 in cells cultured under SB conditions increased steadily after day 6, indicating generation of cortical lineage cells. This effect was completely blocked by BMP2 administration (FIG. 3c). Correspondingly, expression of retinal lineage genes PAX6, SIX6 and VSX2 was increased (FIG. 11a). These results indicated that BMP2 blocked commitment of cells cultured under these conditions to the neuroepithelial fate and promoted acquisition of the retinal fate after day 6.

Next, these cells were treated for 4 days with BMP2 starting from day 4, 5, 6 or 7 and retinal gene expression examined at day 12 to determine whether BMP specified retinal fate. Treating these cells with BMP2 at day 4 was found to inhibit generation of retinal cells, which was indicated by low expression levels of VSX2. This is likely due to impairment of neuroectoderm or ectoderm induction processes, because the BMP pathway was necessary for neuroectoderm differentiation in this early stage (Tao, Y. & Zhang, S.C., 2016, Cell Stem Cell 19:573-586), which was also indicated by low expression levels for SOX1 (FIG. 3d). The most effective window for this BMP2 effect of retinal cell differentiation appeared at around day 5˜ 6, in which time expression levels of retinal lineage genes such as VSX2, PAX6, RAX and SIX6 peaked (FIG. 11b). Thereafter, e.g., at day 7, enhancement of retinal gene expression by BMP2 diminished (FIG. 11b). Thus, induction of retinal fate by BMP occurred at the primitive neuroepithelial stage when these cells were negative for SOX1.

Analysis of the dose effect in these experiments indicated that VSX2 and PAX6 expression levels increased with higher doses of BMP2. BMP2 administration (10 ng/ml) induced maximum levels of VSX2 and PAX6 expression while reducing expression of the definitive neuroepithelial marker SOX1 (FIG. 3e, FIG. 11c). Immunostaining confirmed enriched VSX2 and PAX6 expression in the differentiating cells at day 20 of differentiation when using 10 ng/ml BMP2 (FIG. 11d). Interestingly, other BMP ligands such as BMP4 and BMP7 had a similar effect in regulating these retina transcription factors (FIG. 11e). Together, these results indicated that activation of the BMP pathway specifies the early neuroectoderm to the retinal lineage.

Cellular and scRNA-Seq analysis on the differentiating cells demonstrated that BMP inhibition is not required for inducing early primitive neuroepithelia nor the eye field cells. BMP inhibition hinders the specification of RPCs from the primitive neuroepithelia due to the progression to the dorsal telencephalon cells, as indicated by the expression of GLI3, LHX2, PAX6, SIX3 and OTX2 but not RAX. Treatment with multiple BMP ligands converts the early neuroepithelia to RPCs, as indicated by expression of RAX, PAX6, SIX6, VSX2 and MITF. These results further showed that the BMP effect occurred only on the primitive neuroepithelia but not before (day 4) or after (day 7).

### Example 4

### BMP2 Maintains the EFTF Expression During Retina Specification

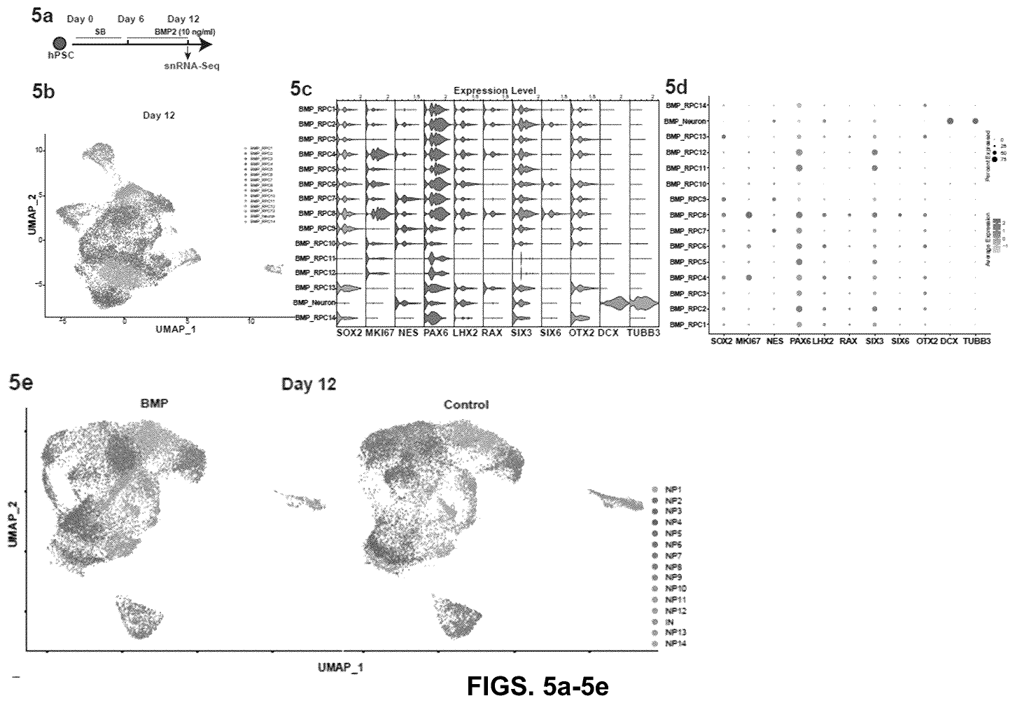

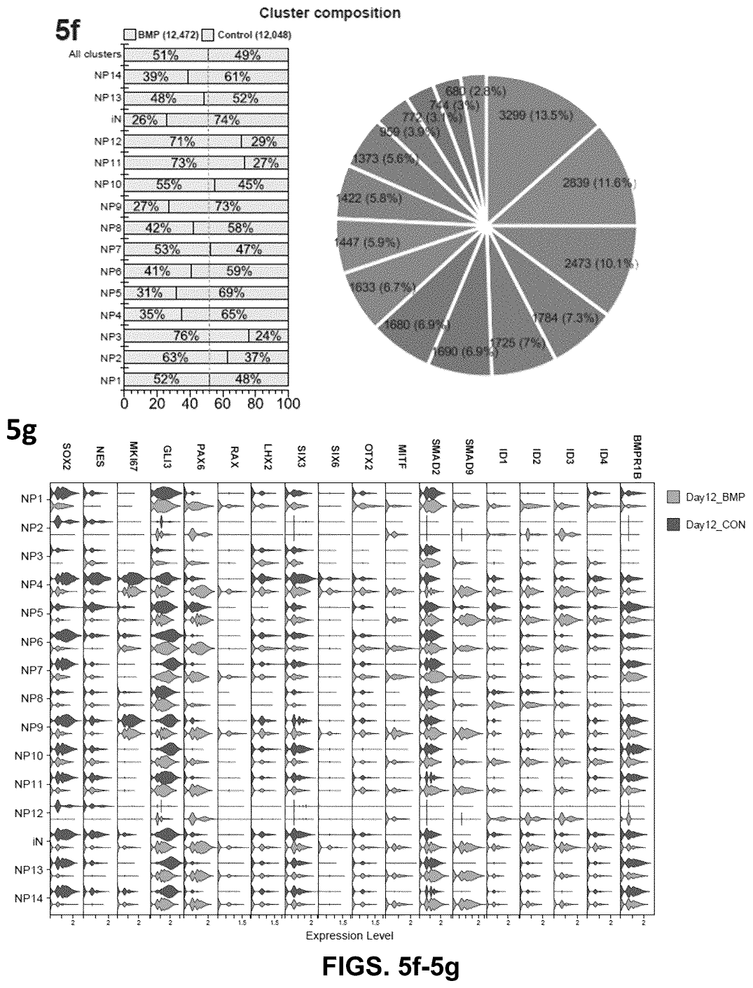

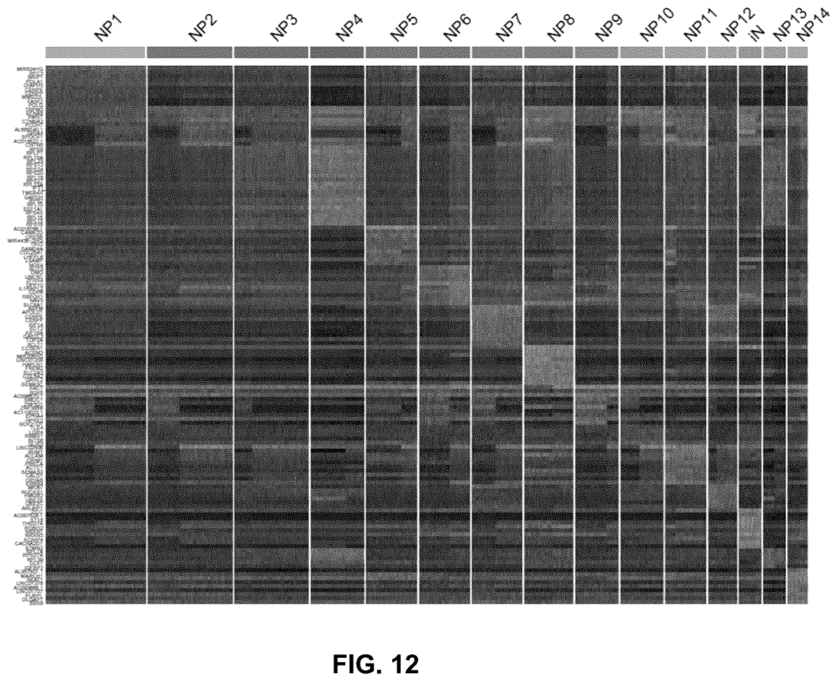

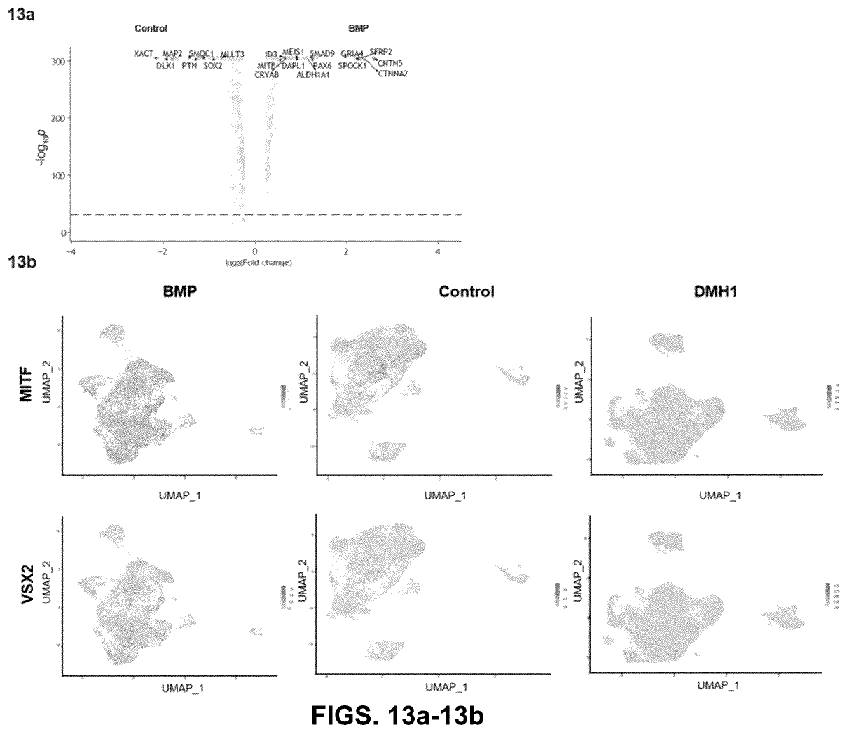

To further illuminate how BMP2 regulated retina specification at a single cell level, snRNA-Seq was performed to profile the differentiating cells under treatment with BMP2 (10 ng/ml) at day 12 (FIGS. 5a-5b). UMAP clustering identified 15 clusters in the population. These cells in most of the clusters co-expressed SOX2, PAX6, SIX3 and OTX2. RAX and LHX2 expression were also detected in multiple clusters (FIGS. 5c-5d). Like the differentiating cells under other conditions (Control or DMH1) at day 12 (FIGS. 2h-2j), there was one cluster comprised of cells expressing DCX and TUBB3, suggesting the presence of newborn immature neurons. Importantly, retina specific marker SIX6 began to express in a few clusters (BMP neural progenitor 2 (BMP_NP2), 6 and 8) in BMP2 condition which was not observed in the control or DMH1 condition (FIG. 2j), confirming the positive regulation of BMP2 in specifying retina fate. The single cell profile was compared between BMP2 (BMP condition) and the control condition (Control) (FIGS. 5e and 12). Integration of these two datasets revealed 15 clusters in the population (FIG. 12). The DEGs were identified among all the clusters between BMP and Control condition (FIG. 13a). The retina lineage genes such as SFRP2, PAX6, DALP1, MITF were up regulated. Specifically, all retinal fate determinant factors in each cluster were compared and up-regulation of PAX6, RAX, SIX3, OTX2, MITF and SIX6 was found at the subpopulation levels (FIG. 5g) indicating the essential role of BMP in maintaining their expression after day 6. The upregulation of ID1/2/3/4 confirmed the activation of BMP pathway by BMP2. In addition, the optic vesicle markers, VSX2 and MITF, were upregulated by BMP as compared to the Control and DMH1 conditions (FIG. 13b).

### Example 5

### PAX6 is Required for BMP Signaling

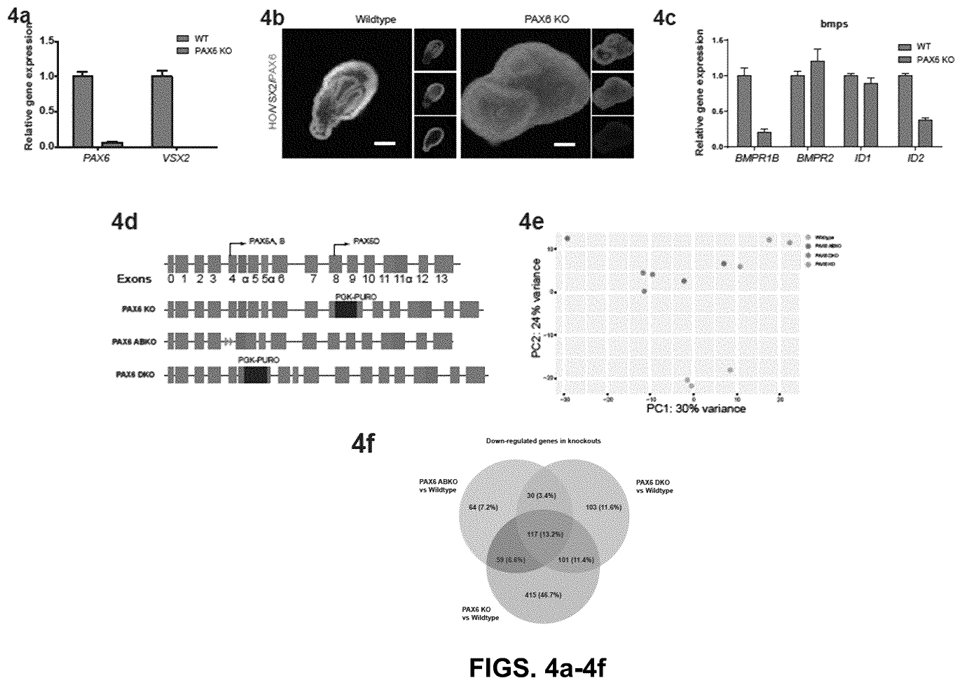

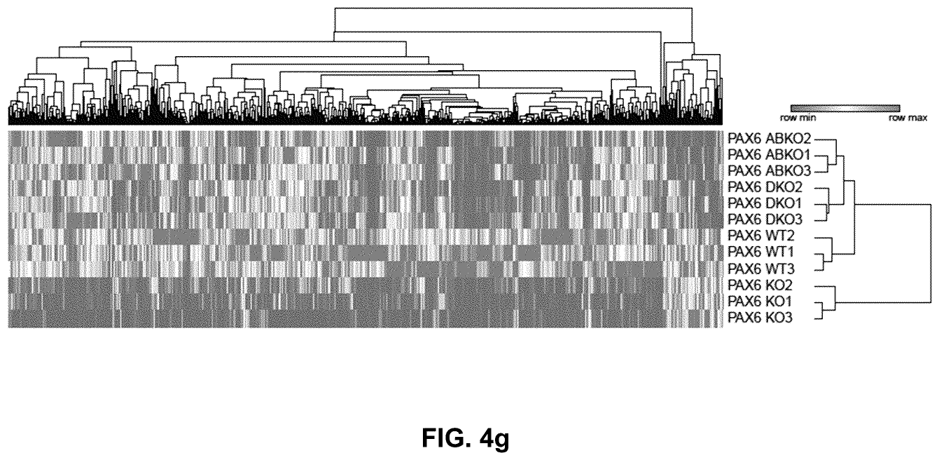

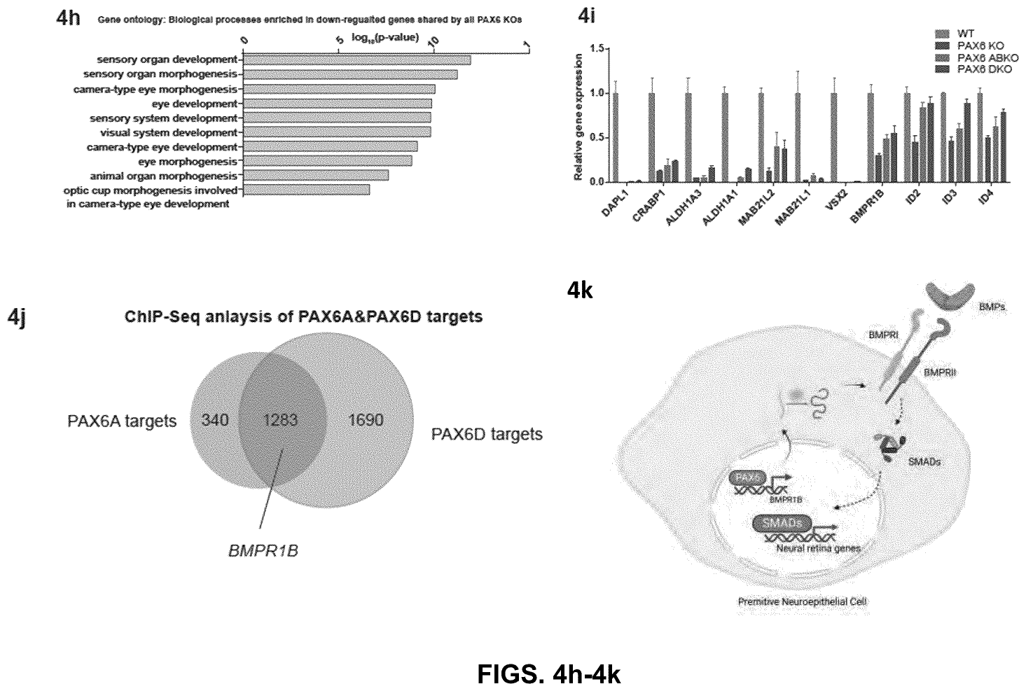

Retina development is tightly regulated by the intrinsic factors such as EFTFs. Results set forth herein demonstrated that BMP signaling was necessary for RPC specification by regulating EFTFs such as RAX, PAX6 and SIX6. In particular, one of the EFTFs, PAX6, was also found to be essential for retinal development at multiple stages (Shaham, O., et al., 2012, Prog Retin Eye Res 31:351-376). How EFTFs interacted with the BMP signaling pathway to regulate retinal differentiation was investigated by the disclosed methods (SB→BMP) to determine whether PAX6 mediated the effects of BMP by differentiating hPSCs without all PAX6 isoforms (PAX6 KO) (Tao, Y. et al., 2020, EMBO reports 21). No VSX2 expression was found in PAX6 KO hPSC-differentiated progenitors (FIGS. 4a-4b), demonstrating the necessity of PAX6 in mediating the BMP effect on RPC specification. Next, how PAX6 modulated BMP signaling in RPC specification was tested. These experiments showed that BMPR1B and ID2 were significantly decreased in PAX6 KO cells while there were no differences in BMPR2 and ID1 expression in cells expressing PAX6 (FIG. 4c), suggesting that PAX6 regulated BMP signaling by modulating its receptors.

To further elucidate how PAX6 regulated the BMP signal pathway and which PAX6 isoform(s) were required (because PAX6 has multiple isoforms expressed in retina cells; Tao, Y. et al., 2020, EMBO reports 21), joint bulk RNA-Seq analysis was performed with RNA-Seq datasets of day 20 progenitors generated from PAX6 KO, PAX6D KO (an isoform uniquely expressed in retina lineage cells) (Tao, Y. et al., 2020, EMBO reports 21) and another PAX6 isoform A&B knockout (PAX6AB KO) (Chen, Y. et al., 2015, Cell Stem Cell 17:233-244) cell lines (FIG. 4c). All samples from PAX6 knockouts (PAX6 KO, PAX6D KO and PAX6AB KO) were grouped together and clearly separated from wildtype samples by PC2 principal variables (FIG. 4e). No clear clustering was found within PAX6 KOs. Among all the pairwise comparisons between PAX6 KO/ABKO/DKO and wildtype, 117 down-regulated genes were shared by all PAX6 KOs (FIG. 4f). Hierarchical clustering of all these differentially regulated genes among wildtype, PAX6 KO, PAX6AB KO and PAX6D KO showed similar gene expression trends in PAX6 KOs (FIG. 4g). Gene ontology analysis of the 117 shared down-regulated genes indicated converged function of PAX6 isoforms in “camera-type eye morphogenesis,” “eye development,” “visual system development,” and “optic cup morphogenesis involved in camera-type eye development” (FIG. 4h). Interestingly, BMPR1B gene expression levels in PAX6 KO, PAX6 ABKO or PAX6D KO cells were significantly lower than in cells expressing wildtype PAX6 (FIG. 4i), suggesting positive regulation of BMPR1B by both PAX6 AB and D isoforms.

To further examine whether PAX6 isoforms directly regulated BMPR1B transcription, ChIP-Seq data was revisited to investigate both PAX6A and PAX6D targets during the retina specification process (Tao, Y. et al., 2020, EMBO reports 21). PAX6A and PAX6D shared 1283 target genes in the process of retina differentiation. Consistent with an essential role of each isoform in BMPR1B expression, BMPR1B was found to be one of the common downstream targets regulated by both PAX6A and PAX6D (FIG. 4j). These results identified a previously unappreciated regulatory mechanism between PAX6 and BMP signaling that linked both intrinsic and extrinsic factors required for retina fate specification processes (FIG. 4k).

Treatment of the primitive neuroepithelia with BMPs up-regulated PAX6 expression, which can be the consequence of retina specification or the direct regulation by BMPs. Knock-out of PAX6, either the A&B, D isoform, or both blocks the generation of RPCs despite the treatment with BMPs, demonstrating the necessity of PAX6 in mediating the effect of BMPs. Furthermore, PAX6 modulates the BMP signaling by regulating BMPR1B transcription during retina specification, as indicated by ChIP-Seq analysis validating the direct targeting of BMPR1B by both PAX6A and PAX6D.

### Example 6

### Regulation of BMP Signaling Enabled Generation of Pure RPCs

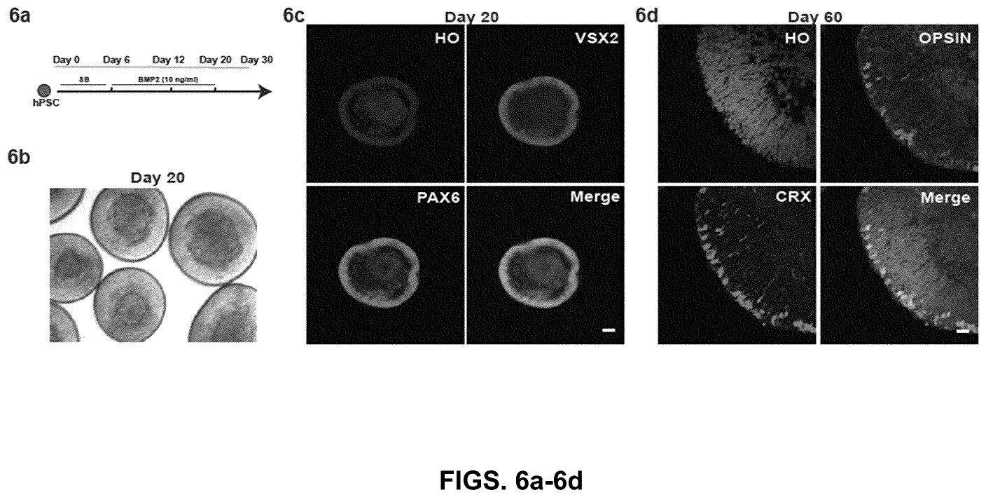

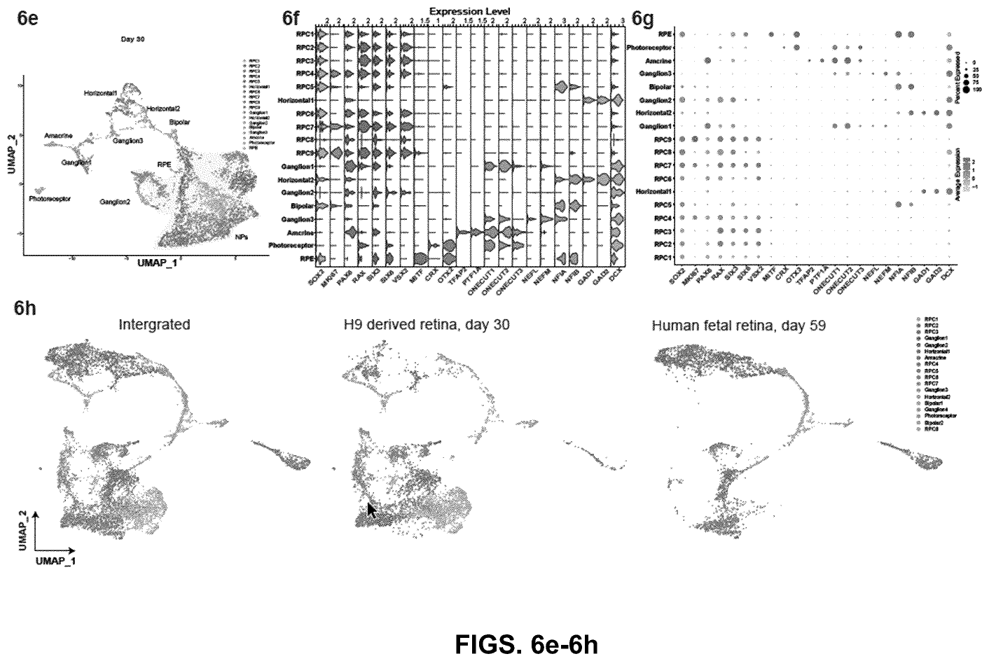

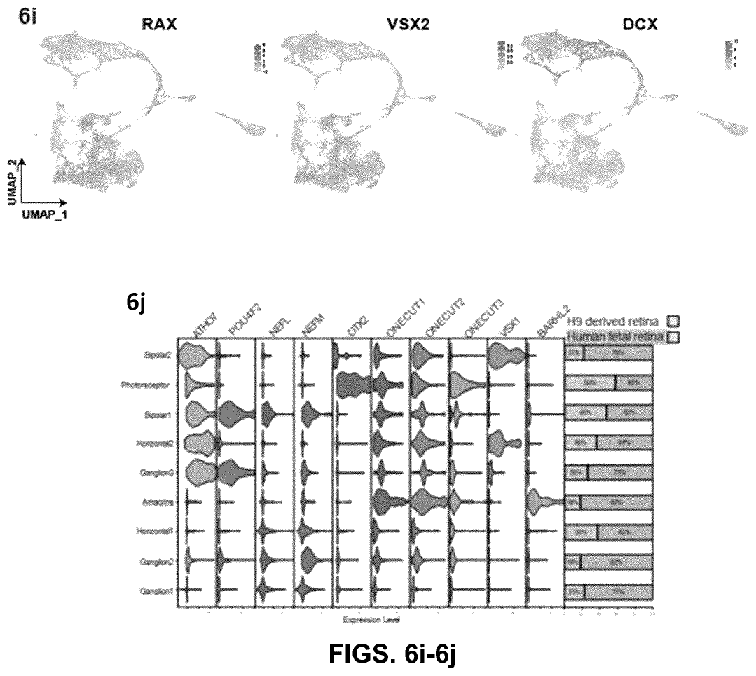

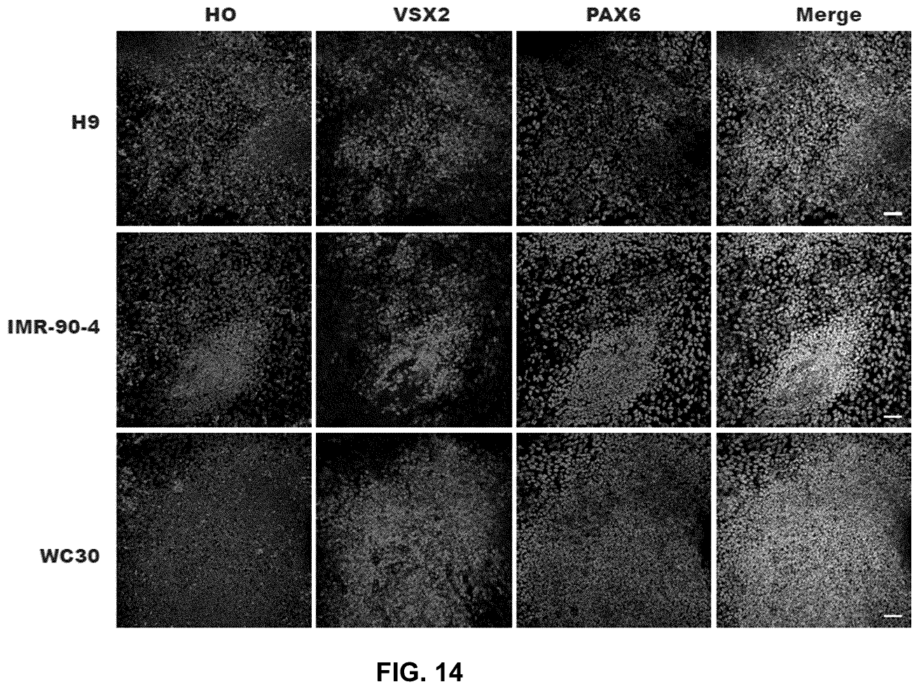

The Examples set forth above resulted in an effective retinal differentiation method performed by sequentially treating hPSCs with SB (day 0-day 5), induction of primitive neuroepithelia and BMP2 (day 6-day 20, 10 ng/ml, specification of eye-field cells and subsequent retinal differentiation) (FIG. 6a), which generated more than 95% PAX6 and VSX2 positive RPCs among all cells after day 15 without manual selection in H9 ESC line. Similar efficiencies have been observed in another 2 iPSC lines (˜87% in IMR90-4 & ˜92% in WC30) (FIG. 14). These conditions permitted homogenous optic vesicle-like structures to be produced in almost all organoids after day 15 when the cells were detached from the solid culture dish substrate and cultured in suspension on day 4 (FIG. 6b). Whole-mount immunostaining of VSX2 and PAX6 confirmed that neural retina progenitors were evenly distributed in a stratified pattern in the organoids (FIG. 6c). The layer structures were largely retained in the organoids after a longer period (until day 60), the generation of photoreceptor cell expressing CRX and OPSIN (FIG. 6d) were detected, confirming the potency of the retinal progenitors in the new condition.

To further validate the identity of retina progenitors specified by sequential treatment of SB and BMP2 (FIG. 6a), snRNA-Seq was performed for these differentiating cells at day 30 (FIGS. 6e-6g). RA was added into the culture medium on day 20 to boost retinogenesis. At day 30, both retinal progenitors (SOX2, MKI67, 80% of all cells) and neurons (DCX, 20% of all cells) were identified in the population (FIG. 6e-6g). Most of the progenitor clusters (77% of all cells) co-expressed retina progenitor markers such as PAX6, RAX, SIX3, SIX6 and VSX2 (FIGS. 6f-6g). Microphthalmia-associated transcription factor (MITF) expression was restricted to one cluster (retina pigment epithelial, RPE, 0.4% of all cells) and segregated from VSX2-expressing cells, which represented retina progenitors at a later stage of retina development. Retina lineage cells such as ganglion (NEFL, NEFM, 8.9% of all cells), horizontal (GAD1, GAD2, 8.7% of all cells), bipolar (NFIA, NFIB, 1.8% of all cells), amacrine (PAX6, TFAP2, PTF1A, 1.6% of all cells) and photoreceptor neurons (CRX, OTX2, 1.5% of all cells) were identified in the population, confirming the multipotency of retina progenitors specified by the new condition.

Mapping the snRNA-Seq dataset (day 30) to the scRNA-Seq datasets from human fetal retina (day 59, GSM4231316) (Sridhar, A. et al., 2020, Cell Rep 30:1644-1659) revealed that all major retinal cells in these cultures merged well with their in vivo counterparts in DCX positive clusters (FIGS. 6h-6j). Fewer retina progenitor (RAX and VSX2 positive) populations were found in the fetal retina samples (FIG. 6h), which is likely due to the more advanced developmental stage in these in vivo samples. In summary, the joint scRNA-seq analysis with the in vivo retina atlas confirmed that the cells derived from the new condition are authentic retina lineage cells.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

### REFERENCES

1 Diacou, R. et al. Cell fate decisions, transcription factors and signaling during early retinal development. Prog Retin Eye Res 91, 101093 (2022). https://doi.org/10.1016/j.preteyeres.2022.101093
2 Giger, F. A. & Houart, C. The Birth of the Eye Vesicle: When Fate Decision Equals Morphogenesis. Front Neurosci 12, 87 (2018). https://doi.org/10.3389/fnins.2018.00087
3 Zuber, M. E., Gestri, G., Viczian, A. S., Barsacchi, G. & Harris, W. A. Specification of the vertebrate eye by a network of eye field transcription factors. Development 130, 5155-5167 (2003). https://doi.org/10.1242/dev.00723
4 Maden, M. Retinoid signalling in the development of the central nervous system. Nat Rev Neurosci 3, 843-853 (2002). https://doi.org/10.1038/nrn963
5 Niehrs, C. Regionally specific induction by the Spemann-Mangold organizer. Nat Rev Genet 5, 425-434 (2004). https://doi.org/10.1038/nrg1347
6 Mason, I. Initiation to end point: the multiple roles of fibroblast growth factors in neural development. Nat Rev Neurosci 8, 583-596 (2007). https://doi.org/10.1038/nrn2189
7 Bielen, H. & Houart, C. The Wnt cries many: Wnt regulation of neurogenesis through tissue patterning, proliferation, and asymmetric cell division. Dev Neurobiol 74, 772-780 (2014). https://doi.org/10.1002/dneu.22168
8 Lagutin, O. V. et al. Six3 repression of Wnt signaling in the anterior neuroectoderm is essential for vertebrate forebrain development. Genes Dev 17, 368-379 (2003). https://doi.org/10.1101/gad.1059403
9 Ando, H. et al. Lhx2 mediates the activity of Six3 in zebrafish forebrain growth. Dev Biol 287, 456-468 (2005). https://doi.org/10.1016/j.ydbio.2005.09.023
10 Georgala, P. A., Carr, C. B. & Price, D. J. The role of Pax6 in forebrain development. Dev Neurobiol 71, 690-709 (2011). https://doi.org/10.1002/dneu.20895
11 Hoch, R. V., Lindtner, S., Price, J. D. & Rubenstein, J. L. OTX2 Transcription Factor Controls Regional Patterning within the Medial Ganglionic Eminence and Regional Identity of the Septum. Cell Rep 12, 482-494 (2015). https://doi.org/10.1016/j.celrep.2015.06.043
12 Matsuo, I., Kuratani, S., Kimura, C., Takeda, N. & Aizawa, S. Mouse Otx2 functions in the formation and patterning of rostral head. Genes Dev 9, 2646-2658 (1995). https://doi.org/10.1101/gad.9.21.2646
13 Orquera, D. P., Nasif, S., Low, M. J., Rubinstein, M. & de Souza, F. S. J. Essential function of the transcription factor Rax in the early patterning of the mammalian hypothalamus. Dev Biol 416, 212-224 (2016). https://doi.org/10.1016/j.ydbio.2016.05.021
14 Jean, D., Bernier, G. & Gruss, P. Six6 (Optx2) is a novel murine Six3-related homeobox gene that demarcates the presumptive pituitary/hypothalamic axis and the ventral optic stalk. Mech Dev 84, 31-40 (1999). https://doi.org/10.1016/s0925-4773 (99) 00068-4
15 Niehrs, C., Kazanskaya, O., Wu, W. & Glinka, A. Dickkopf1 and the Spemann-Mangold head organizer. Int J Dev Biol 45, 237-240 (2001).
16 Houart, C. et al. Establishment of the telencephalon during gastrulation by local antagonism of Wnt signaling. Neuron 35, 255-265 (2002). https://doi.org/10.1016/s0896-6273(02)00751-1
17 Tendeng, C. & Houart, C. Cloning and embryonic expression of five distinct sfrp genes in the zebrafish Danio rerio. Gene Expr Patterns 6, 761-771 (2006). https://doi.org/10.1016/j.modgep.2006.01.006
18 Esteve, P. & Bovolenta, P. Secreted inducers in vertebrate eye development: more functions for old morphogens. Curr Opin Neurobiol 16, 13-19 (2006). https://doi.org/10.1016/j.conb.2006.01.001
19 Moore, K. B., Mood, K., Daar, I. O. & Moody, S. A. Morphogenetic movements underlying eye field formation require interactions between the FGF and ephrinB1 signaling pathways. Dev Cell 6, 55-67 (2004). https://doi.org/10.1016/s1534-5807(03)00395-2
20 Cavodeassi, F. et al. Early stages of zebrafish eye formation require the coordinated activity of Wnt11, Fz5, and the Wnt/beta-catenin pathway. Neuron 47, 43-56 (2005). https://doi.org/10.1016/j.neuron.2005.05.026
21 Teraoka, M. E., Paschaki, M., Muta, Y. & Ladher, R. K. Rostral paraxial mesoderm regulates refinement of the eye field through the bone morphogenetic protein (BMP) pathway. Dev Biol 330, 389-398 (2009). https://doi.org/10.1016/j.ydbio.2009.04.008
22 Bielen, H. & Houart, C. BMP signaling protects telencephalic fate by repressing eye identity and its Cxcr4-dependent morphogenesis. Dev Cell 23, 812-822 (2012). https://doi.org/10.1016/j.devcel.2012.09.006
23 Zhao, C., Wang, Q. & Temple, S. Stem cell therapies for retinal diseases: recapitulating development to replace degenerated cells. Development 144, 1368-1381 (2017). https://doi.org/10.1242/dev.133108
24 Zhong, X. et al. Generation of three-dimensional retinal tissue with functional photoreceptors from human iPSCs. Nat Commun 5, 4047 (2014). https://doi.org/10.1038/ncomms5047
25 Capowski, E. E. et al. Reproducibility and staging of 3D human retinal organoids across multiple pluripotent stem cell lines. Development 146 (2019). https://doi.org/10.1242/dev.171686
26 Gonzalez-Cordero, A. et al. Recapitulation of Human Retinal Development from Human Pluripotent Stem Cells Generates Transplantable Populations of Cone Photoreceptors. Stem Cell Reports 9, 820-837 (2017). https://doi.org/10.1016/j.stemcr.2017.07.022
27 Bielen, H. & Houart, C. BMP Signaling Protects Telencephalic Fate by Repressing Eye Identity and Its Cxcr4-Dependent Morphogenesis. Dev Cell 23, 812-822 (2012). https://doi.org/10.1016/j.devcel.2012.09.006
28 Mao, X. et al. Single-Cell RNA Sequencing of hESC-Derived 3D Retinal Organoids Reveals Novel Genes Regulating RPC Commitment in Early Human Retinogenesis. Stem Cell Reports 13, 747-760 (2019). https://doi.org/10.1016/j.stemcr.2019.08.012
29 Fiddes, I. T. et al. Human-Specific NOTCH2NL Genes Affect Notch Signaling and Cortical Neurogenesis. Cell 173, 1356-1369 e1322 (2018). https://doi.org/10.1016/j.cell.2018.03.051
30 Tao, Y. et al. PAX6D instructs neural retinal specification from human embryonic stem cell-derived neuroectoderm. EMBO Rep 21, e50000 (2020). https://doi.org/10.15252/embr.202050000
31 Stuart, T. et al. Comprehensive Integration of Single-Cell Data. Cell 177, 1888-1902 e1821 (2019). https://doi.org/10.1016/j.cell.2019.05.031
32 Kamachi, Y., Uchikawa, M., Collignon, J., Lovell-Badge, R. & Kondoh, H. Involvement of Sox1, 2 and 3 in the early and subsequent molecular events of lens induction. Development 125, 2521-2532 (1998). https://doi.org/10.1242/dev.125.13.2521
33 Chen, J. et al. Analysis of expression of transcription factors in early human retina. Int J Dev Neurosci 60, 94-102 (2017). https://doi.org/10.1016/j.ijdevneu.2017.01.015
34 Chambers, S. M. et al. Highly efficient neural conversion of human ES and iPS cells by dual inhibition of SMAD signaling. Nat Biotechnol 27, 275-280 (2009). https://doi.org/10.1038/nbt.1529
35 Tao, Y. & Zhang, S. C. Neural Subtype Specification from Human Pluripotent Stem Cells. Cell Stem Cell 19, 573-586 (2016).
36 Shaham, O., Menuchin, Y., Farhy, C. & Ashery-Padan, R. Pax6: a multi-level regulator of ocular development. Prog Retin Eye Res 31, 351-376 (2012). https://doi.org/10.1016/j.preteyeres.2012.04.002
37 Chen, Y. et al. Engineering Human Stem Cell Lines with Inducible Gene Knockout using CRISPR/Cas9. Cell Stem Cell 17, 233-244 (2015). https://doi.org/10.1016/j.stem.2015.06.001
38 Sridhar, A. et al. Single-Cell Transcriptomic Comparison of Human Fetal Retina, hPSC-Derived Retinal Organoids, and Long-Term Retinal Cultures. Cell Rep 30, 1644-1659 e1644 (2020). https://doi.org/10.1016/j.celrep.2020.01.007
39 Hemmati-Brivanlou, A. & Melton, D. Vertebrate embryonic cells will become nerve cells unless told otherwise. Cell 88, 13-17 (1997). https://doi.org/10.1016/s0092-8674 (00) 81853-x
40 Munoz-Sanjuan, I. & Brivanlou, A. H. Neural induction, the default model and embryonic stem cells. Nat Rev Neurosci 3, 271-280 (2002). https://doi.org/10.1038/nrn786
41 Harland, R. Neural induction. Curr Opin Genet Dev 10, 357-362 (2000). https://doi.org/10.1016/s0959-437x(00)00096-4
42 Heeg-Truesdell, E. & LaBonne, C. Neural induction in Xenopus requires inhibition of Wnt-beta-catenin signaling. Dev Biol 298, 71-86 (2006). https://doi.org/10.1016/j.ydbio.2006.06.015
43 Delaune, E., Lemaire, P. & Kodjabachian, L. Neural induction in Xenopus requires early FGF signalling in addition to BMP inhibition. Development 132, 299-310 (2005).
44 Linker, C. & Stern, C. D. Neural induction requires BMP inhibition only as a late step, and involves signals other than FGF and Wnt antagonists. Development 131, 5671-5681 (2004).
45 Li, X. J. et al. Specification of motoneurons from human embryonic stem cells. Nat.Biotechnol. 23, 215-221 (2005).
46 Pankratz, M. T. et al. Directed neural differentiation of human embryonic stem cells via an obligated primitive anterior stage. Stem Cells 25, 1511-1520 (2007). https://doi.org/10.1634/stemcells.2006-0707
47 Zhang, X. et al. Pax6 is a human neuroectoderm cell fate determinant. Cell Stem Cell 7, 90-100 (2010). https://doi.org/10.1016/j.stem.2010.04.017
48 Chambers, S. M. et al. Highly efficient neural conversion of human ES and iPS cells by dual inhibition of SMAD signaling. Nat.Biotechnol. 27, 275-280 (2009).
49 Murali, D. et al. Distinct developmental programs require different levels of Bmp signaling during mouse retinal development. Development 132, 913-923 (2005). https://doi.org/10.1242/dev.01673
50 Kuwahara, A. et al. Generation of a ciliary margin-like stem cell niche from self-organizing human retinal tissue. Nat Commun 6, 6286 (2015). https://doi.org/10.1038/ncomms7286
51 Harkin, J. et al. A highly reproducible and efficient method for retinal organoid differentiation from human pluripotent stem cells. Proc Natl Acad Sci USA 121, e2317285121 (2024). https://doi.org/10.1073/pnas.2317285121
52 Hogan, B. L., Hirst, E. M., Horsburgh, G. & Hetherington, C. M. Small eye (Sey): a mouse model for the genetic analysis of craniofacial abnormalities. Development 103 Suppl, 115-119 (1988).
53 Hill, R. E. et al. Mouse small eye results from mutations in a paired-like homeobox-containing gene. Nature 354, 522-525 (1991). https://doi.org/10.1038/354522a0
54 Grindley, J. C., Davidson, D. R. & Hill, R. E. The role of Pax-6 in eye and nasal development. Development 121, 1433-1442 (1995).
55 Hogan, B. L. et al. Small eyes (Sey): a homozygous lethal mutation on chromosome 2 which affects the differentiation of both lens and nasal placodes in the mouse. J Embryol Exp Morphol 97, 95-110 (1986).
56 Meyer, J. S. et al. Modeling early retinal development with human embryonic and induced pluripotent stem cells. Proc Natl Acad Sci USA 106, 16698-16703 (2009). https://doi.org/10.1073/pnas.0905245106
57 Zhang, S. C., Wernig, M., Duncan, I. D., Brustle, O. & Thomson, J. A. In vitro differentiation of transplantable neural precursors from human embryonic stem cells. Nat Biotechnol 19, 1129-1133 (2001). https://doi.org/10.1038/nbt1201-1129
58 Meyer, J. S. et al. Modeling early retinal development with human embryonic and induced pluripotent stem cells. P Natl Acad Sci USA 106, 16698-16703 (2009). https://doi.org/10.1073/pnas.0905245106
59 Mellough, C. B., Sernagor, E., Moreno-Gimeno, I., Steel, D. H. & Lako, M. Efficient stage-specific differentiation of human pluripotent stem cells toward retinal photoreceptor cells. Stem Cells 30, 673-686 (2012). https://doi.org/10.1002/stem.1037
60 Nakano, T. et al. Self-formation of optic cups and storable stratified neural retina from human ESCs. Cell Stem Cell 10, 771-785 (2012). https://doi.org/10.1016/j.stem.2012.05.009
61 Reichman, S. et al. From confluent human iPS cells to self-forming neural retina and retinal pigmented epithelium. Proc Natl Acad Sci USA 111, 8518-8523 (2014). https://doi.org/10.1073/pnas. 1324212111
62 Zhong, X. F. et al. Generation of three-dimensional retinal tissue with functional photoreceptors from human iPSCs. Nat Commun 5 (2014). https://doi.org/ARTN 4047 10.1038/ncomms5047
63 Kuwahara, A. et al. Generation of a ciliary margin-like stem cell niche from self-organizing human retinal tissue. Nat Commun 6 (2015). https://doi.org/ARTN 6286 10.1038/ncomms7286
64 Tao, Y. et al. Generation of locus coeruleus norepinephrine neurons from human pluripotent stem cells. Nat Biotechnol (2023). https://doi.org/10.1038/s41587-023-01977-4
65 Choudhary, S. & Satija, R. Comparison and evaluation of statistical error models for scRNA-seq. Genome Biol 23, 27 (2022). https://doi.org/10.1186/s13059-021-02584-9
66 Huang, C. T. et al. Time-Course Gene Expression Profiling Reveals a Novel Role of Non-Canonical WNT Signaling During Neural Induction. Sci Rep 6, 32600 (2016). https://doi.org/10.1038/srep32600
67 Tao, Y. et al. Autologous transplant therapy alleviates motor and depressive behaviors in parkinsonian monkeys. Nat Med 27, 632-639 (2021). https://doi.org/10.1038/s41591-021-01257

TABLE 1

| Genes | p_val | avg_log2FC | pct. 1 | pct. 2 | p_val_adj |
|---|---|---|---|---|---|
| RPS17 | 2.79E−246 | 2.37620976 | 0.984 | 0 | 5.88E−242 |
| CALM2 | 2.99E−239 | 3.17505675 | 0.977 | 0.011 | 6.28E−235 |
| MYL6 | 1.42E−225 | 1.89383878 | 0.938 | 0.012 | 2.98E−221 |
| GABARAP | 1.03E−224 | 1.64116264 | 0.917 | 0 | 2.17E−220 |
| LRRC75A-AS1 | 1.44E−224 | 1.69525534 | 0.917 | 0 | 3.04E−220 |
| SIX6 | 2.30E−222 | 2.26007247 | 0.912 | 0 | 4.85E−218 |
| CRABP1 | 1.06E−215 | 3.83608392 | 0.979 | 0.079 | 2.22E−211 |
| SRSF1 | 1.11E−209 | 1.69178871 | 0.87 | 0 | 2.34E−205 |
| CTNNB1 | 7.02E−207 | 2.43829555 | 0.969 | 0.112 | 1.48E−202 |
| TUBB3 | 7.40E−207 | 2.40895116 | 0.863 | 0 | 1.56E−202 |
| DAPL1 | 1.89E−205 | 1.89236586 | 0.858 | 0 | 3.97E−201 |
| CD24 | 1.02E−203 | 2.1123112 | 0.852 | 0 | 2.15E−199 |
| PTPRS | 2.58E−203 | 1.91378565 | 0.909 | 0.042 | 5.44E−199 |
| SRSF6 | 8.31E−200 | 2.11702403 | 0.912 | 0.056 | 1.75E−195 |
| CYP1B1 | 4.82E−198 | 3.6716923 | 0.834 | 0 | 1.01E−193 |
| HACD3 | 6.90E−197 | 1.56615265 | 0.829 | 0 | 1.45E−192 |
| MEIS1 | 1.11E−190 | 1.89728862 | 0.878 | 0.049 | 2.34E−186 |
| HNRNPH1 | 2.11E−186 | 1.77843534 | 0.977 | 0.191 | 4.45E−182 |
| MAB21L1 | 9.56E−185 | 2.13581146 | 0.803 | 0.007 | 2.01E−180 |
| LRP2 | 6.88E−184 | 1.91281342 | 0.808 | 0.013 | 1.45E−179 |
| BCL11B | 6.56E−179 | −3.56436637 | 0.005 | 1 | 1.38E−174 |
| RPS17L | 1.14E−178 | −3.44298404 | 0 | 0.999 | 2.41E−174 |
| PPDPF | 1.36E−177 | −3.55046985 | 0.047 | 0.999 | 2.86E−173 |
| C17orf76-AS1 | 2.61E−177 | −3.04471475 | 0 | 0.994 | 5.50E−173 |
| EEF1A1P6 | 3.32E−177 | −3.28223315 | 0 | 0.994 | 6.99E−173 |
| EEF1G | 5.11E−177 | 2.15227342 | 0.977 | 0.282 | 1.08E−172 |
| RPS10 | 1.81E−175 | −2.92847265 | 0.01 | 0.989 | 3.81E−171 |
| VSX2 | 2.48E−175 | 1.56867485 | 0.756 | 0 | 5.23E−171 |
| PFN2 | 3.37E−174 | 1.29476358 | 0.751 | 0 | 7.10E−170 |
| RPS2 | 2.98E−173 | −4.01329326 | 0.819 | 1 | 6.27E−169 |
| DPYSL2 | 5.18E−173 | 1.68419542 | 0.886 | 0.097 | 1.09E−168 |
| SLC38A2 | 1.62E−171 | 1.6662799 | 0.852 | 0.071 | 3.41E−167 |
| SFRP2 | 7.84E−171 | 4.2799988 | 0.964 | 0.356 | 1.65E−166 |
| YBX1 | 1.94E−170 | −2.88701581 | 0.446 | 1 | 4.09E−166 |
| RPL15 | 3.13E−170 | −2.5003093 | 0.972 | 1 | 6.60E−166 |
| COL2A1 | 5.80E−170 | 2.2100324 | 0.847 | 0.076 | 1.22E−165 |
| RPL6P27 | 4.89E−169 | −2.36946136 | 0 | 0.966 | 1.03E−164 |
| RPL13AP5 | 9.12E−169 | −2.52823969 | 0.003 | 0.966 | 1.92E−164 |
| RPL13P12 | 1.07E−168 | −2.39818443 | 0 | 0.965 | 2.26E−164 |
| FZD5 | 3.53E−168 | 2.32167831 | 0.78 | 0.029 | 7.43E−164 |
| CDH11 | 3.76E−168 | 1.59842372 | 0.744 | 0.006 | 7.91E−164 |
| RPL13 | 5.03E−168 | −2.6506454 | 0.948 | 1 | 1.06E−163 |
| RPS27A | 1.09E−167 | −2.55226358 | 0.733 | 0.998 | 2.29E−163 |
| H3F3A | 1.81E−166 | −2.40186009 | 0 | 0.957 | 3.81E−162 |
| RPL14 | 3.91E−166 | −2.50836899 | 0.583 | 0.999 | 8.24E−162 |
| MARCH6 | 4.77E−166 | 1.76683424 | 0.92 | 0.181 | 1.00E−161 |
| RPS3AP47 | 3.76E−165 | −2.48012604 | 0 | 0.954 | 7.91E−161 |
| MT-CO1 | 1.18E−164 | −3.55241075 | 0 | 0.954 | 2.49E−160 |
| NNAT | 2.41E−163 | −4.49098634 | 0.044 | 0.957 | 5.07E−159 |
| MSI1 | 8.64E−163 | 1.23928172 | 0.832 | 0.065 | 1.82E−158 |
| MT-RNR2 | 1.03E−162 | −2.76750458 | 0 | 0.946 | 2.17E−158 |
| CBX5 | 2.33E−162 | 2.2174268 | 0.977 | 0.396 | 4.90E−158 |
| RPS4X | 2.87E−162 | −2.38589591 | 0.977 | 1 | 6.04E−158 |
| CDON | 8.64E−162 | 2.01484635 | 0.821 | 0.076 | 1.82E−157 |
| RPL13A | 1.38E−161 | −2.31268453 | 0.961 | 1 | 2.90E−157 |
| RPL9 | 2.64E−161 | −2.50952956 | 0.78 | 0.996 | 5.56E−157 |
| RPL13AP25 | 1.63E−160 | −2.19209382 | 0 | 0.936 | 3.44E−156 |
| CDH6 | 1.05E−159 | 3.50637489 | 0.79 | 0.06 | 2.22E−155 |
| RPL17 | 3.72E−159 | −2.21048607 | 0.003 | 0.936 | 7.82E−155 |
| RPS3 | 3.88E−158 | −2.0544991 | 0.972 | 1 | 8.17E−154 |
| RPL19 | 7.74E−158 | −1.95673972 | 0.964 | 0.999 | 1.63E−153 |
| SARAF | 1.87E−157 | 1.13886199 | 0.692 | 0 | 3.94E−153 |
| EFNA5 | 4.37E−157 | 1.68101962 | 0.803 | 0.062 | 9.21E−153 |
| RPS18 | 1.69E−156 | −2.13623017 | 0.974 | 1 | 3.55E−152 |
| PNMA1 | 1.98E−156 | 1.27327623 | 0.689 | 0 | 4.17E−152 |
| DLK1 | 2.06E−156 | −4.8352563 | 0.114 | 0.949 | 4.35E−152 |
| EGR1 | 2.78E−156 | 3.1819037 | 0.749 | 0.035 | 5.86E−152 |
| RPL18 | 7.49E−156 | −2.0236944 | 0.927 | 0.998 | 1.58E−151 |
| RPL24 | 1.02E−153 | −2.05372205 | 0.824 | 0.994 | 2.15E−149 |
| RPL36A | 1.29E−153 | −2.01664738 | 0.018 | 0.918 | 2.72E−149 |
| RP11-122C9.1 | 1.83E−153 | −2.29270976 | 0 | 0.913 | 3.85E−149 |
| MT-ND4 | 6.37E−153 | −2.50177393 | 0 | 0.912 | 1.34E−148 |
| FBN2 | 1.08E−152 | 1.4151526 | 0.702 | 0.012 | 2.28E−148 |
| RPL12 | 1.98E−152 | −2.1520167 | 0.865 | 0.994 | 4.17E−148 |
| RPL11 | 4.37E−152 | −1.7981019 | 0.99 | 1 | 9.19E−148 |
| FTH1P20 | 8.43E−152 | −1.93322171 | 0 | 0.905 | 1.77E−147 |
| RPL18A | 1.70E−151 | −1.88714434 | 0.948 | 0.999 | 3.58E−147 |
| RPL8 | 2.09E−151 | −1.67297638 | 0.99 | 1 | 4.39E−147 |
| CTC-575D19.1 | 9.19E−151 | −1.9014718 | 0 | 0.901 | 1.94E−146 |
| RPS14 | 1.38E−150 | −1.97704841 | 0.992 | 1 | 2.91E−146 |
| SOX11 | 2.56E−150 | 1.57174142 | 0.873 | 0.139 | 5.38E−146 |
| RPL26 | 3.34E−150 | −1.95847278 | 0.948 | 0.994 | 7.04E−146 |
| RPL32 | 3.81E−150 | −1.87904091 | 0.99 | 1 | 8.02E−146 |
| RPS19 | 1.46E−149 | −1.93339656 | 0.99 | 1 | 3.08E−145 |
| SEMA5A | 4.25E−149 | 1.62406838 | 0.681 | 0.009 | 8.95E−145 |
| TSPYL1 | 6.15E−149 | 1.2244426 | 0.712 | 0.023 | 1.29E−144 |
| RPL21 | 1.40E−148 | −1.96234728 | 0.946 | 0.999 | 2.94E−144 |
| RPL29 | 2.19E−148 | −1.90274141 | 0.953 | 1 | 4.62E−144 |
| MT-CO3 | 2.79E−148 | −2.77602674 | 0 | 0.896 | 5.88E−144 |
| NACA | 3.89E−148 | −1.70457508 | 0.917 | 0.999 | 8.20E−144 |
| RPL27A | 1.22E−147 | −1.86437098 | 0.946 | 0.999 | 2.56E−143 |
| PAFAH1B2 | 1.82E−147 | 1.25414365 | 0.801 | 0.081 | 3.84E−143 |
| FXYD6 | 2.08E−147 | 1.05859465 | 0.655 | 0 | 4.38E−143 |
| RAB31 | 7.69E−147 | 1.52355829 | 0.775 | 0.064 | 1.62E−142 |
| DCTN1 | 4.88E−146 | 1.1992459 | 0.674 | 0.01 | 1.03E−141 |
| TMSB4XP8 | 1.08E−145 | −2.25920802 | 0 | 0.885 | 2.28E−141 |
| PXDN | 2.32E−145 | 1.53380108 | 0.772 | 0.072 | 4.89E−141 |
| FABP7 | 2.42E−145 | −2.89411177 | 0.041 | 0.906 | 5.09E−141 |
| CHCHD3 | 5.22E−145 | −1.79686112 | 0.495 | 0.977 | 1.10E−140 |
| FAU | 1.11E−144 | −1.67725636 | 0.769 | 0.994 | 2.34E−140 |
| RPS2P46 | 1.64E−144 | −1.88676239 | 0 | 0.879 | 3.44E−140 |
| C6orf62 | 1.86E−144 | 1.32165973 | 0.803 | 0.083 | 3.91E−140 |
| MARCKSL1 | 2.69E−144 | −2.41351997 | 0.676 | 0.977 | 5.65E−140 |
| COL13A1 | 2.88E−144 | 1.29628621 | 0.645 | 0 | 6.06E−140 |
| HIF1A | 1.07E−143 | 1.30985491 | 0.777 | 0.073 | 2.25E−139 |
| RPL31 | 6.05E−143 | −1.86243933 | 0.899 | 0.996 | 1.27E−138 |
| RPS16 | 8.30E−143 | −1.918087 | 0.699 | 0.985 | 1.75E−138 |
| IGF2BP1 | 2.52E−141 | 1.55148638 | 0.839 | 0.152 | 5.30E−137 |
| YWHAZ | 1.81E−140 | 1.72735619 | 0.961 | 0.456 | 3.81E−136 |
| SPP1 | 2.11E−140 | 1.64224684 | 0.648 | 0.006 | 4.45E−136 |
| AC007969.5 | 2.18E−140 | −1.68104506 | 0 | 0.862 | 4.59E−136 |
| UBA1 | 7.07E−140 | 1.80859548 | 0.922 | 0.347 | 1.49E−135 |
| TMEM66 | 1.28E−139 | −1.80987228 | 0 | 0.861 | 2.69E−135 |
| DDX5 | 2.45E−139 | 1.81854858 | 0.961 | 0.664 | 5.15E−135 |
| FOXP1 | 2.90E−139 | 1.23517665 | 0.769 | 0.079 | 6.10E−135 |
| SNRNP200 | 2.99E−139 | 1.50222443 | 0.855 | 0.167 | 6.29E−135 |
| MT-ND1 | 3.05E−139 | −1.99469922 | 0 | 0.861 | 6.42E−135 |
| RPLP2 | 3.70E−139 | −1.72628616 | 0.782 | 0.993 | 7.78E−135 |
| PLXDC2 | 5.26E−139 | 1.2672308 | 0.663 | 0.017 | 1.11E−134 |
| EIF1 | 3.57E−138 | −1.8636779 | 0.889 | 0.999 | 7.51E−134 |
| HUWE1 | 4.81E−138 | 1.21941901 | 0.803 | 0.104 | 1.01E−133 |
| CRB2 | 1.39E−137 | 1.25530082 | 0.707 | 0.043 | 2.93E−133 |
| USP9X | 1.54E−137 | 1.38641479 | 0.759 | 0.075 | 3.24E−133 |
| RPL30 | 2.09E−137 | −1.65445341 | 0.847 | 0.995 | 4.40E−133 |
| ZNF516 | 3.27E−137 | 1.14597871 | 0.668 | 0.022 | 6.88E−133 |
| MT-CO2 | 4.13E−137 | −2.69581961 | 0 | 0.855 | 8.68E−133 |
| FAM195B | 6.56E−137 | 0.8169339 | 0.614 | 0 | 1.38E−132 |
| CLEC19A | 7.76E−137 | 1.11317535 | 0.617 | 0 | 1.63E−132 |
| PPAP2B | 4.80E−136 | 1.53485573 | 0.777 | 0.097 | 1.01E−131 |
| ID3 | 1.59E−135 | 2.12311883 | 0.679 | 0.033 | 3.35E−131 |
| RPL3 | 1.75E−135 | −1.41569279 | 0.99 | 1 | 3.68E−131 |
| RPS13 | 2.66E−135 | −1.61883731 | 0.876 | 0.993 | 5.61E−131 |
| FLNA | 2.73E−135 | 1.30473798 | 0.762 | 0.082 | 5.74E−131 |
| ANKRD10 | 3.50E−135 | 1.33438097 | 0.894 | 0.237 | 7.37E−131 |
| RPL35A | 3.14E−134 | −1.49346711 | 0.974 | 1 | 6.60E−130 |
| SHISA2 | 3.52E−134 | 1.26293752 | 0.622 | 0.006 | 7.42E−130 |
| FAT1 | 1.87E−133 | 1.3422818 | 0.764 | 0.09 | 3.94E−129 |
| RPL35 | 1.94E−133 | −1.51080901 | 0.927 | 0.995 | 4.08E−129 |
| RPS6 | 2.99E−133 | −1.60624753 | 0.997 | 1 | 6.30E−129 |
| KIAA1217 | 4.65E−132 | 1.34332874 | 0.622 | 0.01 | 9.78E−128 |
| RPLP1 | 7.69E−132 | −1.43385066 | 0.982 | 0.998 | 1.62E−127 |
| EIF2S3 | 1.48E−131 | 1.44656058 | 0.946 | 0.41 | 3.13E−127 |
| FOS | 1.54E−131 | 3.62773224 | 0.756 | 0.117 | 3.24E−127 |
| PLEKHG1 | 1.86E−131 | 1.29583875 | 0.699 | 0.049 | 3.91E−127 |
| TULP4 | 7.61E−131 | 1.06486246 | 0.689 | 0.042 | 1.60E−126 |
| GAS5 | 2.21E−130 | −2.04620791 | 0.215 | 0.911 | 4.64E−126 |
| ID1 | 2.24E−130 | 1.38464502 | 0.666 | 0.032 | 4.71E−126 |
| SRCAP | 2.67E−130 | 0.98716566 | 0.642 | 0.021 | 5.62E−126 |
| MT-ND2 | 1.21E−129 | −1.91743409 | 0 | 0.824 | 2.55E−125 |
| ADGRV1 | 2.50E−129 | 0.98497179 | 0.588 | 0 | 5.26E−125 |
| RPSA | 2.63E−129 | −1.47602986 | 0.904 | 0.996 | 5.53E−125 |
| ALDH1A1 | 5.58E−129 | 4.63606106 | 0.588 | 0 | 1.18E−124 |
| ILF3 | 5.61E−129 | 1.50130075 | 0.943 | 0.403 | 1.18E−124 |
| RPL28 | 1.02E−128 | −1.47544262 | 0.883 | 0.996 | 2.16E−124 |
| HNRNPA1P48 | 4.17E−128 | −1.72385493 | 0 | 0.817 | 8.78E−124 |
| TNRC6B | 8.42E−128 | 1.48225241 | 0.829 | 0.165 | 1.77E−123 |
| SCD | 1.49E−127 | 1.81861479 | 0.769 | 0.114 | 3.14E−123 |
| LAMB2 | 1.86E−127 | 1.09722371 | 0.635 | 0.023 | 3.91E−123 |
| CNOT1 | 2.29E−127 | 1.07362975 | 0.72 | 0.066 | 4.81E−123 |
| RPS8 | 8.89E−127 | −1.29597419 | 0.992 | 1 | 1.87E−122 |
| ERH | 1.89E−126 | −1.61519786 | 0.28 | 0.917 | 3.98E−122 |
| RPL10A | 2.33E−126 | −1.44862438 | 0.977 | 1 | 4.91E−122 |
| RPS9 | 3.69E−126 | −1.37389453 | 0.964 | 1 | 7.77E−122 |
| RPL4P4 | 3.77E−126 | −1.54078883 | 0 | 0.807 | 7.93E−122 |
| RPS15 | 6.91E−126 | −1.38326494 | 0.984 | 1 | 1.46E−121 |
| PAN3 | 1.66E−125 | 0.97723061 | 0.671 | 0.039 | 3.49E−121 |
| PLCG1 | 3.02E−125 | 0.98396432 | 0.635 | 0.026 | 6.35E−121 |
| HCFC1 | 3.99E−125 | 1.0966155 | 0.707 | 0.065 | 8.40E−121 |
| GNB2L1 | 7.28E−125 | −1.31541471 | 0.974 | 1 | 1.53E−120 |
| RPL7A | 8.39E−125 | −1.33798291 | 0.979 | 0.999 | 1.77E−120 |
| CHP1 | 2.17E−124 | 1.03670578 | 0.648 | 0.034 | 4.58E−120 |
| TPT1 | 2.19E−123 | −1.46902483 | 0.811 | 0.995 | 4.62E−119 |
| ATP5E | 9.88E−123 | −1.5549919 | 0.005 | 0.797 | 2.08E−118 |
| RPL10 | 7.01E−122 | −1.15984169 | 0.992 | 1 | 1.48E−117 |
| GPI | 7.87E−122 | 1.62397396 | 0.788 | 0.148 | 1.66E−117 |
| DTX4 | 8.10E−122 | 1.24841898 | 0.604 | 0.018 | 1.70E−117 |
| PLXNB2 | 2.08E−121 | 1.17349679 | 0.676 | 0.059 | 4.39E−117 |
| CTTN | 2.76E−121 | 0.95056116 | 0.65 | 0.04 | 5.81E−117 |
| DDX6 | 3.72E−121 | 1.24265965 | 0.896 | 0.276 | 7.83E−117 |
| RPS3A | 3.83E−121 | −1.46199182 | 0.99 | 1 | 8.06E−117 |
| RPL6 | 1.15E−120 | −1.21595187 | 0.992 | 1 | 2.42E−116 |
| S1PR3 | 1.69E−120 | 1.30084449 | 0.554 | 0 | 3.56E−116 |
| RPL14P1 | 2.13E−120 | −1.43713395 | 0 | 0.782 | 4.47E−116 |
| NDUFA13 | 5.29E−120 | 0.73845984 | 0.549 | 0 | 1.11E−115 |
| TPPP3 | 7.12E−120 | 1.05679914 | 0.552 | 0 | 1.50E−115 |
| RPS7 | 8.20E−120 | −1.39931719 | 0.972 | 1 | 1.73E−115 |
| KDM5C | 1.53E−119 | 0.9494071 | 0.619 | 0.028 | 3.22E−115 |
| ETNK1 | 2.12E−119 | 1.20006837 | 0.744 | 0.109 | 4.46E−115 |
| PTMA | 2.37E−119 | −1.27629426 | 0.99 | 1 | 4.98E−115 |
| MDM4 | 3.50E−119 | 0.99179006 | 0.723 | 0.086 | 7.36E−115 |
| ZNF460 | 6.32E−119 | 0.93377965 | 0.611 | 0.024 | 1.33E−114 |
| RPL23A | 6.85E−119 | −1.39319202 | 0.886 | 0.991 | 1.44E−114 |
| SERF2 | 9.35E−119 | 0.8800702 | 0.624 | 0.031 | 1.97E−114 |
| SDK2 | 1.79E−118 | 1.12418783 | 0.676 | 0.062 | 3.77E−114 |
| MAT2A | 2.97E−118 | 1.12163513 | 0.712 | 0.084 | 6.25E−114 |
| SMS | 3.24E−118 | −1.76636835 | 0.298 | 0.905 | 6.81E−114 |
| LGR5 | 5.00E−118 | 0.9408727 | 0.544 | 0 | 1.05E−113 |
| LMAN1 | 6.84E−118 | 1.25375144 | 0.803 | 0.161 | 1.44E−113 |
| ZFAS1 | 1.01E−117 | −1.64424407 | 0.189 | 0.866 | 2.13E−113 |
| PRPF8 | 1.45E−117 | 1.26355151 | 0.806 | 0.17 | 3.06E−113 |
| NPM1 | 1.67E−117 | −1.34833514 | 0.943 | 0.998 | 3.51E−113 |
| SPHK1 | 7.88E−117 | 1.05228663 | 0.604 | 0.027 | 1.66E−112 |
| GANAB | 9.55E−117 | 1.05787988 | 0.728 | 0.094 | 2.01E−112 |
| EFR3B | 1.69E−116 | 0.9019993 | 0.573 | 0.013 | 3.55E−112 |
| IGFBP2 | 2.66E−116 | −1.79331975 | 0.132 | 0.841 | 5.60E−112 |
| SH3BGRL2 | 1.23E−115 | 1.09880274 | 0.601 | 0.027 | 2.59E−111 |
| NDN | 3.48E−115 | −1.5250654 | 0.181 | 0.872 | 7.32E−111 |
| UBE2L3 | 9.39E−115 | 0.84627478 | 0.573 | 0.016 | 1.98E−110 |
| CHCHD2 | 1.43E−114 | −1.52675479 | 0.365 | 0.913 | 3.01E−110 |
| MT-CYB | 2.73E−114 | −1.84023545 | 0 | 0.762 | 5.75E−110 |
| CCND1 | 3.52E−114 | 2.17536466 | 0.86 | 0.308 | 7.40E−110 |
| NGRN | 7.38E−114 | 0.98274428 | 0.606 | 0.033 | 1.55E−109 |
| SNU13 | 9.50E−114 | 0.77167762 | 0.526 | 0 | 2.00E−109 |
| DDX17 | 1.23E−113 | 1.23405967 | 0.902 | 0.355 | 2.59E−109 |
| RPL37A | 1.39E−113 | −1.31797278 | 0.832 | 0.983 | 2.93E−109 |
| DDB1 | 2.42E−113 | 1.28847194 | 0.782 | 0.169 | 5.09E−109 |
| EIF4EBP2 | 5.68E−113 | 1.06486428 | 0.751 | 0.125 | 1.20E−108 |
| MIAT | 6.88E−113 | 1.64833154 | 0.63 | 0.05 | 1.45E−108 |
| PFDN5 | 8.68E−113 | −1.35509511 | 0.521 | 0.941 | 1.83E−108 |
| TCF12 | 1.48E−112 | 1.36564322 | 0.79 | 0.177 | 3.11E−108 |
| RBM14 | 2.18E−112 | 0.76289406 | 0.521 | 0 | 4.59E−108 |
| PSMA1 | 3.50E−112 | 0.81034137 | 0.63 | 0.043 | 7.36E−108 |
| SET | 9.00E−112 | 1.30211449 | 0.964 | 0.617 | 1.90E−107 |
| DPYSL5 | 2.71E−111 | 1.25680288 | 0.749 | 0.131 | 5.71E−107 |
| RAC1 | 8.11E−111 | −1.34276476 | 0.269 | 0.889 | 1.71E−106 |
| SOX2 | 1.05E−110 | −1.28455727 | 0.35 | 0.94 | 2.20E−106 |
| UBA52 | 1.41E−110 | −1.2867543 | 0.689 | 0.951 | 2.98E−106 |
| GJA1 | 2.43E−110 | 1.50068788 | 0.567 | 0.022 | 5.12E−106 |
| G3BP1 | 4.76E−110 | 1.24532573 | 0.878 | 0.323 | 1.00E−105 |
| DHCR24 | 7.33E−110 | 1.55993804 | 0.635 | 0.064 | 1.54E−105 |
| PFKM | 8.75E−110 | 1.33840921 | 0.798 | 0.213 | 1.84E−105 |
| SMAD2 | 8.81E−110 | 1.04177287 | 0.718 | 0.106 | 1.85E−105 |
| ZEB2 | 9.05E−110 | 1.03972759 | 0.637 | 0.056 | 1.91E−105 |
| ITFG3 | 9.55E−110 | 0.77386518 | 0.51 | 0 | 2.01E−105 |
| RMST | 1.04E−109 | 0.9697897 | 0.661 | 0.064 | 2.19E−105 |
| GCN1 | 1.12E−109 | 0.79986039 | 0.51 | 0 | 2.36E−105 |
| MALAT1 | 1.22E−109 | −1.30358771 | 1 | 0.998 | 2.57E−105 |
| RPS20 | 8.25E−109 | −1.36178064 | 0.824 | 0.971 | 1.74E−104 |
| CRKL | 1.02E−108 | 0.91773382 | 0.627 | 0.05 | 2.15E−104 |
| CIRBP | 1.31E−108 | −1.25956488 | 0.842 | 0.988 | 2.76E−104 |
| ZNF428 | 7.38E−108 | −1.41921409 | 0.028 | 0.752 | 1.55E−103 |
| PTBP1 | 9.10E−108 | 1.19213346 | 0.749 | 0.144 | 1.92E−103 |
| PHPT1 | 1.17E−107 | 0.87502995 | 0.526 | 0.009 | 2.45E−103 |
| EIF4G2 | 1.89E−107 | 1.38272705 | 0.948 | 0.747 | 3.97E−103 |
| POLR2A | 2.08E−107 | 1.01901127 | 0.707 | 0.105 | 4.37E−103 |
| PRKDC | 2.08E−107 | 1.36609955 | 0.845 | 0.285 | 4.37E−103 |
| POLD1 | 6.03E−107 | 0.9525074 | 0.5 | 0 | 1.27E−102 |
| SEMA3A | 1.10E−106 | 0.89332402 | 0.549 | 0.02 | 2.31E−102 |
| ZKSCAN8 | 1.20E−106 | 0.86687606 | 0.541 | 0.016 | 2.52E−102 |
| HNRNPA1 | 3.86E−106 | −1.23151385 | 0.977 | 1 | 8.12E−102 |
| RPS5 | 1.12E−105 | −1.1604406 | 0.969 | 1 | 2.36E−101 |
| SRSF9 | 1.38E−105 | −1.4005643 | 0.168 | 0.819 | 2.90E−101 |
| TUBB2B | 2.01E−105 | −1.64198973 | 0.891 | 0.993 | 4.24E−101 |
| PTN | 2.18E−105 | −2.04385557 | 0.052 | 0.763 | 4.60E−101 |
| NHSL1 | 2.77E−105 | 0.93750501 | 0.58 | 0.033 | 5.83E−101 |
| NOL4L | 3.48E−105 | 0.82960773 | 0.492 | 0 | 7.34E−101 |
| PAX6 | 5.10E−105 | 1.46591168 | 0.899 | 0.389 | 1.07E−100 |
| COX4I1 | 5.14E−105 | −1.17105402 | 0.769 | 0.963 | 1.08E−100 |
| COX6A1 | 7.84E−105 | −1.35167437 | 0.135 | 0.818 | 1.65E−100 |
| COPA | 3.48E−104 | 1.1053484 | 0.764 | 0.166 | 7.33E−100 |
| PRRC2A | 2.02E−103 | 0.89542765 | 0.676 | 0.086 | 4.25E−99 |
| CPSF1 | 2.85E−103 | 0.94798503 | 0.614 | 0.055 | 6.01E−99 |
| CPAMD8 | 3.94E−103 | 1.06102496 | 0.552 | 0.027 | 8.30E−99 |
| LIG3 | 5.03E−103 | 0.88330981 | 0.57 | 0.034 | 1.06E−98 |
| CRB1 | 8.31E−103 | 1.18168039 | 0.565 | 0.034 | 1.75E−98 |
| NUP188 | 9.61E−103 | 0.78567477 | 0.523 | 0.015 | 2.02E−98 |
| SPOCK1 | 1.03E−102 | 1.04325241 | 0.531 | 0.018 | 2.16E−98 |
| GTF3C1 | 1.05E−102 | 0.80866089 | 0.593 | 0.044 | 2.21E−98 |
| ACAA2 | 2.48E−102 | 0.85994692 | 0.513 | 0.011 | 5.21E−98 |
| SKIL | 8.34E−102 | 1.45254069 | 0.71 | 0.142 | 1.75E−97 |
| WDR6 | 1.37E−101 | 1.10451778 | 0.731 | 0.139 | 2.88E−97 |
| TENM4 | 1.61E−101 | 0.85706074 | 0.518 | 0.015 | 3.40E−97 |
| KANSL3 | 2.39E−101 | 0.83578639 | 0.552 | 0.028 | 5.03E−97 |
| AASS | 3.02E−101 | 0.9052056 | 0.64 | 0.075 | 6.36E−97 |
| CASP2 | 3.95E−101 | 0.83711037 | 0.627 | 0.062 | 8.32E−97 |
| TMEM2 | 5.72E−101 | 1.14399579 | 0.593 | 0.054 | 1.20E−96 |
| BAZ2B | 9.60E−101 | 1.41269328 | 0.769 | 0.203 | 2.02E−96 |
| MT-TK | 2.33E−100 | −1.83678343 | 0 | 0.7 | 4.91E−96 |
| ATP5G2 | 2.84E−100 | −1.1613501 | 0.876 | 0.982 | 5.98E−96 |
| COL25A1 | 4.15E−100 | 0.99015881 | 0.508 | 0.013 | 8.73E−96 |
| NDST1 | 8.32E−100 | 0.78898736 | 0.518 | 0.017 | 1.75E−95 |
| STRA6 | 1.22E−99 | 1.07667066 | 0.547 | 0.031 | 2.56E−95 |
| NONO | 4.00E−99 | 1.12767783 | 0.93 | 0.506 | 8.42E−95 |
| PLCH1 | 4.72E−99 | 0.9510813 | 0.596 | 0.054 | 9.94E−95 |
| RPS15A | 5.11E−99 | −1.14321076 | 0.987 | 0.999 | 1.08E−94 |
| FIGN | 5.36E−99 | 0.84795264 | 0.583 | 0.045 | 1.13E−94 |
| OGT | 5.51E−99 | 0.83260615 | 0.565 | 0.038 | 1.16E−94 |
| TP53I11 | 6.16E−99 | 1.18249341 | 0.622 | 0.072 | 1.30E−94 |
| SLC35E2B | 9.07E−99 | 0.78367609 | 0.534 | 0.024 | 1.91E−94 |
| CCDC47 | 1.37E−98 | 0.72513495 | 0.508 | 0.015 | 2.89E−94 |
| RPL27 | 2.37E−98 | −1.19200376 | 0.915 | 0.979 | 4.99E−94 |
| BTF3 | 3.59E−98 | −1.12369226 | 0.855 | 0.988 | 7.55E−94 |
| RP11-632C17--A.1 | 1.22E−97 | −1.18512987 | 0 | 0.682 | 2.58E−93 |
| MED12L | 1.25E−97 | 0.72721508 | 0.461 | 0 | 2.64E−93 |
| DNAJC9 | 1.30E−97 | 0.74516487 | 0.461 | 0 | 2.74E−93 |
| HES4 | 2.02E−97 | −1.68354939 | 0.057 | 0.736 | 4.25E−93 |
| RPSAP58 | 2.31E−97 | −1.25456344 | 0.052 | 0.729 | 4.87E−93 |
| CASC3 | 2.77E−97 | 0.96103635 | 0.645 | 0.089 | 5.83E−93 |
| U2AF1 | 3.96E−97 | −1.30044983 | 0.003 | 0.685 | 8.35E−93 |
| ACTN4 | 4.89E−97 | 0.87564851 | 0.606 | 0.064 | 1.03E−92 |
| RPLP0 | 5.75E−97 | −0.97738225 | 0.982 | 1 | 1.21E−92 |
| CTB-33G10.1 | 5.88E−97 | −1.15930389 | 0 | 0.678 | 1.24E−92 |
| TMEM33 | 1.58E−96 | 0.87150168 | 0.674 | 0.1 | 3.33E−92 |
| ALDH2 | 1.96E−96 | 0.70216371 | 0.456 | 0 | 4.13E−92 |
| SYT11 | 3.14E−96 | 1.29173435 | 0.762 | 0.208 | 6.62E−92 |
| STT3A | 4.22E−96 | 0.794907 | 0.609 | 0.065 | 8.88E−92 |
| LDHA | 6.94E−96 | −1.73635805 | 0.438 | 0.939 | 1.46E−91 |
| GLG1 | 7.92E−96 | 0.92860419 | 0.71 | 0.137 | 1.67E−91 |
| STON2 | 8.89E−96 | 0.8471298 | 0.539 | 0.033 | 1.87E−91 |
| CNTN5 | 9.77E−96 | 0.76729252 | 0.453 | 0 | 2.06E−91 |
| IGSF9 | 9.82E−96 | 0.75809971 | 0.5 | 0.017 | 2.07E−91 |
| RAB5C | 2.62E−95 | 0.6459477 | 0.451 | 0 | 5.52E−91 |
| NHP2L1 | 2.64E−95 | −1.2120251 | 0 | 0.672 | 5.56E−91 |
| C14orf23 | 4.11E−95 | −1.3793254 | 0 | 0.674 | 8.66E−91 |
| DGCR2 | 4.36E−95 | 0.79418129 | 0.554 | 0.042 | 9.19E−91 |
| TIMELESS | 4.97E−95 | 1.08838773 | 0.606 | 0.073 | 1.05E−90 |
| CLASP1 | 5.58E−95 | 0.85304237 | 0.578 | 0.051 | 1.18E−90 |
| PCDH7 | 7.90E−95 | 0.82080864 | 0.521 | 0.027 | 1.66E−90 |
| RQCD1 | 1.17E−94 | 0.81698502 | 0.671 | 0.105 | 2.46E−90 |
| PPP4R3B | 1.25E−94 | 0.69063132 | 0.448 | 0 | 2.64E−90 |
| SLF1 | 1.41E−94 | 0.72269253 | 0.448 | 0 | 2.96E−90 |
| ABLIM1 | 4.77E−94 | 0.75943616 | 0.526 | 0.029 | 1.00E−89 |
| MT-RNR1 | 5.80E−94 | −1.46742618 | 0 | 0.669 | 1.22E−89 |
| TIMM10B | 9.02E−94 | 0.6893211 | 0.466 | 0.007 | 1.90E−89 |
| UBAP2L | 1.38E−93 | 0.96902744 | 0.715 | 0.15 | 2.91E−89 |
| TSPYL4 | 4.42E−93 | 0.96236356 | 0.624 | 0.082 | 9.31E−89 |
| TOB1 | 1.05E−92 | 0.8297434 | 0.503 | 0.023 | 2.21E−88 |
| RPL36 | 1.15E−92 | 0.79930402 | 0.712 | 0.132 | 2.41E−88 |
| PKDCC | 1.27E−92 | 0.73621789 | 0.44 | 0 | 2.67E−88 |
| TIAM1 | 1.67E−92 | 0.82555447 | 0.528 | 0.033 | 3.52E−88 |
| PUM1 | 2.25E−92 | 0.99970706 | 0.71 | 0.153 | 4.73E−88 |
| B4GAT1 | 3.12E−92 | 0.61211411 | 0.438 | 0 | 6.58E−88 |
| PKP4 | 3.68E−92 | 0.66383105 | 0.482 | 0.015 | 7.74E−88 |
| USP22 | 3.74E−92 | 0.85986505 | 0.731 | 0.158 | 7.88E−88 |
| P3H4 | 4.18E−92 | 0.67198556 | 0.438 | 0 | 8.80E−88 |
| MTOR | 5.06E−92 | 0.76803657 | 0.492 | 0.02 | 1.06E−87 |
| TBC1D9B | 7.31E−92 | 0.77724201 | 0.521 | 0.031 | 1.54E−87 |
| XPO5 | 1.07E−91 | 0.73987043 | 0.531 | 0.035 | 2.26E−87 |
| EDF1 | 1.11E−91 | −1.10075992 | 0.622 | 0.932 | 2.33E−87 |
| FASN | 1.11E−91 | 0.99384135 | 0.557 | 0.051 | 2.33E−87 |
| TRO | 1.22E−91 | 0.83515303 | 0.554 | 0.049 | 2.57E−87 |
| ISYNA1 | 1.68E−91 | 1.34670504 | 0.891 | 0.452 | 3.54E−87 |
| NUP98 | 2.51E−91 | 0.72824583 | 0.513 | 0.029 | 5.28E−87 |
| RPS12 | 3.16E−91 | −1.00112675 | 0.969 | 0.998 | 6.65E−87 |
| APLP2 | 3.34E−91 | 1.14949755 | 0.845 | 0.347 | 7.04E−87 |
| CSNK2B | 4.62E−91 | 0.62428882 | 0.433 | 0 | 9.73E−87 |
| PDLIM5 | 5.42E−91 | 0.83826466 | 0.573 | 0.057 | 1.14E−86 |
| PTPLAD1 | 6.16E−91 | −1.18668033 | 0 | 0.652 | 1.30E−86 |
| PTMAP4 | 1.87E−90 | −1.15851402 | 0 | 0.649 | 3.94E−86 |
| LRP1 | 1.96E−90 | 1.01035232 | 0.604 | 0.082 | 4.12E−86 |
| CFAP20 | 2.31E−90 | 0.62428882 | 0.43 | 0 | 4.87E−86 |
| FOXN4 | 4.45E−90 | 0.90738885 | 0.43 | 0 | 9.36E−86 |
| PTMS | 5.76E−90 | −1.3867839 | 0.451 | 0.878 | 1.21E−85 |
| TGFB2 | 7.03E−90 | 0.9420171 | 0.477 | 0.017 | 1.48E−85 |
| SLC27A4 | 1.27E−89 | −1.28912936 | 0.028 | 0.672 | 2.67E−85 |
| ARHGEF26 | 1.28E−89 | 0.86594836 | 0.497 | 0.026 | 2.68E−85 |
| CLDN1 | 1.69E−89 | 0.91929562 | 0.427 | 0 | 3.56E−85 |
| HMGN2 | 3.28E−89 | 1.92314035 | 0.816 | 0.348 | 6.91E−85 |
| FUS | 5.36E−89 | 1.24782336 | 0.92 | 0.586 | 1.13E−84 |
| DKK3 | 6.67E−89 | 1.35956911 | 0.705 | 0.18 | 1.41E−84 |
| LOC283731 | 7.30E−89 | 0.95636487 | 0.425 | 0 | 1.54E−84 |
| RBP1 | 1.22E−88 | 1.13357928 | 0.85 | 0.27 | 2.57E−84 |
| TP53BP1 | 2.31E−88 | 1.29680454 | 0.627 | 0.095 | 4.87E−84 |
| SF3B3 | 3.20E−88 | 0.9527697 | 0.661 | 0.123 | 6.74E−84 |
| CHD7 | 3.23E−88 | 1.07740495 | 0.837 | 0.323 | 6.80E−84 |
| IPO9 | 3.76E−88 | 0.94263746 | 0.681 | 0.137 | 7.92E−84 |
| EIF3F | 7.34E−88 | −1.15300433 | 0.484 | 0.89 | 1.55E−83 |
| ICE2 | 7.58E−88 | 0.65071695 | 0.42 | 0 | 1.60E−83 |
| PSMA6 | 9.61E−88 | 0.73782844 | 0.575 | 0.065 | 2.02E−83 |
| FOSB | 1.13E−87 | 0.89135874 | 0.42 | 0 | 2.38E−83 |
| TRPM3 | 1.27E−87 | 1.03317841 | 0.503 | 0.033 | 2.68E−83 |
| EXOSC10 | 2.47E−87 | 0.75993341 | 0.583 | 0.067 | 5.20E−83 |
| ZBTB16 | 2.82E−87 | 1.05623313 | 0.614 | 0.095 | 5.93E−83 |
| VAT1 | 7.07E−87 | 0.87011882 | 0.661 | 0.12 | 1.49E−82 |
| IFT22 | 9.36E−87 | 0.66963781 | 0.415 | 0 | 1.97E−82 |
| SUPT5H | 1.29E−86 | 0.88268099 | 0.585 | 0.075 | 2.72E−82 |
| RPS27 | 1.34E−86 | −1.0890315 | 0.909 | 0.973 | 2.82E−82 |
| SCUBE3 | 1.51E−86 | 0.64667889 | 0.433 | 0.006 | 3.19E−82 |
| GLUL | 1.69E−86 | 1.13533893 | 0.912 | 0.524 | 3.56E−82 |
| RORB | 1.87E−86 | 0.94107172 | 0.508 | 0.037 | 3.93E−82 |
| MED16 | 2.11E−86 | 0.84913042 | 0.591 | 0.081 | 4.45E−82 |
| COMMD6 | 4.28E−86 | −1.17558723 | 0.233 | 0.792 | 9.02E−82 |
| NEMP1 | 4.96E−86 | 0.63395516 | 0.412 | 0 | 1.04E−81 |
| RPS24 | 5.27E−86 | −1.01623857 | 0.966 | 0.993 | 1.11E−81 |
| NBEA | 7.04E−86 | 0.77586029 | 0.513 | 0.039 | 1.48E−81 |
| WDR83OS | 1.33E−85 | −1.13236438 | 0.117 | 0.733 | 2.80E−81 |
| GPX1 | 1.60E−85 | 0.70216371 | 0.409 | 0 | 3.37E−81 |
| REC8 | 1.60E−85 | 1.08068772 | 0.731 | 0.194 | 3.38E−81 |
| PAGR1 | 1.73E−85 | 0.60229982 | 0.409 | 0 | 3.65E−81 |
| SREBF2 | 2.60E−85 | 0.91669097 | 0.635 | 0.108 | 5.48E−81 |
| RC3H1 | 3.66E−85 | 0.68200903 | 0.503 | 0.035 | 7.70E−81 |
| PTK2 | 4.35E−85 | 0.86845987 | 0.671 | 0.134 | 9.16E−81 |
| MINOS1 | 5.15E−85 | −1.14317978 | 0.282 | 0.804 | 1.08E−80 |
| CAPZA1 | 5.20E−85 | 0.87090853 | 0.723 | 0.178 | 1.09E−80 |
| AMOTL2 | 7.17E−85 | 0.8761284 | 0.482 | 0.028 | 1.51E−80 |
| FTO | 9.45E−85 | 0.67970725 | 0.513 | 0.04 | 1.99E−80 |
| ALDH18A1 | 1.03E−84 | 0.94114707 | 0.591 | 0.086 | 2.16E−80 |
| AXL | 1.26E−84 | 0.69941543 | 0.448 | 0.015 | 2.64E−80 |
| COL9A1 | 1.67E−84 | 0.85998485 | 0.516 | 0.044 | 3.52E−80 |
| PDPN | 2.71E−84 | 0.74439676 | 0.479 | 0.026 | 5.71E−80 |
| JUP | 9.57E−84 | 0.79399338 | 0.49 | 0.033 | 2.01E−79 |
| PRPSAP1 | 1.12E−83 | 1.04948596 | 0.746 | 0.211 | 2.35E−79 |
| VPS72 | 1.62E−83 | 0.5611427 | 0.42 | 0.006 | 3.41E−79 |
| RPL5 | 2.12E−83 | −0.85509946 | 0.992 | 1 | 4.47E−79 |
| TXLNA | 2.32E−83 | 0.67018101 | 0.472 | 0.026 | 4.89E−79 |
| SF3B14 | 3.09E−83 | −1.05959899 | 0 | 0.612 | 6.51E−79 |
| H3F3B | 4.81E−83 | −1.0527189 | 0.969 | 1 | 1.01E−78 |
| ADGRL2 | 4.88E−83 | 0.61211411 | 0.399 | 0 | 1.03E−78 |
| FTH1 | 8.42E−83 | −0.88769433 | 0.987 | 1 | 1.77E−78 |
| ARID2 | 1.03E−82 | 0.76566466 | 0.562 | 0.071 | 2.17E−78 |
| ANKRD36BP1 | 1.50E−82 | 0.53682598 | 0.396 | 0 | 3.16E−78 |
| TFRC | 1.60E−82 | 0.86421227 | 0.495 | 0.039 | 3.37E−78 |
| DDX47 | 1.79E−82 | 0.57496111 | 0.396 | 0 | 3.76E−78 |
| YTHDF3 | 2.66E−82 | 0.70916647 | 0.523 | 0.05 | 5.60E−78 |
| PSAP | 3.58E−82 | 1.2177819 | 0.896 | 0.606 | 7.53E−78 |
| PPP1CB | 3.66E−82 | 0.86843789 | 0.759 | 0.225 | 7.71E−78 |
| MCM2 | 4.07E−82 | 1.15495052 | 0.583 | 0.094 | 8.56E−78 |
| ATP2B4 | 5.06E−82 | 0.7811563 | 0.549 | 0.062 | 1.07E−77 |
| PRRT2 | 5.41E−82 | 0.73588026 | 0.526 | 0.053 | 1.14E−77 |
| MT-TG | 5.66E−82 | −1.39483478 | 0 | 0.61 | 1.19E−77 |
| RSRP1 | 7.35E−82 | 0.5849625 | 0.394 | 0 | 1.55E−77 |
| RP11-864N7.2 | 8.38E−82 | −1.05026113 | 0 | 0.605 | 1.76E−77 |
| ANP32E | 1.12E−81 | 1.32317041 | 0.749 | 0.248 | 2.36E−77 |
| NDUFA4 | 1.34E−81 | −0.96997541 | 0.806 | 0.961 | 2.82E−77 |
| NR2F1 | 1.41E−81 | 0.71721832 | 0.438 | 0.015 | 2.96E−77 |
| SMAD9 | 1.99E−81 | 0.73511535 | 0.487 | 0.035 | 4.19E−77 |
| MRPL57 | 2.44E−81 | 0.54965002 | 0.391 | 0 | 5.13E−77 |
| RPS25 | 2.47E−81 | −1.02987519 | 0.868 | 0.957 | 5.19E−77 |
| DYNC1H1 | 2.61E−81 | 1.1037278 | 0.798 | 0.315 | 5.49E−77 |
| CASZ1 | 3.00E−81 | 0.59983578 | 0.391 | 0 | 6.31E−77 |
| PCBP4 | 3.13E−81 | 0.72042592 | 0.391 | 0 | 6.58E−77 |
| MYH9 | 4.53E−81 | 0.77755062 | 0.472 | 0.029 | 9.53E−77 |
| MAGED4 | 9.61E−81 | −1.08143298 | 0 | 0.6 | 2.02E−76 |
| STK26 | 1.14E−80 | 0.65547048 | 0.389 | 0 | 2.41E−76 |
| EIF3C | 1.77E−80 | −1.07976512 | 0 | 0.599 | 3.72E−76 |
| HSPB1 | 1.83E−80 | −1.25671754 | 0.282 | 0.798 | 3.86E−76 |
| FTLP2 | 1.85E−80 | −1.24811315 | 0 | 0.6 | 3.89E−76 |
| CHCHD2P9 | 2.20E−80 | −0.98046783 | 0 | 0.597 | 4.64E−76 |
| DUSP1 | 2.27E−80 | 1.60725574 | 0.64 | 0.145 | 4.78E−76 |
| ATP5L | 2.39E−80 | −1.05467367 | 0.448 | 0.851 | 5.04E−76 |
| SMG1 | 2.42E−80 | 0.72836589 | 0.503 | 0.044 | 5.10E−76 |
| MZT2B | 4.44E−80 | −1.04598808 | 0.028 | 0.632 | 9.36E−76 |
| SLF2 | 4.46E−80 | 0.62186208 | 0.386 | 0 | 9.40E−76 |
| CEP131 | 5.15E−80 | 0.60966679 | 0.386 | 0 | 1.08E−75 |
| DCAF8 | 7.45E−80 | 0.78045193 | 0.573 | 0.079 | 1.57E−75 |
| SLC35F6 | 8.85E−80 | 0.62861298 | 0.433 | 0.016 | 1.86E−75 |
| ACLY | 1.45E−79 | 0.91256717 | 0.565 | 0.082 | 3.05E−75 |
| RAX | 1.85E−79 | 0.61699633 | 0.383 | 0 | 3.90E−75 |
| JADE1 | 1.90E−79 | 0.60721531 | 0.383 | 0 | 4.00E−75 |
| SLC7A8 | 1.90E−79 | 0.89134908 | 0.435 | 0.018 | 4.00E−75 |
| KIAA0101 | 2.40E−79 | 0.69063132 | 0.383 | 0 | 5.06E−75 |
| CORO1C | 2.41E−79 | 0.91043529 | 0.611 | 0.114 | 5.07E−75 |
| VPS13B | 3.62E−79 | 0.6145456 | 0.435 | 0.017 | 7.61E−75 |
| LGR4 | 4.94E−79 | 0.63301366 | 0.404 | 0.007 | 1.04E−74 |
| ARID1A | 5.17E−79 | 0.69672076 | 0.591 | 0.09 | 1.09E−74 |
| SORCS1 | 5.27E−79 | 0.72141138 | 0.495 | 0.044 | 1.11E−74 |
| PEX19 | 5.38E−79 | 0.76866748 | 0.508 | 0.05 | 1.13E−74 |
| LINC01420 | 6.48E−79 | 0.56236107 | 0.381 | 0 | 1.36E−74 |
| GNS | 6.96E−79 | 0.69950153 | 0.461 | 0.029 | 1.46E−74 |
| ZHX1 | 7.39E−79 | 0.58745205 | 0.381 | 0 | 1.56E−74 |
| CENPU | 7.96E−79 | 0.60229982 | 0.381 | 0 | 1.68E−74 |
| POLE | 8.23E−79 | 0.65499295 | 0.407 | 0.009 | 1.73E−74 |
| CTDSP2 | 1.09E−78 | 0.80088483 | 0.588 | 0.092 | 2.28E−74 |
| RPL4 | 2.96E−78 | −0.79054167 | 0.984 | 1 | 6.24E−74 |
| GLB1L2 | 3.00E−78 | 0.58745205 | 0.378 | 0 | 6.31E−74 |
| DOCK1 | 3.77E−78 | 0.68721013 | 0.451 | 0.026 | 7.94E−74 |
| PFKL | 4.51E−78 | 0.92466641 | 0.694 | 0.176 | 9.50E−74 |
| TBL1XR1 | 5.01E−78 | 0.86389197 | 0.655 | 0.142 | 1.05E−73 |
| FAT3 | 5.36E−78 | 0.77845053 | 0.523 | 0.06 | 1.13E−73 |
| CAPRIN1 | 6.68E−78 | 1.0302401 | 0.821 | 0.356 | 1.41E−73 |
| RNF138 | 6.87E−78 | 0.55047842 | 0.422 | 0.015 | 1.45E−73 |
| ARFGAP3 | 1.17E−77 | 0.57997047 | 0.376 | 0 | 2.46E−73 |
| MT-ND5 | 1.34E−77 | −1.09305429 | 0 | 0.584 | 2.82E−73 |
| SPAG5 | 1.56E−77 | 0.81268443 | 0.376 | 0 | 3.28E−73 |
| SAR1A | 1.88E−77 | 0.8419864 | 0.746 | 0.209 | 3.97E−73 |
| CD47 | 1.90E−77 | 0.77508219 | 0.622 | 0.116 | 3.99E−73 |
| FANCD2 | 2.31E−77 | 0.86766076 | 0.446 | 0.027 | 4.87E−73 |
| BRK1 | 2.84E−77 | −0.97723341 | 0.58 | 0.908 | 5.98E−73 |
| BCL11A | 4.46E−77 | 0.74494737 | 0.466 | 0.034 | 9.38E−73 |
| MCM4 | 5.12E−77 | 1.23907693 | 0.528 | 0.073 | 1.08E−72 |
| ARL6IP4 | 5.13E−77 | 0.53990793 | 0.396 | 0.007 | 1.08E−72 |
| SENP5 | 6.51E−77 | 0.67886725 | 0.528 | 0.062 | 1.37E−72 |
| NCL | 6.96E−77 | 1.03580972 | 0.925 | 0.645 | 1.46E−72 |
| COX8A | 8.73E−77 | 0.58332658 | 0.399 | 0.009 | 1.84E−72 |
| RTN4 | 1.29E−76 | −1.18157614 | 0.347 | 0.825 | 2.71E−72 |
| MYH10 | 1.32E−76 | 0.99628508 | 0.754 | 0.254 | 2.78E−72 |
| SNRPB | 1.44E−76 | 0.9238897 | 0.687 | 0.181 | 3.03E−72 |
| HGS | 1.63E−76 | 0.78218507 | 0.549 | 0.078 | 3.44E−72 |
| HDLBP | 1.87E−76 | 0.91835779 | 0.749 | 0.238 | 3.93E−72 |
| LENG8 | 2.07E−76 | 0.6821064 | 0.534 | 0.067 | 4.35E−72 |
| COX7A2L | 2.59E−76 | −1.06523078 | 0.503 | 0.874 | 5.46E−72 |
| TRRAP | 2.60E−76 | 0.6355742 | 0.453 | 0.031 | 5.48E−72 |
| RPLP0P2 | 2.87E−76 | −0.93574233 | 0.008 | 0.587 | 6.04E−72 |
| POLA1 | 3.63E−76 | 0.80576454 | 0.505 | 0.055 | 7.63E−72 |
| NUP205 | 4.52E−76 | 0.62493721 | 0.412 | 0.015 | 9.51E−72 |
| GREB1 | 4.95E−76 | 0.72557313 | 0.443 | 0.027 | 1.04E−71 |
| INSR | 5.61E−76 | 0.5938803 | 0.402 | 0.011 | 1.18E−71 |
| AGO1 | 5.71E−76 | 0.76406863 | 0.503 | 0.053 | 1.20E−71 |
| CD99L2 | 1.60E−75 | 0.6684625 | 0.49 | 0.048 | 3.38E−71 |
| FOXP2 | 2.09E−75 | 0.73573451 | 0.412 | 0.016 | 4.39E−71 |
| TOMM5 | 2.29E−75 | 0.55982781 | 0.365 | 0 | 4.81E−71 |
| NF2 | 2.53E−75 | 0.65374677 | 0.479 | 0.044 | 5.33E−71 |
| NDUFC2 | 2.78E−75 | −1.05694781 | 0.142 | 0.7 | 5.85E−71 |
| CPXM1 | 5.43E−75 | 0.91207765 | 0.65 | 0.153 | 1.14E−70 |
| ARHGAP33 | 7.31E−75 | 0.68385595 | 0.43 | 0.023 | 1.54E−70 |
| GUK1 | 8.38E−75 | −1.11427214 | 0.332 | 0.798 | 1.76E−70 |
| MT-ATP6 | 1.04E−74 | −1.26729036 | 0 | 0.571 | 2.20E−70 |
| ZBTB33 | 1.10E−74 | 0.59148472 | 0.417 | 0.018 | 2.31E−70 |
| DENND1A | 2.09E−74 | 0.62057243 | 0.531 | 0.068 | 4.41E−70 |
| IFT122 | 2.96E−74 | 0.63306784 | 0.425 | 0.022 | 6.23E−70 |
| TRIM59 | 6.05E−74 | 0.75850094 | 0.378 | 0.006 | 1.27E−69 |
| PTMAP2 | 8.74E−74 | −0.95885402 | 0 | 0.562 | 1.84E−69 |
| PRDX5 | 8.79E−74 | −1.00346648 | 0.562 | 0.89 | 1.85E−69 |
| CCDC8 | 1.15E−73 | 0.489692 | 0.358 | 0 | 2.43E−69 |
| RPA1 | 1.26E−73 | 0.90649627 | 0.681 | 0.182 | 2.65E−69 |
| TSC2 | 1.30E−73 | 0.71242845 | 0.453 | 0.034 | 2.74E−69 |
| SNHG6 | 1.37E−73 | −1.07234982 | 0.065 | 0.63 | 2.88E−69 |
| FAAP100 | 1.38E−73 | 0.52648404 | 0.358 | 0 | 2.91E−69 |
| RPL7 | 1.50E−73 | −1.12256144 | 0.992 | 0.998 | 3.17E−69 |
| KMT2D | 1.54E−73 | 0.53990793 | 0.378 | 0.006 | 3.24E−69 |
| MSH6 | 1.63E−73 | 0.93460281 | 0.593 | 0.121 | 3.42E−69 |
| CBX6 | 1.70E−73 | 0.63168265 | 0.513 | 0.062 | 3.58E−69 |
| UTRN | 1.73E−73 | 0.71996312 | 0.464 | 0.04 | 3.63E−69 |
| SLC16A1 | 1.73E−73 | 1.05791724 | 0.733 | 0.246 | 3.65E−69 |
| PYGB | 3.34E−73 | 0.70187717 | 0.521 | 0.068 | 7.02E−69 |
| PCBP2 | 5.16E−73 | −0.98109837 | 0.642 | 0.896 | 1.09E−68 |
| C19orf43 | 7.02E−73 | −1.0203249 | 0.155 | 0.704 | 1.48E−68 |
| CIT | 7.19E−73 | 0.71588199 | 0.355 | 0 | 1.51E−68 |
| RAF1 | 8.39E−73 | 0.76705595 | 0.596 | 0.116 | 1.77E−68 |
| ENAH | 1.25E−72 | 0.79320351 | 0.707 | 0.188 | 2.63E−68 |
| EYA3 | 1.31E−72 | 0.63283859 | 0.479 | 0.048 | 2.76E−68 |
| CSE1L | 1.82E−72 | 0.76977238 | 0.591 | 0.115 | 3.83E−68 |
| ZNF445 | 3.08E−72 | 0.64304736 | 0.477 | 0.048 | 6.48E−68 |
| AC068137.13 | 4.30E−72 | −0.94792446 | 0 | 0.554 | 9.04E−68 |
| LLGL1 | 4.81E−72 | 0.79629928 | 0.56 | 0.095 | 1.01E−67 |
| HNRNPA2B1 | 5.51E−72 | 0.98813396 | 0.972 | 0.884 | 1.16E−67 |
| TTYH1 | 5.60E−72 | −1.37419574 | 0.497 | 0.858 | 1.18E−67 |
| FADS2 | 5.88E−72 | 0.83981854 | 0.604 | 0.13 | 1.24E−67 |
| RP11-170M17.2 | 5.89E−72 | −0.93322171 | 0 | 0.553 | 1.24E−67 |
| EML1 | 1.23E−71 | 0.69938439 | 0.51 | 0.067 | 2.58E−67 |
| MOV10 | 1.24E−71 | 0.62643928 | 0.412 | 0.022 | 2.61E−67 |
| ATF7IP | 1.32E−71 | 0.82579903 | 0.64 | 0.153 | 2.78E−67 |
| NUMA1 | 1.51E−71 | 0.7682588 | 0.472 | 0.046 | 3.17E−67 |
| ZFX | 2.00E−71 | 0.65795806 | 0.484 | 0.05 | 4.21E−67 |
| SF3B6 | 2.76E−71 | 0.50293999 | 0.347 | 0 | 5.80E−67 |
| EIF6 | 2.76E−71 | 0.51867863 | 0.347 | 0 | 5.80E−67 |
| VCP | 3.13E−71 | 1.04502585 | 0.873 | 0.549 | 6.59E−67 |
| ESYT1 | 3.44E−71 | 0.6605573 | 0.459 | 0.043 | 7.24E−67 |
| NAP1L4 | 3.62E−71 | 0.91214304 | 0.769 | 0.289 | 7.62E−67 |
| SPTAN1 | 3.76E−71 | 0.71740857 | 0.497 | 0.059 | 7.92E−67 |
| RMND5A | 4.16E−71 | 0.67729986 | 0.49 | 0.056 | 8.77E−67 |
| ADNP | 4.71E−71 | 0.7058983 | 0.64 | 0.149 | 9.91E−67 |
| UBR4 | 6.90E−71 | 0.62937514 | 0.435 | 0.032 | 1.45E−66 |
| SRRM2 | 8.26E−71 | 0.90930666 | 0.842 | 0.457 | 1.74E−66 |
| RDH11 | 9.63E−71 | 0.58670653 | 0.42 | 0.026 | 2.03E−66 |
| RPS23 | 9.94E−71 | −0.76083157 | 0.99 | 0.998 | 2.09E−66 |
| CHST15 | 1.19E−70 | 0.54196928 | 0.345 | 0 | 2.51E−66 |
| IFT140 | 1.76E−70 | 0.60985034 | 0.453 | 0.04 | 3.72E−66 |
| CHD1L | 2.49E−70 | 0.61966832 | 0.472 | 0.049 | 5.24E−66 |
| NCAM1 | 2.55E−70 | 0.81507904 | 0.619 | 0.138 | 5.36E−66 |
| AC072062.1 | 2.89E−70 | −0.8622319 | 0 | 0.542 | 6.09E−66 |
| GRWD1 | 3.86E−70 | 0.57997047 | 0.342 | 0 | 8.12E−66 |
| SGPL1 | 4.42E−70 | 0.52588798 | 0.402 | 0.02 | 9.30E−66 |
| MDC1 | 7.27E−70 | 0.66733583 | 0.438 | 0.034 | 1.53E−65 |
| VIM | 8.07E−70 | −1.25460253 | 0.969 | 0.984 | 1.70E−65 |
| RANBP2 | 9.54E−70 | 0.63030816 | 0.531 | 0.078 | 2.01E−65 |
| CASC4 | 1.09E−69 | 0.72266627 | 0.624 | 0.138 | 2.30E−65 |
| RP11-622K12.1 | 1.42E−69 | −0.96066759 | 0 | 0.54 | 2.98E−65 |
| PYURF | 1.43E−69 | −0.89674319 | 0 | 0.538 | 3.02E−65 |
| GPAT4 | 1.57E−69 | 0.51083077 | 0.339 | 0 | 3.30E−65 |
| MIR4426 | 1.72E−69 | −0.85542443 | 0 | 0.537 | 3.62E−65 |
| PITPNM1 | 1.79E−69 | 0.70891454 | 0.425 | 0.032 | 3.77E−65 |
| GRB10 | 2.11E−69 | 0.88546429 | 0.503 | 0.073 | 4.44E−65 |
| BNIP3 | 2.13E−69 | −1.73683701 | 0.368 | 0.829 | 4.48E−65 |
| ABAT | 2.17E−69 | 0.74921896 | 0.552 | 0.097 | 4.58E−65 |
| DCLRE1C | 2.37E−69 | 0.57788093 | 0.433 | 0.033 | 4.98E−65 |
| IGF1R | 2.62E−69 | 0.66790772 | 0.479 | 0.056 | 5.52E−65 |
| PAK3 | 2.69E−69 | −1.2005449 | 0.06 | 0.6 | 5.67E−65 |
| ANKFY1 | 3.09E−69 | 0.62532599 | 0.464 | 0.048 | 6.51E−65 |
| MSN | 5.26E−69 | 0.89907543 | 0.684 | 0.205 | 1.11E−64 |
| TRAPPC5 | 5.75E−69 | 0.4950058 | 0.337 | 0 | 1.21E−64 |
| TMEM263 | 5.94E−69 | 0.51345147 | 0.337 | 0 | 1.25E−64 |
| EML4 | 5.96E−69 | 0.60240809 | 0.474 | 0.053 | 1.25E−64 |
| MEGF8 | 6.14E−69 | 0.60570099 | 0.378 | 0.013 | 1.29E−64 |
| TTF2 | 6.94E−69 | 0.61269923 | 0.417 | 0.028 | 1.46E−64 |
| HOMER2 | 7.17E−69 | 0.58865629 | 0.378 | 0.013 | 1.51E−64 |
| OGDH | 9.20E−69 | 0.55715766 | 0.417 | 0.028 | 1.94E−64 |
| IER2 | 1.22E−68 | 1.94582292 | 0.733 | 0.352 | 2.57E−64 |
| RPAP1 | 1.48E−68 | 0.60186928 | 0.396 | 0.021 | 3.12E−64 |
| COX7A2 | 1.63E−68 | −1.01332516 | 0.282 | 0.771 | 3.44E−64 |
| FAM120B | 1.89E−68 | 0.63010869 | 0.474 | 0.055 | 3.99E−64 |
| CTNNA2 | 2.15E−68 | 0.65433217 | 0.446 | 0.042 | 4.53E−64 |
| EBLN3 | 2.31E−68 | 0.54709432 | 0.334 | 0 | 4.86E−64 |
| LDOC1L | 2.41E−68 | 0.58701268 | 0.404 | 0.024 | 5.08E−64 |
| AC018730.1 | 2.79E−68 | −1.26141679 | 0 | 0.535 | 5.87E−64 |
| ANKRD52 | 3.00E−68 | 0.5261828 | 0.368 | 0.011 | 6.33E−64 |
| VCL | 3.01E−68 | 0.65596171 | 0.433 | 0.037 | 6.34E−64 |
| SEC16A | 4.52E−68 | 0.62974539 | 0.451 | 0.045 | 9.52E−64 |
| COX6B1 | 4.53E−68 | −0.8901641 | 0.316 | 0.79 | 9.53E−64 |
| SF3A1 | 4.56E−68 | 0.6765358 | 0.534 | 0.086 | 9.60E−64 |
| SLC39A10 | 4.59E−68 | 0.74308816 | 0.565 | 0.109 | 9.67E−64 |
| TUBGCP6 | 4.82E−68 | 0.55993804 | 0.37 | 0.012 | 1.01E−63 |
| YWHAE | 6.72E−68 | 0.84361062 | 0.969 | 0.828 | 1.41E−63 |
| LOXL3 | 8.80E−68 | 0.64305008 | 0.404 | 0.026 | 1.85E−63 |
| RASGEF1B | 9.40E−68 | 0.57496111 | 0.332 | 0 | 1.98E−63 |
| FLII | 1.23E−67 | 0.69883615 | 0.521 | 0.079 | 2.58E−63 |
| DBN1 | 1.63E−67 | 0.94516437 | 0.793 | 0.35 | 3.43E−63 |
| ATXN7L3B | 1.71E−67 | 0.92997471 | 0.777 | 0.309 | 3.59E−63 |
| HEPH | 2.02E−67 | 0.68739431 | 0.443 | 0.043 | 4.25E−63 |
| CCL2 | 2.13E−67 | 0.97064666 | 0.352 | 0.007 | 4.49E−63 |
| PNMAL1 | 2.28E−67 | 0.62115678 | 0.474 | 0.057 | 4.81E−63 |
| AP2B1 | 2.42E−67 | 0.85027613 | 0.697 | 0.219 | 5.09E−63 |
| C1orf198 | 3.62E−67 | 0.67906034 | 0.451 | 0.048 | 7.62E−63 |
| HINT1 | 4.52E−67 | −0.83746898 | 0.788 | 0.923 | 9.52E−63 |
| KIAA0195 | 5.71E−67 | 0.62640852 | 0.422 | 0.034 | 1.20E−62 |
| COX7C | 6.40E−67 | −0.90673292 | 0.549 | 0.869 | 1.35E−62 |
| TIA1 | 6.80E−67 | 0.84749388 | 0.712 | 0.23 | 1.43E−62 |
| MCM7 | 6.91E−67 | 1.43838983 | 0.694 | 0.27 | 1.46E−62 |
| SMC1A | 8.52E−67 | 0.93979194 | 0.65 | 0.186 | 1.79E−62 |
| LTBP4 | 9.52E−67 | 0.72787598 | 0.505 | 0.076 | 2.00E−62 |
| CDR1 | 9.76E−67 | 0.74200226 | 0.365 | 0.012 | 2.05E−62 |
| POLDIP3 | 1.10E−66 | 0.70482824 | 0.534 | 0.092 | 2.31E−62 |
| PRELP | 1.35E−66 | 0.57244989 | 0.326 | 0 | 2.83E−62 |
| EGR2 | 1.51E−66 | 0.78040802 | 0.326 | 0 | 3.18E−62 |
| FAT4 | 1.54E−66 | 0.67541526 | 0.36 | 0.011 | 3.24E−62 |
| NOTCH2 | 2.11E−66 | 0.57160096 | 0.412 | 0.031 | 4.44E−62 |
| HDAC1 | 2.22E−66 | 0.67683811 | 0.567 | 0.114 | 4.68E−62 |
| MTPAP | 2.49E−66 | 0.59020732 | 0.415 | 0.032 | 5.24E−62 |
| DPF2 | 3.54E−66 | 0.70899442 | 0.637 | 0.155 | 7.45E−62 |
| CREBZF | 3.66E−66 | 0.63885084 | 0.497 | 0.07 | 7.71E−62 |
| ANKRD12 | 3.72E−66 | 0.57253808 | 0.44 | 0.043 | 7.82E−62 |
| POMK | 3.90E−66 | 0.45739151 | 0.324 | 0 | 8.22E−62 |
| ZNF271P | 4.53E−66 | 0.5003001 | 0.324 | 0 | 9.53E−62 |
| TBX5 | 5.58E−66 | 0.70216371 | 0.324 | 0 | 1.18E−61 |
| PLEC | 7.36E−66 | 0.66203623 | 0.35 | 0.009 | 1.55E−61 |
| SYT7 | 8.34E−66 | 0.54970226 | 0.368 | 0.015 | 1.76E−61 |
| SPATA13 | 8.50E−66 | 0.59461347 | 0.383 | 0.021 | 1.79E−61 |
| TXN | 1.19E−65 | −0.98855047 | 0.459 | 0.83 | 2.51E−61 |
| GLTSCR2 | 1.39E−65 | −0.95894585 | 0.197 | 0.709 | 2.92E−61 |
| PDK3 | 1.46E−65 | 0.66899144 | 0.539 | 0.094 | 3.08E−61 |
| TOMM6 | 1.58E−65 | 0.50293999 | 0.321 | 0 | 3.32E−61 |
| PSMA3-AS1 | 1.61E−65 | 0.46553469 | 0.321 | 0 | 3.39E−61 |
| FADS1 | 2.18E−65 | 1.13244647 | 0.788 | 0.399 | 4.59E−61 |
| SPG11 | 2.33E−65 | 0.6277734 | 0.477 | 0.062 | 4.91E−61 |
| C19orf10 | 2.35E−65 | −0.86126137 | 0 | 0.515 | 4.94E−61 |
| PLXNB1 | 2.45E−65 | 0.53946776 | 0.373 | 0.017 | 5.15E−61 |
| ACVR2B | 2.46E−65 | 0.61579127 | 0.477 | 0.062 | 5.19E−61 |
| TMSB10 | 4.67E−65 | −1.12749847 | 0.933 | 0.979 | 9.82E−61 |
| ODF2 | 5.01E−65 | 0.85550487 | 0.526 | 0.098 | 1.05E−60 |
| MBNL2 | 5.42E−65 | 0.67232218 | 0.461 | 0.056 | 1.14E−60 |
| DACH1 | 5.65E−65 | 0.61301005 | 0.49 | 0.067 | 1.19E−60 |
| CNP | 8.10E−65 | 0.74694857 | 0.583 | 0.125 | 1.70E−60 |
| TMTC4 | 1.20E−64 | 0.79606028 | 0.552 | 0.109 | 2.53E−60 |
| CLRN1 | 2.16E−64 | 0.44920211 | 0.316 | 0 | 4.55E−60 |
| XPOT | 2.25E−64 | 0.79416175 | 0.604 | 0.141 | 4.74E−60 |
| IRGQ | 2.30E−64 | 0.53482832 | 0.394 | 0.026 | 4.84E−60 |
| MCFD2 | 2.30E−64 | 0.67659038 | 0.598 | 0.132 | 4.84E−60 |
| DAPK1 | 2.63E−64 | 0.55874685 | 0.37 | 0.018 | 5.53E−60 |
| NISCH | 2.73E−64 | 0.60039822 | 0.459 | 0.055 | 5.75E−60 |
| FAM84A | 3.01E−64 | 1.10782933 | 0.472 | 0.07 | 6.33E−60 |
| HIPK2 | 3.36E−64 | 0.72465244 | 0.578 | 0.128 | 7.08E−60 |
| TTI1 | 3.54E−64 | 0.6343837 | 0.461 | 0.056 | 7.45E−60 |
| EFNA2 | 4.09E−64 | 0.4423736 | 0.339 | 0.007 | 8.60E−60 |
| ASTN1 | 4.11E−64 | 0.53188127 | 0.37 | 0.018 | 8.66E−60 |
| ARNT | 5.26E−64 | 0.56401133 | 0.391 | 0.027 | 1.11E−59 |
| SPICE1 | 5.50E−64 | 0.6112671 | 0.415 | 0.037 | 1.16E−59 |
| THBS3 | 6.25E−64 | 0.59530938 | 0.378 | 0.022 | 1.31E−59 |
| EIF3FP3 | 9.38E−64 | −0.80785874 | 0 | 0.505 | 1.97E−59 |
| LIG1 | 9.41E−64 | 0.86703905 | 0.479 | 0.072 | 1.98E−59 |
| BROX | 1.08E−63 | 0.57680455 | 0.438 | 0.046 | 2.28E−59 |
| PPP3CA | 1.11E−63 | 0.56650009 | 0.417 | 0.038 | 2.33E−59 |
| CCND2 | 1.58E−63 | 1.15239508 | 0.692 | 0.26 | 3.33E−59 |
| MRP63 | 1.64E−63 | −0.78756734 | 0 | 0.502 | 3.45E−59 |
| NEK9 | 1.66E−63 | 0.6035522 | 0.386 | 0.026 | 3.50E−59 |
| ODC1 | 1.72E−63 | −0.9303247 | 0.536 | 0.9 | 3.62E−59 |
| EFTUD2 | 1.82E−63 | 0.69128287 | 0.518 | 0.09 | 3.84E−59 |
| SEC61A1 | 2.39E−63 | 0.66795027 | 0.578 | 0.122 | 5.03E−59 |
| SNRPD2 | 2.60E−63 | −0.88459076 | 0.609 | 0.878 | 5.48E−59 |
| CELF1 | 2.76E−63 | 0.68709117 | 0.596 | 0.142 | 5.81E−59 |
| CENPBD1P1 | 2.82E−63 | 0.44371658 | 0.311 | 0 | 5.94E−59 |
| LOC100131564 | 3.26E−63 | 0.48168443 | 0.311 | 0 | 6.86E−59 |
| NCAPG2 | 3.29E−63 | 0.64159163 | 0.407 | 0.035 | 6.92E−59 |
| LRBA | 3.47E−63 | 0.6684625 | 0.42 | 0.04 | 7.31E−59 |
| GRIA4 | 4.42E−63 | 0.5063009 | 0.352 | 0.012 | 9.31E−59 |
| IVNS1ABP | 4.92E−63 | 0.97252417 | 0.671 | 0.222 | 1.04E−58 |
| KMT2C | 6.51E−63 | 0.74246308 | 0.531 | 0.099 | 1.37E−58 |
| PHIP | 1.05E−62 | 0.88488819 | 0.699 | 0.241 | 2.22E−58 |
| PFAS | 1.06E−62 | 0.5851841 | 0.352 | 0.013 | 2.23E−58 |
| P3H1 | 1.09E−62 | 0.44096596 | 0.308 | 0 | 2.31E−58 |
| EDC4 | 1.18E−62 | 0.5082053 | 0.308 | 0 | 2.49E−58 |
| OSBPL3 | 1.21E−62 | 0.49765538 | 0.308 | 0 | 2.54E−58 |
| TMPO | 1.41E−62 | 0.99388047 | 0.661 | 0.224 | 2.97E−58 |
| MT-TH | 1.43E−62 | −0.89104834 | 0 | 0.5 | 3.01E−58 |
| IDE | 1.46E−62 | 0.48988303 | 0.373 | 0.022 | 3.08E−58 |
| EPB41 | 1.75E−62 | 0.67001128 | 0.567 | 0.125 | 3.69E−58 |
| AZIN1 | 1.82E−62 | 0.8570878 | 0.78 | 0.35 | 3.84E−58 |
| CCNYL1 | 1.87E−62 | 0.61401471 | 0.391 | 0.029 | 3.94E−58 |
| AFF1 | 3.60E−62 | 0.53416141 | 0.383 | 0.027 | 7.58E−58 |
| SGTA | 3.79E−62 | 0.70424376 | 0.617 | 0.158 | 7.98E−58 |
| OLFM3 | 4.14E−62 | 0.56296937 | 0.355 | 0.016 | 8.71E−58 |
| CLTC | 4.58E−62 | 0.79587802 | 0.565 | 0.127 | 9.64E−58 |
| BIN1 | 5.05E−62 | 0.88672268 | 0.567 | 0.138 | 1.06E−57 |
| SKA2 | 5.92E−62 | 0.78908135 | 0.58 | 0.138 | 1.25E−57 |
| HDAC6 | 5.96E−62 | 0.61681187 | 0.451 | 0.056 | 1.25E−57 |
| PLEKHG2 | 6.27E−62 | 0.51136154 | 0.373 | 0.022 | 1.32E−57 |
| PCNT | 7.03E−62 | 0.59566667 | 0.378 | 0.026 | 1.48E−57 |
| TLE3 | 8.04E−62 | 0.51982285 | 0.376 | 0.023 | 1.69E−57 |
| BABAM1 | 1.00E−61 | 0.566235 | 0.391 | 0.031 | 2.11E−57 |
| PTEN | 1.02E−61 | 0.65440841 | 0.503 | 0.086 | 2.15E−57 |
| ZNF410 | 1.06E−61 | 0.52187 | 0.339 | 0.011 | 2.24E−57 |
| PARP1 | 1.21E−61 | 1.00064306 | 0.837 | 0.501 | 2.55E−57 |
| RBM6 | 1.34E−61 | 0.73894234 | 0.666 | 0.197 | 2.81E−57 |
| ZFP91 | 1.38E−61 | 0.40754296 | 0.303 | 0 | 2.91E−57 |
| CTNNA1 | 1.81E−61 | 0.85240088 | 0.725 | 0.262 | 3.81E−57 |
| RP11-71N10.1 | 2.51E−61 | −1.05026113 | 0 | 0.494 | 5.28E−57 |
| PFDN2 | 3.02E−61 | −0.87615454 | 0.067 | 0.576 | 6.36E−57 |
| BEX1 | 3.19E−61 | −1.06825938 | 0.132 | 0.628 | 6.71E−57 |
| MFN2 | 3.37E−61 | 0.54922189 | 0.412 | 0.04 | 7.10E−57 |
| SYNPR | 4.75E−61 | 0.53655082 | 0.321 | 0.006 | 1.00E−56 |
| MAP1B | 4.86E−61 | 1.05596024 | 0.966 | 0.889 | 1.02E−56 |
| RPS11 | 4.88E−61 | −0.83631993 | 0.904 | 0.95 | 1.03E−56 |
| NCAPD2 | 5.28E−61 | 1.11662878 | 0.42 | 0.05 | 1.11E−56 |
| GATSL2 | 5.66E−61 | 0.62671148 | 0.301 | 0 | 1.19E−56 |
| TGOLN2 | 6.52E−61 | 0.64429619 | 0.505 | 0.087 | 1.37E−56 |
| CD99 | 6.91E−61 | −0.85347352 | 0 | 0.489 | 1.46E−56 |
| PRELID1 | 9.35E−61 | −0.88929363 | 0.155 | 0.653 | 1.97E−56 |
| ABHD16A | 1.26E−60 | 0.58843412 | 0.453 | 0.062 | 2.65E−56 |
| ACACA | 1.39E−60 | 0.72231247 | 0.487 | 0.083 | 2.92E−56 |
| PDCD11 | 1.47E−60 | 0.56457626 | 0.409 | 0.04 | 3.09E−56 |
| CBX2 | 1.55E−60 | 0.75533995 | 0.446 | 0.059 | 3.26E−56 |
| SFI1 | 1.98E−60 | 0.52235611 | 0.378 | 0.028 | 4.16E−56 |
| PLEKHM3 | 2.23E−60 | 0.49765538 | 0.298 | 0 | 4.69E−56 |
| PPP6R3 | 2.58E−60 | 0.63885084 | 0.479 | 0.076 | 5.42E−56 |
| EXOC4 | 2.78E−60 | 0.64134637 | 0.531 | 0.105 | 5.86E−56 |
| ZNF286A | 3.46E−60 | 0.63000366 | 0.399 | 0.037 | 7.29E−56 |
| SMPD4 | 4.87E−60 | 0.48735709 | 0.37 | 0.024 | 1.03E−55 |
| ADD1 | 5.64E−60 | 0.73990011 | 0.562 | 0.127 | 1.19E−55 |
| AP1M1 | 5.91E−60 | 0.73202763 | 0.598 | 0.153 | 1.24E−55 |
| MDM2 | 7.58E−60 | 0.70266207 | 0.534 | 0.105 | 1.60E−55 |
| MRC2 | 7.91E−60 | 0.55681054 | 0.358 | 0.021 | 1.67E−55 |
| APCDD1 | 7.93E−60 | 0.50557505 | 0.295 | 0 | 1.67E−55 |
| WFIKKN1 | 8.17E−60 | 0.54453408 | 0.295 | 0 | 1.72E−55 |
| ESPL1 | 8.86E−60 | 0.61211411 | 0.295 | 0 | 1.87E−55 |
| IQGAP3 | 8.94E−60 | 0.60475966 | 0.295 | 0 | 1.88E−55 |
| PRRC2B | 9.35E−60 | 0.81325982 | 0.744 | 0.29 | 1.97E−55 |
| GLYR1 | 1.15E−59 | 0.53378834 | 0.389 | 0.032 | 2.42E−55 |
| KIRREL | 1.28E−59 | 0.53386353 | 0.329 | 0.011 | 2.70E−55 |
| CYFIP1 | 1.62E−59 | 0.69797747 | 0.477 | 0.079 | 3.41E−55 |
| AKAP13 | 1.68E−59 | 0.6967254 | 0.49 | 0.086 | 3.53E−55 |
| UNC13B | 1.78E−59 | 0.49681487 | 0.363 | 0.023 | 3.74E−55 |
| ZIC1 | 2.04E−59 | −1.03266635 | 0.166 | 0.652 | 4.30E−55 |
| BRD3 | 2.62E−59 | 0.58934868 | 0.601 | 0.145 | 5.53E−55 |
| CPSF6 | 2.95E−59 | 0.7451298 | 0.71 | 0.257 | 6.21E−55 |
| RP11-761E20.1 | 3.01E−59 | −0.83875702 | 0 | 0.479 | 6.34E−55 |
| MZT2A | 3.08E−59 | −0.78474142 | 0.023 | 0.516 | 6.48E−55 |
| CDK6 | 3.62E−59 | 0.71976373 | 0.456 | 0.068 | 7.62E−55 |
| TRPM7 | 4.19E−59 | 0.65814521 | 0.376 | 0.029 | 8.83E−55 |
| PGD | 5.34E−59 | 0.81344206 | 0.842 | 0.449 | 1.12E−54 |
| KPNA6 | 8.25E−59 | 0.5916992 | 0.417 | 0.046 | 1.74E−54 |
| JUN | 8.85E−59 | 1.24968531 | 0.624 | 0.196 | 1.86E−54 |
| NAPRT | 9.01E−59 | 0.44646195 | 0.29 | 0 | 1.90E−54 |
| RPL37 | 9.10E−59 | −0.80848967 | 0.692 | 0.889 | 1.92E−54 |
| NXF1 | 9.16E−59 | 0.57963592 | 0.459 | 0.067 | 1.93E−54 |
| MAF | 1.45E−58 | 0.55162781 | 0.324 | 0.011 | 3.05E−54 |
| MGA | 1.81E−58 | 0.57906593 | 0.43 | 0.054 | 3.80E−54 |
| ENTPD4 | 2.34E−58 | 0.47881535 | 0.383 | 0.033 | 4.94E−54 |
| TUBB2A | 2.50E−58 | −1.48377327 | 0.187 | 0.644 | 5.25E−54 |
| GPM6A | 2.56E−58 | 0.94771656 | 0.824 | 0.455 | 5.39E−54 |
| MED12 | 2.69E−58 | 0.48121802 | 0.355 | 0.022 | 5.66E−54 |
| ADAM10 | 2.82E−58 | 0.70467403 | 0.531 | 0.114 | 5.94E−54 |
| MATR3 | 2.84E−58 | 0.78834081 | 0.946 | 0.709 | 5.97E−54 |
| RIF1 | 2.87E−58 | 0.7397751 | 0.588 | 0.154 | 6.04E−54 |
| WFDC2 | 3.05E−58 | −1.04356747 | 0.013 | 0.491 | 6.41E−54 |
| SRRT | 3.24E−58 | 0.74501336 | 0.554 | 0.131 | 6.83E−54 |
| TMX2 | 3.30E−58 | 0.39906481 | 0.288 | 0 | 6.95E−54 |
| LRPPRC | 3.35E−58 | 0.64650635 | 0.57 | 0.136 | 7.05E−54 |
| SUGP2 | 3.36E−58 | 0.68363293 | 0.562 | 0.133 | 7.08E−54 |
| UBR2 | 3.57E−58 | 0.63036745 | 0.407 | 0.045 | 7.53E−54 |
| CCNL2 | 3.66E−58 | 0.70318011 | 0.58 | 0.15 | 7.70E−54 |
| CD63 | 3.75E−58 | −0.88706435 | 0.811 | 0.933 | 7.88E−54 |
| COLGALT1 | 3.94E−58 | 0.50452912 | 0.363 | 0.026 | 8.29E−54 |
| HIST1H4I | 4.40E−58 | 1.1183494 | 0.288 | 0 | 9.27E−54 |
| PPP1R9A | 4.55E−58 | 0.56302622 | 0.446 | 0.062 | 9.58E−54 |
| COX5B | 4.86E−58 | −0.8516042 | 0.345 | 0.763 | 1.02E−53 |
| RPL18AP3 | 5.29E−58 | −0.7195771 | 0 | 0.469 | 1.11E−53 |
| HERC2P2 | 5.58E−58 | 0.46360371 | 0.347 | 0.02 | 1.17E−53 |
| ANKRD40 | 8.50E−58 | 0.40577695 | 0.321 | 0.011 | 1.79E−53 |
| RAB36 | 1.13E−57 | 0.52912582 | 0.36 | 0.026 | 2.39E−53 |
| MYDGF | 1.20E−57 | 0.39906481 | 0.285 | 0 | 2.54E−53 |
| DNAJC14 | 1.23E−57 | 0.4018964 | 0.285 | 0 | 2.58E−53 |
| ADGRG1 | 1.38E−57 | 0.52648404 | 0.285 | 0 | 2.92E−53 |
| LMNB2 | 1.60E−57 | 0.60503712 | 0.42 | 0.051 | 3.38E−53 |
| DHX8 | 1.94E−57 | 0.58340215 | 0.389 | 0.038 | 4.08E−53 |
| MED1 | 1.95E−57 | 0.52488494 | 0.36 | 0.026 | 4.11E−53 |
| SEC24C | 2.00E−57 | 0.5684452 | 0.404 | 0.045 | 4.21E−53 |
| TCF3 | 2.04E−57 | 0.556107 | 0.44 | 0.061 | 4.30E−53 |
| PCDHB5 | 2.10E−57 | 0.54753156 | 0.386 | 0.037 | 4.41E−53 |
| TNPO2 | 2.10E−57 | 0.53432582 | 0.399 | 0.042 | 4.43E−53 |
| GOLIM4 | 2.34E−57 | 0.71108213 | 0.554 | 0.13 | 4.92E−53 |
| DNAJB1 | 3.07E−57 | 0.6273753 | 0.534 | 0.115 | 6.45E−53 |
| ASAP3 | 3.08E−57 | 0.55127896 | 0.378 | 0.034 | 6.48E−53 |
| CLDND1 | 3.92E−57 | 0.49515486 | 0.396 | 0.04 | 8.26E−53 |
| RHOT2 | 4.14E−57 | 0.67892444 | 0.518 | 0.109 | 8.72E−53 |
| SGSM3 | 4.14E−57 | 0.6292919 | 0.487 | 0.089 | 8.72E−53 |
| TEKT2 | 4.29E−57 | 0.50198892 | 0.345 | 0.021 | 9.03E−53 |
| RAVER1 | 4.39E−57 | 0.40472244 | 0.282 | 0 | 9.24E−53 |
| HAUS5 | 4.40E−57 | 0.51462877 | 0.381 | 0.035 | 9.27E−53 |
| NIPBL-AS1 | 4.60E−57 | 0.44646195 | 0.282 | 0 | 9.68E−53 |
| FANCI | 4.68E−57 | 0.7351228 | 0.412 | 0.051 | 9.86E−53 |
| UCHL1 | 4.73E−57 | −1.17686028 | 0.497 | 0.826 | 9.95E−53 |
| PCDHGC3 | 4.81E−57 | 0.4382101 | 0.282 | 0 | 1.01E−52 |
| DAG1 | 4.83E−57 | 0.63930504 | 0.461 | 0.073 | 1.02E−52 |
| TNPO1 | 5.09E−57 | 0.73457898 | 0.655 | 0.209 | 1.07E−52 |
| RBM12 | 5.80E−57 | 0.46852493 | 0.389 | 0.038 | 1.22E−52 |
| CCNI | 6.06E−57 | −0.91723439 | 0.751 | 0.932 | 1.27E−52 |
| IST1 | 6.40E−57 | 0.71316727 | 0.611 | 0.167 | 1.35E−52 |
| CDK5RAP3 | 7.28E−57 | 0.69402991 | 0.676 | 0.224 | 1.53E−52 |
| BRD8 | 8.51E−57 | 0.77750526 | 0.583 | 0.156 | 1.79E−52 |
| SKP2 | 1.02E−56 | 0.60948281 | 0.531 | 0.114 | 2.16E−52 |
| TPX2 | 1.07E−56 | 1.77827759 | 0.456 | 0.083 | 2.25E−52 |
| NYNRIN | 1.21E−56 | 0.54589868 | 0.352 | 0.024 | 2.56E−52 |
| DDX1 | 1.23E−56 | 0.75957417 | 0.71 | 0.263 | 2.59E−52 |
| RNF182 | 1.23E−56 | 0.48005807 | 0.308 | 0.009 | 2.59E−52 |
| CREB3L2 | 1.24E−56 | 0.51665229 | 0.326 | 0.015 | 2.60E−52 |
| RHNO1 | 1.34E−56 | 0.51769245 | 0.301 | 0.006 | 2.82E−52 |
| CAD | 1.42E−56 | 0.57455233 | 0.36 | 0.028 | 3.00E−52 |
| CBLB | 1.57E−56 | 0.58410435 | 0.383 | 0.038 | 3.30E−52 |
| PARG | 1.58E−56 | 0.3933849 | 0.28 | 0 | 3.33E−52 |
| PPP2R1B | 1.91E−56 | 0.5474471 | 0.417 | 0.051 | 4.01E−52 |
| SS18 | 2.06E−56 | 0.67383385 | 0.611 | 0.178 | 4.33E−52 |
| DCHS1 | 2.07E−56 | 0.5856983 | 0.365 | 0.031 | 4.36E−52 |
| KLHL11 | 2.40E−56 | 0.43591846 | 0.303 | 0.007 | 5.05E−52 |
| USP11 | 3.89E−56 | 0.89226851 | 0.858 | 0.584 | 8.18E−52 |
| ZFP36L1 | 4.02E−56 | 1.11731828 | 0.829 | 0.476 | 8.46E−52 |
| FAM13C | 4.83E−56 | 0.63237363 | 0.528 | 0.115 | 1.02E−51 |
| MRPL38 | 5.45E−56 | 0.38482289 | 0.277 | 0 | 1.15E−51 |
| PGK1 | 5.57E−56 | −1.22559158 | 0.754 | 0.951 | 1.17E−51 |
| RANGAP1 | 5.70E−56 | 0.68356939 | 0.391 | 0.044 | 1.20E−51 |
| P4HB | 5.79E−56 | 0.87688816 | 0.803 | 0.414 | 1.22E−51 |
| ANKRD17 | 6.07E−56 | 0.62602723 | 0.516 | 0.105 | 1.28E−51 |
| CENPV | 6.17E−56 | −0.98757617 | 0.215 | 0.675 | 1.30E−51 |
| DNAAF3 | 6.58E−56 | 0.52388692 | 0.277 | 0 | 1.39E−51 |
| CDS2 | 6.59E−56 | 0.60626774 | 0.562 | 0.134 | 1.39E−51 |
| XRCC5 | 7.39E−56 | 0.88759513 | 0.873 | 0.587 | 1.56E−51 |
| VPS39 | 8.35E−56 | 0.49000586 | 0.373 | 0.034 | 1.76E−51 |
| RCC2 | 9.24E−56 | 0.52075223 | 0.43 | 0.06 | 1.95E−51 |
| PLD3 | 1.16E−55 | 0.92585795 | 0.873 | 0.577 | 2.45E−51 |
| ZNF687 | 1.52E−55 | 0.46081209 | 0.332 | 0.018 | 3.19E−51 |
| HSPA4 | 1.56E−55 | 0.74295622 | 0.596 | 0.171 | 3.28E−51 |
| SLC4A8 | 1.74E−55 | 0.59175066 | 0.422 | 0.057 | 3.67E−51 |
| UBR7 | 1.84E−55 | 0.44626343 | 0.311 | 0.011 | 3.88E−51 |
| KIAA1467 | 1.86E−55 | 0.51413435 | 0.313 | 0.012 | 3.91E−51 |
| WAPL | 2.02E−55 | 0.40754296 | 0.275 | 0 | 4.25E−51 |
| CFAP36 | 2.05E−55 | 0.42435125 | 0.275 | 0 | 4.32E−51 |
| PASK | 2.08E−55 | 0.46520932 | 0.319 | 0.013 | 4.39E−51 |
| NT5C2 | 3.32E−55 | 0.64968529 | 0.531 | 0.12 | 7.00E−51 |
| SRSF5 | 3.74E−55 | 0.77653795 | 0.915 | 0.669 | 7.87E−51 |
| TM9SF1 | 3.83E−55 | 0.49939497 | 0.347 | 0.024 | 8.06E−51 |
| DDX23 | 4.02E−55 | 0.53183506 | 0.425 | 0.057 | 8.46E−51 |
| C1orf61 | 4.34E−55 | −1.07454754 | 0.003 | 0.462 | 9.15E−51 |
| TCOF1 | 5.16E−55 | 0.62119325 | 0.443 | 0.071 | 1.09E−50 |
| CHTF18 | 5.28E−55 | 0.51385829 | 0.352 | 0.027 | 1.11E−50 |
| VPRBP | 5.35E−55 | 0.43117586 | 0.329 | 0.018 | 1.13E−50 |
| SMIM10L1 | 7.17E−55 | 0.40754296 | 0.272 | 0 | 1.51E−50 |
| SSR2 | 7.58E−55 | −0.8578241 | 0.51 | 0.806 | 1.60E−50 |
| TTC37 | 7.84E−55 | 0.55041527 | 0.43 | 0.062 | 1.65E−50 |
| FLRT2 | 9.00E−55 | 0.62772213 | 0.334 | 0.021 | 1.89E−50 |
| CS | 9.75E−55 | 0.45625769 | 0.321 | 0.016 | 2.05E−50 |
| TANC1 | 1.02E−54 | 0.42416916 | 0.303 | 0.01 | 2.16E−50 |
| TRPC4AP | 1.25E−54 | 0.64033476 | 0.508 | 0.108 | 2.62E−50 |
| USP5 | 1.30E−54 | 0.63247264 | 0.513 | 0.112 | 2.75E−50 |
| WDFY1 | 1.46E−54 | 0.57852999 | 0.44 | 0.07 | 3.08E−50 |
| CCDC23 | 1.48E−54 | −0.76387385 | 0 | 0.45 | 3.12E−50 |
| FOXK1 | 1.69E−54 | 0.56530651 | 0.383 | 0.042 | 3.55E−50 |
| FGF19 | 1.92E−54 | 0.7721712 | 0.474 | 0.079 | 4.03E−50 |
| DVL2 | 2.07E−54 | 0.46385566 | 0.352 | 0.028 | 4.36E−50 |
| DTL | 2.17E−54 | 0.67130525 | 0.35 | 0.028 | 4.57E−50 |
| HNRNPK | 2.20E−54 | 0.68933964 | 0.972 | 0.929 | 4.62E−50 |
| SYNCRIP | 2.35E−54 | 0.6030555 | 0.523 | 0.117 | 4.95E−50 |
| RAB24 | 2.56E−54 | 0.37908652 | 0.269 | 0 | 5.39E−50 |
| GNB2 | 2.63E−54 | −0.79222364 | 0.075 | 0.545 | 5.54E−50 |
| MPDZ | 2.68E−54 | 0.60057731 | 0.513 | 0.11 | 5.63E−50 |
| SAFB | 2.75E−54 | 0.75077037 | 0.645 | 0.218 | 5.79E−50 |
| BRD2 | 2.79E−54 | 0.70925772 | 0.666 | 0.226 | 5.87E−50 |
| USP28 | 2.81E−54 | 0.54402027 | 0.352 | 0.028 | 5.92E−50 |
| HERC1 | 2.98E−54 | 0.53828814 | 0.425 | 0.061 | 6.28E−50 |
| PEAK1 | 3.00E−54 | 0.52720684 | 0.36 | 0.032 | 6.32E−50 |
| COG4 | 3.03E−54 | 0.54752879 | 0.448 | 0.075 | 6.37E−50 |
| DHX9 | 3.24E−54 | 0.81609105 | 0.694 | 0.273 | 6.82E−50 |
| EMC6 | 3.38E−54 | −0.7408405 | 0 | 0.447 | 7.11E−50 |
| CERK | 3.55E−54 | 0.64788797 | 0.42 | 0.061 | 7.48E−50 |
| BASP1 | 3.97E−54 | −1.06168941 | 0.111 | 0.579 | 8.37E−50 |
| CTSD | 4.31E−54 | 0.77367452 | 0.619 | 0.193 | 9.07E−50 |
| LRP5 | 4.61E−54 | 0.50627314 | 0.329 | 0.02 | 9.71E−50 |
| UBBP1 | 6.01E−54 | −0.70342133 | 0 | 0.445 | 1.27E−49 |
| KNTC1 | 7.98E−54 | 0.57831925 | 0.347 | 0.028 | 1.68E−49 |
| MTERF4 | 9.39E−54 | 0.50293999 | 0.267 | 0 | 1.98E−49 |
| SCRN1 | 1.09E−53 | 0.70024006 | 0.482 | 0.095 | 2.29E−49 |
| NUP214 | 1.10E−53 | 0.56455467 | 0.472 | 0.087 | 2.32E−49 |
| MGST3 | 1.11E−53 | −0.92603731 | 0.238 | 0.677 | 2.34E−49 |
| ATP6V1B1 | 1.12E−53 | 0.79340536 | 0.267 | 0 | 2.36E−49 |
| MUM1 | 1.29E−53 | 0.62406838 | 0.513 | 0.112 | 2.71E−49 |
| TAPBP | 1.61E−53 | 0.6744409 | 0.438 | 0.071 | 3.38E−49 |
| ACIN1 | 1.74E−53 | 0.5800016 | 0.567 | 0.142 | 3.66E−49 |
| WDR48 | 1.88E−53 | 0.76866316 | 0.578 | 0.161 | 3.95E−49 |
| CREBBP | 2.19E−53 | 0.55280186 | 0.42 | 0.06 | 4.61E−49 |
| NUP62 | 2.38E−53 | 0.63066939 | 0.541 | 0.13 | 5.00E−49 |
| C6orf48 | 2.47E−53 | −0.96201466 | 0.269 | 0.692 | 5.20E−49 |
| USP54 | 2.50E−53 | 0.45262166 | 0.311 | 0.015 | 5.26E−49 |
| SEC61B | 3.12E−53 | −0.8083512 | 0.205 | 0.666 | 6.58E−49 |
| RBM7 | 3.29E−53 | 0.3933849 | 0.264 | 0 | 6.93E−49 |
| ADGRB3 | 3.34E−53 | 0.37908652 | 0.264 | 0 | 7.02E−49 |
| GFRA1 | 3.59E−53 | 0.62749736 | 0.44 | 0.072 | 7.55E−49 |
| NUP50 | 3.61E−53 | 0.54836096 | 0.409 | 0.056 | 7.60E−49 |
| ANLN | 4.62E−53 | 0.58732097 | 0.321 | 0.02 | 9.74E−49 |
| SMU1 | 4.67E−53 | 0.61965851 | 0.541 | 0.131 | 9.84E−49 |
| ADARB1 | 5.07E−53 | 0.49155951 | 0.306 | 0.013 | 1.07E−48 |
| DDX11 | 5.42E−53 | 0.44375621 | 0.316 | 0.017 | 1.14E−48 |
| MMS22L | 5.64E−53 | 0.55037643 | 0.389 | 0.046 | 1.19E−48 |
| ADAR | 6.45E−53 | 0.62652371 | 0.549 | 0.138 | 1.36E−48 |
| TSC1 | 6.81E−53 | 0.5352323 | 0.37 | 0.038 | 1.43E−48 |
| DMXL2 | 6.82E−53 | 0.54277749 | 0.368 | 0.038 | 1.44E−48 |
| SNHG5 | 7.23E−53 | −0.78219891 | 0.124 | 0.588 | 1.52E−48 |
| MTMR9LP | 8.07E−53 | 0.4601564 | 0.324 | 0.02 | 1.70E−48 |
| NBR1 | 8.19E−53 | 0.55716885 | 0.425 | 0.064 | 1.72E−48 |
| ATP6V1A | 8.48E−53 | 0.57007776 | 0.459 | 0.081 | 1.79E−48 |
| HS2ST1 | 8.53E−53 | 0.41689604 | 0.298 | 0.011 | 1.79E−48 |
| PHC3 | 9.51E−53 | 0.50021994 | 0.386 | 0.046 | 2.00E−48 |
| EGFEM1P | 9.60E−53 | 0.48660729 | 0.365 | 0.037 | 2.02E−48 |
| IPO5P1 | 1.13E−52 | 0.37332725 | 0.262 | 0 | 2.39E−48 |
| MRPL12 | 1.16E−52 | 0.38768254 | 0.262 | 0 | 2.44E−48 |
| MARS | 1.26E−52 | 0.63971356 | 0.552 | 0.143 | 2.65E−48 |
| MID1 | 1.86E−52 | 0.64665545 | 0.474 | 0.095 | 3.92E−48 |
| YBX1P2 | 2.03E−52 | −0.67939406 | 0 | 0.435 | 4.27E−48 |
| THRAP3 | 2.46E−52 | 0.70950318 | 0.824 | 0.435 | 5.18E−48 |
| EFHD2 | 2.53E−52 | 0.4646515 | 0.337 | 0.026 | 5.33E−48 |
| XPR1 | 3.46E−52 | 0.6318275 | 0.477 | 0.095 | 7.28E−48 |
| ALMS1 | 3.69E−52 | 0.52442736 | 0.378 | 0.044 | 7.77E−48 |
| FAM227A | 3.99E−52 | 0.51783525 | 0.368 | 0.039 | 8.40E−48 |
| SUMO2 | 4.02E−52 | −0.7040821 | 0.876 | 0.947 | 8.45E−48 |
| RNASEH1 | 4.04E−52 | 0.37908652 | 0.259 | 0 | 8.52E−48 |
| F2RL2 | 4.13E−52 | 0.38195755 | 0.259 | 0 | 8.69E−48 |
| SMG5 | 4.24E−52 | 0.43487886 | 0.316 | 0.018 | 8.94E−48 |
| DNMT3B | 4.34E−52 | 0.46043289 | 0.345 | 0.029 | 9.14E−48 |
| DCUN1D1 | 4.45E−52 | 0.5397149 | 0.448 | 0.077 | 9.37E−48 |
| PSMB7 | 4.81E−52 | −0.84618071 | 0.28 | 0.694 | 1.01E−47 |
| SF3B1 | 5.42E−52 | 0.77276516 | 0.806 | 0.424 | 1.14E−47 |
| IARS | 5.91E−52 | 0.74617845 | 0.655 | 0.227 | 1.24E−47 |
| SACS | 6.05E−52 | 0.51515859 | 0.394 | 0.051 | 1.27E−47 |
| POLR1A | 6.30E−52 | 0.46254506 | 0.308 | 0.016 | 1.33E−47 |
| MAP4 | 6.55E−52 | 0.70081426 | 0.666 | 0.235 | 1.38E−47 |
| TOP2A | 6.94E−52 | 2.58583929 | 0.477 | 0.116 | 1.46E−47 |
| TARDBP | 6.98E−52 | 0.68550417 | 0.666 | 0.244 | 1.47E−47 |
| KHDRBS1 | 7.02E−52 | −0.80631709 | 0.459 | 0.784 | 1.48E−47 |
| SPPL2B | 7.10E−52 | 0.57374384 | 0.464 | 0.088 | 1.50E−47 |
| RPL36AL | 7.31E−52 | −0.81324125 | 0.197 | 0.65 | 1.54E−47 |
| TMEM109 | 9.37E−52 | 0.4774142 | 0.386 | 0.048 | 1.97E−47 |
| HLA-DRB1 | 1.01E−51 | 0.61071199 | 0.337 | 0.028 | 2.12E−47 |
| PARP4 | 1.11E−51 | 0.43781126 | 0.326 | 0.023 | 2.34E−47 |
| PALLD | 1.12E−51 | 0.70017136 | 0.552 | 0.148 | 2.35E−47 |
| UGGT1 | 1.17E−51 | 0.47983406 | 0.373 | 0.043 | 2.46E−47 |
| BUB1B | 1.19E−51 | 0.73261764 | 0.326 | 0.024 | 2.50E−47 |
| MT-TT | 1.40E−51 | −0.77215927 | 0 | 0.432 | 2.94E−47 |
| PRAF2 | 1.42E−51 | 0.37043897 | 0.256 | 0 | 2.99E−47 |
| CABIN1 | 1.43E−51 | 0.52524029 | 0.453 | 0.081 | 3.01E−47 |
| CLN3 | 1.43E−51 | 0.364645 | 0.256 | 0 | 3.01E−47 |
| CALR | 1.44E−51 | 0.99120315 | 0.793 | 0.44 | 3.03E−47 |
| CNN2 | 1.44E−51 | 0.6516066 | 0.536 | 0.136 | 3.03E−47 |
| RDH10 | 1.54E−51 | 0.52095879 | 0.293 | 0.011 | 3.24E−47 |
| MMS19 | 1.65E−51 | 0.530064 | 0.389 | 0.05 | 3.47E−47 |
| BCL9 | 1.77E−51 | 0.51843705 | 0.396 | 0.054 | 3.73E−47 |
| BTAF1 | 1.80E−51 | 0.48600314 | 0.396 | 0.053 | 3.78E−47 |
| ECE1 | 1.82E−51 | 0.48377612 | 0.319 | 0.021 | 3.82E−47 |
| PDS5B | 1.82E−51 | 0.62326398 | 0.453 | 0.084 | 3.83E−47 |
| HERC2 | 2.38E−51 | 0.50757748 | 0.417 | 0.064 | 5.01E−47 |
| SLIT2 | 2.42E−51 | 0.50835179 | 0.396 | 0.053 | 5.09E−47 |
| CYP51A1 | 2.50E−51 | 0.72576377 | 0.56 | 0.158 | 5.26E−47 |
| LAMTOR5 | 2.50E−51 | −0.79753717 | 0.269 | 0.696 | 5.27E−47 |
| TSPAN14 | 3.24E−51 | 0.66052468 | 0.57 | 0.165 | 6.81E−47 |
| CNN3 | 3.70E−51 | 0.78581945 | 0.943 | 0.835 | 7.80E−47 |
| MOB3B | 3.82E−51 | 0.64922994 | 0.422 | 0.07 | 8.04E−47 |
| CDK10 | 4.03E−51 | 0.56892682 | 0.469 | 0.093 | 8.49E−47 |
| NAA40 | 4.04E−51 | 0.50099733 | 0.417 | 0.064 | 8.50E−47 |
| NINL | 4.17E−51 | 0.50450786 | 0.339 | 0.029 | 8.79E−47 |
| SIK2 | 4.19E−51 | 0.40334076 | 0.298 | 0.013 | 8.83E−47 |
| RBM15 | 4.64E−51 | 0.45900913 | 0.363 | 0.039 | 9.78E−47 |
| WHSC1 | 4.81E−51 | 0.68392455 | 0.547 | 0.152 | 1.01E−46 |
| STRADA | 4.89E−51 | 0.34123362 | 0.254 | 0 | 1.03E−46 |
| ARPC4 | 4.90E−51 | 0.38109162 | 0.282 | 0.009 | 1.03E−46 |
| LDAH | 5.13E−51 | 0.38482289 | 0.254 | 0 | 1.08E−46 |
| MGARP | 5.29E−51 | 0.44646195 | 0.254 | 0 | 1.11E−46 |
| SLC5A3 | 5.38E−51 | 0.39622765 | 0.254 | 0 | 1.13E−46 |
| MAN2C1 | 5.65E−51 | 0.44683503 | 0.326 | 0.024 | 1.19E−46 |
| IKBKAP | 6.09E−51 | 0.44304238 | 0.347 | 0.032 | 1.28E−46 |
| TMSB4X | 6.22E−51 | −0.89455889 | 0.995 | 0.996 | 1.31E−46 |
| ARCN1 | 6.39E−51 | 0.53806586 | 0.484 | 0.101 | 1.35E−46 |
| CKAP2 | 6.68E−51 | 1.03135769 | 0.539 | 0.145 | 1.41E−46 |
| LSS | 6.92E−51 | 0.59509888 | 0.412 | 0.065 | 1.46E−46 |
| GOT2 | 7.31E−51 | 0.66987207 | 0.63 | 0.215 | 1.54E−46 |
| VPS53 | 7.33E−51 | 0.50183509 | 0.412 | 0.062 | 1.54E−46 |
| PLK2 | 7.52E−51 | 0.97038592 | 0.446 | 0.087 | 1.58E−46 |
| CASC5 | 8.54E−51 | 0.61848901 | 0.332 | 0.028 | 1.80E−46 |
| MCM3 | 8.94E−51 | 1.05129877 | 0.585 | 0.2 | 1.88E−46 |
| ZNF608 | 1.09E−50 | 0.61619809 | 0.554 | 0.15 | 2.29E−46 |
| HNRNPR | 1.10E−50 | 0.7721353 | 0.909 | 0.658 | 2.31E−46 |
| DCTN4 | 1.26E−50 | 0.51366941 | 0.466 | 0.09 | 2.66E−46 |
| PROS1 | 1.40E−50 | 0.44587855 | 0.319 | 0.022 | 2.95E−46 |
| MAU2 | 1.55E−50 | 0.54111728 | 0.37 | 0.043 | 3.26E−46 |
| PSME3 | 1.62E−50 | 0.59193403 | 0.523 | 0.127 | 3.41E−46 |
| NELFCD | 1.71E−50 | 0.66065357 | 0.575 | 0.164 | 3.60E−46 |
| PLEKHA1 | 1.73E−50 | 0.75636433 | 0.451 | 0.088 | 3.65E−46 |
| VKORC1 | 1.79E−50 | 0.37620976 | 0.251 | 0 | 3.78E−46 |
| SPIN4 | 1.80E−50 | 0.364645 | 0.251 | 0 | 3.80E−46 |
| SNX19 | 1.81E−50 | 0.48327074 | 0.358 | 0.037 | 3.82E−46 |
| TM9SF4 | 1.84E−50 | 0.61377186 | 0.487 | 0.109 | 3.87E−46 |
| JUNB | 1.85E−50 | 0.66901834 | 0.386 | 0.054 | 3.89E−46 |
| KMT2E-AS1 | 1.87E−50 | 0.37908652 | 0.251 | 0 | 3.93E−46 |
| GIGYF1 | 2.02E−50 | 0.39955575 | 0.319 | 0.022 | 4.26E−46 |
| TPP2 | 2.04E−50 | 0.47835961 | 0.389 | 0.051 | 4.30E−46 |
| HIST1H3I | 2.11E−50 | 0.68599242 | 0.251 | 0 | 4.44E−46 |
| RBM10 | 2.25E−50 | 0.59901889 | 0.484 | 0.104 | 4.74E−46 |
| FAM134C | 2.46E−50 | 0.44454985 | 0.339 | 0.031 | 5.19E−46 |
| SRGAP3 | 2.67E−50 | 0.72315521 | 0.611 | 0.21 | 5.61E−46 |
| FBN1 | 3.06E−50 | 0.40822672 | 0.275 | 0.007 | 6.44E−46 |
| NFATC4 | 3.21E−50 | 0.45987588 | 0.303 | 0.017 | 6.75E−46 |
| SEPW1 | 3.34E−50 | −0.81413829 | 0.238 | 0.667 | 7.04E−46 |
| LIN7C | 3.37E−50 | 0.70450525 | 0.448 | 0.086 | 7.10E−46 |
| SPIN1 | 4.53E−50 | 0.63474188 | 0.598 | 0.186 | 9.54E−46 |
| C1orf122 | 4.69E−50 | −0.70791338 | 0.028 | 0.468 | 9.86E−46 |
| SNX3 | 4.97E−50 | −0.80185744 | 0.197 | 0.632 | 1.05E−45 |
| C16orf62 | 5.22E−50 | 0.48949236 | 0.415 | 0.065 | 1.10E−45 |
| STAT2 | 5.27E−50 | 0.5249784 | 0.319 | 0.023 | 1.11E−45 |
| POGK | 5.97E−50 | 0.50525553 | 0.37 | 0.045 | 1.26E−45 |
| POLR2J | 6.04E−50 | −0.74668209 | 0.088 | 0.532 | 1.27E−45 |
| KIF15 | 7.10E−50 | 0.69128778 | 0.355 | 0.04 | 1.49E−45 |
| BUD31 | 8.01E−50 | −0.76629691 | 0.114 | 0.561 | 1.69E−45 |
| CNOT10 | 9.58E−50 | 0.48720949 | 0.448 | 0.082 | 2.02E−45 |
| CELSR2 | 1.09E−49 | 0.50355557 | 0.381 | 0.051 | 2.29E−45 |
| WDR19 | 1.13E−49 | 0.45701823 | 0.334 | 0.029 | 2.37E−45 |
| MT-TA | 1.25E−49 | −0.73237271 | 0 | 0.418 | 2.63E−45 |
| NGFRAP1 | 1.28E−49 | −0.73657683 | 0.865 | 0.94 | 2.70E−45 |
| FANCA | 1.43E−49 | 0.42921865 | 0.319 | 0.023 | 3.02E−45 |
| NDUFB2 | 1.47E−49 | −0.77023613 | 0.137 | 0.582 | 3.08E−45 |
| COMT | 1.49E−49 | −0.72280836 | 0.021 | 0.447 | 3.13E−45 |
| ARFGAP1 | 1.57E−49 | 0.44644898 | 0.339 | 0.032 | 3.30E−45 |
| HELZ | 1.66E−49 | 0.58086517 | 0.44 | 0.083 | 3.49E−45 |
| SETX | 1.80E−49 | 0.55298528 | 0.448 | 0.084 | 3.79E−45 |
| HEY1 | 1.97E−49 | 0.68047787 | 0.308 | 0.021 | 4.15E−45 |
| CALCRL | 2.09E−49 | 0.42389094 | 0.267 | 0.006 | 4.40E−45 |
| ZC3H7B | 2.17E−49 | 0.4612206 | 0.329 | 0.028 | 4.56E−45 |
| PI4KB | 2.24E−49 | 0.50511764 | 0.409 | 0.065 | 4.71E−45 |
| UBXN2B | 2.62E−49 | 0.51888614 | 0.404 | 0.061 | 5.52E−45 |
| MED24 | 2.74E−49 | 0.58823188 | 0.474 | 0.1 | 5.77E−45 |
| KIAA0100 | 2.94E−49 | 0.50440382 | 0.368 | 0.045 | 6.19E−45 |
| MAGI3 | 2.95E−49 | 0.48039015 | 0.324 | 0.027 | 6.22E−45 |
| SEMA6A | 3.20E−49 | 0.75229409 | 0.495 | 0.123 | 6.74E−45 |
| TSPAN3 | 3.46E−49 | 0.70940213 | 0.93 | 0.633 | 7.27E−45 |
| CMTR1 | 3.50E−49 | 0.43706219 | 0.321 | 0.026 | 7.36E−45 |
| PGAP1 | 4.59E−49 | 0.583381 | 0.477 | 0.104 | 9.67E−45 |
| ABCD4 | 4.98E−49 | 0.61805754 | 0.513 | 0.128 | 1.05E−44 |
| NCOA2 | 5.21E−49 | 0.48830557 | 0.342 | 0.034 | 1.10E−44 |
| MSH2 | 5.22E−49 | 0.65704989 | 0.56 | 0.165 | 1.10E−44 |
| MSANTD4 | 5.32E−49 | 0.70389619 | 0.487 | 0.112 | 1.12E−44 |
| NASP | 6.18E−49 | 0.9085965 | 0.744 | 0.379 | 1.30E−44 |
| WDR11 | 7.91E−49 | 0.55409547 | 0.383 | 0.054 | 1.66E−44 |
| PLOD1 | 1.13E−48 | 0.57601625 | 0.394 | 0.061 | 2.38E−44 |
| ZNF574 | 1.13E−48 | 0.50776675 | 0.365 | 0.045 | 2.38E−44 |
| RNF168 | 1.21E−48 | 0.63690646 | 0.461 | 0.098 | 2.54E−44 |
| KIF11 | 1.27E−48 | 0.90846593 | 0.391 | 0.064 | 2.68E−44 |
| SEC11A | 1.31E−48 | −0.83819501 | 0.313 | 0.704 | 2.76E−44 |
| SRP14 | 1.38E−48 | −0.74318319 | 0.956 | 0.985 | 2.91E−44 |
| DSEL | 1.48E−48 | 0.60870775 | 0.311 | 0.023 | 3.11E−44 |
| DNMT3A | 1.60E−48 | 0.53878696 | 0.487 | 0.11 | 3.38E−44 |
| POTEF | 1.61E−48 | 0.3914889 | 0.293 | 0.016 | 3.38E−44 |
| PUM2 | 1.79E−48 | 0.58398271 | 0.495 | 0.116 | 3.77E−44 |
| SF3B5 | 2.31E−48 | −0.75335892 | 0.194 | 0.636 | 4.85E−44 |
| TTN | 2.61E−48 | 0.39875138 | 0.262 | 0.006 | 5.49E−44 |
| MON2 | 2.71E−48 | 0.45114701 | 0.339 | 0.034 | 5.71E−44 |
| C7orf50 | 2.72E−48 | −0.50082541 | 0.44 | 0.781 | 5.74E−44 |
| HNRNPU | 3.19E−48 | 0.71373024 | 0.756 | 0.358 | 6.71E−44 |
| EDC3 | 3.46E−48 | 0.46952905 | 0.355 | 0.042 | 7.29E−44 |
| RABAC1 | 3.82E−48 | −0.74346501 | 0.36 | 0.736 | 8.03E−44 |
| RFC5 | 4.00E−48 | 0.75259528 | 0.477 | 0.114 | 8.41E−44 |
| LRP6 | 4.03E−48 | 0.47469075 | 0.352 | 0.04 | 8.48E−44 |
| USP34 | 4.08E−48 | 0.55262501 | 0.433 | 0.081 | 8.58E−44 |
| IFT80 | 4.75E−48 | 0.58978656 | 0.402 | 0.066 | 1.00E−43 |
| RBM5 | 5.18E−48 | 0.59717214 | 0.539 | 0.149 | 1.09E−43 |
| PCIF1 | 5.23E−48 | 0.53847624 | 0.36 | 0.045 | 1.10E−43 |
| TBCB | 6.35E−48 | −0.78871315 | 0.497 | 0.784 | 1.34E−43 |
| KIF22 | 7.00E−48 | 0.94874048 | 0.534 | 0.158 | 1.47E−43 |
| SULF2 | 7.70E−48 | 0.50632885 | 0.288 | 0.016 | 1.62E−43 |
| TOR1AIP2 | 8.11E−48 | 0.55316457 | 0.443 | 0.089 | 1.71E−43 |
| C14orf132 | 8.97E−48 | 0.55736798 | 0.461 | 0.099 | 1.89E−43 |
| PPP2R4 | 9.65E−48 | 0.46499445 | 0.43 | 0.077 | 2.03E−43 |
| C2orf68 | 1.05E−47 | 0.58799912 | 0.438 | 0.084 | 2.22E−43 |
| QRICH1 | 1.13E−47 | 0.49113271 | 0.409 | 0.068 | 2.37E−43 |
| KIF2C | 1.14E−47 | 0.89771646 | 0.363 | 0.05 | 2.40E−43 |
| KRT10 | 1.15E−47 | −0.72484071 | 0.07 | 0.504 | 2.42E−43 |
| FAM162A | 1.15E−47 | −1.10412701 | 0.233 | 0.653 | 2.42E−43 |
| PRKARIA | 1.16E−47 | 0.77043188 | 0.821 | 0.494 | 2.43E−43 |
| PSMD1 | 1.16E−47 | 0.65329605 | 0.609 | 0.202 | 2.45E−43 |
| CNDP2 | 1.19E−47 | 0.6526239 | 0.624 | 0.221 | 2.50E−43 |
| OAZ1 | 1.30E−47 | −0.74600057 | 0.878 | 0.954 | 2.73E−43 |
| MIS18BP1 | 1.50E−47 | 0.93983768 | 0.451 | 0.098 | 3.15E−43 |
| HSDL1 | 1.55E−47 | 0.60889794 | 0.536 | 0.148 | 3.26E−43 |
| SIPA1L3 | 1.59E−47 | 0.41936728 | 0.262 | 0.007 | 3.34E−43 |
| PTCD3 | 1.62E−47 | 0.54653178 | 0.523 | 0.137 | 3.41E−43 |
| KCTD20 | 1.76E−47 | 0.52322678 | 0.448 | 0.09 | 3.70E−43 |
| RTN1 | 1.97E−47 | −1.30131249 | 0.06 | 0.482 | 4.15E−43 |
| MOB4 | 2.08E−47 | −0.64484352 | 0 | 0.402 | 4.38E−43 |
| EEF2 | 2.28E−47 | 0.76104928 | 0.972 | 0.988 | 4.81E−43 |
| CALU | 2.49E−47 | 0.78773765 | 0.769 | 0.406 | 5.24E−43 |
| ZNF451 | 3.55E−47 | 0.53990513 | 0.448 | 0.09 | 7.48E−43 |
| MEST | 3.96E−47 | −0.87956607 | 0.845 | 0.977 | 8.33E−43 |
| PLK4 | 4.02E−47 | 0.42625543 | 0.308 | 0.024 | 8.46E−43 |
| PTPN13 | 4.15E−47 | 0.59396337 | 0.464 | 0.104 | 8.74E−43 |
| ACO1 | 4.43E−47 | 0.49772949 | 0.433 | 0.082 | 9.33E−43 |
| ERCC6L2 | 4.45E−47 | 0.58169203 | 0.381 | 0.057 | 9.38E−43 |
| HSPA8 | 4.47E−47 | 1.0067367 | 0.889 | 0.787 | 9.40E−43 |
| RFWD3 | 4.54E−47 | 0.46798003 | 0.298 | 0.021 | 9.56E−43 |
| LUC7L3 | 4.82E−47 | 0.69781131 | 0.795 | 0.423 | 1.01E−42 |
| MRPL23 | 5.16E−47 | −0.63592842 | 0.005 | 0.41 | 1.09E−42 |
| MDN1 | 5.35E−47 | 0.3764956 | 0.267 | 0.01 | 1.13E−42 |
| COPB1 | 6.37E−47 | 0.72360405 | 0.575 | 0.189 | 1.34E−42 |
| LAMA5 | 6.63E−47 | 0.46705926 | 0.275 | 0.012 | 1.40E−42 |
| MANF | 7.27E−47 | −0.74993882 | 0.049 | 0.474 | 1.53E−42 |
| C16orf45 | 8.11E−47 | 0.71462149 | 0.591 | 0.193 | 1.71E−42 |
| HIST1H3G | 8.44E−47 | 0.65141681 | 0.254 | 0.006 | 1.78E−42 |
| PTPRF | 9.25E−47 | 0.71309549 | 0.539 | 0.16 | 1.95E−42 |
| RP11-344E13.3 | 1.07E−46 | −0.69254943 | 0 | 0.399 | 2.25E−42 |
| RPS6KB1 | 1.19E−46 | 0.36103397 | 0.262 | 0.009 | 2.50E−42 |
| DPY19L3 | 1.25E−46 | 0.48920512 | 0.345 | 0.04 | 2.64E−42 |
| RLIM | 1.35E−46 | 0.50485818 | 0.464 | 0.101 | 2.84E−42 |
| POLR3E | 1.41E−46 | 0.65221134 | 0.37 | 0.054 | 2.96E−42 |
| MCM6 | 1.65E−46 | 0.78057286 | 0.461 | 0.112 | 3.48E−42 |
| PSKH1 | 1.76E−46 | 0.404396 | 0.306 | 0.024 | 3.70E−42 |
| SEH1L | 1.92E−46 | 0.41129061 | 0.293 | 0.02 | 4.04E−42 |
| RPS21 | 2.46E−46 | −0.75611435 | 0.301 | 0.692 | 5.18E−42 |
| C19orf53 | 2.47E−46 | −0.67649657 | 0.223 | 0.644 | 5.21E−42 |
| TUBGCP4 | 2.54E−46 | 0.47071381 | 0.422 | 0.078 | 5.35E−42 |
| SUMO2P1 | 3.07E−46 | −0.62782372 | 0 | 0.395 | 6.46E−42 |
| CSDE1 | 3.67E−46 | 0.76358543 | 0.876 | 0.644 | 7.73E−42 |
| PDXDC1 | 3.69E−46 | 0.45637508 | 0.376 | 0.055 | 7.76E−42 |
| CCNF | 3.73E−46 | 0.64648916 | 0.298 | 0.023 | 7.85E−42 |
| ATAD2 | 4.03E−46 | 0.64098176 | 0.407 | 0.073 | 8.49E−42 |
| HM13 | 4.09E−46 | 0.67183328 | 0.715 | 0.3 | 8.61E−42 |
| RBBP4 | 4.34E−46 | 0.68541563 | 0.855 | 0.555 | 9.13E−42 |
| ZNF462 | 4.38E−46 | 0.68230615 | 0.528 | 0.147 | 9.23E−42 |
| CDCA7 | 4.54E−46 | 0.61783642 | 0.368 | 0.055 | 9.56E−42 |
| TKT | 4.65E−46 | 0.90066373 | 0.777 | 0.471 | 9.79E−42 |
| H6PD | 4.71E−46 | 0.39282805 | 0.269 | 0.012 | 9.92E−42 |
| RBM23 | 4.93E−46 | 0.60856755 | 0.617 | 0.218 | 1.04E−41 |
| UBQLN2 | 5.44E−46 | 0.62424396 | 0.598 | 0.205 | 1.15E−41 |
| LRIG2 | 5.93E−46 | 0.4441874 | 0.334 | 0.037 | 1.25E−41 |
| KIF18B | 6.57E−46 | 0.4165902 | 0.254 | 0.007 | 1.38E−41 |
| PROM1 | 6.79E−46 | 0.42206836 | 0.358 | 0.046 | 1.43E−41 |
| FTL | 6.88E−46 | −1.11154209 | 0.974 | 0.999 | 1.45E−41 |
| CD320 | 7.73E−46 | 0.34244331 | 0.254 | 0.007 | 1.63E−41 |
| STIP1 | 8.96E−46 | 0.70033512 | 0.697 | 0.307 | 1.89E−41 |
| ZNF638 | 9.25E−46 | 0.6733119 | 0.738 | 0.342 | 1.95E−41 |
| EP300 | 1.23E−45 | 0.47888544 | 0.329 | 0.035 | 2.60E−41 |
| BTBD3 | 1.45E−45 | 0.59124114 | 0.412 | 0.078 | 3.06E−41 |
| BCLAF1 | 1.49E−45 | 0.60857075 | 0.741 | 0.341 | 3.13E−41 |
| UHRF1BP1 | 1.71E−45 | 0.42526161 | 0.301 | 0.024 | 3.60E−41 |
| KPNA1 | 1.80E−45 | 0.56783594 | 0.503 | 0.128 | 3.79E−41 |
| TRAFD1 | 1.90E−45 | 0.4985375 | 0.368 | 0.055 | 4.01E−41 |
| TOMM7 | 1.96E−45 | −0.7224062 | 0.091 | 0.515 | 4.12E−41 |
| GPC4 | 1.99E−45 | 0.62968727 | 0.552 | 0.164 | 4.18E−41 |
| PPP4C | 2.05E−45 | −0.75127762 | 0.308 | 0.694 | 4.33E−41 |
| TRIP12 | 2.12E−45 | 0.5177098 | 0.427 | 0.086 | 4.47E−41 |
| ZC3H11A | 2.20E−45 | 0.60651641 | 0.477 | 0.115 | 4.62E−41 |
| MCM5 | 2.34E−45 | 0.87159065 | 0.451 | 0.108 | 4.93E−41 |
| SMARCAL1 | 2.37E−45 | 0.46932171 | 0.321 | 0.033 | 4.99E−41 |
| PTPRK | 2.53E−45 | 0.45516409 | 0.355 | 0.048 | 5.33E−41 |
| WDHD1 | 3.21E−45 | 0.50691068 | 0.345 | 0.044 | 6.76E−41 |
| CALCOCO1 | 3.54E−45 | 0.63397412 | 0.391 | 0.068 | 7.45E−41 |
| LSM7 | 3.65E−45 | −0.72601368 | 0.215 | 0.627 | 7.69E−41 |
| SCAP | 3.82E−45 | 0.50093999 | 0.433 | 0.088 | 8.03E−41 |
| C1orf86 | 3.87E−45 | −0.5767084 | 0 | 0.386 | 8.15E−41 |
| EEF1B2 | 3.96E−45 | −0.75892307 | 0.668 | 0.852 | 8.33E−41 |
| HNRNPH2 | 5.15E−45 | −0.61290904 | 0 | 0.386 | 1.08E−40 |
| HDAC5 | 5.16E−45 | 0.5476081 | 0.422 | 0.083 | 1.09E−40 |
| AP1G1 | 5.56E−45 | 0.44980183 | 0.352 | 0.048 | 1.17E−40 |
| DCUN1D4 | 5.74E−45 | 0.46303881 | 0.363 | 0.053 | 1.21E−40 |
| CPNE1 | 7.30E−45 | 0.53037358 | 0.552 | 0.16 | 1.54E−40 |
| DHCR7 | 7.88E−45 | 0.68129531 | 0.523 | 0.152 | 1.66E−40 |
| APEH | 8.00E−45 | 0.56849559 | 0.526 | 0.147 | 1.69E−40 |
| SPCS3 | 8.05E−45 | 0.56704627 | 0.567 | 0.18 | 1.69E−40 |
| ACKR3 | 9.67E−45 | 0.59196436 | 0.326 | 0.038 | 2.03E−40 |
| LANCL1 | 9.83E−45 | 0.56800232 | 0.415 | 0.081 | 2.07E−40 |
| NDUFB10 | 1.01E−44 | −0.70603365 | 0.262 | 0.66 | 2.14E−40 |
| ECT2 | 1.05E−44 | 0.77189904 | 0.36 | 0.055 | 2.22E−40 |
| EHD1 | 1.10E−44 | 0.49513089 | 0.324 | 0.037 | 2.32E−40 |
| TACC3 | 1.13E−44 | 0.72755539 | 0.35 | 0.05 | 2.38E−40 |
| INTS7 | 1.19E−44 | 0.4617517 | 0.334 | 0.04 | 2.51E−40 |
| ITPRIP | 1.26E−44 | 0.37300661 | 0.256 | 0.01 | 2.65E−40 |
| STK36 | 1.30E−44 | 0.38371195 | 0.267 | 0.013 | 2.74E−40 |
| STUB1 | 1.31E−44 | −0.62091801 | 0.01 | 0.4 | 2.76E−40 |
| POLQ | 1.32E−44 | 0.45625769 | 0.269 | 0.015 | 2.77E−40 |
| SHC1 | 1.33E−44 | 0.49092431 | 0.337 | 0.042 | 2.79E−40 |
| RP11-742N3.1 | 1.57E−44 | −0.58613781 | 0 | 0.383 | 3.30E−40 |
| FAM117B | 1.64E−44 | 0.5111817 | 0.332 | 0.04 | 3.44E−40 |
| DCP2 | 1.68E−44 | 0.61864616 | 0.479 | 0.122 | 3.53E−40 |
| MRPL14 | 1.69E−44 | 0.40334076 | 0.269 | 0.015 | 3.55E−40 |
| GOLGA3 | 1.77E−44 | 0.41457518 | 0.321 | 0.034 | 3.73E−40 |
| MAVS | 1.78E−44 | 0.45997391 | 0.373 | 0.057 | 3.75E−40 |
| UQCRH | 1.89E−44 | −0.73050944 | 0.492 | 0.778 | 3.97E−40 |
| BARD1 | 1.93E−44 | 0.54955523 | 0.402 | 0.075 | 4.06E−40 |
| SSR4 | 1.95E−44 | −0.81543063 | 0.308 | 0.677 | 4.11E−40 |
| NACAP1 | 2.00E−44 | −0.56959567 | 0 | 0.381 | 4.21E−40 |
| HSP90B1 | 2.05E−44 | 0.9747493 | 0.821 | 0.553 | 4.31E−40 |
| AP3M1 | 2.12E−44 | 0.57708062 | 0.552 | 0.174 | 4.47E−40 |
| PTPRO | 2.30E−44 | 0.44209022 | 0.316 | 0.033 | 4.84E−40 |
| NUP160 | 2.34E−44 | 0.57563185 | 0.464 | 0.112 | 4.93E−40 |
| LCA5 | 2.47E−44 | 0.58250854 | 0.383 | 0.066 | 5.19E−40 |
| ZIC4 | 2.52E−44 | −0.76965862 | 0.041 | 0.443 | 5.31E−40 |
| HECTD4 | 2.55E−44 | 0.46344203 | 0.324 | 0.035 | 5.37E−40 |
| ANGEL1 | 2.94E−44 | 0.37126528 | 0.262 | 0.012 | 6.18E−40 |
| WFS1 | 2.99E−44 | 0.35060407 | 0.254 | 0.01 | 6.30E−40 |
| TXNRD1 | 3.08E−44 | 0.54650282 | 0.459 | 0.109 | 6.48E−40 |
| INPP5F | 3.08E−44 | 0.59480546 | 0.391 | 0.07 | 6.49E−40 |
| MBTPS1 | 3.10E−44 | 0.50098897 | 0.391 | 0.068 | 6.53E−40 |
| LBR | 3.34E−44 | 0.89624558 | 0.681 | 0.318 | 7.04E−40 |
| NDUFS5 | 3.55E−44 | −0.81362803 | 0.446 | 0.751 | 7.48E−40 |
| HMGCR | 3.88E−44 | 0.8786802 | 0.57 | 0.202 | 8.16E−40 |
| PTK7 | 4.19E−44 | 0.47129266 | 0.396 | 0.071 | 8.82E−40 |
| CHD4 | 4.88E−44 | 0.64944527 | 0.666 | 0.278 | 1.03E−39 |
| RPL22 | 5.23E−44 | −0.69886425 | 0.645 | 0.842 | 1.10E−39 |
| EP400 | 5.25E−44 | 0.44878446 | 0.404 | 0.075 | 1.11E−39 |
| CUL1 | 5.67E−44 | 0.60956881 | 0.464 | 0.117 | 1.19E−39 |
| ALKBH7 | 6.43E−44 | −0.71072187 | 0.264 | 0.653 | 1.35E−39 |
| KIF18A | 6.68E−44 | 0.634029 | 0.316 | 0.035 | 1.41E−39 |
| COG8 | 6.90E−44 | 0.37984404 | 0.303 | 0.028 | 1.45E−39 |
| SLC38A1 | 6.93E−44 | 0.76990776 | 0.707 | 0.335 | 1.46E−39 |
| EPB41L2 | 7.35E−44 | 0.55206689 | 0.363 | 0.055 | 1.55E−39 |
| CCAR1 | 7.64E−44 | 0.56147316 | 0.573 | 0.188 | 1.61E−39 |
| LINC00632 | 7.78E−44 | −0.74422272 | 0.078 | 0.483 | 1.64E−39 |
| CDH2 | 7.91E−44 | 0.73768487 | 0.826 | 0.521 | 1.67E−39 |
| MIEF1 | 8.48E−44 | 0.60956881 | 0.43 | 0.092 | 1.78E−39 |
| WDR90 | 8.66E−44 | 0.48625917 | 0.251 | 0.01 | 1.82E−39 |
| TRABD | 8.73E−44 | 0.50598048 | 0.37 | 0.059 | 1.84E−39 |
| MTHFD1 | 9.07E−44 | 0.53424156 | 0.386 | 0.07 | 1.91E−39 |
| C16orf80 | 9.51E−44 | −0.6163646 | 0 | 0.378 | 2.00E−39 |
| NDUFV2 | 9.58E−44 | 0.41647046 | 0.339 | 0.043 | 2.02E−39 |
| DCAF7 | 1.01E−43 | 0.73983012 | 0.689 | 0.32 | 2.12E−39 |
| FLNB | 1.02E−43 | 0.40128043 | 0.282 | 0.021 | 2.14E−39 |
| UNC45A | 1.04E−43 | 0.46639386 | 0.404 | 0.077 | 2.20E−39 |
| USP10 | 1.08E−43 | 0.63393009 | 0.624 | 0.238 | 2.28E−39 |
| FN1 | 1.09E−43 | 0.55037643 | 0.339 | 0.045 | 2.28E−39 |
| TMBIM4 | 1.15E−43 | 0.38327495 | 0.295 | 0.026 | 2.42E−39 |
| ARGLU1 | 1.21E−43 | 0.63545408 | 0.811 | 0.439 | 2.55E−39 |
| SLC5A6 | 1.43E−43 | 0.45681408 | 0.386 | 0.067 | 3.00E−39 |
| YWHAH | 1.61E−43 | −0.85394169 | 0.334 | 0.685 | 3.39E−39 |
| RPS3AP6 | 1.76E−43 | −0.56959567 | 0 | 0.375 | 3.71E−39 |
| ARID1B | 1.80E−43 | 0.45454159 | 0.313 | 0.033 | 3.80E−39 |
| SND1 | 1.97E−43 | 0.59324192 | 0.593 | 0.211 | 4.14E−39 |
| TOP3A | 2.25E−43 | 0.45344961 | 0.306 | 0.031 | 4.74E−39 |
| ELMO2 | 2.26E−43 | 0.45654341 | 0.326 | 0.039 | 4.75E−39 |
| BOC | 2.32E−43 | 0.57883912 | 0.404 | 0.078 | 4.88E−39 |
| KAT6A | 2.63E−43 | 0.51275135 | 0.443 | 0.1 | 5.54E−39 |
| PRRC2C | 3.03E−43 | 0.58705286 | 0.756 | 0.357 | 6.39E−39 |
| RABL5 | 3.16E−43 | −0.60713132 | 0 | 0.374 | 6.66E−39 |
| TTYH3 | 3.45E−43 | 0.45807789 | 0.42 | 0.084 | 7.26E−39 |
| DAP | 3.65E−43 | 0.67791956 | 0.518 | 0.15 | 7.68E−39 |
| NUP93 | 3.84E−43 | 0.67173657 | 0.583 | 0.205 | 8.08E−39 |
| RPL34 | 3.92E−43 | −1.12712542 | 0.977 | 0.944 | 8.24E−39 |
| MCM10 | 5.09E−43 | 0.5120523 | 0.306 | 0.032 | 1.07E−38 |
| AB019441.29 | 5.45E−43 | −0.55165891 | 0 | 0.372 | 1.15E−38 |
| DENND5A | 5.47E−43 | 0.40369512 | 0.272 | 0.018 | 1.15E−38 |
| TBCA | 5.72E−43 | −0.73915406 | 0.35 | 0.702 | 1.20E−38 |
| KIFC1 | 6.08E−43 | 0.84032601 | 0.378 | 0.071 | 1.28E−38 |
| CBL | 6.37E−43 | 0.50828547 | 0.394 | 0.073 | 1.34E−38 |
| FLRT3 | 6.71E−43 | 0.78833297 | 0.417 | 0.092 | 1.41E−38 |
| NFAT5 | 7.47E−43 | 0.57613649 | 0.479 | 0.126 | 1.57E−38 |
| INTS3 | 7.73E−43 | 0.44939809 | 0.358 | 0.055 | 1.63E−38 |
| NAA25 | 8.22E−43 | 0.4314233 | 0.355 | 0.053 | 1.73E−38 |
| PTOV1 | 8.94E−43 | −0.74176056 | 0.176 | 0.582 | 1.88E−38 |
| CYB5B | 9.40E−43 | 0.49699698 | 0.459 | 0.109 | 1.98E−38 |
| MAGEH1 | 9.79E−43 | 0.36597428 | 0.316 | 0.034 | 2.06E−38 |
| CDC7 | 1.07E−42 | 0.46226211 | 0.383 | 0.067 | 2.25E−38 |
| TAGLN2 | 1.08E−42 | 0.50509521 | 0.37 | 0.061 | 2.27E−38 |
| ACVR2A | 1.09E−42 | 0.47406299 | 0.35 | 0.053 | 2.29E−38 |
| RP11-887P2.3 | 1.14E−42 | −0.60597299 | 0 | 0.37 | 2.40E−38 |
| NDRG4 | 1.17E−42 | 0.61389235 | 0.417 | 0.089 | 2.47E−38 |
| MAGT1 | 1.23E−42 | 0.38763961 | 0.352 | 0.051 | 2.59E−38 |
| ATRX | 1.31E−42 | 0.65371591 | 0.707 | 0.339 | 2.76E−38 |
| ATP6AP1 | 1.32E−42 | 0.59040943 | 0.63 | 0.24 | 2.78E−38 |
| TFDP2 | 1.33E−42 | 0.72801934 | 0.666 | 0.278 | 2.80E−38 |
| POFUT1 | 1.35E−42 | 0.39620083 | 0.306 | 0.032 | 2.84E−38 |
| AARS2 | 1.57E−42 | 0.33955642 | 0.256 | 0.013 | 3.31E−38 |
| TBC1D5 | 1.61E−42 | 0.50350375 | 0.368 | 0.062 | 3.39E−38 |
| TMEM184B | 1.67E−42 | 0.48065598 | 0.409 | 0.082 | 3.51E−38 |
| PJA2 | 1.87E−42 | 0.71007289 | 0.609 | 0.235 | 3.94E−38 |
| ZC3H7A | 1.90E−42 | 0.53274092 | 0.448 | 0.106 | 4.00E−38 |
| FSTL1 | 2.03E−42 | 0.56565169 | 0.5 | 0.139 | 4.27E−38 |
| TTLL4 | 2.03E−42 | 0.3837019 | 0.293 | 0.027 | 4.28E−38 |
| HMG20A | 2.07E−42 | 0.50509521 | 0.386 | 0.071 | 4.35E−38 |
| NDUFA7.1 | 2.10E−42 | −0.57434139 | 0 | 0.368 | 4.42E−38 |
| YEATS2 | 2.43E−42 | 0.51080278 | 0.482 | 0.127 | 5.11E−38 |
| PFN1 | 2.48E−42 | −0.68929527 | 0.824 | 0.921 | 5.22E−38 |
| TERF2IP | 2.79E−42 | −0.86566404 | 0.21 | 0.6 | 5.88E−38 |
| KIAA1524 | 2.97E−42 | 0.51683128 | 0.337 | 0.048 | 6.24E−38 |
| VPS13C | 2.98E−42 | 0.50904751 | 0.402 | 0.081 | 6.28E−38 |
| COL4A2 | 3.04E−42 | 0.49935419 | 0.381 | 0.068 | 6.41E−38 |
| GPRC5B | 3.05E−42 | 0.48047278 | 0.391 | 0.075 | 6.42E−38 |
| RRM1 | 3.19E−42 | 0.74961344 | 0.57 | 0.209 | 6.71E−38 |
| NCAPD3 | 3.22E−42 | 0.41155419 | 0.301 | 0.031 | 6.78E−38 |
| NFATC3 | 3.55E−42 | 0.35633888 | 0.262 | 0.016 | 7.47E−38 |
| ABR | 3.84E−42 | 0.45637508 | 0.36 | 0.059 | 8.09E−38 |
| DEPDC1B | 3.85E−42 | 0.64826171 | 0.373 | 0.067 | 8.10E−38 |
| SLC39A9 | 3.86E−42 | 0.36802536 | 0.293 | 0.027 | 8.13E−38 |
| NAA15 | 4.41E−42 | 0.46313721 | 0.394 | 0.075 | 9.29E−38 |
| MAEA | 4.57E−42 | 0.59295989 | 0.51 | 0.15 | 9.62E−38 |
| TLK1 | 4.90E−42 | 0.55920028 | 0.534 | 0.16 | 1.03E−37 |
| EMX2 | 5.68E−42 | −0.66420853 | 0.003 | 0.372 | 1.20E−37 |
| SRP9P1 | 5.90E−42 | −0.55045516 | 0 | 0.364 | 1.24E−37 |
| CLASP2 | 6.01E−42 | 0.62984494 | 0.557 | 0.182 | 1.26E−37 |
| DCTN5 | 6.02E−42 | 0.50676738 | 0.37 | 0.065 | 1.27E−37 |
| TMEM161B-AS1 | 6.09E−42 | −0.67097956 | 0.016 | 0.391 | 1.28E−37 |
| CADM2 | 6.61E−42 | 0.65767331 | 0.56 | 0.189 | 1.39E−37 |
| TMEM258 | 7.14E−42 | −0.63475992 | 0.14 | 0.553 | 1.50E−37 |
| SEC23A | 7.33E−42 | 0.67973361 | 0.578 | 0.211 | 1.54E−37 |
| ZC3H14 | 7.68E−42 | 0.57780746 | 0.583 | 0.204 | 1.62E−37 |
| CXXC1 | 8.15E−42 | 0.54905277 | 0.415 | 0.087 | 1.72E−37 |
| TBC1D13 | 8.55E−42 | 0.34826322 | 0.264 | 0.017 | 1.80E−37 |
| CDC5L | 9.84E−42 | 0.55214224 | 0.5 | 0.143 | 2.07E−37 |
| TMBIM6 | 1.11E−41 | 0.61644911 | 0.93 | 0.869 | 2.34E−37 |
| RP11-556K13.1 | 1.11E−41 | −0.55766264 | 0 | 0.363 | 2.34E−37 |
| TCERG1 | 1.12E−41 | 0.53544819 | 0.547 | 0.172 | 2.35E−37 |
| KDM6A | 1.13E−41 | 0.33963979 | 0.277 | 0.022 | 2.39E−37 |
| OS9 | 1.33E−41 | 0.6312575 | 0.663 | 0.285 | 2.79E−37 |
| KDM4C | 1.39E−41 | 0.41372741 | 0.262 | 0.017 | 2.93E−37 |
| NAT10 | 1.47E−41 | 0.45014601 | 0.342 | 0.051 | 3.10E−37 |
| SRGAP2 | 1.62E−41 | 0.37284916 | 0.259 | 0.016 | 3.42E−37 |
| SARNP | 1.79E−41 | 0.57203273 | 0.469 | 0.12 | 3.77E−37 |
| NELL2 | 1.82E−41 | 0.64832485 | 0.549 | 0.185 | 3.83E−37 |
| SCAF4 | 1.83E−41 | 0.55652068 | 0.484 | 0.131 | 3.85E−37 |
| EIF3K | 1.86E−41 | −0.70159016 | 0.466 | 0.759 | 3.92E−37 |
| TTK | 1.90E−41 | 0.7051573 | 0.295 | 0.031 | 4.00E−37 |
| CKAP5 | 2.10E−41 | 0.68948028 | 0.474 | 0.134 | 4.42E−37 |
| RNF4 | 2.10E−41 | 0.45807789 | 0.42 | 0.09 | 4.42E−37 |
| CACFD1 | 2.11E−41 | 0.48384098 | 0.394 | 0.078 | 4.45E−37 |
| CUTA | 2.27E−41 | −0.73618456 | 0.635 | 0.831 | 4.78E−37 |
| CUX1 | 2.34E−41 | 0.53291629 | 0.474 | 0.126 | 4.93E−37 |
| LARS2 | 2.51E−41 | 0.3557419 | 0.262 | 0.017 | 5.29E−37 |
| CHMP7 | 2.60E−41 | 0.47197389 | 0.363 | 0.06 | 5.47E−37 |
| SSBP2 | 2.73E−41 | 0.62393134 | 0.78 | 0.4 | 5.76E−37 |
| C2CD5 | 3.08E−41 | 0.36692619 | 0.288 | 0.027 | 6.49E−37 |
| ALDH7A1 | 3.11E−41 | 0.58265166 | 0.642 | 0.262 | 6.54E−37 |
| USP33 | 3.21E−41 | 0.5747012 | 0.588 | 0.213 | 6.76E−37 |
| FOCAD | 3.50E−41 | 0.46226789 | 0.347 | 0.054 | 7.37E−37 |
| ZNF215 | 3.89E−41 | 0.40853475 | 0.267 | 0.02 | 8.18E−37 |
| AEBP2 | 3.90E−41 | 0.43287058 | 0.316 | 0.039 | 8.20E−37 |
| CATG00000016283.1 | 4.14E−41 | −0.59316965 | 0 | 0.359 | 8.71E−37 |
| MLLT11 | 5.69E−41 | −1.38271016 | 0.453 | 0.738 | 1.20E−36 |
| RP11-166D19.1 | 7.02E−41 | −0.62553916 | 0 | 0.358 | 1.48E−36 |
| ARFGAP2 | 7.11E−41 | 0.57023896 | 0.482 | 0.132 | 1.50E−36 |
| LHX9 | 7.62E−41 | 0.38723837 | 0.295 | 0.029 | 1.60E−36 |
| LHX2 | 9.14E−41 | 0.65089449 | 0.562 | 0.198 | 1.92E−36 |
| CLSTN3 | 9.14E−41 | 0.42549554 | 0.355 | 0.059 | 1.92E−36 |
| SSR1 | 1.05E−40 | 0.55857508 | 0.622 | 0.236 | 2.21E−36 |
| RACGAP1 | 1.23E−40 | 0.72116463 | 0.352 | 0.062 | 2.60E−36 |
| RBM19 | 1.27E−40 | 0.37222105 | 0.264 | 0.02 | 2.68E−36 |
| NUP107 | 1.27E−40 | 0.54889977 | 0.487 | 0.141 | 2.68E−36 |
| LDLR | 1.44E−40 | 0.49269493 | 0.334 | 0.05 | 3.03E−36 |
| MGAT1 | 1.52E−40 | 0.47003475 | 0.345 | 0.055 | 3.20E−36 |
| LNPEP | 1.54E−40 | 0.41129061 | 0.264 | 0.02 | 3.23E−36 |
| DPY19L2 | 1.61E−40 | 0.40023542 | 0.267 | 0.021 | 3.40E−36 |
| PAICS | 1.68E−40 | 0.83673064 | 0.749 | 0.423 | 3.55E−36 |
| FASTKD2 | 1.93E−40 | 0.48796229 | 0.394 | 0.081 | 4.05E−36 |
| RNU6-585P | 2.15E−40 | −0.53471431 | 0 | 0.353 | 4.53E−36 |
| CDC42 | 2.35E−40 | 0.51764632 | 0.845 | 0.416 | 4.96E−36 |
| CEP41 | 2.45E−40 | 0.62191991 | 0.446 | 0.12 | 5.17E−36 |
| MIF | 2.54E−40 | −0.55766264 | 0 | 0.353 | 5.35E−36 |
| RAI1 | 2.57E−40 | 0.39356794 | 0.324 | 0.045 | 5.40E−36 |
| KPNA2 | 2.58E−40 | 1.37523844 | 0.661 | 0.317 | 5.44E−36 |
| IREB2 | 2.63E−40 | 0.43692715 | 0.402 | 0.086 | 5.54E−36 |
| SMARCC2 | 2.63E−40 | 0.54635813 | 0.552 | 0.187 | 5.55E−36 |
| POLA2 | 2.70E−40 | 0.44113518 | 0.29 | 0.031 | 5.67E−36 |
| FEZ1 | 2.73E−40 | −0.74074901 | 0.199 | 0.587 | 5.74E−36 |
| CYTH2 | 2.73E−40 | 0.44819638 | 0.412 | 0.088 | 5.76E−36 |
| MSI2 | 3.42E−40 | 0.59727145 | 0.632 | 0.253 | 7.20E−36 |
| NUP210 | 3.53E−40 | 0.44134209 | 0.298 | 0.034 | 7.43E−36 |
| NCBP1 | 3.72E−40 | 0.41200074 | 0.368 | 0.066 | 7.83E−36 |
| NRIP1 | 3.80E−40 | 0.61924641 | 0.51 | 0.159 | 8.01E−36 |
| VCAN | 4.03E−40 | 0.65476517 | 0.547 | 0.186 | 8.49E−36 |
| COMMD3 | 4.51E−40 | −0.56740521 | 0.013 | 0.374 | 9.50E−36 |
| RCBTB1 | 4.56E−40 | 0.37331373 | 0.311 | 0.039 | 9.61E−36 |
| SEC24B | 4.64E−40 | 0.40038076 | 0.326 | 0.046 | 9.77E−36 |
| MKL2 | 4.69E−40 | 0.45789551 | 0.337 | 0.051 | 9.87E−36 |
| STRIP1 | 4.83E−40 | 0.50908297 | 0.378 | 0.073 | 1.02E−35 |
| PHKB | 4.89E−40 | 0.48517547 | 0.446 | 0.11 | 1.03E−35 |
| SNX27 | 5.10E−40 | 0.50414954 | 0.402 | 0.088 | 1.07E−35 |
| CTPS1 | 5.50E−40 | 0.58699203 | 0.464 | 0.13 | 1.16E−35 |
| 2-Sep | 5.56E−40 | 0.73205106 | 0.917 | 0.731 | 1.17E−35 |
| PREX2 | 5.93E−40 | 0.37487903 | 0.275 | 0.024 | 1.25E−35 |
| NLRP1 | 6.53E−40 | −0.58508579 | 0.003 | 0.357 | 1.37E−35 |
| IPO5 | 8.17E−40 | 0.67021653 | 0.627 | 0.265 | 1.72E−35 |
| TDP1 | 8.50E−40 | 0.39628744 | 0.316 | 0.043 | 1.79E−35 |
| FBXL20 | 9.36E−40 | 0.44991214 | 0.358 | 0.062 | 1.97E−35 |
| CNTN4 | 9.55E−40 | 0.36876962 | 0.269 | 0.023 | 2.01E−35 |
| INPPL1 | 1.03E−39 | 0.37959642 | 0.256 | 0.018 | 2.17E−35 |
| DAXX | 1.07E−39 | 0.4683357 | 0.394 | 0.082 | 2.26E−35 |
| EIF4G1 | 1.12E−39 | 0.56977822 | 0.5 | 0.155 | 2.36E−35 |
| SNX1 | 1.16E−39 | 0.45707088 | 0.407 | 0.088 | 2.45E−35 |
| GPC6 | 1.31E−39 | 0.54335969 | 0.43 | 0.105 | 2.75E−35 |
| FANCG | 1.36E−39 | 0.45591786 | 0.358 | 0.062 | 2.87E−35 |
| ZBTB17 | 1.37E−39 | 0.40711447 | 0.308 | 0.039 | 2.87E−35 |
| ZNF776 | 1.58E−39 | 0.39123421 | 0.313 | 0.042 | 3.32E−35 |
| FNIP1 | 1.60E−39 | 0.36620144 | 0.295 | 0.033 | 3.38E−35 |
| CENPT | 1.70E−39 | 0.48346383 | 0.433 | 0.106 | 3.58E−35 |
| C2CD3 | 1.76E−39 | 0.36370955 | 0.262 | 0.021 | 3.69E−35 |
| IKZF4 | 1.77E−39 | 0.39649071 | 0.277 | 0.027 | 3.72E−35 |
| RIMKLB | 1.79E−39 | 0.5037987 | 0.497 | 0.148 | 3.76E−35 |
| SURF4 | 1.91E−39 | 0.56833161 | 0.578 | 0.203 | 4.02E−35 |
| JAM3 | 1.93E−39 | 0.49777558 | 0.482 | 0.137 | 4.06E−35 |
| PLAG1 | 1.97E−39 | 0.35333958 | 0.275 | 0.026 | 4.15E−35 |
| AIF1L | 2.07E−39 | 0.46979915 | 0.469 | 0.125 | 4.36E−35 |
| RBL1 | 2.23E−39 | 0.3854143 | 0.28 | 0.028 | 4.70E−35 |
| BACH1 | 2.39E−39 | 0.40542809 | 0.293 | 0.033 | 5.03E−35 |
| YWHAG | 2.42E−39 | 0.53590819 | 0.624 | 0.246 | 5.09E−35 |
| ITGB8 | 2.42E−39 | 0.52655187 | 0.435 | 0.112 | 5.10E−35 |
| NOVA1 | 2.56E−39 | −0.67702176 | 0.049 | 0.421 | 5.38E−35 |
| 10-Sep | 2.57E−39 | 0.48270095 | 0.438 | 0.109 | 5.41E−35 |
| CCDC97 | 2.59E−39 | 0.40814759 | 0.298 | 0.035 | 5.46E−35 |
| CRTAP | 2.77E−39 | 0.50483748 | 0.425 | 0.101 | 5.83E−35 |
| RBM39 | 3.01E−39 | 0.59862582 | 0.86 | 0.551 | 6.33E−35 |
| ATG13 | 3.18E−39 | 0.39405506 | 0.337 | 0.053 | 6.70E−35 |
| BRAT1 | 3.30E−39 | 0.47404293 | 0.389 | 0.081 | 6.94E−35 |
| SUPT6H | 3.60E−39 | 0.42586843 | 0.311 | 0.042 | 7.57E−35 |
| PHF8 | 3.68E−39 | 0.37394986 | 0.256 | 0.02 | 7.75E−35 |
| EMC1 | 3.71E−39 | 0.40104453 | 0.313 | 0.043 | 7.81E−35 |
| SIN3A | 3.76E−39 | 0.46985474 | 0.391 | 0.083 | 7.91E−35 |
| ATP6V1G1 | 3.77E−39 | −0.70678804 | 0.648 | 0.837 | 7.93E−35 |
| INO80D | 3.78E−39 | 0.4652529 | 0.313 | 0.044 | 7.96E−35 |
| FLOT2 | 4.21E−39 | 0.43569093 | 0.345 | 0.057 | 8.86E−35 |
| CDCA5 | 4.28E−39 | 0.48930287 | 0.319 | 0.046 | 9.00E−35 |
| NCAPG | 4.42E−39 | 0.67544633 | 0.36 | 0.071 | 9.32E−35 |
| CENPJ | 4.84E−39 | 0.38135396 | 0.288 | 0.032 | 1.02E−34 |
| TBL3 | 4.85E−39 | 0.5269193 | 0.412 | 0.094 | 1.02E−34 |
| EPB41L4A-AS1 | 4.91E−39 | −0.610073 | 0.036 | 0.403 | 1.03E−34 |
| INTS1 | 4.98E−39 | 0.39070484 | 0.29 | 0.033 | 1.05E−34 |
| MRPL42 | 5.59E−39 | 0.57676266 | 0.57 | 0.204 | 1.18E−34 |
| SPRY1 | 5.83E−39 | −0.69561715 | 0.031 | 0.391 | 1.23E−34 |
| UBE4B | 6.03E−39 | 0.43625322 | 0.306 | 0.04 | 1.27E−34 |
| HK1 | 6.23E−39 | 0.59956742 | 0.495 | 0.152 | 1.31E−34 |
| SNF8 | 6.35E−39 | −0.58128914 | 0.057 | 0.434 | 1.34E−34 |
| NFX1 | 7.22E−39 | 0.40007486 | 0.329 | 0.05 | 1.52E−34 |
| SPIDR | 7.73E−39 | 0.42191621 | 0.35 | 0.061 | 1.63E−34 |
| ATP5O | 7.97E−39 | −0.64111831 | 0.687 | 0.837 | 1.68E−34 |
| GYG1 | 8.10E−39 | 0.88360153 | 0.523 | 0.182 | 1.71E−34 |
| DCAF11 | 8.47E−39 | 0.50880583 | 0.415 | 0.095 | 1.78E−34 |
| TEAD1 | 9.65E−39 | 0.47061879 | 0.381 | 0.079 | 2.03E−34 |
| ATR | 1.04E−38 | 0.41294749 | 0.28 | 0.029 | 2.19E−34 |
| DNAJC10 | 1.11E−38 | 0.58929462 | 0.609 | 0.246 | 2.34E−34 |
| MYBBP1A | 1.20E−38 | 0.32969454 | 0.251 | 0.018 | 2.52E−34 |
| GAK | 1.23E−38 | 0.4023805 | 0.303 | 0.039 | 2.60E−34 |
| NBAS | 1.24E−38 | 0.45487853 | 0.381 | 0.078 | 2.61E−34 |
| CD46 | 1.29E−38 | 0.52590847 | 0.536 | 0.18 | 2.72E−34 |
| GADD45GIP1 | 1.35E−38 | −0.56596691 | 0.181 | 0.575 | 2.85E−34 |
| THADA | 1.46E−38 | 0.34538071 | 0.251 | 0.018 | 3.07E−34 |
| ACTB | 1.54E−38 | 0.53078481 | 0.997 | 0.998 | 3.24E−34 |
| TNPO3 | 1.60E−38 | 0.5423734 | 0.464 | 0.13 | 3.37E−34 |
| FAM96B | 1.61E−38 | −0.67778528 | 0.215 | 0.593 | 3.39E−34 |
| ZNF652 | 1.85E−38 | 0.42790195 | 0.365 | 0.07 | 3.89E−34 |
| GGA3 | 1.93E−38 | 0.35965768 | 0.269 | 0.026 | 4.05E−34 |
| PIK3C2A | 2.19E−38 | 0.42575269 | 0.35 | 0.062 | 4.60E−34 |
| SMARCE1 | 2.20E−38 | 0.621109 | 0.782 | 0.441 | 4.64E−34 |
| SAP130 | 2.23E−38 | 0.32038211 | 0.254 | 0.02 | 4.70E−34 |
| PLEKHG4B | 2.29E−38 | 0.46894705 | 0.321 | 0.049 | 4.83E−34 |
| ELAC2 | 2.34E−38 | 0.43541741 | 0.358 | 0.066 | 4.93E−34 |
| ZNF317 | 2.40E−38 | 0.34939406 | 0.267 | 0.024 | 5.06E−34 |
| MON1B | 2.48E−38 | 0.37839067 | 0.288 | 0.033 | 5.22E−34 |
| DNMT1 | 2.49E−38 | 0.64441702 | 0.484 | 0.154 | 5.24E−34 |
| NSUN2 | 2.61E−38 | 0.5102736 | 0.313 | 0.045 | 5.49E−34 |
| ZNF423 | 2.95E−38 | 0.46301558 | 0.373 | 0.077 | 6.21E−34 |
| STMN2 | 3.28E−38 | −1.68156839 | 0.026 | 0.388 | 6.90E−34 |
| ANAPC5 | 3.46E−38 | 0.57273844 | 0.611 | 0.249 | 7.29E−34 |
| FAM13B | 3.48E−38 | 0.54090353 | 0.409 | 0.095 | 7.34E−34 |
| KMT2A | 3.61E−38 | 0.55546456 | 0.505 | 0.161 | 7.61E−34 |
| PPP2R5D | 3.67E−38 | 0.39834032 | 0.311 | 0.044 | 7.73E−34 |
| RAD23B | 3.69E−38 | −0.58943958 | 0.101 | 0.483 | 7.76E−34 |
| EIF3L | 4.00E−38 | −0.5622033 | 0.85 | 0.936 | 8.41E−34 |
| PDIA4 | 4.09E−38 | 0.63414545 | 0.679 | 0.306 | 8.62E−34 |
| DHX15 | 4.17E−38 | 0.59671789 | 0.609 | 0.252 | 8.78E−34 |
| FAM35DP | 4.24E−38 | −0.53333302 | 0.005 | 0.346 | 8.93E−34 |
| ZNHIT1 | 4.28E−38 | −0.68445211 | 0.163 | 0.534 | 9.02E−34 |
| ILDR2 | 4.50E−38 | 0.37487903 | 0.262 | 0.023 | 9.47E−34 |
| SLC7A6 | 4.93E−38 | 0.35401101 | 0.251 | 0.02 | 1.04E−33 |
| ATP5J | 4.93E−38 | −0.67112076 | 0.461 | 0.735 | 1.04E−33 |
| SNHG7 | 5.02E−38 | −0.57683133 | 0.028 | 0.385 | 1.06E−33 |
| SLC37A4 | 5.15E−38 | 0.47014505 | 0.394 | 0.086 | 1.08E−33 |
| XRCC6 | 5.20E−38 | 0.66099567 | 0.868 | 0.664 | 1.10E−33 |
| PRDX2 | 5.29E−38 | −0.65627006 | 0.935 | 0.962 | 1.11E−33 |
| SLC16A3 | 5.65E−38 | −0.74787181 | 0.07 | 0.438 | 1.19E−33 |
| NUP43 | 5.79E−38 | 0.37964965 | 0.285 | 0.033 | 1.22E−33 |
| PTPRD | 6.51E−38 | 0.39394859 | 0.308 | 0.043 | 1.37E−33 |
| ICMT | 6.78E−38 | 0.51616086 | 0.391 | 0.088 | 1.43E−33 |
| IARS2 | 7.33E−38 | 0.52020929 | 0.443 | 0.119 | 1.54E−33 |
| ZDHHC20 | 8.02E−38 | 0.37316256 | 0.269 | 0.027 | 1.69E−33 |
| NDUFB11 | 8.32E−38 | −0.72564598 | 0.396 | 0.696 | 1.75E−33 |
| KDM5A | 8.38E−38 | 0.57442926 | 0.497 | 0.159 | 1.76E−33 |
| MSRB2 | 9.61E−38 | −0.5719101 | 0.018 | 0.364 | 2.02E−33 |
| GAA | 1.05E−37 | 0.35396657 | 0.254 | 0.021 | 2.21E−33 |
| RC3H2 | 1.10E−37 | 0.44968404 | 0.368 | 0.073 | 2.31E−33 |
| EWSR1 | 1.15E−37 | 0.59064061 | 0.834 | 0.512 | 2.42E−33 |
| SOX3 | 1.17E−37 | −0.63705421 | 0.005 | 0.345 | 2.46E−33 |
| TSPYL2 | 1.24E−37 | 0.52710665 | 0.342 | 0.061 | 2.60E−33 |
| NDUFA11 | 1.36E−37 | −0.71844954 | 0.43 | 0.696 | 2.87E−33 |
| TTLL5 | 1.38E−37 | 0.38392082 | 0.254 | 0.021 | 2.90E−33 |
| TARS | 1.40E−37 | 0.57347324 | 0.549 | 0.193 | 2.95E−33 |
| REEP6 | 1.43E−37 | 0.3777652 | 0.301 | 0.04 | 3.01E−33 |
| C17orf62 | 1.46E−37 | 0.49931712 | 0.44 | 0.117 | 3.08E−33 |
| LUZP1 | 1.54E−37 | 0.40139634 | 0.313 | 0.046 | 3.23E−33 |
| ATOX1 | 1.58E−37 | −0.53049155 | 0.049 | 0.414 | 3.33E−33 |
| PPIP5K2 | 1.63E−37 | 0.44829712 | 0.44 | 0.116 | 3.44E−33 |
| EFCAB14 | 1.70E−37 | 0.38137979 | 0.313 | 0.046 | 3.58E−33 |
| LASP1 | 1.73E−37 | 0.43118828 | 0.345 | 0.061 | 3.64E−33 |
| AURKAIP1 | 1.76E−37 | −0.71408495 | 0.29 | 0.627 | 3.70E−33 |
| RASSF4 | 1.77E−37 | 0.41349537 | 0.301 | 0.04 | 3.72E−33 |
| CCDC104 | 2.03E−37 | −0.53471431 | 0 | 0.333 | 4.28E−33 |
| C1orf54 | 2.56E−37 | −0.55675396 | 0.031 | 0.384 | 5.38E−33 |
| ZMYM3 | 2.77E−37 | 0.40748272 | 0.272 | 0.029 | 5.84E−33 |
| ZFP36L2 | 2.83E−37 | 0.73361393 | 0.503 | 0.172 | 5.95E−33 |
| SEC14L1 | 2.90E−37 | 0.4037896 | 0.36 | 0.07 | 6.10E−33 |
| ASF1B | 3.02E−37 | 0.49283353 | 0.347 | 0.064 | 6.35E−33 |
| B3GALNT2 | 3.05E−37 | 0.35839456 | 0.282 | 0.033 | 6.42E−33 |
| HIF1AN | 3.19E−37 | 0.40642846 | 0.329 | 0.055 | 6.71E−33 |
| RPRD2 | 3.48E−37 | 0.40542809 | 0.339 | 0.06 | 7.34E−33 |
| CATG00000005861.1 | 3.58E−37 | −0.50395206 | 0 | 0.33 | 7.54E−33 |
| RP11-649A16.1 | 3.75E−37 | −0.62668189 | 0 | 0.331 | 7.90E−33 |
| AMPD2 | 3.98E−37 | 0.38410959 | 0.313 | 0.048 | 8.37E−33 |
| TWSG1 | 4.06E−37 | 0.45012037 | 0.389 | 0.086 | 8.54E−33 |
| GTPBP1 | 4.10E−37 | 0.47336177 | 0.383 | 0.083 | 8.64E−33 |
| SMG7 | 4.22E−37 | 0.45756571 | 0.342 | 0.062 | 8.89E−33 |
| HSP90AB3P | 4.26E−37 | −0.47251955 | 0 | 0.329 | 8.97E−33 |
| CD276 | 4.49E−37 | 0.54208521 | 0.44 | 0.119 | 9.45E−33 |
| AMOT | 4.53E−37 | 0.37191972 | 0.272 | 0.029 | 9.54E−33 |
| APC | 5.08E−37 | 0.42852777 | 0.295 | 0.039 | 1.07E−32 |
| TCTN2 | 5.23E−37 | 0.34254039 | 0.264 | 0.026 | 1.10E−32 |
| PCED1A | 6.20E−37 | 0.38689214 | 0.28 | 0.033 | 1.31E−32 |
| EIF3H | 6.48E−37 | −0.72016044 | 0.655 | 0.824 | 1.37E−32 |
| CADM1 | 7.37E−37 | 0.63616545 | 0.565 | 0.209 | 1.55E−32 |
| GBF1 | 7.61E−37 | 0.38221338 | 0.295 | 0.04 | 1.60E−32 |
| CHMP2A | 7.74E−37 | −0.72467581 | 0.28 | 0.617 | 1.63E−32 |
| SLC11A2 | 8.12E−37 | 0.50730771 | 0.389 | 0.087 | 1.71E−32 |
| ZBTB41 | 9.06E−37 | 0.45181676 | 0.319 | 0.051 | 1.91E−32 |
| PSMA7 | 1.04E−36 | −0.5863963 | 0.806 | 0.901 | 2.19E−32 |
| SNORA2 | 1.22E−36 | −0.50891831 | 0 | 0.326 | 2.56E−32 |
| EEF1D | 1.27E−36 | −0.62808372 | 0.78 | 0.881 | 2.67E−32 |
| CPT1C | 1.40E−36 | 0.44642546 | 0.389 | 0.088 | 2.94E−32 |
| SDHA | 1.40E−36 | 0.4913777 | 0.547 | 0.191 | 2.95E−32 |
| MACF1 | 1.45E−36 | 0.53430561 | 0.503 | 0.163 | 3.04E−32 |
| SEC31A | 1.48E−36 | 0.53227313 | 0.464 | 0.137 | 3.12E−32 |
| CDC27 | 1.53E−36 | 0.46361551 | 0.399 | 0.097 | 3.23E−32 |
| PCYOX1 | 1.56E−36 | 0.47646471 | 0.477 | 0.143 | 3.28E−32 |
| GULP1 | 1.57E−36 | 0.46166149 | 0.472 | 0.136 | 3.31E−32 |
| AVL9 | 1.60E−36 | 0.35218097 | 0.285 | 0.035 | 3.37E−32 |
| RTFDC1 | 1.69E−36 | 0.45115536 | 0.474 | 0.142 | 3.57E−32 |
| ANKRD36 | 2.07E−36 | 0.38055922 | 0.324 | 0.054 | 4.37E−32 |
| DLGAP5 | 2.23E−36 | 0.93258204 | 0.316 | 0.055 | 4.70E−32 |
| VPS35 | 2.29E−36 | 0.60117754 | 0.689 | 0.346 | 4.81E−32 |
| EZH2 | 2.51E−36 | 0.58472553 | 0.51 | 0.172 | 5.28E−32 |
| BCL2L13 | 2.66E−36 | 0.49932997 | 0.409 | 0.104 | 5.60E−32 |
| UHRF1 | 2.90E−36 | 0.81664466 | 0.451 | 0.139 | 6.11E−32 |
| FTH1P10 | 3.10E−36 | −0.4813899 | 0 | 0.323 | 6.53E−32 |
| HSPA8P8 | 3.43E−36 | −0.4839143 | 0 | 0.323 | 7.21E−32 |
| STAG1 | 3.70E−36 | 0.43393452 | 0.396 | 0.094 | 7.78E−32 |
| CHMP1A | 3.70E−36 | 0.52376638 | 0.521 | 0.177 | 7.79E−32 |
| RRM2 | 4.15E−36 | 0.77417194 | 0.402 | 0.101 | 8.75E−32 |
| LARP4B | 4.94E−36 | 0.38412571 | 0.277 | 0.034 | 1.04E−31 |
| WNK1 | 4.98E−36 | 0.45998425 | 0.521 | 0.176 | 1.05E−31 |
| NFXL1 | 5.02E−36 | 0.37269356 | 0.251 | 0.023 | 1.06E−31 |
| NUP133 | 5.37E−36 | 0.48559844 | 0.381 | 0.088 | 1.13E−31 |
| PPP4R1 | 5.77E−36 | 0.44808847 | 0.326 | 0.056 | 1.21E−31 |
| CCAR2 | 5.96E−36 | 0.44062892 | 0.402 | 0.097 | 1.25E−31 |
| POR | 6.02E−36 | 0.48696114 | 0.526 | 0.18 | 1.27E−31 |
| SALL2 | 6.30E−36 | 0.51039208 | 0.407 | 0.105 | 1.33E−31 |
| WDR1 | 7.49E−36 | 0.61348261 | 0.567 | 0.218 | 1.58E−31 |
| RBMX | 8.06E−36 | 0.58282979 | 0.933 | 0.834 | 1.70E−31 |
| GALC | 8.72E−36 | 0.38454639 | 0.282 | 0.037 | 1.84E−31 |
| ATP6V1F | 9.05E−36 | −0.67524254 | 0.301 | 0.633 | 1.91E−31 |
| FRYL | 1.04E−35 | 0.4631124 | 0.396 | 0.095 | 2.19E−31 |
| AMPH | 1.35E−35 | 0.45471395 | 0.329 | 0.06 | 2.85E−31 |
| EXOC7 | 1.36E−35 | 0.49133481 | 0.536 | 0.192 | 2.86E−31 |
| KIF23 | 1.41E−35 | 0.67691169 | 0.345 | 0.071 | 2.98E−31 |
| NKAIN3 | 1.49E−35 | −0.60941042 | 0.034 | 0.372 | 3.14E−31 |
| UBXN7 | 1.55E−35 | 0.43393452 | 0.394 | 0.094 | 3.26E−31 |
| TXNDC17 | 1.59E−35 | −0.59247344 | 0.093 | 0.456 | 3.35E−31 |
| NDRG3 | 1.66E−35 | 0.51777371 | 0.373 | 0.084 | 3.50E−31 |
| TMEM160 | 1.69E−35 | −0.56049798 | 0.049 | 0.396 | 3.56E−31 |
| RELN | 2.19E−35 | 0.46973347 | 0.285 | 0.039 | 4.61E−31 |
| MAGED1 | 2.21E−35 | 0.6684625 | 0.733 | 0.388 | 4.66E−31 |
| SLC35A4 | 2.27E−35 | 0.42607524 | 0.36 | 0.076 | 4.79E−31 |
| DIS3L | 2.34E−35 | 0.4985375 | 0.355 | 0.073 | 4.93E−31 |
| CCNA2 | 2.38E−35 | 0.94480736 | 0.383 | 0.097 | 5.02E−31 |
| CFL1 | 2.41E−35 | −0.57482396 | 0.953 | 0.988 | 5.08E−31 |
| TELO2 | 2.62E−35 | 0.40877153 | 0.308 | 0.05 | 5.52E−31 |
| CERS5 | 2.70E−35 | 0.42027044 | 0.407 | 0.104 | 5.69E−31 |
| LRRC41 | 2.98E−35 | 0.54663178 | 0.591 | 0.247 | 6.28E−31 |
| EMD | 3.01E−35 | −0.51815503 | 0.031 | 0.369 | 6.34E−31 |
| PCMTD2 | 3.10E−35 | 0.47980093 | 0.433 | 0.122 | 6.52E−31 |
| NAA50 | 3.18E−35 | 0.58511185 | 0.573 | 0.226 | 6.70E−31 |
| EFNB2 | 3.34E−35 | 0.57330526 | 0.352 | 0.076 | 7.03E−31 |
| ABHD2 | 3.38E−35 | 0.34027541 | 0.272 | 0.033 | 7.11E−31 |
| DRAP1 | 3.43E−35 | −0.66057599 | 0.277 | 0.619 | 7.22E−31 |
| JPX | 4.01E−35 | −0.56548933 | 0.041 | 0.381 | 8.43E−31 |
| LDLRAD3 | 4.14E−35 | 0.3931845 | 0.277 | 0.035 | 8.71E−31 |
| GLI3 | 4.35E−35 | 0.50901229 | 0.368 | 0.083 | 9.15E−31 |
| BRIP1 | 4.58E−35 | 0.3550459 | 0.262 | 0.029 | 9.63E−31 |
| PPIB | 4.93E−35 | −0.74496969 | 0.391 | 0.68 | 1.04E−30 |
| UBTF | 5.40E−35 | 0.43164561 | 0.43 | 0.117 | 1.14E−30 |
| CCNT1 | 5.73E−35 | 0.32489926 | 0.277 | 0.035 | 1.21E−30 |
| EMC10 | 6.14E−35 | −0.54688665 | 0.023 | 0.353 | 1.29E−30 |
| COPE | 6.34E−35 | −0.63306289 | 0.609 | 0.791 | 1.33E−30 |
| SERAC1 | 6.45E−35 | 0.36631701 | 0.262 | 0.029 | 1.36E−30 |
| VAMP3 | 6.57E−35 | 0.4378843 | 0.43 | 0.119 | 1.38E−30 |
| MYO3A | 6.64E−35 | 0.33510705 | 0.256 | 0.027 | 1.40E−30 |
| PRTG | 6.72E−35 | 0.41044617 | 0.306 | 0.05 | 1.41E−30 |
| TRMT112 | 7.18E−35 | −0.74188434 | 0.402 | 0.687 | 1.51E−30 |
| GIT2 | 7.21E−35 | 0.3260703 | 0.277 | 0.035 | 1.52E−30 |
| MAP1S | 7.65E−35 | 0.44482691 | 0.391 | 0.097 | 1.61E−30 |
| BIRC6 | 7.67E−35 | 0.55560503 | 0.412 | 0.114 | 1.62E−30 |
| LUC7L | 8.04E−35 | 0.44237645 | 0.474 | 0.145 | 1.69E−30 |
| NNT | 8.10E−35 | 0.44175147 | 0.293 | 0.044 | 1.71E−30 |
| UBE2E1 | 8.13E−35 | −0.44500924 | 0.044 | 0.39 | 1.71E−30 |
| DCTN3 | 8.36E−35 | −0.62299602 | 0.246 | 0.598 | 1.76E−30 |
| PCM1 | 8.66E−35 | 0.6169101 | 0.681 | 0.331 | 1.82E−30 |
| RHEB | 8.79E−35 | −0.69046876 | 0.482 | 0.738 | 1.85E−30 |
| ATG9A | 8.96E−35 | 0.33626607 | 0.267 | 0.032 | 1.89E−30 |
| SEPP1 | 1.06E−34 | 0.42846626 | 0.373 | 0.083 | 2.23E−30 |
| RP11-785H5.1 | 1.07E−34 | −0.50767835 | 0 | 0.312 | 2.25E−30 |
| RPS23P8 | 1.22E−34 | −0.44428084 | 0 | 0.311 | 2.58E−30 |
| GINS1 | 1.23E−34 | 0.37827935 | 0.321 | 0.057 | 2.59E−30 |
| RNF26 | 1.26E−34 | 0.55092208 | 0.409 | 0.11 | 2.65E−30 |
| HSPE1 | 1.28E−34 | −0.63119644 | 0.163 | 0.521 | 2.69E−30 |
| PMS1 | 1.31E−34 | 0.42835449 | 0.334 | 0.066 | 2.75E−30 |
| NAALAD2 | 1.38E−34 | 0.4084202 | 0.321 | 0.059 | 2.91E−30 |
| CCP110 | 1.41E−34 | 0.50251735 | 0.389 | 0.097 | 2.96E−30 |
| HLA-A | 1.51E−34 | −0.74884365 | 0.373 | 0.669 | 3.18E−30 |
| CTBP1 | 1.52E−34 | 0.38705777 | 0.391 | 0.094 | 3.21E−30 |
| BCKDHA | 1.56E−34 | 0.35767496 | 0.301 | 0.048 | 3.28E−30 |
| CELF2 | 1.60E−34 | 0.5735405 | 0.614 | 0.268 | 3.38E−30 |
| FEN1 | 1.64E−34 | 0.64455252 | 0.495 | 0.171 | 3.44E−30 |
| CIAO1 | 1.77E−34 | 0.50920048 | 0.523 | 0.187 | 3.73E−30 |
| EIF5AL1 | 1.93E−34 | −0.45461353 | 0 | 0.309 | 4.06E−30 |
| ID2 | 1.98E−34 | 0.69620373 | 0.63 | 0.275 | 4.17E−30 |
| KIAA0368 | 2.08E−34 | 0.37346036 | 0.311 | 0.053 | 4.37E−30 |
| DDIT4 | 2.11E−34 | −0.72465354 | 0.456 | 0.795 | 4.43E−30 |
| PPM1L | 2.20E−34 | 0.36499846 | 0.264 | 0.032 | 4.63E−30 |
| KIAA0196 | 2.46E−34 | 0.3305297 | 0.259 | 0.029 | 5.18E−30 |
| SHFM1 | 2.57E−34 | −0.56685692 | 0.225 | 0.589 | 5.40E−30 |
| SESN3 | 2.59E−34 | 0.51758325 | 0.373 | 0.089 | 5.45E−30 |
| NPC2 | 2.87E−34 | −0.76783262 | 0.21 | 0.549 | 6.04E−30 |
| DIP2A | 3.13E−34 | 0.42645134 | 0.326 | 0.061 | 6.59E−30 |
| POLR2D | 3.47E−34 | 0.48441639 | 0.425 | 0.12 | 7.30E−30 |
| CTR9 | 3.47E−34 | 0.46703635 | 0.42 | 0.116 | 7.30E−30 |
| UBAC2 | 3.82E−34 | 0.49357368 | 0.536 | 0.199 | 8.05E−30 |
| RPS27L | 3.92E−34 | −0.7054963 | 0.256 | 0.603 | 8.25E−30 |
| BUB1 | 3.97E−34 | 0.68315901 | 0.301 | 0.051 | 8.36E−30 |
| HIGD2A | 3.99E−34 | −0.5582449 | 0.163 | 0.526 | 8.40E−30 |
| HELLS | 4.32E−34 | 0.5062401 | 0.422 | 0.12 | 9.10E−30 |
| RABGAP1 | 4.33E−34 | 0.46361551 | 0.376 | 0.089 | 9.13E−30 |
| NHLRC2 | 5.17E−34 | 0.4119414 | 0.342 | 0.068 | 1.09E−29 |
| THUMPD1 | 5.32E−34 | 0.47198408 | 0.513 | 0.182 | 1.12E−29 |
| CNTNAP2 | 5.39E−34 | −0.54921564 | 0.052 | 0.392 | 1.13E−29 |
| RANBP1 | 5.55E−34 | −0.74175213 | 0.606 | 0.83 | 1.17E−29 |
| SKIV2L | 5.58E−34 | 0.34995311 | 0.254 | 0.028 | 1.18E−29 |
| ZNF384 | 5.61E−34 | 0.32411311 | 0.254 | 0.028 | 1.18E−29 |
| ABL1 | 5.62E−34 | 0.3737716 | 0.29 | 0.044 | 1.18E−29 |
| BMS1 | 5.69E−34 | 0.40226775 | 0.402 | 0.103 | 1.20E−29 |
| FKBP8 | 5.88E−34 | −0.62190672 | 0.264 | 0.614 | 1.24E−29 |
| PPM1G | 5.88E−34 | 0.5683886 | 0.658 | 0.311 | 1.24E−29 |
| COG1 | 6.00E−34 | 0.38811351 | 0.282 | 0.042 | 1.26E−29 |
| E4F1 | 6.21E−34 | 0.34373577 | 0.298 | 0.048 | 1.31E−29 |
| TUG1 | 6.23E−34 | 0.43596079 | 0.389 | 0.097 | 1.31E−29 |
| PDIA3 | 6.42E−34 | 0.58546702 | 0.891 | 0.692 | 1.35E−29 |
| FKBP9 | 7.45E−34 | 0.34767083 | 0.264 | 0.033 | 1.57E−29 |
| PIP5K1A | 7.95E−34 | 0.4503774 | 0.427 | 0.12 | 1.67E−29 |
| RPRD1B | 9.34E−34 | 0.44156531 | 0.363 | 0.082 | 1.97E−29 |
| DMTF1 | 9.61E−34 | 0.42453691 | 0.42 | 0.114 | 2.02E−29 |
| RP11-423H2.1 | 9.96E−34 | −0.48517485 | 0 | 0.304 | 2.10E−29 |
| SON | 1.02E−33 | 0.54558607 | 0.881 | 0.581 | 2.15E−29 |
| NEK4 | 1.07E−33 | 0.36086775 | 0.311 | 0.054 | 2.26E−29 |
| ARMC6 | 1.15E−33 | 0.38672349 | 0.334 | 0.065 | 2.41E−29 |
| RANBP6 | 1.26E−33 | 0.41440651 | 0.306 | 0.053 | 2.64E−29 |
| BCOR | 1.47E−33 | 0.40339946 | 0.275 | 0.038 | 3.10E−29 |
| ZWINT | 1.56E−33 | 0.69315702 | 0.459 | 0.153 | 3.29E−29 |
| NCSTN | 1.66E−33 | 0.46966042 | 0.5 | 0.17 | 3.49E−29 |
| C21orf58 | 2.07E−33 | 0.4226111 | 0.311 | 0.057 | 4.36E−29 |
| CARHSP1 | 2.16E−33 | −0.58836073 | 0.127 | 0.48 | 4.55E−29 |
| ASPM | 2.20E−33 | 0.73414417 | 0.339 | 0.073 | 4.62E−29 |
| KIF4A | 2.47E−33 | 0.48438923 | 0.293 | 0.048 | 5.20E−29 |
| NUP155 | 2.60E−33 | 0.36668879 | 0.28 | 0.042 | 5.46E−29 |
| C5orf24 | 2.71E−33 | 0.36171193 | 0.275 | 0.039 | 5.70E−29 |
| LSMD1 | 3.67E−33 | −0.44168607 | 0 | 0.3 | 7.72E−29 |
| YLPM1 | 3.73E−33 | 0.38460244 | 0.422 | 0.115 | 7.86E−29 |
| PACS2 | 4.36E−33 | 0.38390768 | 0.272 | 0.038 | 9.17E−29 |
| PPCS | 4.67E−33 | 0.31695129 | 0.262 | 0.033 | 9.84E−29 |
| SOX2-OT | 4.71E−33 | −0.56247107 | 0.036 | 0.361 | 9.92E−29 |
| NENF | 4.73E−33 | −0.4853843 | 0.013 | 0.322 | 9.95E−29 |
| FAM103A1 | 4.73E−33 | −0.50146251 | 0.036 | 0.359 | 9.96E−29 |
| COTL1 | 5.06E−33 | 0.66563922 | 0.746 | 0.407 | 1.07E−28 |
| GOLGB1 | 5.08E−33 | 0.5360122 | 0.562 | 0.231 | 1.07E−28 |
| POGZ | 5.10E−33 | 0.43184171 | 0.482 | 0.155 | 1.07E−28 |
| ZNF692 | 6.20E−33 | 0.31745717 | 0.269 | 0.037 | 1.31E−28 |
| THOC2 | 7.26E−33 | 0.44687538 | 0.508 | 0.175 | 1.53E−28 |
| SMDT1 | 7.61E−33 | −0.58215035 | 0.148 | 0.496 | 1.60E−28 |
| RP11-84A1.1 | 7.79E−33 | −0.44038693 | 0 | 0.297 | 1.64E−28 |
| RPL9P7 | 7.79E−33 | −0.44038693 | 0 | 0.297 | 1.64E−28 |
| ELMOD2 | 8.36E−33 | 0.40444298 | 0.332 | 0.068 | 1.76E−28 |
| RPN1 | 8.54E−33 | 0.50657481 | 0.591 | 0.238 | 1.80E−28 |
| SLC35B4 | 8.81E−33 | 0.44957602 | 0.466 | 0.15 | 1.85E−28 |
| TMEM43 | 9.02E−33 | 0.41682285 | 0.383 | 0.098 | 1.90E−28 |
| SNHG16 | 1.02E−32 | 0.6315962 | 0.637 | 0.303 | 2.15E−28 |
| RP3-340B19.2 | 1.08E−32 | −0.44298404 | 0 | 0.296 | 2.28E−28 |
| EPHB2 | 1.29E−32 | 0.38203876 | 0.329 | 0.066 | 2.71E−28 |
| BAX | 1.33E−32 | −0.5588404 | 0.08 | 0.416 | 2.80E−28 |
| INTS4 | 1.34E−32 | 0.37148076 | 0.288 | 0.046 | 2.82E−28 |
| PTPRG | 1.36E−32 | 0.53647598 | 0.446 | 0.143 | 2.86E−28 |
| ZNF532 | 1.53E−32 | 0.50616678 | 0.427 | 0.128 | 3.22E−28 |
| RNF213 | 1.57E−32 | 0.34484803 | 0.277 | 0.042 | 3.31E−28 |
| LEMD1 | 1.62E−32 | −0.48817681 | 0.023 | 0.337 | 3.41E−28 |
| SMARCD1 | 1.64E−32 | 0.43034158 | 0.456 | 0.144 | 3.45E−28 |
| ABI2 | 1.77E−32 | 0.5322827 | 0.674 | 0.336 | 3.72E−28 |
| GDAP2 | 1.78E−32 | 0.39426217 | 0.321 | 0.062 | 3.76E−28 |
| RRP1B | 1.85E−32 | 0.43856647 | 0.407 | 0.114 | 3.90E−28 |
| COPB2 | 1.88E−32 | 0.55827958 | 0.591 | 0.269 | 3.96E−28 |
| TGFBR1 | 2.16E−32 | 0.3697389 | 0.345 | 0.076 | 4.55E−28 |
| ACADVL | 2.19E−32 | 0.59349446 | 0.567 | 0.233 | 4.62E−28 |
| DEK | 2.26E−32 | 0.74933523 | 0.723 | 0.438 | 4.76E−28 |
| CD164 | 2.34E−32 | 0.5866242 | 0.596 | 0.269 | 4.94E−28 |
| VEZF1 | 2.43E−32 | 0.44447576 | 0.521 | 0.193 | 5.11E−28 |
| USP37 | 3.04E−32 | 0.42053498 | 0.277 | 0.043 | 6.40E−28 |
| AARS | 3.14E−32 | 0.45561385 | 0.37 | 0.093 | 6.62E−28 |
| CASP7 | 3.41E−32 | 0.43859495 | 0.275 | 0.042 | 7.18E−28 |
| HJURP | 3.76E−32 | 0.54836096 | 0.29 | 0.051 | 7.91E−28 |
| MYSM1 | 3.98E−32 | 0.33687283 | 0.259 | 0.034 | 8.39E−28 |
| HNRNPF | 4.32E−32 | 0.6733119 | 0.741 | 0.447 | 9.10E−28 |
| WDR34 | 4.41E−32 | 0.51982513 | 0.596 | 0.253 | 9.29E−28 |
| LIMK2 | 4.69E−32 | 0.33178395 | 0.262 | 0.035 | 9.87E−28 |
| BEX2 | 5.47E−32 | −0.62699339 | 0.187 | 0.529 | 1.15E−27 |
| LZTS2 | 5.58E−32 | 0.38537854 | 0.267 | 0.038 | 1.17E−27 |
| GNG3 | 5.62E−32 | −0.70377068 | 0.034 | 0.347 | 1.18E−27 |
| UBE3B | 5.72E−32 | 0.31695129 | 0.272 | 0.04 | 1.20E−27 |
| NOTCH1 | 5.80E−32 | 0.45770771 | 0.313 | 0.062 | 1.22E−27 |
| ARPC3 | 6.96E−32 | −0.64820116 | 0.459 | 0.711 | 1.46E−27 |
| ILVBL | 7.06E−32 | 0.47162483 | 0.503 | 0.182 | 1.49E−27 |
| CCDC107 | 7.09E−32 | −0.45534219 | 0.01 | 0.311 | 1.49E−27 |
| GIGYF2 | 7.22E−32 | 0.4707925 | 0.433 | 0.136 | 1.52E−27 |
| ARNT2 | 7.36E−32 | 0.30027168 | 0.251 | 0.032 | 1.55E−27 |
| CEP112 | 7.39E−32 | 0.35208324 | 0.295 | 0.051 | 1.56E−27 |
| CLK1 | 7.73E−32 | 0.38563422 | 0.373 | 0.093 | 1.63E−27 |
| FAM65A | 7.85E−32 | 0.31898808 | 0.254 | 0.033 | 1.65E−27 |
| ULK3 | 7.86E−32 | 0.3760116 | 0.295 | 0.053 | 1.65E−27 |
| RAB5B | 8.59E−32 | 0.32424633 | 0.259 | 0.035 | 1.81E−27 |
| FGFR1OP | 1.00E−31 | 0.4314233 | 0.394 | 0.11 | 2.11E−27 |
| ZNF385A | 1.07E−31 | 0.380952 | 0.275 | 0.043 | 2.26E−27 |
| LRCH3 | 1.08E−31 | 0.34764459 | 0.301 | 0.055 | 2.28E−27 |
| IQGAP1 | 1.09E−31 | 0.33517454 | 0.256 | 0.034 | 2.29E−27 |
| FAM53C | 1.09E−31 | 0.45258021 | 0.365 | 0.09 | 2.29E−27 |
| RP11-752G15.4 | 1.10E−31 | −0.45846929 | 0 | 0.289 | 2.32E−27 |
| ZMYND8 | 1.17E−31 | 0.4196109 | 0.368 | 0.092 | 2.47E−27 |
| AGL | 1.20E−31 | 0.36920224 | 0.28 | 0.044 | 2.53E−27 |
| PHF6 | 1.41E−31 | 0.52040227 | 0.526 | 0.204 | 2.96E−27 |
| TUBB4B | 1.46E−31 | 1.29920593 | 0.718 | 0.477 | 3.07E−27 |
| GAPDH | 1.54E−31 | −0.49343921 | 0.995 | 1 | 3.25E−27 |
| HNRNPC | 1.65E−31 | 0.50741243 | 0.956 | 0.856 | 3.46E−27 |
| TMEM135 | 1.69E−31 | 0.31594608 | 0.275 | 0.043 | 3.56E−27 |
| SLC35E2 | 1.75E−31 | 0.3415105 | 0.293 | 0.051 | 3.69E−27 |
| NAA16 | 1.97E−31 | 0.37817254 | 0.334 | 0.073 | 4.16E−27 |
| ATF6B | 2.08E−31 | 0.49625294 | 0.438 | 0.134 | 4.37E−27 |
| CRNKL1 | 2.21E−31 | 0.35092952 | 0.316 | 0.064 | 4.65E−27 |
| SPTLC2 | 2.37E−31 | 0.36145039 | 0.298 | 0.055 | 4.98E−27 |
| UQCR10 | 2.38E−31 | −0.60335855 | 0.56 | 0.757 | 5.01E−27 |
| SIPA1L1 | 2.85E−31 | 0.41929074 | 0.306 | 0.06 | 6.00E−27 |
| CHCHD10 | 2.97E−31 | −0.45473657 | 0.003 | 0.29 | 6.25E−27 |
| CLCC1 | 2.97E−31 | 0.32579197 | 0.293 | 0.053 | 6.26E−27 |
| KIDINS220 | 3.46E−31 | 0.46930027 | 0.453 | 0.15 | 7.27E−27 |
| PIGG | 3.51E−31 | 0.33009164 | 0.262 | 0.038 | 7.38E−27 |
| NCLN | 3.69E−31 | 0.33347825 | 0.262 | 0.038 | 7.76E−27 |
| CDCA8 | 3.85E−31 | 0.6196632 | 0.334 | 0.078 | 8.11E−27 |
| SP4 | 4.06E−31 | 0.37496281 | 0.303 | 0.059 | 8.55E−27 |
| ARMC9 | 4.13E−31 | 0.36505869 | 0.321 | 0.067 | 8.68E−27 |
| UACA | 4.31E−31 | 0.35041634 | 0.293 | 0.053 | 9.07E−27 |
| COG5 | 4.47E−31 | 0.36542871 | 0.326 | 0.07 | 9.42E−27 |
| MELK | 4.57E−31 | 0.38882612 | 0.293 | 0.053 | 9.63E−27 |
| CASP3 | 5.17E−31 | 0.66123817 | 0.474 | 0.171 | 1.09E−26 |
| ATAD2B | 6.81E−31 | 0.37451681 | 0.301 | 0.057 | 1.43E−26 |
| SETD5-AS1 | 6.85E−31 | −0.46359432 | 0 | 0.282 | 1.44E−26 |
| PARK7 | 7.35E−31 | −0.55492157 | 0.78 | 0.856 | 1.55E−26 |
| CAST | 7.70E−31 | 0.35557954 | 0.251 | 0.034 | 1.62E−26 |
| USP4 | 7.87E−31 | 0.38140486 | 0.308 | 0.062 | 1.66E−26 |
| SELK | 7.96E−31 | −0.64859437 | 0.35 | 0.642 | 1.68E−26 |
| MKI67 | 8.32E−31 | 1.03614723 | 0.391 | 0.121 | 1.75E−26 |
| SAE1 | 8.65E−31 | 0.57540626 | 0.653 | 0.323 | 1.82E−26 |
| MYADM | 9.19E−31 | 0.4398435 | 0.36 | 0.093 | 1.93E−26 |
| PCGF3 | 9.50E−31 | 0.39285621 | 0.347 | 0.083 | 2.00E−26 |
| SRPK1 | 9.82E−31 | 0.48789025 | 0.526 | 0.204 | 2.07E−26 |
| SDC3 | 9.85E−31 | 0.33817093 | 0.288 | 0.051 | 2.07E−26 |
| CDC23 | 1.05E−30 | 0.39198972 | 0.376 | 0.1 | 2.21E−26 |
| SEC61G | 1.11E−30 | −0.55683472 | 0.111 | 0.447 | 2.33E−26 |
| TXNIP | 1.18E−30 | −0.59441117 | 0.249 | 0.619 | 2.49E−26 |
| MTX3 | 1.26E−30 | 0.33124967 | 0.267 | 0.042 | 2.65E−26 |
| ITCH | 1.29E−30 | 0.36571046 | 0.251 | 0.035 | 2.72E−26 |
| RB1 | 1.41E−30 | 0.42963391 | 0.391 | 0.109 | 2.97E−26 |
| C21orf33 | 1.42E−30 | −0.43621648 | 0.016 | 0.307 | 2.99E−26 |
| CYBA | 1.48E−30 | −0.49423094 | 0.021 | 0.318 | 3.11E−26 |
| FSCN1 | 1.52E−30 | 0.58277069 | 0.707 | 0.413 | 3.20E−26 |
| CEP250 | 1.57E−30 | 0.32320497 | 0.254 | 0.035 | 3.31E−26 |
| TMED9 | 1.58E−30 | −0.61911409 | 0.461 | 0.72 | 3.32E−26 |
| SNRPE | 1.59E−30 | −0.62820269 | 0.332 | 0.628 | 3.34E−26 |
| TMED4 | 1.59E−30 | 0.48396469 | 0.692 | 0.337 | 3.35E−26 |
| NRCAM | 1.61E−30 | 0.41598093 | 0.355 | 0.089 | 3.39E−26 |
| TROAP | 1.63E−30 | 0.63542658 | 0.251 | 0.037 | 3.43E−26 |
| SLC25A6 | 1.70E−30 | −0.41283121 | 0 | 0.279 | 3.58E−26 |
| SUZ12P | 1.83E−30 | −0.44687096 | 0 | 0.279 | 3.85E−26 |
| GYG2 | 1.90E−30 | 0.39262739 | 0.324 | 0.071 | 4.00E−26 |
| POLR2B | 1.96E−30 | 0.46235516 | 0.451 | 0.149 | 4.13E−26 |
| UBL5 | 2.11E−30 | −0.61187855 | 0.539 | 0.73 | 4.45E−26 |
| CKAP4 | 2.18E−30 | 0.56260383 | 0.474 | 0.167 | 4.58E−26 |
| ANXA6 | 2.19E−30 | 0.44542616 | 0.479 | 0.171 | 4.62E−26 |
| FTSJ3 | 2.37E−30 | 0.36440082 | 0.269 | 0.044 | 5.00E−26 |
| SF1 | 2.42E−30 | 0.46163791 | 0.699 | 0.35 | 5.10E−26 |
| SETD2 | 2.52E−30 | 0.47121151 | 0.474 | 0.169 | 5.31E−26 |
| EXO1 | 2.53E−30 | 0.33062425 | 0.254 | 0.037 | 5.34E−26 |
| NRD1 | 2.67E−30 | 0.59545009 | 0.585 | 0.278 | 5.63E−26 |
| AKAP8 | 3.08E−30 | 0.36450514 | 0.254 | 0.037 | 6.49E−26 |
| C5orf51 | 3.13E−30 | 0.3465344 | 0.277 | 0.048 | 6.60E−26 |
| NEFL | 3.23E−30 | −0.68517446 | 0.041 | 0.345 | 6.80E−26 |
| KIAA1429 | 3.71E−30 | 0.37000225 | 0.308 | 0.064 | 7.80E−26 |
| MMRN1 | 3.76E−30 | 0.36429771 | 0.277 | 0.048 | 7.91E−26 |
| POLR3A | 3.99E−30 | 0.34211065 | 0.298 | 0.057 | 8.41E−26 |
| RP11-796G6.1 | 4.74E−30 | −0.40885136 | 0 | 0.275 | 9.98E−26 |
| POMT1 | 5.04E−30 | 0.33028325 | 0.272 | 0.045 | 1.06E−25 |
| ABCC5 | 5.49E−30 | 0.33317602 | 0.285 | 0.051 | 1.16E−25 |
| BTRC | 5.56E−30 | 0.38597271 | 0.293 | 0.056 | 1.17E−25 |
| CPSF3 | 5.92E−30 | 0.43759094 | 0.461 | 0.156 | 1.25E−25 |
| RP11-12M9.3 | 6.06E−30 | −0.38878573 | 0 | 0.274 | 1.28E−25 |
| CPSF2 | 6.35E−30 | 0.42267548 | 0.352 | 0.089 | 1.34E−25 |
| ACTR2 | 6.50E−30 | 0.51019446 | 0.64 | 0.322 | 1.37E−25 |
| AAGAB | 6.94E−30 | 0.42269157 | 0.425 | 0.134 | 1.46E−25 |
| ATP6V0A1 | 6.95E−30 | 0.4107327 | 0.376 | 0.101 | 1.46E−25 |
| CATG00000089634.1 | 7.04E−30 | −0.42339078 | 0 | 0.274 | 1.48E−25 |
| GABARAPL2 | 7.47E−30 | −0.67845998 | 0.5 | 0.711 | 1.57E−25 |
| NDC1 | 7.61E−30 | 0.43625322 | 0.256 | 0.039 | 1.60E−25 |
| HIST1H4C | 8.38E−30 | 1.87644749 | 0.43 | 0.164 | 1.76E−25 |
| SORBS2 | 8.54E−30 | 0.44486431 | 0.399 | 0.12 | 1.80E−25 |
| RNF10 | 9.22E−30 | 0.56664194 | 0.482 | 0.185 | 1.94E−25 |
| KEAP1 | 1.09E−29 | 0.46237783 | 0.368 | 0.099 | 2.29E−25 |
| ERLIN1 | 1.21E−29 | 0.33704112 | 0.282 | 0.051 | 2.55E−25 |
| SOX4 | 1.31E−29 | 0.61128879 | 0.79 | 0.458 | 2.75E−25 |
| PPP2R5C | 1.31E−29 | 0.47248706 | 0.42 | 0.134 | 2.75E−25 |
| NCAPH2 | 1.32E−29 | 0.44624564 | 0.368 | 0.099 | 2.79E−25 |
| FEM1B | 1.33E−29 | 0.36516422 | 0.298 | 0.059 | 2.79E−25 |
| FAM64A | 1.38E−29 | 0.67902069 | 0.368 | 0.104 | 2.90E−25 |
| ATXN2 | 1.44E−29 | 0.41310988 | 0.459 | 0.158 | 3.04E−25 |
| NBN | 1.47E−29 | 0.42278166 | 0.365 | 0.098 | 3.09E−25 |
| RBBP7 | 1.51E−29 | 0.5672205 | 0.806 | 0.601 | 3.18E−25 |
| SUPT4H1 | 1.55E−29 | −0.5400838 | 0.109 | 0.428 | 3.26E−25 |
| WDR36 | 1.58E−29 | 0.34818414 | 0.303 | 0.062 | 3.32E−25 |
| ANKRD36C | 1.60E−29 | 0.35786241 | 0.334 | 0.079 | 3.38E−25 |
| ATAD5 | 1.63E−29 | 0.39905857 | 0.35 | 0.088 | 3.43E−25 |
| CHD6 | 1.79E−29 | 0.40605115 | 0.399 | 0.115 | 3.76E−25 |
| NEK2 | 1.83E−29 | 0.72729008 | 0.259 | 0.043 | 3.86E−25 |
| FARSA | 1.93E−29 | 0.46308866 | 0.523 | 0.2 | 4.06E−25 |
| EPM2AIP1 | 1.96E−29 | 0.50210411 | 0.5 | 0.192 | 4.12E−25 |
| SPCS2 | 1.99E−29 | −0.63197748 | 0.472 | 0.689 | 4.18E−25 |
| EXTL3 | 2.02E−29 | 0.33692958 | 0.256 | 0.04 | 4.25E−25 |
| PRPF39 | 2.07E−29 | 0.37546818 | 0.358 | 0.092 | 4.35E−25 |
| ZKSCAN1 | 2.66E−29 | 0.62541802 | 0.65 | 0.358 | 5.60E−25 |
| DTNA | 2.74E−29 | 0.52025509 | 0.435 | 0.149 | 5.78E−25 |
| WASH2P | 2.77E−29 | −0.40352775 | 0 | 0.269 | 5.83E−25 |
| CDC42EP4 | 3.03E−29 | 0.45900913 | 0.389 | 0.114 | 6.38E−25 |
| RGS12 | 3.03E−29 | 0.32437799 | 0.251 | 0.038 | 6.39E−25 |
| HSD17B4 | 3.21E−29 | 0.51896021 | 0.469 | 0.166 | 6.77E−25 |
| CKAP2L | 3.36E−29 | 0.50017413 | 0.288 | 0.057 | 7.07E−25 |
| SLC25A24 | 3.54E−29 | 0.40701086 | 0.368 | 0.101 | 7.46E−25 |
| NDUFA2 | 3.59E−29 | −0.50039818 | 0.14 | 0.467 | 7.57E−25 |
| ARHGEF40 | 3.82E−29 | 0.37270646 | 0.303 | 0.065 | 8.04E−25 |
| TAS2R14 | 4.12E−29 | −0.42097259 | 0.003 | 0.273 | 8.66E−25 |
| MAFB | 4.23E−29 | 0.46705307 | 0.251 | 0.039 | 8.90E−25 |
| RNF216 | 4.31E−29 | 0.36636742 | 0.329 | 0.077 | 9.08E−25 |
| C17orf89 | 4.58E−29 | −0.4330072 | 0.036 | 0.334 | 9.64E−25 |
| DYNC2H1 | 4.58E−29 | 0.50657481 | 0.342 | 0.087 | 9.65E−25 |
| NRAS | 4.61E−29 | 0.48225241 | 0.466 | 0.171 | 9.70E−25 |
| MKI67IP | 4.67E−29 | −0.45332598 | 0 | 0.268 | 9.82E−25 |
| TUBB3.1 | 4.71E−29 | −0.47505951 | 0 | 0.268 | 9.92E−25 |
| EDEM3 | 4.83E−29 | 0.33812216 | 0.269 | 0.046 | 1.02E−24 |
| VPS41 | 4.85E−29 | 0.44158161 | 0.391 | 0.116 | 1.02E−24 |
| PRPF40A | 4.91E−29 | 0.52225659 | 0.746 | 0.418 | 1.03E−24 |
| LPP | 5.08E−29 | 0.31020971 | 0.256 | 0.04 | 1.07E−24 |
| TPI1 | 5.13E−29 | −0.56091074 | 0.933 | 0.991 | 1.08E−24 |
| TK1 | 5.30E−29 | 0.40893094 | 0.321 | 0.072 | 1.12E−24 |
| BLOC1S4 | 6.40E−29 | −0.43645972 | 0.028 | 0.318 | 1.35E−24 |
| CHMP5 | 6.81E−29 | −0.52889099 | 0.264 | 0.578 | 1.43E−24 |
| SUN1 | 7.02E−29 | 0.45574256 | 0.443 | 0.155 | 1.48E−24 |
| FZD3 | 7.39E−29 | 0.59157182 | 0.658 | 0.35 | 1.56E−24 |
| AMD1 | 8.45E−29 | 0.49349939 | 0.484 | 0.191 | 1.78E−24 |
| SFXN1 | 9.45E−29 | 0.48064029 | 0.583 | 0.268 | 1.99E−24 |
| EXOC6B | 1.15E−28 | 0.30349949 | 0.269 | 0.048 | 2.42E−24 |
| TAF15 | 1.22E−28 | −0.4981113 | 0.101 | 0.417 | 2.56E−24 |
| GMFB | 1.25E−28 | 0.46642531 | 0.427 | 0.143 | 2.64E−24 |
| COX20 | 1.30E−28 | −0.43256742 | 0 | 0.264 | 2.74E−24 |
| NES | 1.47E−28 | −0.57634568 | 0.145 | 0.463 | 3.09E−24 |
| RNF40 | 1.54E−28 | 0.42524199 | 0.456 | 0.165 | 3.25E−24 |
| BOD1 | 1.55E−28 | −0.55701591 | 0.187 | 0.504 | 3.26E−24 |
| KDM4A | 1.55E−28 | 0.35254678 | 0.303 | 0.066 | 3.26E−24 |
| RPS26 | 1.74E−28 | −0.5369235 | 0.368 | 0.648 | 3.66E−24 |
| MXRA8 | 1.96E−28 | 0.50930084 | 0.277 | 0.055 | 4.12E−24 |
| KIAA1551 | 2.01E−28 | 0.3735907 | 0.347 | 0.092 | 4.24E−24 |
| KRTCAP2 | 2.15E−28 | 0.34314381 | 0.251 | 0.04 | 4.53E−24 |
| CCDC93 | 2.24E−28 | 0.33709592 | 0.288 | 0.057 | 4.71E−24 |
| MAP3K7 | 2.33E−28 | 0.4514456 | 0.49 | 0.189 | 4.90E−24 |
| NUSAP1 | 2.37E−28 | 0.90325047 | 0.435 | 0.154 | 5.00E−24 |
| GTF3C5 | 2.40E−28 | 0.44624042 | 0.412 | 0.134 | 5.06E−24 |
| ZFYVE27 | 2.44E−28 | 0.31494951 | 0.269 | 0.049 | 5.14E−24 |
| DCXR | 2.45E−28 | −0.56829822 | 0.194 | 0.507 | 5.16E−24 |
| STAM2 | 2.49E−28 | 0.31258707 | 0.262 | 0.044 | 5.23E−24 |
| WARS | 2.54E−28 | 0.35328447 | 0.373 | 0.104 | 5.34E−24 |
| SCNM1 | 2.55E−28 | −0.42733078 | 0 | 0.262 | 5.37E−24 |
| BBS2 | 2.59E−28 | 0.43638102 | 0.36 | 0.099 | 5.45E−24 |
| AP1B1 | 2.64E−28 | 0.31562836 | 0.277 | 0.053 | 5.55E−24 |
| UBE4A | 2.81E−28 | 0.38266257 | 0.35 | 0.092 | 5.91E−24 |
| RNF181 | 2.96E−28 | −0.50255753 | 0.104 | 0.417 | 6.22E−24 |
| MAP1LC3A | 3.00E−28 | −0.54194157 | 0.062 | 0.357 | 6.31E−24 |
| TMEM14D | 3.17E−28 | −0.38878573 | 0 | 0.26 | 6.66E−24 |
| RP11-463M16.4 | 3.17E−28 | −0.39013216 | 0 | 0.26 | 6.67E−24 |
| NUFIP2 | 3.43E−28 | 0.4268554 | 0.422 | 0.138 | 7.22E−24 |
| FAN1 | 3.76E−28 | 0.28871583 | 0.262 | 0.045 | 7.91E−24 |
| ANAPC7 | 3.76E−28 | 0.37615317 | 0.373 | 0.108 | 7.91E−24 |
| PRC1 | 3.78E−28 | 0.58845413 | 0.337 | 0.089 | 7.96E−24 |
| MAPRE2 | 3.90E−28 | 0.41944286 | 0.311 | 0.071 | 8.21E−24 |
| FMO5 | 4.07E−28 | −0.36707025 | 0 | 0.259 | 8.57E−24 |
| RP11-620J15.3 | 4.31E−28 | −0.38203469 | 0 | 0.259 | 9.08E−24 |
| DCLK1 | 4.33E−28 | 0.41907295 | 0.347 | 0.093 | 9.11E−24 |
| PNN | 4.55E−28 | 0.47302293 | 0.712 | 0.383 | 9.58E−24 |
| SURF1 | 4.71E−28 | −0.47878003 | 0.041 | 0.331 | 9.91E−24 |
| COG2 | 4.91E−28 | 0.33099156 | 0.288 | 0.059 | 1.03E−23 |
| CCT5 | 5.82E−28 | 0.54611856 | 0.785 | 0.526 | 1.22E−23 |
| LAGE3 | 5.94E−28 | −0.46153632 | 0.034 | 0.319 | 1.25E−23 |
| DYNLL1 | 6.14E−28 | −0.59583246 | 0.736 | 0.881 | 1.29E−23 |
| TRIM27 | 6.21E−28 | 0.47131603 | 0.435 | 0.152 | 1.31E−23 |
| TUBGCP3 | 6.22E−28 | 0.28447956 | 0.269 | 0.049 | 1.31E−23 |
| TUBGCP5 | 6.46E−28 | 0.30830374 | 0.254 | 0.043 | 1.36E−23 |
| GNAI3 | 6.56E−28 | 0.56960351 | 0.666 | 0.359 | 1.38E−23 |
| DDHD2 | 6.57E−28 | 0.41644709 | 0.363 | 0.104 | 1.38E−23 |
| SMEK2 | 6.68E−28 | −0.39952212 | 0 | 0.258 | 1.41E−23 |
| NDUFAB1 | 6.80E−28 | −0.54743793 | 0.298 | 0.599 | 1.43E−23 |
| ATPIF1 | 7.16E−28 | −0.57040986 | 0.404 | 0.655 | 1.51E−23 |
| XBP1 | 7.69E−28 | −0.57446087 | 0.137 | 0.45 | 1.62E−23 |
| LAMP1 | 7.75E−28 | −0.42868507 | 0.018 | 0.29 | 1.63E−23 |
| FKBP1A | 7.87E−28 | −0.61643134 | 0.547 | 0.724 | 1.66E−23 |
| TLDC1 | 8.35E−28 | 0.35208324 | 0.269 | 0.05 | 1.76E−23 |
| HK2 | 9.50E−28 | 0.43422148 | 0.298 | 0.066 | 2.00E−23 |
| SBNO1 | 1.02E−27 | 0.54731301 | 0.557 | 0.252 | 2.15E−23 |
| DDX39A | 1.11E−27 | 0.53172059 | 0.477 | 0.182 | 2.34E−23 |
| MLLT10 | 1.15E−27 | 0.42331416 | 0.474 | 0.175 | 2.42E−23 |
| DNAJA3 | 1.16E−27 | 0.31699013 | 0.316 | 0.073 | 2.43E−23 |
| SPCS1 | 1.19E−27 | −0.5880586 | 0.363 | 0.642 | 2.50E−23 |
| GAPVD1 | 1.25E−27 | 0.38797912 | 0.363 | 0.104 | 2.63E−23 |
| R3HDM1 | 1.56E−27 | 0.40893094 | 0.352 | 0.098 | 3.29E−23 |
| PMPCA | 1.62E−27 | 0.37967852 | 0.44 | 0.154 | 3.41E−23 |
| FEZ2 | 1.71E−27 | −0.40430905 | 0.008 | 0.269 | 3.60E−23 |
| LMNB1 | 1.75E−27 | 0.53947268 | 0.482 | 0.192 | 3.69E−23 |
| ACN9 | 1.79E−27 | −0.3847389 | 0 | 0.254 | 3.77E−23 |
| IGFBPL1 | 1.81E−27 | 0.75948758 | 0.446 | 0.171 | 3.80E−23 |
| ADAM17 | 1.82E−27 | 0.31812752 | 0.326 | 0.081 | 3.84E−23 |
| AKAP12 | 1.85E−27 | 0.69712319 | 0.479 | 0.203 | 3.90E−23 |
| PDS5A | 1.86E−27 | 0.3345843 | 0.311 | 0.072 | 3.93E−23 |
| ITGB1 | 1.98E−27 | 0.41031031 | 0.407 | 0.134 | 4.16E−23 |
| RPL38 | 2.00E−27 | −0.46992693 | 0.622 | 0.773 | 4.22E−23 |
| RNF6 | 2.13E−27 | 0.32716908 | 0.293 | 0.064 | 4.49E−23 |
| WAC | 2.15E−27 | 0.41616337 | 0.435 | 0.148 | 4.53E−23 |
| SF3B4 | 2.63E−27 | 0.31947251 | 0.262 | 0.048 | 5.54E−23 |
| BMPR1A | 3.02E−27 | 0.35552978 | 0.345 | 0.092 | 6.36E−23 |
| MASP1 | 3.24E−27 | −0.53105729 | 0 | 0.253 | 6.82E−23 |
| NDC80 | 3.26E−27 | 0.49128168 | 0.288 | 0.062 | 6.86E−23 |
| DGUOK | 3.37E−27 | −0.55659254 | 0.298 | 0.589 | 7.09E−23 |
| NUF2 | 3.57E−27 | 0.5723388 | 0.308 | 0.075 | 7.52E−23 |
| CDCA4 | 3.62E−27 | 0.48335109 | 0.394 | 0.127 | 7.62E−23 |
| SNRPF | 3.67E−27 | −0.49520352 | 0.166 | 0.48 | 7.72E−23 |
| PTTG1IP | 3.69E−27 | 0.51749858 | 0.692 | 0.401 | 7.76E−23 |
| NDUFB9 | 3.92E−27 | −0.51268976 | 0.275 | 0.576 | 8.25E−23 |
| VPS52 | 4.45E−27 | 0.41741417 | 0.316 | 0.078 | 9.36E−23 |
| LMO1 | 5.10E−27 | −0.48414006 | 0.008 | 0.267 | 1.07E−22 |
| RPL23 | 5.12E−27 | −0.495434 | 0.938 | 0.94 | 1.08E−22 |
| VDAC2 | 5.25E−27 | −0.61912633 | 0.619 | 0.779 | 1.11E−22 |
| AHCY | 5.45E−27 | 0.51668484 | 0.642 | 0.342 | 1.15E−22 |
| EPB41L5 | 5.51E−27 | 0.30004538 | 0.29 | 0.062 | 1.16E−22 |
| PON2 | 5.62E−27 | −0.6555974 | 0.311 | 0.598 | 1.18E−22 |
| MIA3 | 5.76E−27 | 0.36898316 | 0.36 | 0.104 | 1.21E−22 |
| PNP | 6.14E−27 | 0.37300661 | 0.269 | 0.053 | 1.29E−22 |
| PIGQ | 6.58E−27 | 0.31366215 | 0.285 | 0.06 | 1.38E−22 |
| COX5A | 6.70E−27 | −0.52919416 | 0.549 | 0.743 | 1.41E−22 |
| SPTBN1 | 7.25E−27 | 0.39739972 | 0.337 | 0.092 | 1.53E−22 |
| GCSH | 7.27E−27 | −0.50146251 | 0.135 | 0.436 | 1.53E−22 |
| CLCN3 | 7.64E−27 | 0.4042402 | 0.448 | 0.164 | 1.61E−22 |
| EHMT1 | 7.89E−27 | 0.39760859 | 0.417 | 0.141 | 1.66E−22 |
| CSNK1A1 | 8.16E−27 | 0.5003765 | 0.624 | 0.32 | 1.72E−22 |
| DHX40 | 8.20E−27 | 0.40200015 | 0.386 | 0.121 | 1.73E−22 |
| ERAL1 | 8.56E−27 | 0.39819652 | 0.427 | 0.144 | 1.80E−22 |
| PCNXL4 | 9.17E−27 | 0.45180645 | 0.482 | 0.189 | 1.93E−22 |
| ELAVL3 | 9.68E−27 | −0.45192304 | 0.01 | 0.269 | 2.04E−22 |
| MAN1B1 | 9.82E−27 | 0.33032417 | 0.319 | 0.078 | 2.07E−22 |
| TM9SF2 | 1.04E−26 | 0.4357577 | 0.575 | 0.259 | 2.19E−22 |
| ATM | 1.07E−26 | 0.34545817 | 0.332 | 0.088 | 2.24E−22 |
| USP47 | 1.07E−26 | 0.47849494 | 0.528 | 0.237 | 2.24E−22 |
| CHMP4B | 1.07E−26 | −0.41570727 | 0.003 | 0.253 | 2.24E−22 |
| DDX56 | 1.15E−26 | 0.39503292 | 0.451 | 0.165 | 2.42E−22 |
| CCDC117 | 1.18E−26 | 0.40143355 | 0.272 | 0.055 | 2.48E−22 |
| XPC | 1.19E−26 | 0.39078764 | 0.389 | 0.125 | 2.50E−22 |
| MRE11A | 1.22E−26 | 0.32158804 | 0.285 | 0.061 | 2.57E−22 |
| ARHGAP35 | 1.23E−26 | 0.41280762 | 0.396 | 0.127 | 2.59E−22 |
| KIF20B | 1.34E−26 | 0.39634226 | 0.311 | 0.077 | 2.81E−22 |
| ZW10 | 1.59E−26 | 0.31779787 | 0.262 | 0.05 | 3.35E−22 |
| ATXN10 | 1.60E−26 | 0.49952328 | 0.777 | 0.522 | 3.38E−22 |
| ZMIZ1 | 1.64E−26 | 0.34327775 | 0.306 | 0.072 | 3.45E−22 |
| CLTB | 1.65E−26 | −0.43000937 | 0.047 | 0.325 | 3.48E−22 |
| TRAPPC6A | 1.69E−26 | −0.45148901 | 0.067 | 0.357 | 3.55E−22 |
| DYNLRB1 | 1.69E−26 | −0.55177328 | 0.293 | 0.579 | 3.56E−22 |
| DUSP15 | 1.81E−26 | −0.4216092 | 0.01 | 0.265 | 3.80E−22 |
| MAD1L1 | 2.02E−26 | 0.36078606 | 0.285 | 0.062 | 4.25E−22 |
| TRIOBP | 2.03E−26 | 0.36600865 | 0.295 | 0.068 | 4.28E−22 |
| TPM2 | 2.25E−26 | 0.3454143 | 0.251 | 0.045 | 4.74E−22 |
| TRIP13 | 2.32E−26 | 0.31697109 | 0.269 | 0.054 | 4.88E−22 |
| DPCD | 2.33E−26 | −0.50974793 | 0.153 | 0.455 | 4.91E−22 |
| TCEB2 | 2.43E−26 | −0.52951674 | 0.593 | 0.76 | 5.11E−22 |
| THOC1 | 2.43E−26 | 0.40416754 | 0.373 | 0.114 | 5.12E−22 |
| KLHL12 | 2.49E−26 | 0.30122199 | 0.311 | 0.076 | 5.24E−22 |
| ARMC8 | 2.52E−26 | 0.4124634 | 0.396 | 0.13 | 5.31E−22 |
| AP2M1 | 2.78E−26 | 0.51147324 | 0.87 | 0.661 | 5.85E−22 |
| KIF1A | 2.79E−26 | 0.51381719 | 0.609 | 0.304 | 5.87E−22 |
| SHISA5 | 2.86E−26 | 0.48454422 | 0.518 | 0.226 | 6.01E−22 |
| ZNF33B | 2.87E−26 | 0.3077907 | 0.275 | 0.057 | 6.05E−22 |
| EBPL | 2.96E−26 | −0.45783048 | 0.057 | 0.34 | 6.24E−22 |
| CHD8 | 3.00E−26 | 0.36182906 | 0.342 | 0.094 | 6.31E−22 |
| TMEM19 | 3.16E−26 | 0.3068507 | 0.251 | 0.045 | 6.66E−22 |
| CITED2 | 3.23E−26 | −0.5292824 | 0.098 | 0.389 | 6.80E−22 |
| TCEA2 | 3.31E−26 | −0.43039089 | 0.073 | 0.362 | 6.98E−22 |
| PANK4 | 3.42E−26 | 0.29830925 | 0.282 | 0.061 | 7.20E−22 |
| PFDN6 | 3.54E−26 | −0.39131873 | 0.01 | 0.263 | 7.45E−22 |
| C12orf75 | 3.63E−26 | −0.43289822 | 0.018 | 0.278 | 7.64E−22 |
| PSPC1 | 3.69E−26 | 0.43283425 | 0.508 | 0.209 | 7.77E−22 |
| TRMT1 | 3.96E−26 | 0.35244599 | 0.321 | 0.083 | 8.33E−22 |
| SMC4 | 4.01E−26 | 0.69552151 | 0.492 | 0.215 | 8.44E−22 |
| UBR5 | 4.01E−26 | 0.40031215 | 0.339 | 0.097 | 8.45E−22 |
| PTDSS1 | 4.22E−26 | 0.39245024 | 0.389 | 0.126 | 8.89E−22 |
| TPR | 4.70E−26 | 0.5093656 | 0.671 | 0.389 | 9.90E−22 |
| GUSB | 5.24E−26 | 0.33609895 | 0.368 | 0.109 | 1.10E−21 |
| ZFAND5 | 5.31E−26 | 0.46140726 | 0.575 | 0.274 | 1.12E−21 |
| CLSPN | 5.54E−26 | 0.44308876 | 0.339 | 0.095 | 1.17E−21 |
| BAZ2A | 5.62E−26 | 0.40764835 | 0.35 | 0.105 | 1.18E−21 |
| BRCA1 | 6.05E−26 | 0.35956108 | 0.29 | 0.067 | 1.27E−21 |
| NDUFAF3 | 6.91E−26 | −0.55033829 | 0.22 | 0.509 | 1.45E−21 |
| AURKB | 7.06E−26 | 0.60848978 | 0.337 | 0.097 | 1.49E−21 |
| HMGA1 | 7.40E−26 | 0.69493471 | 0.78 | 0.571 | 1.56E−21 |
| RAB3GAP2 | 7.61E−26 | 0.32326899 | 0.308 | 0.076 | 1.60E−21 |
| TBRG4 | 7.71E−26 | 0.36763452 | 0.345 | 0.1 | 1.62E−21 |
| ABCF2 | 7.91E−26 | 0.35339559 | 0.329 | 0.088 | 1.66E−21 |
| CTDSPL2 | 8.02E−26 | 0.4305607 | 0.42 | 0.152 | 1.69E−21 |
| SPDL1 | 8.57E−26 | 0.38843345 | 0.275 | 0.06 | 1.80E−21 |
| IVD | 8.66E−26 | 0.35073745 | 0.358 | 0.106 | 1.82E−21 |
| SHROOM3 | 8.74E−26 | 0.3839963 | 0.42 | 0.147 | 1.84E−21 |
| RAP2B | 9.42E−26 | 0.30109143 | 0.251 | 0.046 | 1.98E−21 |
| SEC23B | 9.49E−26 | 0.43629692 | 0.44 | 0.167 | 2.00E−21 |
| TNFRSF10B | 9.51E−26 | 0.44744244 | 0.365 | 0.115 | 2.00E−21 |
| KRT8 | 9.83E−26 | 0.48559844 | 0.313 | 0.082 | 2.07E−21 |
| ELP3 | 9.96E−26 | 0.31647717 | 0.277 | 0.06 | 2.10E−21 |
| ACOX1 | 1.07E−25 | 0.36028756 | 0.269 | 0.056 | 2.25E−21 |
| GNRH1 | 1.07E−25 | −2.64407054 | 0.008 | 0.254 | 2.26E−21 |
| DDX3X | 1.14E−25 | 0.48853957 | 0.56 | 0.259 | 2.39E−21 |
| CCDC167 | 1.14E−25 | −0.49172549 | 0.127 | 0.428 | 2.40E−21 |
| PLK1 | 1.15E−25 | 0.86525623 | 0.254 | 0.05 | 2.42E−21 |
| ANK2 | 1.20E−25 | 0.37508222 | 0.288 | 0.066 | 2.53E−21 |
| ATN1 | 1.21E−25 | 0.31531567 | 0.277 | 0.06 | 2.54E−21 |
| BCAT2 | 1.26E−25 | 0.34218566 | 0.311 | 0.079 | 2.66E−21 |
| RTKN2 | 1.27E−25 | 0.38182315 | 0.272 | 0.059 | 2.68E−21 |
| ZNF106 | 1.28E−25 | 0.41812849 | 0.404 | 0.137 | 2.69E−21 |
| STAG2 | 1.31E−25 | 0.40208676 | 0.404 | 0.138 | 2.77E−21 |
| C20orf96 | 1.35E−25 | 0.30888963 | 0.311 | 0.077 | 2.85E−21 |
| CEP97 | 1.44E−25 | 0.32313736 | 0.311 | 0.077 | 3.03E−21 |
| UQCRQ | 1.51E−25 | −0.57626436 | 0.386 | 0.643 | 3.19E−21 |
| AQR | 1.61E−25 | 0.34761346 | 0.319 | 0.084 | 3.39E−21 |
| EEF1E1 | 1.62E−25 | −0.40811203 | 0.031 | 0.293 | 3.41E−21 |
| PRR14L | 1.63E−25 | 0.36095632 | 0.37 | 0.115 | 3.43E−21 |
| FUBP1 | 1.64E−25 | 0.49793272 | 0.642 | 0.344 | 3.44E−21 |
| ANKZF1 | 1.66E−25 | 0.42278166 | 0.339 | 0.097 | 3.50E−21 |
| SNHG8 | 1.73E−25 | −0.43587416 | 0.047 | 0.317 | 3.65E−21 |
| TXNL4A | 1.77E−25 | −0.4515866 | 0.054 | 0.329 | 3.73E−21 |
| METTL9 | 1.82E−25 | −0.5374794 | 0.352 | 0.622 | 3.83E−21 |
| FAM13A | 1.82E−25 | 0.32525774 | 0.311 | 0.079 | 3.84E−21 |
| NDUFB5 | 1.91E−25 | −0.55258697 | 0.262 | 0.546 | 4.03E−21 |
| MAGOH | 1.92E−25 | −0.4443186 | 0.062 | 0.34 | 4.05E−21 |
| RANBP3 | 2.00E−25 | 0.42991657 | 0.412 | 0.143 | 4.22E−21 |
| LARS | 2.10E−25 | 0.5140089 | 0.57 | 0.284 | 4.42E−21 |
| LINC00461 | 2.11E−25 | −0.44086798 | 0.008 | 0.252 | 4.45E−21 |
| H2AFY2 | 2.21E−25 | 0.52201893 | 0.785 | 0.543 | 4.66E−21 |
| PITPNB | 2.23E−25 | 0.39341655 | 0.456 | 0.178 | 4.70E−21 |
| SNX14 | 2.27E−25 | 0.39491486 | 0.345 | 0.1 | 4.77E−21 |
| USP39 | 2.30E−25 | 0.35600127 | 0.407 | 0.138 | 4.85E−21 |
| TOMM34 | 2.30E−25 | 0.33503876 | 0.301 | 0.075 | 4.85E−21 |
| PAF1 | 2.34E−25 | 0.33399626 | 0.308 | 0.079 | 4.92E−21 |
| MORC3 | 2.46E−25 | 0.39086198 | 0.282 | 0.064 | 5.18E−21 |
| IP6K1 | 2.57E−25 | 0.31482554 | 0.282 | 0.065 | 5.41E−21 |
| NOP58 | 2.68E−25 | 0.41090322 | 0.521 | 0.225 | 5.63E−21 |
| DHX30 | 2.95E−25 | 0.37481354 | 0.383 | 0.126 | 6.22E−21 |
| POU2F1 | 3.21E−25 | 0.36461676 | 0.365 | 0.114 | 6.76E−21 |
| SHPRH | 3.82E−25 | 0.38460244 | 0.376 | 0.12 | 8.05E−21 |
| ZNF346 | 4.29E−25 | 0.38650081 | 0.311 | 0.081 | 9.03E−21 |
| APBB1 | 4.43E−25 | 0.38828581 | 0.461 | 0.181 | 9.33E−21 |
| CTSB | 4.46E−25 | 0.43834271 | 0.516 | 0.225 | 9.39E−21 |
| AP2S1 | 4.50E−25 | −0.59373678 | 0.36 | 0.604 | 9.47E−21 |
| ACTL6A | 4.52E−25 | 0.44717707 | 0.606 | 0.309 | 9.51E−21 |
| PHYHIPL | 4.52E−25 | 0.52222303 | 0.466 | 0.193 | 9.52E−21 |
| SRSF10 | 4.54E−25 | 0.52338646 | 0.746 | 0.483 | 9.57E−21 |
| ATP5G3 | 4.58E−25 | −0.51567 | 0.64 | 0.781 | 9.64E−21 |
| KIAA0355 | 4.72E−25 | 0.29259559 | 0.269 | 0.057 | 9.95E−21 |
| SEL1L | 4.78E−25 | 0.32300424 | 0.306 | 0.078 | 1.01E−20 |
| CCNG2 | 5.25E−25 | −0.449122 | 0.078 | 0.358 | 1.10E−20 |
| RNF38 | 5.39E−25 | 0.39087978 | 0.324 | 0.089 | 1.13E−20 |
| UBE2E3 | 5.53E−25 | −0.60917632 | 0.482 | 0.691 | 1.16E−20 |
| ZNF827 | 5.53E−25 | 0.35990517 | 0.319 | 0.087 | 1.16E−20 |
| MRPL41 | 5.54E−25 | −0.45888272 | 0.119 | 0.414 | 1.17E−20 |
| BAIAP2 | 5.92E−25 | 0.35718165 | 0.319 | 0.084 | 1.25E−20 |
| DHX16 | 6.13E−25 | 0.36681964 | 0.269 | 0.059 | 1.29E−20 |
| UGGT2 | 6.15E−25 | 0.29456441 | 0.313 | 0.082 | 1.30E−20 |
| U2SURP | 6.53E−25 | 0.44346358 | 0.562 | 0.27 | 1.37E−20 |
| DIDO1 | 6.78E−25 | 0.37356819 | 0.378 | 0.125 | 1.43E−20 |
| TUBA1A | 6.92E−25 | −0.84867704 | 0.979 | 0.995 | 1.46E−20 |
| SCYL2 | 7.54E−25 | 0.44201731 | 0.347 | 0.104 | 1.59E−20 |
| DCAF12 | 7.75E−25 | 0.38037477 | 0.389 | 0.131 | 1.63E−20 |
| RFX3 | 8.04E−25 | 0.41323783 | 0.415 | 0.148 | 1.69E−20 |
| CTNNAL1 | 8.25E−25 | 0.38647315 | 0.396 | 0.136 | 1.74E−20 |
| WEE1 | 8.28E−25 | 0.31385149 | 0.277 | 0.062 | 1.74E−20 |
| TCEAL2 | 8.83E−25 | −0.40638891 | 0.016 | 0.26 | 1.86E−20 |
| MED23 | 9.00E−25 | 0.28571454 | 0.262 | 0.055 | 1.89E−20 |
| CDK16 | 9.00E−25 | 0.48570205 | 0.583 | 0.301 | 1.90E−20 |
| HMGB3 | 9.19E−25 | 0.7282473 | 0.718 | 0.468 | 1.94E−20 |
| CLEC16A | 9.20E−25 | 0.37731738 | 0.293 | 0.072 | 1.94E−20 |
| COPG1 | 9.86E−25 | 0.36617727 | 0.368 | 0.12 | 2.07E−20 |
| SREK1 | 1.15E−24 | 0.44029235 | 0.58 | 0.285 | 2.41E−20 |
| RFC3 | 1.20E−24 | 0.36773948 | 0.355 | 0.109 | 2.52E−20 |
| SOD1 | 1.26E−24 | −0.5059975 | 0.705 | 0.812 | 2.64E−20 |
| TADA1 | 1.27E−24 | 0.29352472 | 0.298 | 0.073 | 2.67E−20 |
| UXT | 1.33E−24 | −0.39171302 | 0.047 | 0.311 | 2.80E−20 |
| CAPN1 | 1.34E−24 | 0.33362429 | 0.313 | 0.084 | 2.81E−20 |
| CCNJ | 1.39E−24 | 0.39322085 | 0.334 | 0.099 | 2.93E−20 |
| KIF1B | 1.42E−24 | 0.48702026 | 0.523 | 0.238 | 2.99E−20 |
| TRPS1 | 1.42E−24 | 0.40446917 | 0.324 | 0.092 | 2.99E−20 |
| POLR2H | 1.47E−24 | −0.46597552 | 0.122 | 0.407 | 3.09E−20 |
| TMEM165 | 1.48E−24 | −0.54560961 | 0.21 | 0.494 | 3.12E−20 |
| PSRC1 | 1.51E−24 | 0.52139411 | 0.275 | 0.064 | 3.17E−20 |
| TIMM8B | 1.63E−24 | −0.41660522 | 0.15 | 0.45 | 3.42E−20 |
| GALNT2 | 1.63E−24 | 0.38306028 | 0.365 | 0.119 | 3.43E−20 |
| ARL4C | 1.68E−24 | 0.31862841 | 0.262 | 0.056 | 3.54E−20 |
| MCRS1 | 1.78E−24 | 0.45268684 | 0.451 | 0.178 | 3.74E−20 |
| HBS1L | 1.80E−24 | 0.36103397 | 0.409 | 0.144 | 3.80E−20 |
| FAM60A | 1.81E−24 | 0.45236731 | 0.689 | 0.394 | 3.81E−20 |
| DNM1L | 1.81E−24 | 0.38395988 | 0.472 | 0.194 | 3.81E−20 |
| UNG | 1.84E−24 | 0.55612806 | 0.451 | 0.187 | 3.87E−20 |
| RTCB | 2.19E−24 | 0.46593631 | 0.635 | 0.352 | 4.60E−20 |
| HEXA | 2.21E−24 | 0.27893829 | 0.301 | 0.077 | 4.66E−20 |
| UBC | 2.30E−24 | −0.58844581 | 0.798 | 0.911 | 4.85E−20 |
| C12orf57 | 2.58E−24 | −0.55231572 | 0.275 | 0.551 | 5.42E−20 |
| MIER1 | 2.61E−24 | 0.48118393 | 0.347 | 0.104 | 5.50E−20 |
| CCNG1 | 2.82E−24 | −0.61811236 | 0.725 | 0.852 | 5.93E−20 |
| PSEN1 | 2.89E−24 | 0.37628175 | 0.417 | 0.153 | 6.08E−20 |
| PHF21A | 2.98E−24 | 0.32425925 | 0.311 | 0.083 | 6.27E−20 |
| NDUFA12 | 3.04E−24 | −0.46932988 | 0.148 | 0.436 | 6.40E−20 |
| RWDD1 | 3.09E−24 | −0.42652827 | 0.135 | 0.427 | 6.51E−20 |
| TRAPPC2L | 3.13E−24 | −0.42142301 | 0.075 | 0.35 | 6.59E−20 |
| LAS1L | 3.14E−24 | 0.34115121 | 0.313 | 0.087 | 6.62E−20 |
| CRY1 | 3.16E−24 | 0.33550035 | 0.277 | 0.066 | 6.66E−20 |
| TET2 | 3.17E−24 | 0.32552122 | 0.28 | 0.067 | 6.68E−20 |
| TBC1D16 | 3.46E−24 | 0.34322167 | 0.269 | 0.061 | 7.28E−20 |
| SYF2 | 3.47E−24 | −0.49386078 | 0.135 | 0.418 | 7.31E−20 |
| TSTD1 | 3.50E−24 | −0.41497372 | 0.039 | 0.293 | 7.36E−20 |
| CCDC85B | 3.58E−24 | −0.44218157 | 0.065 | 0.334 | 7.54E−20 |
| PACSIN2 | 3.68E−24 | 0.30565899 | 0.288 | 0.07 | 7.74E−20 |
| ERC1 | 3.77E−24 | 0.44497422 | 0.28 | 0.067 | 7.95E−20 |
| TOMM40 | 3.80E−24 | −0.47236139 | 0.119 | 0.401 | 8.00E−20 |
| CCT8 | 3.87E−24 | −0.51751045 | 0.725 | 0.866 | 8.15E−20 |
| NRBP1 | 4.36E−24 | 0.44885789 | 0.5 | 0.215 | 9.18E−20 |
| C17orf85 | 4.60E−24 | 0.31307449 | 0.347 | 0.105 | 9.69E−20 |
| AFF4 | 4.98E−24 | 0.52990167 | 0.557 | 0.271 | 1.05E−19 |
| CPSF7 | 5.09E−24 | 0.31326723 | 0.308 | 0.083 | 1.07E−19 |
| MPPED2 | 5.12E−24 | −0.48293647 | 0.07 | 0.341 | 1.08E−19 |
| UHRF1BP1L | 5.18E−24 | 0.28571454 | 0.256 | 0.055 | 1.09E−19 |
| CARD8 | 5.27E−24 | 0.32058893 | 0.277 | 0.066 | 1.11E−19 |
| PSMD2 | 5.34E−24 | 0.527316 | 0.655 | 0.38 | 1.12E−19 |
| NR2C1 | 5.56E−24 | 0.31316958 | 0.326 | 0.093 | 1.17E−19 |
| KCTD10 | 5.64E−24 | 0.32923636 | 0.259 | 0.056 | 1.19E−19 |
| GTSE1 | 5.71E−24 | 0.44715522 | 0.269 | 0.064 | 1.20E−19 |
| FNDC3A | 6.01E−24 | 0.39875409 | 0.5 | 0.214 | 1.26E−19 |
| GSE1 | 6.21E−24 | 0.30612957 | 0.259 | 0.056 | 1.31E−19 |
| OPA1 | 6.21E−24 | 0.35022484 | 0.337 | 0.1 | 1.31E−19 |
| KLHL9 | 6.29E−24 | 0.41496613 | 0.451 | 0.186 | 1.32E−19 |
| DLL1 | 6.61E−24 | 0.40950395 | 0.303 | 0.078 | 1.39E−19 |
| NUP153 | 6.80E−24 | 0.28028956 | 0.254 | 0.053 | 1.43E−19 |
| PIGF | 6.97E−24 | −0.41363486 | 0.054 | 0.318 | 1.47E−19 |
| CEP68 | 7.31E−24 | 0.3964701 | 0.37 | 0.125 | 1.54E−19 |
| FGF9 | 8.12E−24 | 0.37360265 | 0.267 | 0.061 | 1.71E−19 |
| CTPS2 | 8.15E−24 | 0.34495855 | 0.352 | 0.112 | 1.72E−19 |
| ATP5B | 8.70E−24 | 0.42627509 | 0.966 | 0.933 | 1.83E−19 |
| TTC28 | 8.74E−24 | 0.35377894 | 0.363 | 0.12 | 1.84E−19 |
| PCDH9 | 1.01E−23 | 0.44900164 | 0.251 | 0.054 | 2.12E−19 |
| PPP2R2A | 1.01E−23 | −0.43555307 | 0.08 | 0.35 | 2.14E−19 |
| CYSTM1 | 1.08E−23 | −0.45385215 | 0.08 | 0.348 | 2.28E−19 |
| STARD3 | 1.14E−23 | 0.35467088 | 0.37 | 0.123 | 2.40E−19 |
| GTF2A2 | 1.15E−23 | −0.44630769 | 0.174 | 0.465 | 2.41E−19 |
| NOL7 | 1.16E−23 | −0.46626081 | 0.218 | 0.502 | 2.45E−19 |
| IBTK | 1.20E−23 | 0.35987378 | 0.365 | 0.122 | 2.52E−19 |
| RAB3A | 1.22E−23 | −0.54463865 | 0.023 | 0.263 | 2.56E−19 |
| CNOT2 | 1.51E−23 | 0.38679827 | 0.521 | 0.227 | 3.18E−19 |
| KIAA1109 | 1.52E−23 | 0.32760467 | 0.301 | 0.081 | 3.20E−19 |
| C6orf89 | 1.54E−23 | 0.33938007 | 0.267 | 0.061 | 3.23E−19 |
| LSG1 | 1.57E−23 | 0.31580789 | 0.262 | 0.06 | 3.31E−19 |
| MTMR14 | 1.63E−23 | 0.31044495 | 0.326 | 0.095 | 3.43E−19 |
| UBE2G2 | 1.68E−23 | 0.37477972 | 0.412 | 0.152 | 3.54E−19 |
| RABEP1 | 1.75E−23 | 0.41317332 | 0.415 | 0.155 | 3.68E−19 |
| IFRD1 | 1.79E−23 | 0.48604655 | 0.492 | 0.225 | 3.76E−19 |
| ZNHIT3 | 1.81E−23 | −0.48619575 | 0.205 | 0.489 | 3.82E−19 |
| BIRC5 | 1.87E−23 | 0.83381835 | 0.435 | 0.187 | 3.93E−19 |
| KANSL2 | 1.92E−23 | 0.31279794 | 0.352 | 0.11 | 4.05E−19 |
| EIF2B4 | 2.07E−23 | 0.30935794 | 0.295 | 0.077 | 4.36E−19 |
| API5 | 2.19E−23 | 0.43697317 | 0.604 | 0.313 | 4.60E−19 |
| ANKRD13C | 2.33E−23 | 0.31653298 | 0.332 | 0.099 | 4.90E−19 |
| EXD2 | 2.35E−23 | 0.33444778 | 0.277 | 0.068 | 4.94E−19 |
| ATP2C1 | 2.35E−23 | 0.35582217 | 0.368 | 0.126 | 4.95E−19 |
| SKIV2L2 | 2.36E−23 | 0.33681236 | 0.378 | 0.127 | 4.97E−19 |
| ZNF664 | 2.45E−23 | 0.29617726 | 0.295 | 0.077 | 5.17E−19 |
| PDIA6 | 2.58E−23 | −0.56243426 | 0.744 | 0.831 | 5.42E−19 |
| ETV5 | 2.60E−23 | 0.40183184 | 0.391 | 0.139 | 5.46E−19 |
| MRPS18C | 2.72E−23 | −0.35457775 | 0.021 | 0.259 | 5.72E−19 |
| FAM177A1 | 2.75E−23 | −0.40017917 | 0.057 | 0.315 | 5.78E−19 |
| ENTPD6 | 2.80E−23 | 0.31172904 | 0.275 | 0.066 | 5.89E−19 |
| FAM208A | 2.88E−23 | 0.29830925 | 0.256 | 0.056 | 6.06E−19 |
| ZZZ3 | 2.91E−23 | 0.31091049 | 0.319 | 0.092 | 6.13E−19 |
| SZRD1 | 2.94E−23 | 0.31764138 | 0.303 | 0.083 | 6.19E−19 |
| FKBP2 | 3.24E−23 | −0.51795063 | 0.197 | 0.471 | 6.81E−19 |
| BAG4 | 3.41E−23 | 0.27780812 | 0.254 | 0.055 | 7.17E−19 |
| SUPT16H | 3.41E−23 | 0.46585269 | 0.604 | 0.334 | 7.19E−19 |
| PTBP2 | 3.51E−23 | 0.52582482 | 0.583 | 0.292 | 7.40E−19 |
| CDC42SE2 | 3.57E−23 | 0.31169265 | 0.355 | 0.114 | 7.51E−19 |
| NDUFB8 | 3.63E−23 | −0.59056571 | 0.456 | 0.656 | 7.64E−19 |
| LHFP | 3.64E−23 | −0.50033584 | 0.101 | 0.369 | 7.66E−19 |
| FBX07 | 4.06E−23 | 0.47256758 | 0.497 | 0.225 | 8.55E−19 |
| TSC22D2 | 4.14E−23 | 0.31554259 | 0.29 | 0.075 | 8.72E−19 |
| NUP88 | 4.71E−23 | 0.32677115 | 0.285 | 0.073 | 9.91E−19 |
| TOR1AIP1 | 5.02E−23 | 0.36013201 | 0.28 | 0.071 | 1.06E−18 |
| RNF5 | 5.23E−23 | −0.55197421 | 0.43 | 0.656 | 1.10E−18 |
| MARCKS | 5.44E−23 | 0.49894133 | 0.863 | 0.599 | 1.15E−18 |
| NFRKB | 5.63E−23 | 0.36628342 | 0.339 | 0.105 | 1.19E−18 |
| C19orf70 | 5.67E−23 | −0.39382178 | 0.052 | 0.304 | 1.19E−18 |
| RNF141 | 5.69E−23 | 0.32179881 | 0.326 | 0.098 | 1.20E−18 |
| HINT2 | 5.75E−23 | −0.40339473 | 0.065 | 0.328 | 1.21E−18 |
| CCNB1 | 5.78E−23 | 1.00766005 | 0.368 | 0.131 | 1.22E−18 |
| HLTF | 6.31E−23 | 0.40932953 | 0.487 | 0.214 | 1.33E−18 |
| EHMT2 | 6.55E−23 | 0.38562637 | 0.43 | 0.169 | 1.38E−18 |
| PRMT6 | 6.69E−23 | 0.32260166 | 0.358 | 0.116 | 1.41E−18 |
| RPL26L1 | 6.72E−23 | −0.40168937 | 0.098 | 0.373 | 1.42E−18 |
| ALDH1B1 | 6.76E−23 | 0.2645571 | 0.254 | 0.056 | 1.42E−18 |
| PGRMC1 | 6.91E−23 | −0.58838273 | 0.57 | 0.726 | 1.45E−18 |
| CDK1 | 7.28E−23 | 0.62500327 | 0.409 | 0.158 | 1.53E−18 |
| ZNF714 | 7.64E−23 | 0.34341843 | 0.36 | 0.12 | 1.61E−18 |
| ATP5G1 | 7.86E−23 | −0.44394241 | 0.142 | 0.421 | 1.65E−18 |
| LARP1 | 7.90E−23 | 0.30681221 | 0.301 | 0.082 | 1.66E−18 |
| CHTF8 | 8.22E−23 | 0.35300501 | 0.407 | 0.152 | 1.73E−18 |
| ORC3 | 8.82E−23 | 0.33604203 | 0.347 | 0.112 | 1.86E−18 |
| BCAS3 | 8.90E−23 | 0.29931976 | 0.269 | 0.065 | 1.87E−18 |
| BRI3 | 9.00E−23 | −0.33786711 | 0.018 | 0.251 | 1.90E−18 |
| WASF3 | 9.09E−23 | 0.33244865 | 0.293 | 0.078 | 1.91E−18 |
| ABCD3 | 9.28E−23 | 0.38133628 | 0.381 | 0.134 | 1.95E−18 |
| PNRC1 | 9.54E−23 | −0.51475933 | 0.233 | 0.511 | 2.01E−18 |
| ATP5H | 1.03E−22 | −0.4691691 | 0.267 | 0.54 | 2.17E−18 |
| PDK2 | 1.04E−22 | 0.33741188 | 0.308 | 0.088 | 2.18E−18 |
| EXT2 | 1.08E−22 | 0.37872253 | 0.345 | 0.112 | 2.26E−18 |
| DDX41 | 1.15E−22 | 0.35761385 | 0.321 | 0.095 | 2.43E−18 |
| COASY | 1.21E−22 | 0.45077708 | 0.381 | 0.136 | 2.56E−18 |
| H1FX | 1.28E−22 | −0.53365063 | 0.155 | 0.429 | 2.70E−18 |
| PPP1R15A | 1.29E−22 | 0.47960919 | 0.37 | 0.134 | 2.71E−18 |
| UBXN1 | 1.36E−22 | −0.55748842 | 0.373 | 0.604 | 2.87E−18 |
| MGME1 | 1.45E−22 | 0.37578263 | 0.365 | 0.123 | 3.06E−18 |
| SLC20A1 | 1.50E−22 | 0.57965323 | 0.5 | 0.24 | 3.16E−18 |
| LRRN1 | 1.54E−22 | 0.4826189 | 0.487 | 0.225 | 3.24E−18 |
| PRKACB | 1.59E−22 | 0.3341368 | 0.298 | 0.082 | 3.34E−18 |
| SPC25 | 1.64E−22 | 0.42258864 | 0.324 | 0.099 | 3.45E−18 |
| TPM3 | 1.65E−22 | 0.56228355 | 0.694 | 0.424 | 3.48E−18 |
| ASH2L | 1.67E−22 | 0.38784344 | 0.365 | 0.126 | 3.51E−18 |
| RCC1 | 1.72E−22 | 0.2997763 | 0.272 | 0.068 | 3.62E−18 |
| SMC2 | 1.74E−22 | 0.41659588 | 0.402 | 0.153 | 3.67E−18 |
| ANAPC4 | 1.76E−22 | 0.28275537 | 0.254 | 0.057 | 3.71E−18 |
| PHF3 | 1.84E−22 | 0.37090241 | 0.422 | 0.164 | 3.87E−18 |
| ZNF521 | 1.87E−22 | 0.3282108 | 0.301 | 0.084 | 3.94E−18 |
| TJP1 | 1.92E−22 | 0.41868419 | 0.332 | 0.105 | 4.04E−18 |
| KIAA0319L | 1.98E−22 | 0.2995909 | 0.272 | 0.067 | 4.18E−18 |
| RPN2 | 2.08E−22 | 0.4563017 | 0.847 | 0.65 | 4.38E−18 |
| MBD1 | 2.18E−22 | 0.30228793 | 0.332 | 0.101 | 4.60E−18 |
| CCDC50 | 2.22E−22 | 0.40906173 | 0.487 | 0.219 | 4.68E−18 |
| MEETTL3 | 2.27E−22 | 0.33217911 | 0.329 | 0.104 | 4.79E−18 |
| PHF5A | 2.31E−22 | −0.39151196 | 0.075 | 0.334 | 4.85E−18 |
| VIMP | 2.36E−22 | −0.48638944 | 0.15 | 0.421 | 4.96E−18 |
| DMXL1 | 2.48E−22 | 0.31653298 | 0.319 | 0.094 | 5.23E−18 |
| HIST1H1E | 2.50E−22 | 0.57735977 | 0.319 | 0.103 | 5.26E−18 |
| QPRT | 2.50E−22 | 0.48503706 | 0.575 | 0.317 | 5.26E−18 |
| KAT7 | 2.51E−22 | 0.35553253 | 0.391 | 0.143 | 5.28E−18 |
| MRPS21 | 2.72E−22 | −0.51122547 | 0.35 | 0.595 | 5.72E−18 |
| EIF4B | 2.89E−22 | 0.47980093 | 0.666 | 0.391 | 6.09E−18 |
| KLHDC8B | 2.97E−22 | 0.49153411 | 0.614 | 0.342 | 6.25E−18 |
| IDH2 | 3.08E−22 | 0.56257816 | 0.699 | 0.444 | 6.49E−18 |
| TCTN3 | 3.34E−22 | 0.3708212 | 0.477 | 0.207 | 7.03E−18 |
| C21orf59 | 3.52E−22 | 0.43029148 | 0.534 | 0.258 | 7.42E−18 |
| ALDH3A2 | 3.67E−22 | 0.39511782 | 0.412 | 0.156 | 7.73E−18 |
| SSFA2 | 3.70E−22 | 0.33201862 | 0.303 | 0.087 | 7.78E−18 |
| TMEM231 | 3.78E−22 | 0.29529617 | 0.306 | 0.088 | 7.96E−18 |
| AGTPBP1 | 3.79E−22 | 0.35893143 | 0.352 | 0.117 | 7.98E−18 |
| PIGT | 3.85E−22 | 0.39724136 | 0.479 | 0.21 | 8.11E−18 |
| ZFR | 3.89E−22 | 0.49307618 | 0.617 | 0.344 | 8.19E−18 |
| FBLN1 | 4.08E−22 | −0.64132504 | 0.249 | 0.521 | 8.59E−18 |
| C14orf159 | 4.10E−22 | 0.34814725 | 0.293 | 0.082 | 8.62E−18 |
| SAT1 | 4.37E−22 | −0.6173783 | 0.205 | 0.467 | 9.20E−18 |
| PSMB1 | 4.74E−22 | −0.4806039 | 0.749 | 0.815 | 9.98E−18 |
| HAT1 | 5.09E−22 | 0.38665567 | 0.487 | 0.215 | 1.07E−17 |
| RBX1 | 5.51E−22 | −0.43920245 | 0.363 | 0.616 | 1.16E−17 |
| DR1 | 5.81E−22 | 0.45263824 | 0.407 | 0.161 | 1.22E−17 |
| ETF1 | 5.81E−22 | 0.37234872 | 0.451 | 0.189 | 1.22E−17 |
| UQCRC1 | 5.84E−22 | 0.50414614 | 0.736 | 0.471 | 1.23E−17 |
| KDM3A | 6.43E−22 | 0.42077303 | 0.453 | 0.197 | 1.35E−17 |
| SIGMAR1 | 6.91E−22 | 0.38883684 | 0.446 | 0.183 | 1.46E−17 |
| CENPF | 7.25E−22 | 0.98689167 | 0.433 | 0.191 | 1.53E−17 |
| SLC35F5 | 7.32E−22 | 0.32063444 | 0.339 | 0.11 | 1.54E−17 |
| LTN1 | 7.86E−22 | 0.37409012 | 0.404 | 0.156 | 1.65E−17 |
| PRPF3 | 7.86E−22 | 0.32900835 | 0.316 | 0.095 | 1.66E−17 |
| UTP6 | 7.98E−22 | 0.39712387 | 0.319 | 0.098 | 1.68E−17 |
| ARPC2 | 8.48E−22 | −0.53416325 | 0.645 | 0.764 | 1.78E−17 |
| EIF3M | 8.89E−22 | −0.49459796 | 0.44 | 0.65 | 1.87E−17 |
| TMEM115 | 9.17E−22 | 0.31906373 | 0.332 | 0.104 | 1.93E−17 |
| CENPN | 9.66E−22 | 0.36684338 | 0.313 | 0.097 | 2.03E−17 |
| STRN3 | 1.04E−21 | 0.30285492 | 0.277 | 0.073 | 2.19E−17 |
| ATF2 | 1.11E−21 | 0.36950852 | 0.42 | 0.165 | 2.35E−17 |
| PLS3 | 1.13E−21 | 0.42857494 | 0.443 | 0.189 | 2.38E−17 |
| SCAF11 | 1.13E−21 | 0.40056234 | 0.619 | 0.331 | 2.39E−17 |
| CNIH1 | 1.24E−21 | −0.47336082 | 0.117 | 0.375 | 2.60E−17 |
| NEAT1 | 1.28E−21 | 0.51086601 | 0.596 | 0.311 | 2.70E−17 |
| TAF9 | 1.38E−21 | −0.43669773 | 0.142 | 0.41 | 2.90E−17 |
| EIF4H | 1.48E−21 | 0.46483871 | 0.767 | 0.507 | 3.13E−17 |
| NUMB | 1.52E−21 | 0.29005738 | 0.303 | 0.088 | 3.21E−17 |
| KBTBD6 | 1.54E−21 | 0.33503876 | 0.342 | 0.114 | 3.25E−17 |
| NDUFS1 | 1.58E−21 | 0.3242816 | 0.407 | 0.155 | 3.33E−17 |
| RFC4 | 1.63E−21 | 0.37637252 | 0.378 | 0.137 | 3.42E−17 |
| SHKBP1 | 1.64E−21 | 0.3895567 | 0.329 | 0.106 | 3.45E−17 |
| TSPAN13 | 1.68E−21 | −0.47957939 | 0.161 | 0.429 | 3.53E−17 |
| DHTKD1 | 1.86E−21 | 0.33888952 | 0.275 | 0.073 | 3.92E−17 |
| RPAP3 | 1.89E−21 | 0.29922869 | 0.298 | 0.087 | 3.97E−17 |
| CAND1 | 1.95E−21 | 0.5416622 | 0.573 | 0.306 | 4.11E−17 |
| FPGS | 2.14E−21 | 0.26397721 | 0.282 | 0.076 | 4.51E−17 |
| MAGEF1 | 2.24E−21 | −0.45606617 | 0.218 | 0.48 | 4.72E−17 |
| HIST1H1C | 2.56E−21 | 0.97439856 | 0.363 | 0.142 | 5.38E−17 |
| PAM | 2.69E−21 | 0.41960361 | 0.466 | 0.218 | 5.66E−17 |
| ZNF518B | 2.78E−21 | 0.3245081 | 0.306 | 0.092 | 5.85E−17 |
| CHCHD6 | 2.78E−21 | −0.43777922 | 0.127 | 0.389 | 5.85E−17 |
| ITFG2 | 2.92E−21 | 0.31070943 | 0.298 | 0.087 | 6.14E−17 |
| RICTOR | 3.28E−21 | 0.29470868 | 0.282 | 0.078 | 6.91E−17 |
| ZNF512 | 3.45E−21 | 0.37762498 | 0.394 | 0.155 | 7.26E−17 |
| HNRNPA0 | 3.65E−21 | −0.50146251 | 0.782 | 0.857 | 7.69E−17 |
| LINC00662 | 3.70E−21 | −0.38173465 | 0.047 | 0.281 | 7.79E−17 |
| PARVA | 3.78E−21 | 0.29931666 | 0.316 | 0.098 | 7.95E−17 |
| AAAS | 4.22E−21 | 0.38232552 | 0.422 | 0.175 | 8.89E−17 |
| CTTNBP2NL | 4.30E−21 | 0.32812236 | 0.337 | 0.111 | 9.06E−17 |
| CHCHD5 | 5.47E−21 | −0.3708784 | 0.07 | 0.315 | 1.15E−16 |
| CREB1 | 5.62E−21 | 0.37836858 | 0.44 | 0.193 | 1.18E−16 |
| CDKAL1 | 5.73E−21 | 0.35773675 | 0.295 | 0.086 | 1.21E−16 |
| MRPL21 | 6.26E−21 | −0.40361518 | 0.088 | 0.337 | 1.32E−16 |
| ZNF672 | 6.31E−21 | 0.42870294 | 0.321 | 0.105 | 1.33E−16 |
| MAPRE1 | 6.81E−21 | 0.4692597 | 0.689 | 0.427 | 1.43E−16 |
| SLC25A13 | 7.02E−21 | 0.37159869 | 0.352 | 0.125 | 1.48E−16 |
| COL11A1 | 7.14E−21 | 0.40421011 | 0.37 | 0.143 | 1.50E−16 |
| ZFHX4 | 7.51E−21 | 0.37016146 | 0.394 | 0.154 | 1.58E−16 |
| BDP1 | 7.72E−21 | 0.33334048 | 0.376 | 0.139 | 1.63E−16 |
| CBWD1 | 7.89E−21 | −0.33108772 | 0.034 | 0.259 | 1.66E−16 |
| GYS1 | 8.09E−21 | 0.30520296 | 0.293 | 0.086 | 1.70E−16 |
| SMARCAD1 | 8.45E−21 | 0.34554149 | 0.402 | 0.16 | 1.78E−16 |
| HP1BP3 | 9.50E−21 | 0.54997888 | 0.65 | 0.401 | 2.00E−16 |
| FAM179B | 1.18E−20 | 0.30477552 | 0.332 | 0.11 | 2.49E−16 |
| CBX3 | 1.24E−20 | 0.45154002 | 0.736 | 0.488 | 2.62E−16 |
| BUD13 | 1.26E−20 | 0.25158974 | 0.254 | 0.064 | 2.65E−16 |
| CCNB2 | 1.40E−20 | 0.9254491 | 0.376 | 0.154 | 2.95E−16 |
| MRPL51 | 1.42E−20 | −0.48466109 | 0.337 | 0.579 | 2.99E−16 |
| SMARCA4 | 1.79E−20 | 0.5098655 | 0.699 | 0.425 | 3.77E−16 |
| MUL1 | 1.79E−20 | 0.27168041 | 0.285 | 0.082 | 3.78E−16 |
| PPP1R10 | 1.92E−20 | 0.47307751 | 0.459 | 0.211 | 4.03E−16 |
| BPTF | 1.93E−20 | 0.40516016 | 0.516 | 0.248 | 4.06E−16 |
| RBM25 | 1.93E−20 | 0.35216064 | 0.554 | 0.271 | 4.07E−16 |
| LAMP2 | 2.01E−20 | 0.3246149 | 0.487 | 0.22 | 4.23E−16 |
| RIC8A | 2.02E−20 | 0.3693247 | 0.389 | 0.148 | 4.26E−16 |
| TP53BP2 | 2.03E−20 | 0.29878384 | 0.298 | 0.09 | 4.28E−16 |
| FBXO5 | 2.04E−20 | 0.39082368 | 0.376 | 0.142 | 4.30E−16 |
| TOP2B | 2.13E−20 | 0.40869088 | 0.5 | 0.243 | 4.48E−16 |
| ABCE1 | 2.32E−20 | 0.33793938 | 0.365 | 0.134 | 4.88E−16 |
| CEP70 | 2.40E−20 | 0.39623387 | 0.313 | 0.104 | 5.06E−16 |
| PLOD2 | 2.41E−20 | 0.38019798 | 0.35 | 0.126 | 5.07E−16 |
| C16orf59 | 2.41E−20 | 0.25633659 | 0.251 | 0.062 | 5.08E−16 |
| RAB8A | 2.43E−20 | 0.35256788 | 0.487 | 0.226 | 5.13E−16 |
| HSF2 | 2.65E−20 | 0.34850194 | 0.404 | 0.164 | 5.58E−16 |
| RFX5 | 2.66E−20 | 0.31506406 | 0.272 | 0.076 | 5.60E−16 |
| EIF4G3 | 2.72E−20 | 0.30611425 | 0.337 | 0.114 | 5.73E−16 |
| ILK | 2.75E−20 | 0.42446588 | 0.508 | 0.244 | 5.78E−16 |
| MCCC1 | 2.82E−20 | 0.36313267 | 0.288 | 0.086 | 5.93E−16 |
| NDUFB4 | 2.83E−20 | −0.39910079 | 0.249 | 0.518 | 5.96E−16 |
| DDX20 | 2.84E−20 | 0.26482976 | 0.293 | 0.087 | 5.98E−16 |
| DPYSL3 | 2.92E−20 | −0.40532541 | 0.07 | 0.309 | 6.14E−16 |
| STAU1 | 2.93E−20 | 0.40407281 | 0.518 | 0.244 | 6.18E−16 |
| ZNF74 | 2.94E−20 | 0.37241303 | 0.272 | 0.077 | 6.18E−16 |
| HNRNPM | 3.00E−20 | 0.45674485 | 0.751 | 0.494 | 6.31E−16 |
| SPARC | 3.00E−20 | 0.68663674 | 0.767 | 0.564 | 6.32E−16 |
| NBEAL1 | 3.16E−20 | −0.40283447 | 0.155 | 0.416 | 6.66E−16 |
| CAPN7 | 3.26E−20 | 0.30383681 | 0.298 | 0.092 | 6.86E−16 |
| RAD21 | 3.30E−20 | 0.53479503 | 0.642 | 0.397 | 6.96E−16 |
| FNTA | 3.43E−20 | −0.39599225 | 0.122 | 0.375 | 7.23E−16 |
| CENPC | 3.49E−20 | 0.34388968 | 0.313 | 0.103 | 7.36E−16 |
| TARS2 | 3.89E−20 | 0.290387 | 0.262 | 0.07 | 8.18E−16 |
| INTS10 | 3.94E−20 | 0.31997619 | 0.339 | 0.119 | 8.29E−16 |
| APLP1 | 4.06E−20 | −0.57988447 | 0.342 | 0.573 | 8.54E−16 |
| GNAI1 | 4.59E−20 | 0.28576308 | 0.29 | 0.087 | 9.67E−16 |
| DDX10 | 5.21E−20 | 0.29189915 | 0.272 | 0.076 | 1.10E−15 |
| CDC37 | 5.48E−20 | 0.39384012 | 0.671 | 0.412 | 1.15E−15 |
| VPS13A | 5.95E−20 | 0.28428212 | 0.259 | 0.07 | 1.25E−15 |
| MED10 | 6.20E−20 | −0.45706839 | 0.148 | 0.4 | 1.31E−15 |
| PNRC2 | 6.35E−20 | 0.35801987 | 0.56 | 0.284 | 1.34E−15 |
| TNKS | 6.47E−20 | 0.29555047 | 0.378 | 0.143 | 1.36E−15 |
| PDCD5 | 6.89E−20 | −0.42406581 | 0.212 | 0.473 | 1.45E−15 |
| MAD2L1 | 7.02E−20 | 0.57424716 | 0.477 | 0.231 | 1.48E−15 |
| SMG6 | 7.12E−20 | 0.27655164 | 0.264 | 0.072 | 1.50E−15 |
| ALDH6A1 | 7.14E−20 | 0.33956244 | 0.402 | 0.161 | 1.50E−15 |
| CYR61 | 7.17E−20 | 0.47904132 | 0.262 | 0.075 | 1.51E−15 |
| CTNNBIP1 | 7.35E−20 | −0.36455256 | 0.047 | 0.27 | 1.55E−15 |
| C9orf16 | 7.43E−20 | −0.47121213 | 0.236 | 0.49 | 1.56E−15 |
| UBE2S | 7.45E−20 | −0.45739823 | 0.054 | 0.282 | 1.57E−15 |
| IL6ST | 7.84E−20 | 0.25559937 | 0.251 | 0.065 | 1.65E−15 |
| HIP1 | 8.15E−20 | 0.2988775 | 0.311 | 0.101 | 1.72E−15 |
| TCEAL3 | 8.47E−20 | −0.41650782 | 0.137 | 0.389 | 1.78E−15 |
| TULP3 | 8.61E−20 | 0.28573622 | 0.352 | 0.126 | 1.81E−15 |
| NCOR1 | 9.71E−20 | 0.39959565 | 0.72 | 0.461 | 2.04E−15 |
| TMED5 | 1.07E−19 | 0.27410593 | 0.264 | 0.073 | 2.25E−15 |
| RCN2 | 1.13E−19 | −0.43637396 | 0.824 | 0.933 | 2.39E−15 |
| ACAD9 | 1.13E−19 | 0.31476637 | 0.301 | 0.097 | 2.39E−15 |
| NDFIP1 | 1.23E−19 | −0.48985589 | 0.479 | 0.65 | 2.59E−15 |
| TCEB1 | 1.31E−19 | −0.42073123 | 0.223 | 0.483 | 2.75E−15 |
| EZR | 1.31E−19 | 0.36674762 | 0.352 | 0.13 | 2.76E−15 |
| BLMH | 1.42E−19 | 0.33875705 | 0.35 | 0.131 | 2.98E−15 |
| ING3 | 1.42E−19 | 0.30544721 | 0.337 | 0.12 | 2.99E−15 |
| BRWD1 | 1.70E−19 | 0.39392412 | 0.531 | 0.275 | 3.57E−15 |
| GPAA1 | 1.73E−19 | 0.35846461 | 0.549 | 0.281 | 3.65E−15 |
| MGRN1 | 1.85E−19 | 0.25219275 | 0.267 | 0.075 | 3.89E−15 |
| TSPAN6 | 1.92E−19 | 0.35847102 | 0.702 | 0.427 | 4.04E−15 |
| IPO7 | 2.01E−19 | 0.3393389 | 0.43 | 0.186 | 4.23E−15 |
| AIFM1 | 2.02E−19 | 0.27695353 | 0.267 | 0.075 | 4.26E−15 |
| SCAND1 | 2.06E−19 | −0.39241975 | 0.394 | 0.621 | 4.33E−15 |
| CACUL1 | 2.10E−19 | 0.32641828 | 0.415 | 0.174 | 4.42E−15 |
| NOP10 | 2.13E−19 | −0.38206225 | 0.197 | 0.458 | 4.49E−15 |
| ATP6V1B2 | 2.28E−19 | 0.35680939 | 0.461 | 0.213 | 4.81E−15 |
| GPR108 | 2.29E−19 | 0.26844203 | 0.277 | 0.082 | 4.82E−15 |
| DNAJC3 | 2.30E−19 | 0.28878651 | 0.301 | 0.097 | 4.84E−15 |
| GRIPAP1 | 2.30E−19 | 0.27120715 | 0.256 | 0.071 | 4.85E−15 |
| UQCR11 | 2.41E−19 | −0.39954418 | 0.396 | 0.617 | 5.07E−15 |
| NDUFS6 | 2.43E−19 | −0.41672383 | 0.212 | 0.466 | 5.12E−15 |
| GGA2 | 2.49E−19 | 0.32239046 | 0.358 | 0.136 | 5.25E−15 |
| NEDD1 | 2.77E−19 | 0.2983703 | 0.329 | 0.114 | 5.83E−15 |
| CDK17 | 2.87E−19 | 0.33032417 | 0.277 | 0.083 | 6.03E−15 |
| ARIH2 | 2.89E−19 | 0.30281302 | 0.427 | 0.185 | 6.08E−15 |
| SDR39U1 | 2.90E−19 | −0.35689006 | 0.075 | 0.307 | 6.10E−15 |
| RPE | 3.01E−19 | 0.26748494 | 0.326 | 0.111 | 6.34E−15 |
| USP1 | 3.04E−19 | 0.43373284 | 0.456 | 0.213 | 6.40E−15 |
| LEO1 | 3.10E−19 | 0.27533068 | 0.264 | 0.075 | 6.53E−15 |
| WDR35 | 3.13E−19 | 0.27614507 | 0.28 | 0.083 | 6.58E−15 |
| OCIAD2 | 3.16E−19 | −0.4930195 | 0.166 | 0.411 | 6.65E−15 |
| PCBD1 | 3.16E−19 | −0.39177888 | 0.096 | 0.333 | 6.66E−15 |
| KCTD6 | 3.20E−19 | −0.40887613 | 0.093 | 0.334 | 6.74E−15 |
| ARMCX6 | 3.50E−19 | −0.33510412 | 0.041 | 0.257 | 7.37E−15 |
| LAMTOR2 | 3.53E−19 | −0.38660441 | 0.142 | 0.392 | 7.43E−15 |
| NEMF | 3.55E−19 | 0.33798433 | 0.376 | 0.148 | 7.48E−15 |
| ZMPSTE24 | 3.66E−19 | 0.29643209 | 0.311 | 0.104 | 7.71E−15 |
| ASPH | 3.80E−19 | 0.40437848 | 0.534 | 0.285 | 7.99E−15 |
| RWDD3 | 4.13E−19 | −0.36157633 | 0.044 | 0.259 | 8.69E−15 |
| SSU72 | 4.14E−19 | −0.44796496 | 0.189 | 0.438 | 8.72E−15 |
| HIBADH | 4.27E−19 | 0.32439016 | 0.355 | 0.133 | 8.98E−15 |
| MTMR4 | 4.45E−19 | 0.29177659 | 0.269 | 0.078 | 9.36E−15 |
| SUGP1 | 4.52E−19 | 0.28694883 | 0.29 | 0.092 | 9.52E−15 |
| RFC1 | 4.54E−19 | 0.37388489 | 0.474 | 0.22 | 9.56E−15 |
| SERBP1 | 4.60E−19 | 0.43604949 | 0.899 | 0.721 | 9.69E−15 |
| ARMC10 | 4.66E−19 | −0.32525906 | 0.041 | 0.256 | 9.82E−15 |
| UBA6 | 4.69E−19 | 0.31522045 | 0.345 | 0.127 | 9.87E−15 |
| PKN2 | 4.83E−19 | 0.3089784 | 0.347 | 0.128 | 1.02E−14 |
| CYFIP2 | 5.06E−19 | 0.42270608 | 0.417 | 0.183 | 1.06E−14 |
| DYNC1LI2 | 5.27E−19 | 0.34048731 | 0.469 | 0.215 | 1.11E−14 |
| ZNF24 | 5.33E−19 | 0.35098031 | 0.516 | 0.249 | 1.12E−14 |
| KIAA1324L | 5.56E−19 | 0.39332861 | 0.306 | 0.104 | 1.17E−14 |
| SRPR | 5.71E−19 | 0.3693247 | 0.373 | 0.148 | 1.20E−14 |
| PSMD11 | 5.86E−19 | 0.34385814 | 0.648 | 0.373 | 1.23E−14 |
| TRDMT1 | 5.89E−19 | 0.30128464 | 0.269 | 0.078 | 1.24E−14 |
| BEX4 | 6.05E−19 | −0.49451644 | 0.573 | 0.71 | 1.27E−14 |
| OAZ2 | 6.22E−19 | 0.44692908 | 0.811 | 0.609 | 1.31E−14 |
| TMEM30A | 6.22E−19 | 0.32067623 | 0.363 | 0.138 | 1.31E−14 |
| KATNB1 | 6.53E−19 | 0.29940838 | 0.264 | 0.076 | 1.37E−14 |
| SF3A3 | 6.64E−19 | 0.35371305 | 0.557 | 0.285 | 1.40E−14 |
| FBXO3 | 6.89E−19 | 0.30078081 | 0.339 | 0.125 | 1.45E−14 |
| FECH | 7.21E−19 | 0.36822947 | 0.251 | 0.07 | 1.52E−14 |
| HADHA | 8.12E−19 | 0.46291359 | 0.624 | 0.38 | 1.71E−14 |
| UBE2W | 8.63E−19 | 0.34603701 | 0.381 | 0.152 | 1.82E−14 |
| RCN1 | 8.85E−19 | 0.3674401 | 0.894 | 0.716 | 1.86E−14 |
| EIF2B1 | 9.68E−19 | 0.31180386 | 0.433 | 0.186 | 2.04E−14 |
| TET1 | 1.01E−18 | 0.39934659 | 0.459 | 0.214 | 2.13E−14 |
| CYP20A1 | 1.01E−18 | 0.26425793 | 0.303 | 0.099 | 2.13E−14 |
| TMEM208 | 1.02E−18 | −0.37654151 | 0.091 | 0.326 | 2.16E−14 |
| HSPA9 | 1.07E−18 | 0.45279908 | 0.699 | 0.438 | 2.26E−14 |
| DAD1 | 1.16E−18 | −0.45790304 | 0.446 | 0.641 | 2.44E−14 |
| KNSTRN | 1.18E−18 | 0.47811795 | 0.293 | 0.099 | 2.49E−14 |
| WDR43 | 1.21E−18 | 0.29572061 | 0.301 | 0.099 | 2.54E−14 |
| DTD1 | 1.21E−18 | −0.49044951 | 0.316 | 0.543 | 2.54E−14 |
| PDCD2 | 1.22E−18 | −0.38404934 | 0.114 | 0.352 | 2.57E−14 |
| SPATA6 | 1.23E−18 | 0.26025306 | 0.262 | 0.075 | 2.59E−14 |
| CALCOCO2 | 1.24E−18 | 0.30228793 | 0.311 | 0.106 | 2.62E−14 |
| ZNF91 | 1.27E−18 | 0.31016201 | 0.376 | 0.15 | 2.67E−14 |
| AHI1 | 1.28E−18 | 0.31235901 | 0.345 | 0.13 | 2.70E−14 |
| NUCKS1 | 1.32E−18 | 0.58793116 | 0.759 | 0.562 | 2.79E−14 |
| CAMK2D | 1.33E−18 | 0.3049553 | 0.254 | 0.072 | 2.80E−14 |
| TMEM14B | 1.34E−18 | −0.48052593 | 0.285 | 0.52 | 2.82E−14 |
| ABCF1 | 1.34E−18 | 0.30115167 | 0.37 | 0.145 | 2.82E−14 |
| ZCCHC11 | 1.42E−18 | 0.38048173 | 0.552 | 0.296 | 3.00E−14 |
| SP3 | 1.43E−18 | 0.35395187 | 0.383 | 0.158 | 3.02E−14 |
| RCBTB2 | 1.49E−18 | 0.33290914 | 0.324 | 0.117 | 3.15E−14 |
| MRPL55 | 1.53E−18 | −0.39219796 | 0.153 | 0.396 | 3.22E−14 |
| GPBP1L1 | 1.60E−18 | 0.26817876 | 0.303 | 0.101 | 3.36E−14 |
| MYEOV2 | 1.66E−18 | −0.37593162 | 0.142 | 0.391 | 3.49E−14 |
| ELMSAN1 | 1.68E−18 | 0.37528692 | 0.404 | 0.174 | 3.53E−14 |
| POLR2L | 1.68E−18 | −0.41447385 | 0.269 | 0.516 | 3.54E−14 |
| ATRAID | 1.73E−18 | −0.47323857 | 0.422 | 0.617 | 3.64E−14 |
| RNF20 | 1.74E−18 | 0.25474918 | 0.376 | 0.148 | 3.66E−14 |
| FGD5-AS1 | 1.82E−18 | 0.37701968 | 0.503 | 0.251 | 3.83E−14 |
| SAP18 | 1.85E−18 | −0.47603401 | 0.655 | 0.754 | 3.89E−14 |
| CARKD | 1.93E−18 | 0.26339779 | 0.254 | 0.072 | 4.06E−14 |
| SEC11C | 1.94E−18 | −0.45552622 | 0.181 | 0.425 | 4.09E−14 |
| RBM33 | 1.94E−18 | 0.253425 | 0.267 | 0.078 | 4.09E−14 |
| FAM168B | 2.16E−18 | 0.28176962 | 0.28 | 0.088 | 4.55E−14 |
| PSMA5 | 2.19E−18 | −0.43410724 | 0.21 | 0.451 | 4.62E−14 |
| VPS4A | 2.20E−18 | 0.35513043 | 0.422 | 0.183 | 4.63E−14 |
| INSIG2 | 2.24E−18 | −0.49710391 | 0.127 | 0.363 | 4.72E−14 |
| XRCC1 | 2.34E−18 | 0.34146341 | 0.35 | 0.132 | 4.92E−14 |
| AGBL5 | 2.51E−18 | 0.28583847 | 0.262 | 0.077 | 5.28E−14 |
| ZNF292 | 2.60E−18 | 0.43391178 | 0.668 | 0.422 | 5.47E−14 |
| C8orf33 | 2.70E−18 | 0.32214496 | 0.459 | 0.216 | 5.68E−14 |
| NT5DC2 | 2.75E−18 | 0.27816311 | 0.269 | 0.082 | 5.80E−14 |
| DPY19L4 | 2.89E−18 | 0.25886708 | 0.256 | 0.075 | 6.08E−14 |
| RAP1GDS1 | 2.90E−18 | 0.30409914 | 0.319 | 0.114 | 6.10E−14 |
| TPM4 | 2.92E−18 | 0.49907015 | 0.668 | 0.455 | 6.16E−14 |
| CHD2 | 3.44E−18 | 0.39985322 | 0.503 | 0.262 | 7.24E−14 |
| DNTTIP1 | 3.53E−18 | −0.36030774 | 0.091 | 0.318 | 7.43E−14 |
| APPL2 | 3.53E−18 | 0.26546279 | 0.303 | 0.103 | 7.43E−14 |
| SMC3 | 3.68E−18 | 0.34693244 | 0.604 | 0.346 | 7.74E−14 |
| USP14 | 4.08E−18 | 0.33021722 | 0.477 | 0.225 | 8.59E−14 |
| SAT2 | 4.18E−18 | −0.47857 | 0.236 | 0.472 | 8.80E−14 |
| SEC23IP | 4.25E−18 | 0.29966046 | 0.324 | 0.117 | 8.94E−14 |
| AKAP11 | 4.35E−18 | 0.27494499 | 0.285 | 0.092 | 9.17E−14 |
| DYM | 4.66E−18 | 0.3103854 | 0.311 | 0.11 | 9.82E−14 |
| COL6A1 | 4.77E−18 | 0.29111174 | 0.298 | 0.1 | 1.00E−13 |
| PHYH | 4.77E−18 | −0.37776052 | 0.104 | 0.334 | 1.00E−13 |
| GLRX5 | 4.94E−18 | −0.31174478 | 0.073 | 0.293 | 1.04E−13 |
| TCEB3 | 5.15E−18 | 0.2587703 | 0.277 | 0.087 | 1.08E−13 |
| CERS2 | 5.18E−18 | 0.25536351 | 0.29 | 0.094 | 1.09E−13 |
| SOCS4 | 5.42E−18 | 0.29233711 | 0.254 | 0.073 | 1.14E−13 |
| COPS8 | 5.70E−18 | −0.46508317 | 0.417 | 0.627 | 1.20E−13 |
| SMC5 | 5.90E−18 | 0.29231401 | 0.389 | 0.16 | 1.24E−13 |
| MXD4 | 6.05E−18 | 0.292647 | 0.262 | 0.079 | 1.27E−13 |
| SLC39A1 | 6.13E−18 | 0.40787818 | 0.477 | 0.238 | 1.29E−13 |
| HNRNPH3 | 6.28E−18 | 0.39303802 | 0.782 | 0.542 | 1.32E−13 |
| AKAP9 | 6.36E−18 | 0.32187961 | 0.534 | 0.275 | 1.34E−13 |
| MBOAT2 | 6.61E−18 | 0.28785849 | 0.311 | 0.11 | 1.39E−13 |
| DSCR3 | 6.91E−18 | 0.29846596 | 0.345 | 0.133 | 1.45E−13 |
| CNOT8 | 7.01E−18 | 0.29226913 | 0.303 | 0.104 | 1.48E−13 |
| TPD52L2 | 7.12E−18 | 0.3022343 | 0.365 | 0.148 | 1.50E−13 |
| NDUFS4 | 7.20E−18 | −0.40770918 | 0.241 | 0.485 | 1.52E−13 |
| ATP6V0B | 7.67E−18 | −0.57894662 | 0.435 | 0.606 | 1.61E−13 |
| PARP2 | 8.10E−18 | 0.26786163 | 0.308 | 0.106 | 1.70E−13 |
| PRMT5 | 8.40E−18 | 0.30954635 | 0.44 | 0.199 | 1.77E−13 |
| CAPZA2 | 9.53E−18 | 0.37620733 | 0.598 | 0.344 | 2.01E−13 |
| RBM3 | 9.63E−18 | −0.52151573 | 0.487 | 0.667 | 2.03E−13 |
| TRIM37 | 9.67E−18 | 0.28450855 | 0.288 | 0.097 | 2.03E−13 |
| SELENBP1 | 1.03E−17 | 0.27210956 | 0.264 | 0.081 | 2.17E−13 |
| PHF14 | 1.07E−17 | −0.42602437 | 0.174 | 0.41 | 2.25E−13 |
| DIS3 | 1.08E−17 | 0.30743458 | 0.324 | 0.117 | 2.28E−13 |
| ELOVL5 | 1.09E−17 | 0.35268663 | 0.495 | 0.244 | 2.30E−13 |
| RPRD1A | 1.12E−17 | 0.33836864 | 0.425 | 0.194 | 2.36E−13 |
| MKLN1 | 1.18E−17 | 0.27006031 | 0.259 | 0.078 | 2.48E−13 |
| GART | 1.18E−17 | 0.40049976 | 0.402 | 0.18 | 2.48E−13 |
| LINC00493 | 1.31E−17 | −0.31684099 | 0.104 | 0.337 | 2.76E−13 |
| FHL1 | 1.33E−17 | 0.39302501 | 0.456 | 0.216 | 2.81E−13 |
| HLA-C | 1.35E−17 | 0.27128478 | 0.269 | 0.086 | 2.85E−13 |
| CDC42EP3 | 1.43E−17 | 0.29251909 | 0.262 | 0.081 | 3.01E−13 |
| ACTR1A | 1.50E−17 | 0.40051074 | 0.472 | 0.231 | 3.16E−13 |
| ANAPC16 | 1.51E−17 | −0.45380389 | 0.5 | 0.664 | 3.18E−13 |
| MRPS27 | 1.68E−17 | 0.3383139 | 0.474 | 0.236 | 3.54E−13 |
| DNAJC15 | 1.69E−17 | −0.34582589 | 0.093 | 0.317 | 3.55E−13 |
| SPG20 | 1.70E−17 | 0.36161873 | 0.521 | 0.271 | 3.58E−13 |
| FLYWCH2 | 1.71E−17 | −0.36609223 | 0.101 | 0.325 | 3.61E−13 |
| GTF2I | 1.91E−17 | 0.25967446 | 0.352 | 0.137 | 4.01E−13 |
| OSBPL2 | 2.11E−17 | 0.26550991 | 0.256 | 0.078 | 4.44E−13 |
| NUPL1 | 2.19E−17 | 0.33720924 | 0.399 | 0.181 | 4.60E−13 |
| GDI1 | 2.35E−17 | 0.37552031 | 0.591 | 0.329 | 4.94E−13 |
| ZNF3 | 2.40E−17 | 0.30227957 | 0.378 | 0.16 | 5.05E−13 |
| AGPAT4 | 2.51E−17 | 0.28719442 | 0.342 | 0.132 | 5.29E−13 |
| LMAN2L | 2.54E−17 | 0.28200097 | 0.321 | 0.12 | 5.34E−13 |
| TMTC3 | 2.59E−17 | 0.29922869 | 0.277 | 0.092 | 5.45E−13 |
| SPINT2 | 2.67E−17 | 0.37160321 | 0.487 | 0.243 | 5.62E−13 |
| MRPL20 | 2.94E−17 | −0.45582349 | 0.202 | 0.43 | 6.20E−13 |
| RHOA | 3.02E−17 | 0.37405666 | 0.847 | 0.628 | 6.36E−13 |
| MLH3 | 3.12E−17 | 0.26850217 | 0.275 | 0.089 | 6.57E−13 |
| SSNA1 | 3.12E−17 | −0.38162274 | 0.168 | 0.4 | 6.57E−13 |
| ATP5D | 3.24E−17 | −0.45752758 | 0.438 | 0.623 | 6.82E−13 |
| AGO3 | 3.47E−17 | 0.27857181 | 0.259 | 0.081 | 7.31E−13 |
| PRKAB1 | 3.63E−17 | 0.33615526 | 0.324 | 0.122 | 7.65E−13 |
| NUDCD3 | 3.73E−17 | 0.30362581 | 0.29 | 0.1 | 7.85E−13 |
| CDKN2AIP | 3.80E−17 | 0.30633368 | 0.321 | 0.12 | 7.99E−13 |
| DYNC1I2 | 4.00E−17 | 0.36901112 | 0.443 | 0.214 | 8.42E−13 |
| APMAP | 4.18E−17 | 0.40421828 | 0.588 | 0.345 | 8.79E−13 |
| C4orf3 | 4.19E−17 | −0.42491899 | 0.604 | 0.762 | 8.83E−13 |
| MAPK1IP1L | 4.42E−17 | 0.38376135 | 0.785 | 0.553 | 9.31E−13 |
| TMEM39B | 4.51E−17 | 0.32599362 | 0.269 | 0.087 | 9.49E−13 |
| RDX | 4.59E−17 | 0.35273622 | 0.617 | 0.351 | 9.66E−13 |
| NDUFA9 | 5.07E−17 | 0.36370026 | 0.306 | 0.111 | 1.07E−12 |
| ITSN1 | 5.08E−17 | 0.40515612 | 0.466 | 0.225 | 1.07E−12 |
| CRIPT | 5.37E−17 | 0.33347825 | 0.456 | 0.221 | 1.13E−12 |
| NAP1L1 | 5.49E−17 | −0.38071604 | 0.935 | 0.951 | 1.16E−12 |
| FAM172A | 5.50E−17 | 0.27210956 | 0.269 | 0.087 | 1.16E−12 |
| ARHGEF7 | 5.55E−17 | 0.31117913 | 0.267 | 0.086 | 1.17E−12 |
| STAU2 | 5.56E−17 | 0.3465344 | 0.42 | 0.197 | 1.17E−12 |
| PHACTR4 | 5.63E−17 | 0.33067253 | 0.427 | 0.198 | 1.19E−12 |
| MOB3A | 5.88E−17 | 0.33244865 | 0.256 | 0.081 | 1.24E−12 |
| SYT1 | 5.89E−17 | 0.27900246 | 0.549 | 0.284 | 1.24E−12 |
| SENP7 | 6.28E−17 | 0.28003899 | 0.308 | 0.112 | 1.32E−12 |
| CDK2 | 6.38E−17 | 0.26643776 | 0.308 | 0.11 | 1.34E−12 |
| PBK | 6.55E−17 | 0.36324828 | 0.295 | 0.106 | 1.38E−12 |
| ZNF281 | 6.91E−17 | 0.32708664 | 0.337 | 0.132 | 1.46E−12 |
| ARF5 | 7.18E−17 | 0.3115194 | 0.412 | 0.186 | 1.51E−12 |
| GALNT7 | 7.28E−17 | 0.29649372 | 0.37 | 0.155 | 1.53E−12 |
| RAB3GAP1 | 7.65E−17 | 0.33275902 | 0.301 | 0.11 | 1.61E−12 |
| PRDX3 | 8.95E−17 | 0.37931349 | 0.754 | 0.537 | 1.88E−12 |
| RFXANK | 8.99E−17 | −0.35442139 | 0.109 | 0.329 | 1.89E−12 |
| SCG5 | 9.07E−17 | −0.44806625 | 0.088 | 0.297 | 1.91E−12 |
| SMARCB1 | 1.03E−16 | 0.37772902 | 0.788 | 0.571 | 2.16E−12 |
| AP2A1 | 1.07E−16 | 0.31407763 | 0.324 | 0.125 | 2.25E−12 |
| ENY2 | 1.08E−16 | −0.39766535 | 0.347 | 0.572 | 2.27E−12 |
| ISOC2 | 1.09E−16 | −0.3534262 | 0.075 | 0.282 | 2.29E−12 |
| ANKRD54 | 1.09E−16 | 0.29674404 | 0.282 | 0.097 | 2.30E−12 |
| MYL6B | 1.17E−16 | −0.4235526 | 0.394 | 0.584 | 2.46E−12 |
| ZSCAN18 | 1.20E−16 | −0.38460906 | 0.127 | 0.348 | 2.53E−12 |
| ARAF | 1.20E−16 | 0.26717815 | 0.308 | 0.112 | 2.53E−12 |
| NSD1 | 1.23E−16 | 0.41729765 | 0.453 | 0.222 | 2.58E−12 |
| NDUFB3 | 1.29E−16 | −0.33627542 | 0.148 | 0.379 | 2.71E−12 |
| HECTD1 | 1.33E−16 | 0.31387207 | 0.355 | 0.148 | 2.79E−12 |
| PGAM1 | 1.44E−16 | 0.38057416 | 0.487 | 0.26 | 3.02E−12 |
| COX6C | 1.50E−16 | −0.45024584 | 0.575 | 0.697 | 3.16E−12 |
| SMARCA1 | 1.59E−16 | 0.41216587 | 0.604 | 0.377 | 3.36E−12 |
| SHMT1 | 1.70E−16 | 0.26613605 | 0.277 | 0.095 | 3.57E−12 |
| SART3 | 1.77E−16 | 0.28910879 | 0.29 | 0.104 | 3.72E−12 |
| HDDC2 | 1.83E−16 | −0.3644816 | 0.171 | 0.399 | 3.84E−12 |
| GMNN | 1.88E−16 | 0.28525612 | 0.402 | 0.18 | 3.96E−12 |
| LSAMP | 1.90E−16 | 0.37024547 | 0.44 | 0.216 | 4.00E−12 |
| EID1 | 2.06E−16 | −0.37743835 | 0.876 | 0.929 | 4.33E−12 |
| SLC2A1 | 2.09E−16 | 0.9224267 | 0.772 | 0.626 | 4.39E−12 |
| SRM | 2.17E−16 | −0.3145721 | 0.075 | 0.282 | 4.57E−12 |
| HAX1 | 2.38E−16 | −0.37840411 | 0.212 | 0.438 | 5.01E−12 |
| WBP11 | 2.45E−16 | 0.33010211 | 0.513 | 0.265 | 5.16E−12 |
| DBNL | 2.49E−16 | 0.31130591 | 0.474 | 0.233 | 5.25E−12 |
| VDAC1 | 2.57E−16 | −0.43431965 | 0.676 | 0.782 | 5.40E−12 |
| UBE2C | 2.60E−16 | 0.55747162 | 0.363 | 0.158 | 5.48E−12 |
| ADI1 | 2.67E−16 | 0.34401441 | 0.342 | 0.142 | 5.62E−12 |
| KLF6 | 2.67E−16 | 0.2652198 | 0.321 | 0.121 | 5.62E−12 |
| CPSF3L | 2.69E−16 | 0.34747152 | 0.446 | 0.221 | 5.66E−12 |
| ORC6 | 2.74E−16 | 0.30977585 | 0.389 | 0.167 | 5.76E−12 |
| PRMT2 | 2.77E−16 | 0.39233809 | 0.611 | 0.369 | 5.84E−12 |
| DFFA | 2.84E−16 | 0.25828454 | 0.36 | 0.15 | 5.97E−12 |
| EIF1B | 2.94E−16 | −0.46929918 | 0.487 | 0.654 | 6.20E−12 |
| ADCK4 | 3.01E−16 | 0.25431627 | 0.267 | 0.089 | 6.34E−12 |
| CLIC4 | 3.22E−16 | 0.37066927 | 0.474 | 0.246 | 6.79E−12 |
| CEP78 | 3.26E−16 | 0.31466635 | 0.407 | 0.182 | 6.87E−12 |
| JTB | 3.33E−16 | −0.35765657 | 0.269 | 0.493 | 7.01E−12 |
| ACTN1 | 3.41E−16 | 0.29817689 | 0.381 | 0.163 | 7.18E−12 |
| TMA7 | 3.46E−16 | −0.44973702 | 0.593 | 0.699 | 7.29E−12 |
| NCOA4 | 3.56E−16 | 0.41207136 | 0.448 | 0.226 | 7.49E−12 |
| PRDX6 | 3.65E−16 | −0.42206665 | 0.756 | 0.812 | 7.68E−12 |
| BLCAP | 3.73E−16 | −0.40504063 | 0.345 | 0.564 | 7.85E−12 |
| MPHOSPH9 | 3.97E−16 | 0.26644081 | 0.259 | 0.086 | 8.36E−12 |
| NDUFB7 | 4.12E−16 | −0.25530192 | 0.337 | 0.556 | 8.68E−12 |
| SERINC1 | 4.32E−16 | 0.39511194 | 0.58 | 0.347 | 9.09E−12 |
| VRK1 | 4.37E−16 | 0.32394579 | 0.389 | 0.174 | 9.20E−12 |
| EIF4EBP1 | 4.57E−16 | −0.57166412 | 0.218 | 0.425 | 9.63E−12 |
| RRN3 | 4.71E−16 | 0.27851598 | 0.282 | 0.099 | 9.91E−12 |
| ARL5A | 4.73E−16 | −0.38918427 | 0.176 | 0.399 | 9.95E−12 |
| BSDC1 | 4.78E−16 | 0.3398063 | 0.399 | 0.188 | 1.01E−11 |
| UBA3 | 4.79E−16 | 0.31289681 | 0.407 | 0.186 | 1.01E−11 |
| ATF6 | 4.95E−16 | 0.25916967 | 0.29 | 0.105 | 1.04E−11 |
| DAAM1 | 5.43E−16 | 0.36091013 | 0.56 | 0.318 | 1.14E−11 |
| CCDC109B | 5.46E−16 | −0.38822989 | 0.145 | 0.362 | 1.15E−11 |
| IMMT | 5.52E−16 | 0.33815046 | 0.492 | 0.267 | 1.16E−11 |
| CSNK2A1 | 5.59E−16 | 0.35203684 | 0.539 | 0.304 | 1.18E−11 |
| NLN | 5.61E−16 | 0.25520106 | 0.267 | 0.09 | 1.18E−11 |
| RBM12B | 5.72E−16 | 0.25289469 | 0.28 | 0.099 | 1.20E−11 |
| DKC1 | 5.88E−16 | 0.36240081 | 0.446 | 0.224 | 1.24E−11 |
| GOSR2 | 6.02E−16 | 0.41066474 | 0.383 | 0.174 | 1.27E−11 |
| DSN1 | 6.05E−16 | 0.27998101 | 0.301 | 0.11 | 1.27E−11 |
| CANX | 6.18E−16 | 0.39236691 | 0.86 | 0.693 | 1.30E−11 |
| TSEN34 | 6.20E−16 | −0.34411756 | 0.174 | 0.401 | 1.31E−11 |
| PLIN2 | 6.27E−16 | 0.3290414 | 0.44 | 0.219 | 1.32E−11 |
| ORAI2 | 6.79E−16 | 0.28841036 | 0.316 | 0.125 | 1.43E−11 |
| SUPT20H | 7.25E−16 | 0.28427096 | 0.422 | 0.198 | 1.53E−11 |
| RFC2 | 7.61E−16 | 0.29970855 | 0.389 | 0.176 | 1.60E−11 |
| TMEM67 | 8.08E−16 | 0.25854126 | 0.277 | 0.098 | 1.70E−11 |
| APP | 8.54E−16 | 0.27426925 | 0.347 | 0.144 | 1.80E−11 |
| TCF25 | 8.55E−16 | 0.33439384 | 0.484 | 0.257 | 1.80E−11 |
| MGEA5 | 9.21E−16 | 0.34213594 | 0.484 | 0.259 | 1.94E−11 |
| SLC1A5 | 9.93E−16 | 0.39173382 | 0.363 | 0.161 | 2.09E−11 |
| DNER | 1.06E−15 | −0.42070801 | 0.127 | 0.346 | 2.24E−11 |
| TOX | 1.09E−15 | 0.28922909 | 0.311 | 0.121 | 2.31E−11 |
| PPT1 | 1.11E−15 | 0.39380037 | 0.565 | 0.353 | 2.34E−11 |
| MUTYH | 1.14E−15 | 0.27927118 | 0.298 | 0.111 | 2.40E−11 |
| LRRC49 | 1.15E−15 | 0.34703369 | 0.355 | 0.154 | 2.42E−11 |
| SRI | 1.22E−15 | −0.40482887 | 0.554 | 0.685 | 2.56E−11 |
| MKS1 | 1.37E−15 | 0.29398817 | 0.259 | 0.087 | 2.88E−11 |
| BTBD17 | 1.39E−15 | −0.40991962 | 0.135 | 0.35 | 2.94E−11 |
| CES2 | 1.42E−15 | 0.2900544 | 0.303 | 0.116 | 2.99E−11 |
| KLHL24 | 1.43E−15 | 0.31751945 | 0.347 | 0.147 | 3.02E−11 |
| MPZL1 | 1.45E−15 | 0.41340615 | 0.477 | 0.244 | 3.05E−11 |
| UMPS | 1.46E−15 | 0.25171504 | 0.321 | 0.126 | 3.08E−11 |
| SPEN | 1.48E−15 | 0.26867708 | 0.285 | 0.104 | 3.12E−11 |
| FASTK | 1.51E−15 | 0.26049457 | 0.342 | 0.142 | 3.18E−11 |
| POLR1D | 1.55E−15 | −0.35152709 | 0.124 | 0.337 | 3.26E−11 |
| ISOC1 | 1.56E−15 | 0.30288993 | 0.355 | 0.156 | 3.29E−11 |
| PMF1 | 1.61E−15 | −0.32581414 | 0.122 | 0.337 | 3.39E−11 |
| SNRPC | 1.66E−15 | −0.4188496 | 0.376 | 0.565 | 3.49E−11 |
| MAP2K1 | 1.72E−15 | −0.34623695 | 0.073 | 0.268 | 3.63E−11 |
| PRPF4B | 1.82E−15 | 0.33134524 | 0.293 | 0.11 | 3.83E−11 |
| ZNF33A | 1.90E−15 | 0.28151825 | 0.319 | 0.128 | 4.00E−11 |
| HAUS6 | 1.90E−15 | 0.25653761 | 0.303 | 0.115 | 4.00E−11 |
| LSM1 | 1.94E−15 | −0.34562054 | 0.14 | 0.356 | 4.09E−11 |
| FBXO42 | 2.03E−15 | 0.26933139 | 0.269 | 0.094 | 4.27E−11 |
| MTCH1 | 2.18E−15 | −0.42018677 | 0.37 | 0.57 | 4.58E−11 |
| PCCA | 2.25E−15 | 0.2831422 | 0.365 | 0.159 | 4.73E−11 |
| AP3M2 | 2.30E−15 | 0.30431138 | 0.282 | 0.104 | 4.84E−11 |
| USMG5 | 2.42E−15 | −0.32472382 | 0.13 | 0.347 | 5.09E−11 |
| CDK12 | 2.49E−15 | 0.30790501 | 0.293 | 0.109 | 5.25E−11 |
| UBQLN1 | 2.62E−15 | 0.3344265 | 0.443 | 0.224 | 5.51E−11 |
| PDLIM7 | 2.65E−15 | 0.34974396 | 0.648 | 0.435 | 5.59E−11 |
| VWA9 | 2.86E−15 | 0.35779352 | 0.319 | 0.13 | 6.02E−11 |
| TRAPPC4 | 2.89E−15 | −0.3700789 | 0.218 | 0.439 | 6.08E−11 |
| SUPT7L | 3.05E−15 | 0.29295336 | 0.347 | 0.149 | 6.42E−11 |
| GRB2 | 3.09E−15 | 0.32799234 | 0.425 | 0.203 | 6.51E−11 |
| CDC20 | 3.10E−15 | 0.59304866 | 0.306 | 0.123 | 6.52E−11 |
| RB1CC1 | 3.24E−15 | 0.3515973 | 0.438 | 0.222 | 6.82E−11 |
| HSPH1 | 3.32E−15 | 0.30672322 | 0.378 | 0.17 | 6.98E−11 |
| HTATSF1 | 3.48E−15 | 0.33851824 | 0.528 | 0.296 | 7.32E−11 |
| IFT81 | 3.52E−15 | 0.30065788 | 0.464 | 0.238 | 7.40E−11 |
| NKTR | 3.53E−15 | 0.47488623 | 0.508 | 0.281 | 7.42E−11 |
| RPAIN | 3.54E−15 | −0.42052405 | 0.275 | 0.483 | 7.45E−11 |
| RAB11A | 3.72E−15 | 0.5223318 | 0.687 | 0.458 | 7.83E−11 |
| FGF13 | 3.80E−15 | −0.43842471 | 0.176 | 0.388 | 8.00E−11 |
| KANSL1 | 3.83E−15 | 0.25674254 | 0.275 | 0.098 | 8.06E−11 |
| IQCB1 | 4.03E−15 | 0.26549783 | 0.324 | 0.132 | 8.49E−11 |
| N4BP2 | 4.32E−15 | 0.34935217 | 0.412 | 0.207 | 9.08E−11 |
| POLR2F | 4.38E−15 | −0.36010666 | 0.207 | 0.424 | 9.23E−11 |
| PSMB3 | 4.59E−15 | −0.416929 | 0.492 | 0.638 | 9.66E−11 |
| LAMTOR4 | 4.63E−15 | −0.34336735 | 0.233 | 0.455 | 9.74E−11 |
| APOPT1 | 4.78E−15 | −0.29712616 | 0.161 | 0.384 | 1.01E−10 |
| PDCD6IP | 5.04E−15 | 0.32198369 | 0.355 | 0.155 | 1.06E−10 |
| UBE2E2 | 5.05E−15 | −0.30913109 | 0.101 | 0.304 | 1.06E−10 |
| EMC7 | 5.29E−15 | −0.37809783 | 0.212 | 0.428 | 1.11E−10 |
| G3BP2 | 5.29E−15 | 0.34692917 | 0.57 | 0.346 | 1.11E−10 |
| PRPF6 | 5.37E−15 | 0.39141995 | 0.495 | 0.268 | 1.13E−10 |
| IFT20 | 5.38E−15 | −0.29474547 | 0.065 | 0.254 | 1.13E−10 |
| NDUFA8 | 5.41E−15 | −0.29385516 | 0.181 | 0.395 | 1.14E−10 |
| CDK5RAP2 | 5.47E−15 | 0.27692302 | 0.301 | 0.117 | 1.15E−10 |
| SNRPB2 | 5.57E−15 | −0.37759085 | 0.202 | 0.418 | 1.17E−10 |
| DAPK3 | 5.73E−15 | 0.27536755 | 0.306 | 0.123 | 1.21E−10 |
| KIAA1715 | 5.76E−15 | 0.28204568 | 0.363 | 0.16 | 1.21E−10 |
| GSTP1 | 5.84E−15 | −0.34747672 | 0.943 | 0.972 | 1.23E−10 |
| C1orf174 | 5.84E−15 | 0.27768395 | 0.311 | 0.123 | 1.23E−10 |
| POLR2G | 6.22E−15 | −0.43213533 | 0.482 | 0.638 | 1.31E−10 |
| GNPAT | 6.26E−15 | 0.33038289 | 0.412 | 0.2 | 1.32E−10 |
| FKBP5 | 6.38E−15 | 0.31345481 | 0.251 | 0.087 | 1.34E−10 |
| DLAT | 6.49E−15 | 0.26731183 | 0.313 | 0.127 | 1.37E−10 |
| LSM2 | 7.28E−15 | −0.367503 | 0.288 | 0.493 | 1.53E−10 |
| PDDC1 | 7.60E−15 | 0.2620973 | 0.264 | 0.094 | 1.60E−10 |
| SMARCA5 | 8.13E−15 | 0.37007728 | 0.715 | 0.5 | 1.71E−10 |
| MED19 | 8.18E−15 | −0.3187815 | 0.091 | 0.287 | 1.72E−10 |
| FRZB | 8.65E−15 | −0.46488789 | 0.233 | 0.43 | 1.82E−10 |
| WASF2 | 8.75E−15 | 0.28322059 | 0.412 | 0.2 | 1.84E−10 |
| LSM4 | 9.71E−15 | −0.40120386 | 0.731 | 0.846 | 2.04E−10 |
| ZNF655 | 9.96E−15 | 0.25607945 | 0.264 | 0.094 | 2.10E−10 |
| DNAJA2 | 1.03E−14 | 0.35665553 | 0.495 | 0.275 | 2.16E−10 |
| DIABLO | 1.05E−14 | 0.28204568 | 0.365 | 0.163 | 2.22E−10 |
| MED4 | 1.06E−14 | −0.39803292 | 0.223 | 0.427 | 2.24E−10 |
| FH | 1.07E−14 | 0.32087545 | 0.438 | 0.219 | 2.26E−10 |
| GLB1 | 1.09E−14 | 0.28348705 | 0.42 | 0.205 | 2.29E−10 |
| RAB4A | 1.10E−14 | −0.31769558 | 0.119 | 0.322 | 2.32E−10 |
| TOMM22 | 1.18E−14 | −0.38128486 | 0.324 | 0.528 | 2.49E−10 |
| MAP2K4 | 1.27E−14 | −0.29290127 | 0.073 | 0.264 | 2.68E−10 |
| UQCC2 | 1.49E−14 | −0.30352313 | 0.163 | 0.378 | 3.14E−10 |
| GPX4 | 1.52E−14 | −0.38776015 | 0.821 | 0.875 | 3.19E−10 |
| SLC16A9 | 1.54E−14 | −0.34561269 | 0.155 | 0.366 | 3.25E−10 |
| USO1 | 1.62E−14 | 0.35249596 | 0.35 | 0.156 | 3.41E−10 |
| GARS | 1.67E−14 | 0.32042719 | 0.541 | 0.309 | 3.51E−10 |
| VPS26A | 1.80E−14 | 0.34076628 | 0.482 | 0.26 | 3.79E−10 |
| UBE2D2 | 1.89E−14 | −0.36531873 | 0.539 | 0.689 | 3.98E−10 |
| POLR2I | 1.96E−14 | −0.3540449 | 0.205 | 0.417 | 4.13E−10 |
| ARFIP2 | 2.01E−14 | 0.25932491 | 0.332 | 0.142 | 4.24E−10 |
| ETFB | 2.06E−14 | −0.4995754 | 0.288 | 0.478 | 4.33E−10 |
| ADH5 | 2.06E−14 | −0.40591817 | 0.394 | 0.576 | 4.34E−10 |
| TMEM261 | 2.43E−14 | −0.37944282 | 0.215 | 0.427 | 5.11E−10 |
| DDX54 | 2.47E−14 | 0.25638723 | 0.288 | 0.112 | 5.19E−10 |
| SRPRB | 2.47E−14 | 0.32899696 | 0.321 | 0.136 | 5.20E−10 |
| PRRC1 | 2.51E−14 | 0.29291192 | 0.313 | 0.13 | 5.28E−10 |
| ORC4 | 2.86E−14 | 0.337575 | 0.415 | 0.208 | 6.02E−10 |
| SUCO | 3.03E−14 | 0.31513921 | 0.399 | 0.198 | 6.37E−10 |
| CTNNBL1 | 3.07E−14 | 0.28807114 | 0.415 | 0.202 | 6.47E−10 |
| RNF7 | 3.14E−14 | −0.38123605 | 0.451 | 0.627 | 6.62E−10 |
| CXXC5 | 3.27E−14 | −0.397669 | 0.264 | 0.467 | 6.88E−10 |
| EIF4A3 | 3.28E−14 | 0.36341662 | 0.653 | 0.427 | 6.91E−10 |
| PBX1 | 3.35E−14 | 0.41672611 | 0.409 | 0.21 | 7.05E−10 |
| TMEM14C | 3.45E−14 | −0.41526977 | 0.459 | 0.625 | 7.25E−10 |
| TCEAL8 | 3.68E−14 | −0.29852712 | 0.285 | 0.496 | 7.74E−10 |
| TCEAL4 | 3.72E−14 | −0.28918862 | 0.352 | 0.553 | 7.83E−10 |
| HSP90AB1 | 3.85E−14 | 0.31965082 | 0.964 | 0.983 | 8.11E−10 |
| MTMR2 | 3.98E−14 | 0.30567927 | 0.355 | 0.164 | 8.37E−10 |
| ANAPC11 | 4.18E−14 | −0.42219138 | 0.479 | 0.622 | 8.80E−10 |
| FARSB | 4.38E−14 | 0.2924797 | 0.358 | 0.163 | 9.22E−10 |
| FAM219B | 4.58E−14 | 0.3043519 | 0.308 | 0.128 | 9.64E−10 |
| SLC25A5 | 4.67E−14 | −0.42448312 | 0.648 | 0.715 | 9.83E−10 |
| MPDU1 | 4.68E−14 | 0.27614507 | 0.363 | 0.164 | 9.84E−10 |
| IWS1 | 4.83E−14 | 0.27302573 | 0.282 | 0.11 | 1.02E−09 |
| CNOT7 | 4.94E−14 | 0.33981223 | 0.596 | 0.359 | 1.04E−09 |
| DYNLT1 | 4.99E−14 | −0.43259717 | 0.469 | 0.611 | 1.05E−09 |
| MLF1 | 5.21E−14 | −0.29080371 | 0.13 | 0.329 | 1.10E−09 |
| LMO4 | 5.56E−14 | −0.37062867 | 0.145 | 0.344 | 1.17E−09 |
| DPP3 | 5.80E−14 | 0.27418356 | 0.28 | 0.11 | 1.22E−09 |
| AK4 | 5.88E−14 | −0.35174931 | 0.117 | 0.315 | 1.24E−09 |
| HMGN1 | 5.98E−14 | −0.39565609 | 0.915 | 0.94 | 1.26E−09 |
| MTG1 | 6.32E−14 | 0.30422937 | 0.37 | 0.174 | 1.33E−09 |
| KCNQ1OT1 | 6.35E−14 | 0.48469859 | 0.536 | 0.336 | 1.34E−09 |
| CCDC88A | 6.37E−14 | 0.3032812 | 0.412 | 0.209 | 1.34E−09 |
| PGLS | 6.54E−14 | −0.38549649 | 0.383 | 0.566 | 1.38E−09 |
| HERPUD1 | 6.56E−14 | −0.55892653 | 0.262 | 0.455 | 1.38E−09 |
| CCDC130 | 6.73E−14 | 0.26910943 | 0.28 | 0.109 | 1.42E−09 |
| SH3BGRL | 7.02E−14 | −0.34043756 | 0.503 | 0.645 | 1.48E−09 |
| DST | 7.09E−14 | 0.34605438 | 0.368 | 0.176 | 1.49E−09 |
| TIMM13 | 7.46E−14 | −0.37640503 | 0.355 | 0.54 | 1.57E−09 |
| IFT57 | 7.55E−14 | 0.41370408 | 0.541 | 0.324 | 1.59E−09 |
| BTG2 | 7.92E−14 | 0.34288662 | 0.267 | 0.103 | 1.67E−09 |
| SERPINH1 | 7.98E−14 | 0.31829211 | 0.627 | 0.396 | 1.68E−09 |
| ZMYND11 | 8.34E−14 | 0.28250057 | 0.259 | 0.098 | 1.76E−09 |
| MRPL17 | 8.36E−14 | −0.32111186 | 0.101 | 0.291 | 1.76E−09 |
| G2E3 | 8.41E−14 | 0.29015949 | 0.28 | 0.112 | 1.77E−09 |
| EIF5 | 8.77E−14 | 0.38037477 | 0.826 | 0.647 | 1.85E−09 |
| FRMD4A | 8.85E−14 | 0.25794048 | 0.324 | 0.138 | 1.86E−09 |
| GOLGA4 | 9.13E−14 | 0.31280882 | 0.363 | 0.172 | 1.92E−09 |
| ZNF593 | 9.17E−14 | 0.27837892 | 0.345 | 0.158 | 1.93E−09 |
| ALG8 | 9.22E−14 | 0.25846115 | 0.365 | 0.169 | 1.94E−09 |
| LARP4 | 9.78E−14 | 0.29773389 | 0.264 | 0.099 | 2.06E−09 |
| BLOC1S2 | 1.07E−13 | −0.34283754 | 0.174 | 0.377 | 2.26E−09 |
| ENC1 | 1.13E−13 | 0.32823359 | 0.298 | 0.121 | 2.39E−09 |
| VTA1 | 1.14E−13 | 0.2502246 | 0.453 | 0.233 | 2.41E−09 |
| PRCP | 1.22E−13 | 0.28740519 | 0.391 | 0.186 | 2.56E−09 |
| ZNF207 | 1.22E−13 | 0.31271969 | 0.645 | 0.413 | 2.58E−09 |
| EFHC1 | 1.23E−13 | 0.27578793 | 0.399 | 0.202 | 2.59E−09 |
| DZIP3 | 1.28E−13 | 0.26858772 | 0.332 | 0.147 | 2.70E−09 |
| AATF | 1.34E−13 | 0.2508062 | 0.28 | 0.111 | 2.82E−09 |
| GPS1 | 1.38E−13 | 0.28161501 | 0.446 | 0.236 | 2.90E−09 |
| PPP2R5E | 1.48E−13 | −0.33350491 | 0.163 | 0.367 | 3.12E−09 |
| PSD3 | 1.55E−13 | 0.34550427 | 0.293 | 0.122 | 3.27E−09 |
| TRIM28 | 1.56E−13 | 0.26636405 | 0.285 | 0.115 | 3.28E−09 |
| PPIF | 1.58E−13 | 0.25394034 | 0.295 | 0.121 | 3.32E−09 |
| PCBP1 | 1.76E−13 | −0.40020571 | 0.513 | 0.65 | 3.71E−09 |
| NELFA | 1.77E−13 | 0.25203042 | 0.272 | 0.104 | 3.72E−09 |
| NUDT15 | 1.85E−13 | 0.29947758 | 0.329 | 0.143 | 3.90E−09 |
| PEPD | 1.97E−13 | 0.3392296 | 0.373 | 0.185 | 4.14E−09 |
| CCDC90B | 1.99E−13 | −0.37871944 | 0.37 | 0.554 | 4.19E−09 |
| WDR33 | 2.39E−13 | 0.26437858 | 0.482 | 0.259 | 5.04E−09 |
| CALM1 | 2.45E−13 | −0.4542974 | 0.692 | 0.79 | 5.16E−09 |
| ATXN2L | 2.46E−13 | 0.32708664 | 0.321 | 0.143 | 5.18E−09 |
| TMOD3 | 2.80E−13 | 0.29015377 | 0.321 | 0.143 | 5.89E−09 |
| CHRAC1 | 2.84E−13 | 0.27428281 | 0.345 | 0.159 | 5.99E−09 |
| MEA1 | 2.87E−13 | −0.35755309 | 0.197 | 0.397 | 6.05E−09 |
| C11orf31 | 2.92E−13 | −0.32717231 | 0.541 | 0.674 | 6.14E−09 |
| FAM229B | 2.93E−13 | −0.31265949 | 0.228 | 0.436 | 6.16E−09 |
| ZFAND3 | 2.96E−13 | −0.32713232 | 0.202 | 0.402 | 6.23E−09 |
| SNRNP70 | 3.11E−13 | −0.36597815 | 0.225 | 0.416 | 6.55E−09 |
| CCNT2 | 3.12E−13 | 0.26871487 | 0.402 | 0.199 | 6.57E−09 |
| SRSF4 | 3.12E−13 | 0.35525849 | 0.588 | 0.383 | 6.57E−09 |
| GORASP2 | 3.34E−13 | 0.28143937 | 0.479 | 0.265 | 7.03E−09 |
| GDE1 | 3.36E−13 | 0.26004177 | 0.36 | 0.17 | 7.07E−09 |
| DDIT3 | 3.37E−13 | −0.4305627 | 0.078 | 0.253 | 7.09E−09 |
| FBXW5 | 3.64E−13 | 0.26847523 | 0.311 | 0.136 | 7.67E−09 |
| LINC00094 | 3.71E−13 | 0.27725555 | 0.352 | 0.165 | 7.80E−09 |
| ATIC | 3.71E−13 | 0.32843499 | 0.404 | 0.211 | 7.81E−09 |
| MRPL52 | 3.85E−13 | −0.26195082 | 0.109 | 0.3 | 8.10E−09 |
| CNPY2 | 3.87E−13 | 0.26319379 | 0.448 | 0.24 | 8.14E−09 |
| SCYL1 | 3.97E−13 | 0.25689052 | 0.272 | 0.109 | 8.36E−09 |
| CHIC2 | 3.99E−13 | −0.28271738 | 0.075 | 0.251 | 8.40E−09 |
| ZNF268 | 4.00E−13 | 0.27143295 | 0.282 | 0.116 | 8.43E−09 |
| LTA4H | 4.16E−13 | 0.44541134 | 0.567 | 0.357 | 8.77E−09 |
| PREP | 4.18E−13 | 0.25623453 | 0.262 | 0.103 | 8.79E−09 |
| GOLGA7 | 4.22E−13 | −0.33263004 | 0.244 | 0.44 | 8.89E−09 |
| GCA | 4.23E−13 | −0.3331576 | 0.148 | 0.339 | 8.91E−09 |
| TUSC3 | 4.38E−13 | −0.39411203 | 0.422 | 0.593 | 9.23E−09 |
| HN1 | 4.54E−13 | 0.42127237 | 0.663 | 0.479 | 9.57E−09 |
| EMC4 | 4.63E−13 | −0.40285491 | 0.383 | 0.55 | 9.75E−09 |
| IFITM3 | 4.65E−13 | 0.34808861 | 0.541 | 0.333 | 9.80E−09 |
| OAT | 4.79E−13 | 0.29369328 | 0.378 | 0.185 | 1.01E−08 |
| CCT7 | 4.83E−13 | 0.36605385 | 0.764 | 0.598 | 1.02E−08 |
| TRIB2 | 5.15E−13 | 0.32780719 | 0.37 | 0.183 | 1.08E−08 |
| PUF60 | 5.28E−13 | 0.38808085 | 0.746 | 0.534 | 1.11E−08 |
| JAK1 | 5.37E−13 | 0.94747087 | 0.332 | 0.148 | 1.13E−08 |
| SDHC | 5.57E−13 | −0.40117169 | 0.373 | 0.544 | 1.17E−08 |
| SGCE | 5.57E−13 | −0.26731506 | 0.111 | 0.301 | 1.17E−08 |
| NHP2 | 5.70E−13 | −0.42525194 | 0.453 | 0.597 | 1.20E−08 |
| ISCU | 5.96E−13 | −0.38987081 | 0.282 | 0.469 | 1.26E−08 |
| ATG4B | 6.11E−13 | 0.2662948 | 0.363 | 0.172 | 1.29E−08 |
| WDR18 | 6.22E−13 | 0.42073222 | 0.583 | 0.366 | 1.31E−08 |
| POLD3 | 6.28E−13 | 0.27917913 | 0.319 | 0.147 | 1.32E−08 |
| TRAF7 | 6.40E−13 | 0.28561575 | 0.259 | 0.1 | 1.35E−08 |
| TMEM98 | 6.43E−13 | 0.3402539 | 0.694 | 0.469 | 1.35E−08 |
| OCIAD1 | 6.51E−13 | −0.33126257 | 0.513 | 0.653 | 1.37E−08 |
| DHX32 | 6.71E−13 | 0.2665819 | 0.288 | 0.12 | 1.41E−08 |
| H2AFX | 6.93E−13 | 0.40313426 | 0.399 | 0.21 | 1.46E−08 |
| GFPT1 | 7.53E−13 | 0.28984888 | 0.306 | 0.136 | 1.59E−08 |
| OSBPL9 | 7.63E−13 | 0.31235424 | 0.461 | 0.26 | 1.61E−08 |
| URI1 | 7.67E−13 | 0.36537291 | 0.262 | 0.105 | 1.61E−08 |
| EIF3A | 7.79E−13 | 0.29896652 | 0.57 | 0.352 | 1.64E−08 |
| ASNA1 | 9.85E−13 | −0.27056072 | 0.106 | 0.289 | 2.07E−08 |
| SNX2 | 9.93E−13 | 0.31581037 | 0.518 | 0.308 | 2.09E−08 |
| C19orf48 | 1.03E−12 | 0.35719981 | 0.503 | 0.303 | 2.17E−08 |
| SERPINF1 | 1.03E−12 | −0.36330007 | 0.122 | 0.303 | 2.17E−08 |
| TMSB15A | 1.06E−12 | 0.39077724 | 0.653 | 0.445 | 2.23E−08 |
| NUP62CL | 1.14E−12 | 0.34852227 | 0.313 | 0.142 | 2.41E−08 |
| KLHDC3 | 1.17E−12 | 0.28937385 | 0.534 | 0.318 | 2.46E−08 |
| DDOST | 1.23E−12 | 0.25720124 | 0.699 | 0.476 | 2.58E−08 |
| TENM3 | 1.26E−12 | 0.48508754 | 0.251 | 0.105 | 2.65E−08 |
| CLTA | 1.33E−12 | −0.36463519 | 0.583 | 0.7 | 2.80E−08 |
| PPP1R12A | 1.42E−12 | 0.31477638 | 0.464 | 0.26 | 2.99E−08 |
| C4orf48 | 1.46E−12 | −0.41603873 | 0.319 | 0.504 | 3.07E−08 |
| PIAS1 | 1.54E−12 | 0.28322059 | 0.404 | 0.214 | 3.24E−08 |
| TAF12 | 1.58E−12 | −0.26625794 | 0.083 | 0.256 | 3.33E−08 |
| DAZAP2 | 1.65E−12 | −0.26554298 | 0.212 | 0.413 | 3.48E−08 |
| ELP2 | 1.69E−12 | 0.27009603 | 0.313 | 0.141 | 3.55E−08 |
| MIS12 | 1.79E−12 | 0.28390847 | 0.347 | 0.166 | 3.76E−08 |
| GPM6B | 1.96E−12 | −0.3705653 | 0.541 | 0.732 | 4.13E−08 |
| ZBTB20 | 2.23E−12 | 0.32941022 | 0.368 | 0.189 | 4.70E−08 |
| C22orf39 | 2.35E−12 | −0.29112099 | 0.161 | 0.35 | 4.94E−08 |
| AASDHPPT | 2.39E−12 | 0.31760722 | 0.554 | 0.346 | 5.03E−08 |
| AAR2 | 2.48E−12 | 0.2983703 | 0.285 | 0.121 | 5.22E−08 |
| RNF34 | 2.62E−12 | 0.25359355 | 0.316 | 0.143 | 5.52E−08 |
| DUT | 2.81E−12 | −0.41469204 | 0.407 | 0.598 | 5.93E−08 |
| HPS4 | 3.32E−12 | 0.25759777 | 0.319 | 0.147 | 7.00E−08 |
| WNK3 | 3.34E−12 | 0.26585776 | 0.303 | 0.134 | 7.04E−08 |
| SNRPD1 | 3.49E−12 | −0.27400266 | 0.503 | 0.672 | 7.35E−08 |
| MRPL47 | 3.51E−12 | −0.30660911 | 0.184 | 0.378 | 7.39E−08 |
| UFC1 | 4.02E−12 | −0.33294022 | 0.259 | 0.45 | 8.46E−08 |
| TUSC2 | 4.04E−12 | −0.28764509 | 0.124 | 0.302 | 8.51E−08 |
| SENP6 | 4.33E−12 | 0.27541699 | 0.347 | 0.169 | 9.13E−08 |
| DDR1 | 4.35E−12 | 0.32087911 | 0.604 | 0.403 | 9.15E−08 |
| FNBP4 | 4.36E−12 | 0.26132687 | 0.425 | 0.227 | 9.19E−08 |
| SNW1 | 4.38E−12 | 0.26600306 | 0.526 | 0.315 | 9.21E−08 |
| MICU2 | 4.47E−12 | 0.28663052 | 0.5 | 0.292 | 9.42E−08 |
| RAD50 | 4.64E−12 | 0.29051432 | 0.396 | 0.214 | 9.76E−08 |
| SLC25A39 | 4.85E−12 | 0.36890222 | 0.526 | 0.318 | 1.02E−07 |
| WRB | 4.90E−12 | −0.36692038 | 0.407 | 0.565 | 1.03E−07 |
| NPEPPS | 5.21E−12 | 0.25633659 | 0.355 | 0.176 | 1.10E−07 |
| FAM92A1 | 5.48E−12 | 0.26444278 | 0.505 | 0.29 | 1.15E−07 |
| TTC14 | 5.86E−12 | 0.30131401 | 0.402 | 0.216 | 1.23E−07 |
| GNL2 | 5.86E−12 | 0.3437759 | 0.412 | 0.221 | 1.23E−07 |
| STMN4 | 5.92E−12 | −0.56640618 | 0.218 | 0.401 | 1.25E−07 |
| FSIP2 | 6.07E−12 | 0.29652353 | 0.298 | 0.138 | 1.28E−07 |
| NDUFA5 | 6.11E−12 | −0.35351084 | 0.425 | 0.578 | 1.29E−07 |
| IGBP1 | 6.28E−12 | −0.33887162 | 0.176 | 0.361 | 1.32E−07 |
| C14orf166 | 6.35E−12 | −0.39771755 | 0.624 | 0.7 | 1.34E−07 |
| RNMT | 6.35E−12 | 0.25503031 | 0.365 | 0.182 | 1.34E−07 |
| CD2BP2 | 6.40E−12 | 0.26293158 | 0.422 | 0.225 | 1.35E−07 |
| TMEM106C | 6.41E−12 | 0.26919231 | 0.459 | 0.257 | 1.35E−07 |
| PSMD8 | 6.85E−12 | −0.33729477 | 0.635 | 0.699 | 1.44E−07 |
| BIRC2 | 6.96E−12 | 0.25913284 | 0.345 | 0.167 | 1.46E−07 |
| SPAG7 | 7.15E−12 | −0.32857205 | 0.233 | 0.418 | 1.51E−07 |
| DOCK7 | 7.18E−12 | 0.25130495 | 0.251 | 0.101 | 1.51E−07 |
| GNG5 | 7.20E−12 | −0.38158767 | 0.526 | 0.632 | 1.52E−07 |
| SRP68 | 7.89E−12 | 0.27203643 | 0.321 | 0.152 | 1.66E−07 |
| CHTOP | 8.12E−12 | 0.26189459 | 0.534 | 0.323 | 1.71E−07 |
| CCNL1 | 8.39E−12 | 0.26088534 | 0.466 | 0.265 | 1.77E−07 |
| CLSTN1 | 8.93E−12 | 0.41083933 | 0.321 | 0.152 | 1.88E−07 |
| PCMT1 | 9.38E−12 | −0.36265034 | 0.492 | 0.636 | 1.98E−07 |
| C14orf1 | 1.06E−11 | −0.40324496 | 0.355 | 0.52 | 2.24E−07 |
| DCP1A | 1.09E−11 | 0.25820459 | 0.329 | 0.155 | 2.30E−07 |
| CIRH1A | 1.11E−11 | 0.27042084 | 0.298 | 0.134 | 2.34E−07 |
| DCTN6 | 1.14E−11 | −0.30657059 | 0.168 | 0.352 | 2.39E−07 |
| HSD17B10 | 1.16E−11 | −0.3161504 | 0.308 | 0.489 | 2.45E−07 |
| CAMLG | 1.23E−11 | −0.31476776 | 0.29 | 0.472 | 2.60E−07 |
| RNF11 | 1.25E−11 | −0.28153704 | 0.091 | 0.256 | 2.63E−07 |
| FTX | 1.29E−11 | −0.32356563 | 0.117 | 0.286 | 2.71E−07 |
| ZC3H13 | 1.41E−11 | 0.31861322 | 0.479 | 0.285 | 2.97E−07 |
| METTL5 | 1.43E−11 | −0.26792661 | 0.096 | 0.263 | 3.02E−07 |
| MRS2 | 1.47E−11 | 0.25820459 | 0.326 | 0.158 | 3.10E−07 |
| SMIM20 | 1.51E−11 | −0.30137553 | 0.117 | 0.286 | 3.18E−07 |
| HBP1 | 1.89E−11 | 0.272066 | 0.326 | 0.158 | 3.98E−07 |
| POLD2 | 1.89E−11 | 0.3155551 | 0.63 | 0.429 | 3.99E−07 |
| CLPP | 1.95E−11 | −0.3191004 | 0.192 | 0.372 | 4.10E−07 |
| DPY30 | 2.19E−11 | −0.31101298 | 0.184 | 0.367 | 4.61E−07 |
| ASAP1 | 2.26E−11 | 0.27062807 | 0.259 | 0.11 | 4.75E−07 |
| FIS1 | 2.64E−11 | −0.30176993 | 0.334 | 0.504 | 5.56E−07 |
| NIPBL | 2.73E−11 | 0.25778454 | 0.396 | 0.215 | 5.75E−07 |
| AP3B1 | 2.84E−11 | 0.27231414 | 0.394 | 0.205 | 5.98E−07 |
| VPS51 | 2.95E−11 | 0.28995087 | 0.345 | 0.175 | 6.21E−07 |
| SH3GLB1 | 3.04E−11 | 0.27197342 | 0.409 | 0.222 | 6.41E−07 |
| XPO1 | 3.25E−11 | 0.29121792 | 0.544 | 0.348 | 6.84E−07 |
| PDCD4 | 3.47E−11 | 0.2585867 | 0.407 | 0.224 | 7.30E−07 |
| 11-Sep | 3.53E−11 | 0.29576446 | 0.707 | 0.506 | 7.42E−07 |
| MRPL32 | 3.71E−11 | −0.28382 | 0.117 | 0.285 | 7.80E−07 |
| PODXL2 | 3.81E−11 | −0.34465471 | 0.218 | 0.394 | 8.02E−07 |
| TRAF4 | 4.26E−11 | −0.26998936 | 0.096 | 0.257 | 8.96E−07 |
| INSIG1 | 4.72E−11 | 0.39195156 | 0.342 | 0.182 | 9.94E−07 |
| ZNF644 | 5.11E−11 | 0.25107589 | 0.521 | 0.32 | 1.08E−06 |
| WDR73 | 5.13E−11 | 0.30291963 | 0.402 | 0.221 | 1.08E−06 |
| SPATC1L | 6.07E−11 | −0.27099596 | 0.119 | 0.286 | 1.28E−06 |
| NUDT21 | 6.09E−11 | 0.279339 | 0.484 | 0.292 | 1.28E−06 |
| XRN2 | 6.19E−11 | 0.29408091 | 0.604 | 0.396 | 1.30E−06 |
| NAA20 | 7.10E−11 | −0.30807853 | 0.184 | 0.356 | 1.50E−06 |
| HSPA13 | 7.20E−11 | 0.25808346 | 0.433 | 0.246 | 1.52E−06 |
| SNX17 | 7.32E−11 | 0.27582558 | 0.492 | 0.301 | 1.54E−06 |
| CAP1 | 7.33E−11 | 0.32391361 | 0.622 | 0.402 | 1.54E−06 |
| COMMD1 | 7.60E−11 | −0.2530041 | 0.142 | 0.313 | 1.60E−06 |
| RERE | 7.82E−11 | 0.25598631 | 0.422 | 0.238 | 1.65E−06 |
| RBPJ | 8.76E−11 | 0.31133112 | 0.614 | 0.416 | 1.85E−06 |
| SRRM1 | 9.42E−11 | 0.28635223 | 0.65 | 0.45 | 1.98E−06 |
| MANBAL | 9.87E−11 | −0.2935851 | 0.197 | 0.372 | 2.08E−06 |
| ADSS | 1.03E−10 | 0.26298917 | 0.376 | 0.204 | 2.17E−06 |
| SSR3 | 1.06E−10 | 0.33595447 | 0.624 | 0.436 | 2.24E−06 |
| CDK2AP2 | 1.07E−10 | 0.29459883 | 0.324 | 0.164 | 2.26E−06 |
| SMAD5 | 1.08E−10 | 0.27266916 | 0.272 | 0.123 | 2.28E−06 |
| ERP29 | 1.12E−10 | −0.38372487 | 0.503 | 0.619 | 2.37E−06 |
| PDAP1 | 1.19E−10 | −0.37187944 | 0.389 | 0.527 | 2.52E−06 |
| MED17 | 1.30E−10 | 0.25260102 | 0.332 | 0.169 | 2.74E−06 |
| PCDHB2 | 1.36E−10 | 0.28419735 | 0.275 | 0.128 | 2.86E−06 |
| KDM5B | 1.48E−10 | 0.33800759 | 0.557 | 0.394 | 3.13E−06 |
| CCDC115 | 1.50E−10 | −0.26961954 | 0.197 | 0.37 | 3.17E−06 |
| TCF4 | 1.59E−10 | 0.30609586 | 0.365 | 0.197 | 3.34E−06 |
| ACTR1B | 1.67E−10 | 0.27504727 | 0.342 | 0.182 | 3.53E−06 |
| KLHL7 | 1.75E−10 | 0.25824787 | 0.453 | 0.273 | 3.69E−06 |
| C12orf76 | 1.78E−10 | −0.25582959 | 0.135 | 0.3 | 3.74E−06 |
| GPC3 | 1.84E−10 | −0.38520944 | 0.148 | 0.311 | 3.87E−06 |
| HSPA5 | 1.91E−10 | 0.44333511 | 0.808 | 0.688 | 4.01E−06 |
| ATP5F1 | 1.93E−10 | −0.33734313 | 0.547 | 0.655 | 4.07E−06 |
| CD200 | 2.16E−10 | −0.28066864 | 0.142 | 0.303 | 4.54E−06 |
| TMEM179B | 2.21E−10 | −0.27597006 | 0.13 | 0.291 | 4.65E−06 |
| METRN | 2.40E−10 | −0.29956839 | 0.153 | 0.319 | 5.06E−06 |
| PRPF4 | 2.53E−10 | 0.25725642 | 0.306 | 0.149 | 5.33E−06 |
| LAMTOR1 | 2.53E−10 | −0.33186007 | 0.373 | 0.526 | 5.34E−06 |
| PEBP1 | 2.60E−10 | −0.34110386 | 0.93 | 0.927 | 5.48E−06 |
| NDUFV3 | 3.01E−10 | −0.27676815 | 0.158 | 0.323 | 6.34E−06 |
| TMEM219 | 3.11E−10 | −0.28467791 | 0.21 | 0.377 | 6.56E−06 |
| AP3S1 | 3.15E−10 | −0.27412196 | 0.251 | 0.418 | 6.64E−06 |
| EAPP | 3.26E−10 | −0.30335028 | 0.238 | 0.406 | 6.86E−06 |
| PJA1 | 3.69E−10 | 0.28192879 | 0.412 | 0.243 | 7.77E−06 |
| CFDP1 | 3.71E−10 | −0.33948626 | 0.37 | 0.521 | 7.80E−06 |
| NTPCR | 3.83E−10 | −0.31395319 | 0.168 | 0.331 | 8.06E−06 |
| ZCCHC17 | 4.13E−10 | −0.31335356 | 0.246 | 0.406 | 8.70E−06 |
| PAPOLA | 4.35E−10 | 0.31612155 | 0.715 | 0.553 | 9.17E−06 |
| LSM3 | 4.60E−10 | −0.2690998 | 0.207 | 0.373 | 9.69E−06 |
| COL4A5 | 5.06E−10 | 0.27205619 | 0.389 | 0.218 | 1.07E−05 |
| CRABP2 | 5.16E−10 | −0.48487009 | 0.342 | 0.494 | 1.09E−05 |
| MDH2 | 5.33E−10 | −0.36007555 | 0.573 | 0.661 | 1.12E−05 |
| IK | 5.77E−10 | 0.2538294 | 0.516 | 0.324 | 1.21E−05 |
| UNC119 | 5.92E−10 | −0.27889734 | 0.174 | 0.341 | 1.25E−05 |
| EFNB1 | 6.24E−10 | −0.28389432 | 0.184 | 0.355 | 1.31E−05 |
| RMI1 | 7.00E−10 | 0.25609418 | 0.295 | 0.145 | 1.47E−05 |
| EIF4E | 7.15E−10 | −0.32526945 | 0.308 | 0.461 | 1.51E−05 |
| WBP5 | 7.76E−10 | −0.3373763 | 0.303 | 0.467 | 1.63E−05 |
| CSTB | 7.80E−10 | −0.30921296 | 0.301 | 0.457 | 1.64E−05 |
| MCL1 | 7.85E−10 | 0.28061855 | 0.474 | 0.302 | 1.65E−05 |
| YES1 | 8.13E−10 | 0.25773304 | 0.311 | 0.16 | 1.71E−05 |
| PBRM1 | 8.88E−10 | 0.28703339 | 0.404 | 0.233 | 1.87E−05 |
| SYNE2 | 9.12E−10 | 0.34521897 | 0.619 | 0.482 | 1.92E−05 |
| TRAM1 | 9.34E−10 | 0.25547866 | 0.575 | 0.384 | 1.97E−05 |
| YY1 | 9.53E−10 | −0.25627085 | 0.148 | 0.308 | 2.01E−05 |
| PAFAH1B3 | 9.64E−10 | −0.38418679 | 0.438 | 0.559 | 2.03E−05 |
| ECI2 | 1.06E−09 | −0.29888831 | 0.212 | 0.37 | 2.24E−05 |
| NAE1 | 1.07E−09 | 0.28016933 | 0.562 | 0.375 | 2.26E−05 |
| SH3BGRL3 | 1.11E−09 | −0.35404148 | 0.443 | 0.573 | 2.34E−05 |
| HSDL2 | 1.19E−09 | 0.27690021 | 0.298 | 0.148 | 2.50E−05 |
| NDUFS8 | 1.19E−09 | −0.29079784 | 0.484 | 0.593 | 2.51E−05 |
| BCAS2 | 1.21E−09 | −0.30999916 | 0.313 | 0.474 | 2.55E−05 |
| TMEM14A | 1.27E−09 | −0.25007846 | 0.124 | 0.278 | 2.68E−05 |
| CCDC34 | 1.37E−09 | 0.26866349 | 0.358 | 0.194 | 2.89E−05 |
| RTF1 | 1.40E−09 | −0.33538423 | 0.303 | 0.452 | 2.95E−05 |
| AIMP1 | 1.42E−09 | −0.25229075 | 0.119 | 0.27 | 2.98E−05 |
| TUBG1 | 1.61E−09 | 0.25234462 | 0.378 | 0.211 | 3.40E−05 |
| MTR | 1.83E−09 | 0.25792438 | 0.324 | 0.174 | 3.85E−05 |
| EIF2S2 | 1.92E−09 | −0.34865869 | 0.324 | 0.471 | 4.04E−05 |
| LINC00674 | 2.01E−09 | 0.25205946 | 0.412 | 0.248 | 4.23E−05 |
| ZNF706 | 2.03E−09 | −0.27219208 | 0.342 | 0.504 | 4.27E−05 |
| CCDC12 | 2.06E−09 | −0.26588506 | 0.174 | 0.333 | 4.33E−05 |
| ARHGDIA | 2.25E−09 | −0.27209145 | 0.161 | 0.314 | 4.74E−05 |
| NT5C3B | 2.27E−09 | −0.30795483 | 0.223 | 0.38 | 4.79E−05 |
| MRPL18 | 2.29E−09 | −0.26207937 | 0.218 | 0.375 | 4.82E−05 |
| SRSF7 | 2.43E−09 | 0.30217985 | 0.847 | 0.729 | 5.11E−05 |
| ACTR10 | 2.57E−09 | −0.29924797 | 0.249 | 0.406 | 5.42E−05 |
| SHMT2 | 2.63E−09 | −0.3283308 | 0.332 | 0.48 | 5.54E−05 |
| ATP6V0E1 | 2.63E−09 | −0.26503535 | 0.244 | 0.406 | 5.55E−05 |
| SEC62 | 2.71E−09 | −0.29996898 | 0.295 | 0.455 | 5.70E−05 |
| MRPS23 | 2.84E−09 | −0.25635001 | 0.153 | 0.306 | 5.99E−05 |
| TRH | 3.45E−09 | 0.41196106 | 0.415 | 0.256 | 7.25E−05 |
| STARD3NL | 4.11E−09 | −0.32955442 | 0.339 | 0.484 | 8.65E−05 |
| AMZ2 | 4.13E−09 | −0.26987612 | 0.202 | 0.361 | 8.69E−05 |
| TUBGCP2 | 4.16E−09 | 0.27614507 | 0.337 | 0.187 | 8.75E−05 |
| SUMO1 | 4.89E−09 | −0.25477095 | 0.782 | 0.819 | 0.000103 |
| HMGA2 | 4.90E−09 | −0.31064016 | 0.155 | 0.3 | 0.000103 |
| STRA13 | 5.20E−09 | −0.28846878 | 0.215 | 0.367 | 0.000109 |
| PDZD11 | 5.44E−09 | −0.25867463 | 0.184 | 0.339 | 0.000115 |
| EI24 | 5.58E−09 | −0.30005676 | 0.301 | 0.452 | 0.000117 |
| FAM32A | 5.80E−09 | 0.25117816 | 0.658 | 0.493 | 0.000122 |
| GAP43 | 8.33E−09 | −0.80655711 | 0.171 | 0.306 | 0.000175 |
| EIF1AX | 9.49E−09 | −0.26519501 | 0.301 | 0.444 | 0.0002 |
| LGALS3BP | 9.67E−09 | 0.26027659 | 0.412 | 0.257 | 0.000204 |
| SRSF3 | 9.67E−09 | 0.26945286 | 0.927 | 0.813 | 0.000204 |
| MYL12B | 1.06E−08 | −0.33507987 | 0.552 | 0.637 | 0.000224 |
| KARS | 1.09E−08 | 0.27889569 | 0.598 | 0.416 | 0.00023 |
| CST3 | 1.21E−08 | −0.3516143 | 0.521 | 0.609 | 0.000255 |
| TUBA1B | 1.21E−08 | 0.79945757 | 0.85 | 0.949 | 0.000255 |
| MRPL22 | 1.24E−08 | −0.25704378 | 0.176 | 0.325 | 0.000262 |
| SSBP1 | 1.32E−08 | −0.36776797 | 0.438 | 0.538 | 0.000278 |
| RNPS1 | 1.49E−08 | 0.30624468 | 0.785 | 0.632 | 0.000313 |
| FAM136A | 1.51E−08 | 0.30037119 | 0.443 | 0.279 | 0.000318 |
| EIF3I | 1.82E−08 | −0.31448234 | 0.596 | 0.655 | 0.000383 |
| TRAP1 | 1.84E−08 | 0.3832223 | 0.44 | 0.284 | 0.000386 |
| YIF1A | 1.84E−08 | −0.3259239 | 0.293 | 0.433 | 0.000388 |
| CNIH4 | 2.18E−08 | −0.27412934 | 0.174 | 0.317 | 0.000459 |
| GSTA4 | 2.19E−08 | −0.316816 | 0.396 | 0.522 | 0.000462 |
| MRPL4 | 2.70E−08 | −0.28650284 | 0.22 | 0.367 | 0.000569 |
| C1QBP | 2.79E−08 | −0.31497104 | 0.479 | 0.59 | 0.000587 |
| ERI3 | 2.84E−08 | −0.25454777 | 0.308 | 0.454 | 0.000598 |
| PHGDH | 3.22E−08 | −0.55540131 | 0.474 | 0.546 | 0.000677 |
| ZIC3 | 3.48E−08 | −0.33021478 | 0.477 | 0.592 | 0.000732 |
| MFSD10 | 4.09E−08 | 0.27784376 | 0.407 | 0.258 | 0.000861 |
| THYN1 | 4.33E−08 | −0.26891847 | 0.29 | 0.432 | 0.000913 |
| TMEM230 | 4.51E−08 | −0.32421659 | 0.415 | 0.527 | 0.000949 |
| HMGCS1 | 4.63E−08 | 0.46599595 | 0.565 | 0.438 | 0.000976 |
| PSMB6 | 4.63E−08 | −0.32686606 | 0.635 | 0.665 | 0.000976 |
| KIF5B | 5.94E−08 | 0.27700408 | 0.746 | 0.588 | 0.001251 |
| CALD1 | 6.20E−08 | 0.37101806 | 0.492 | 0.326 | 0.001304 |
| RSL24D1 | 7.59E−08 | −0.3656163 | 0.482 | 0.575 | 0.001598 |
| CSRP2 | 7.64E−08 | −0.31504938 | 0.492 | 0.599 | 0.001608 |
| PSMA2 | 8.31E−08 | −0.30043776 | 0.443 | 0.543 | 0.001748 |
| ST13 | 8.45E−08 | −0.26584162 | 0.832 | 0.829 | 0.00178 |
| SNAP47 | 9.16E−08 | 0.253425 | 0.256 | 0.131 | 0.001928 |
| EXOSC7 | 9.46E−08 | −0.25558865 | 0.197 | 0.336 | 0.001992 |
| MPLKIP | 1.14E−07 | −0.25502461 | 0.223 | 0.364 | 0.002398 |
| NDUFA6 | 1.19E−07 | −0.29096376 | 0.44 | 0.555 | 0.002495 |
| PRKCSH | 1.21E−07 | −0.2530452 | 0.244 | 0.384 | 0.002543 |
| PSMB5 | 1.26E−07 | −0.29173472 | 0.619 | 0.659 | 0.002655 |
| HMGB1 | 1.27E−07 | −0.46794958 | 0.868 | 0.94 | 0.002682 |
| HPRT1 | 1.39E−07 | −0.27176528 | 0.176 | 0.309 | 0.002919 |
| TPM1 | 1.62E−07 | −0.31830635 | 0.295 | 0.429 | 0.003408 |
| ASNS | 1.68E−07 | −0.2855407 | 0.189 | 0.32 | 0.003535 |
| UPF3A | 1.70E−07 | −0.27026655 | 0.236 | 0.372 | 0.003582 |
| EIF2S1 | 1.75E−07 | 0.28470709 | 0.492 | 0.337 | 0.003686 |
| PSIP1 | 1.77E−07 | −0.29683226 | 0.611 | 0.674 | 0.003723 |
| TUBB | 1.96E−07 | 0.25254741 | 0.977 | 0.973 | 0.004118 |
| ASNSD1 | 2.08E−07 | 0.25526634 | 0.368 | 0.227 | 0.004373 |
| ATP2B1 | 2.24E−07 | −0.30081892 | 0.321 | 0.447 | 0.004715 |
| CWC15 | 2.54E−07 | −0.25694048 | 0.381 | 0.498 | 0.005353 |
| BCAT1 | 2.95E−07 | 0.25578042 | 0.402 | 0.259 | 0.006213 |
| WDR13 | 2.99E−07 | 0.25629033 | 0.324 | 0.193 | 0.006286 |
| ECH1 | 3.36E−07 | −0.2688819 | 0.415 | 0.537 | 0.007063 |
| RPA2 | 3.61E−07 | 0.25355437 | 0.51 | 0.369 | 0.007591 |
| TTC3 | 4.49E−07 | −0.31845943 | 0.829 | 0.866 | 0.009443 |
| TGIF1 | 5.28E−07 | −0.30446926 | 0.316 | 0.434 | 0.011111 |
| VBP1 | 6.41E−07 | −0.26309015 | 0.404 | 0.517 | 0.013499 |
| EIF4A2 | 6.79E−07 | −0.3222835 | 0.842 | 0.879 | 0.014284 |
| TSG101 | 7.05E−07 | −0.26742257 | 0.329 | 0.45 | 0.014843 |
| ATP5C1 | 7.34E−07 | −0.267944 | 0.573 | 0.631 | 0.015457 |
| UBE2L6 | 8.13E−07 | −0.25941519 | 0.272 | 0.401 | 0.017112 |
| CCT6A | 8.66E−07 | 0.26451805 | 0.751 | 0.621 | 0.018224 |
| CDO1 | 1.02E−06 | −0.28810974 | 0.342 | 0.457 | 0.021447 |
| CCT2 | 1.06E−06 | 0.26249639 | 0.775 | 0.664 | 0.022275 |
| CSNK1D | 1.08E−06 | 0.27787804 | 0.358 | 0.229 | 0.022645 |
| TTC19 | 1.15E−06 | −0.25150999 | 0.264 | 0.395 | 0.024126 |
| PSMB2 | 1.27E−06 | −0.27576746 | 0.56 | 0.612 | 0.026753 |
| TAGLN3 | 1.40E−06 | −0.39250816 | 0.14 | 0.254 | 0.029493 |
| GDI2 | 1.48E−06 | −0.26139268 | 0.689 | 0.757 | 0.031157 |
| FKBP4 | 1.63E−06 | 0.26114291 | 0.619 | 0.499 | 0.034366 |
| PRSS23 | 1.74E−06 | −0.27574973 | 0.256 | 0.397 | 0.036576 |
| MEAF6 | 1.81E−06 | −0.2776565 | 0.466 | 0.549 | 0.03805 |
| NME4 | 1.81E−06 | −0.26187119 | 0.378 | 0.496 | 0.038084 |
| CKS2 | 2.07E−06 | 0.3807143 | 0.358 | 0.247 | 0.043566 |
| 15-Sep | 2.26E−06 | −0.25817486 | 0.415 | 0.517 | 0.047638 |
| ARRDC3 | 2.43E−06 | 0.35767496 | 0.477 | 0.359 | 0.051051 |
| ARPC1A | 3.27E−06 | 0.25518031 | 0.614 | 0.454 | 0.068786 |
| CPE | 3.63E−06 | −0.28068836 | 0.508 | 0.61 | 0.076444 |
| FKBP3 | 4.55E−06 | −0.27475451 | 0.51 | 0.581 | 0.095692 |
| LCMT1 | 4.79E−06 | −0.25175843 | 0.205 | 0.315 | 0.100768 |
| TMEM147 | 4.86E−06 | −0.27035626 | 0.417 | 0.509 | 0.102337 |
| C11orf49 | 7.31E−06 | 0.2565425 | 0.422 | 0.29 | 0.153802 |
| MAPK10 | 7.35E−06 | 0.26154342 | 0.422 | 0.3 | 0.154661 |
| C1orf43 | 3.65E−05 | −0.28047107 | 0.49 | 0.56 | 0.767773 |
| PEF1 | 3.99E−05 | 0.5596965 | 0.42 | 0.298 | 0.840271 |
| AP1S2 | 4.12E−05 | 0.26102707 | 0.575 | 0.411 | 0.866319 |
| MAP1LC3B | 5.18E−05 | −0.38330718 | 0.518 | 0.571 | 1 |
| PRDX4 | 0.000157 | −0.25020527 | 0.606 | 0.65 | 1 |
| PIN1 | 0.000479 | −0.25087912 | 0.49 | 0.534 | 1 |
| CLNS1A | 0.000615 | −0.26359202 | 0.63 | 0.62 | 1 |
| NCBP2 | 0.001982 | 0.27760063 | 0.635 | 0.521 | 1 |
| PCSK1N | 0.00357 | −0.5004887 | 0.21 | 0.268 | 1 |
| TYMS | 0.009399 | −0.39064102 | 0.358 | 0.388 | 1 |
| ARL6IP1 | 0.037303 | 0.43548009 | 0.769 | 0.721 | 1 |
| CKB | 0.187262 | 0.27498009 | 0.909 | 0.968 | 1 |
| FAM63B | 0.402853 | 0.25298089 | 0.282 | 0.251 | 1 |
| REEP5 | 0.50385 | 0.53869462 | 0.303 | 0.276 | 1 |

Table 1 shows the percentage of cells expressing the gene in retina cells (Pct. 1), the percentage of cells expressing the gene in cortical cells (Pct.2), and the fold change (FC) for the expression levels of the gene in retina versus cortical cells. P_val is p-value; avg_log 2FC is the log fold-change of the average expression between the two groups. Positive values indicate that the gene is more highly expressed in the first group. P_val_adjust is the adjusted p-value, based on boneferroni correction using all genes in the dataset.

1. A method for producing neuroectoderm cells from human pluripotent stem cells (hPSCs) comprising the steps of treating hPSCs in cell culture in vitro with inhibitors of BMP or TGF-beta, alone or in combinations thereof, for no more than 6 days in chemically defined media.

2. The method of claim 1, wherein the BMP inhibitor is DMH1 and the neuroectoderm cells produced thereby are definitive neuroectoderm cells.

3. The method of claim 1, wherein the TGF-beta inhibitor is SB-431542 and the neuroectoderm cells produced thereby are primitive neuroectoderm cells (PNCs).

4. The method of claim 1, wherein RAX or VSX2 expression is induced and SOX1 expression repressed by treatment with the TGF-beta inhibitor.

5. The method of claim 3, wherein the PNCs produced by the method are characterized by expression of PAX6 and non-expression of SOX1.

6. The method of claim 4, wherein hPSCs are treated with the TGF-beta inhibitor for up to 6 days.

7. The method of claim 1, wherein hPSCs are treated simultaneously with a BMP inhibitor and a TGF-beta inhibitor.

8. A method for producing retinal progenitor cells from primitive neuroectoderm cells (PNCs) comprising the step of treating the PNCs in cell culture in vitro with BMP2, BMP4, or BMP7 for about 20 days.

9. The method of claim 8 wherein the PNCs are produced by treating hPSCs in cell culture in vitro with inhibitors of BMP and TGF-beta, alone or in combinations thereof, for 5 to 6 days in chemically defined media.

10. The method of claim 9, wherein PNCs are produced by treating the cells with BMP2.

11. The method of claim 10, wherein BMP2 is administered in a concentration of 2-10 ng/mL.

12. The method of claim 11, wherein PNCs are treated with BMP2 after day 4 and before day 7 of cell culture.

13. A composition of retinal progenitor cells produced by the method of claim 8.

14. The retinal progenitor cells of claim 13 wherein the progenitor cells are about 50% pure to about 100% pure.

15. A composition of neuroectoderm cells produced by the method of claim 1.

16. A method for producing retinal progenitor cells from human pluripotent stem cells (hPSCs) comprising the steps of treating hPSCs in cell culture with a TGF-beta inhibitor for five days followed by treatment with BMP activator from day 6 to day 20.

17. The method of claim 16, wherein the TGF-beta inhibitor is A 77-01, A 83-01, AZ 12799734, D 4476, disitertide, galunisertib, GW 788388, IN 1130, LY 2109761, R 268712, RepSox, SB 431542, SB 505124, SB 525334, SD 208, or SM 16.

18. The method of claim 16, wherein the BMP activator is BMP2.

19. The method of claim 17, wherein the TGF-beta inhibitor is SB 431542.

20. The method of claim 18, wherein the concentration of BMP2 is 10 ng/ml.

21. A composition of retinal progenitor cells produced by the method of claim 16.

22. The retinal progenitor cells of claim 20 wherein the progenitor cells are about 50% pure to about 100% pure.
